SEQUENCE LISTING (27 sequences)

SEQ ID NO: 1 - protein, 186 aa, Artificial Sequence
  DHFR and DFR1 amino acid sequence
VRPLNCIVAVSQNMGIGKNGDLPWPPLRNEFKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 2 - protein, 211 aa, Artificial Sequence
  DHFR and DFR1 amino acid sequence
MAGGKIPIVGIVACLQPEMGIGFRGGLPWRLPSEMKYFRQVTSLTKDPNKKNALIMGRKTWESIPPKFRPLPNRMNVIISRSFKDDFVHDKERSIVQSNSLANAIMNLESNFKEHLERIYVIGGGEVYSQIFSITDHWLITKINPLDKNATPAMDTFLDAKKLEEVFSEQDPAQLKEFLPPKVELPETDCDQRYSLEEKGYCFEFTLYNRK

SEQ ID NO: 3 - protein, 232 aa, Artificial Sequence
  wFc (wild type human Fc)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 232 aa, Artificial Sequence
  mFc (mutant human Fc)
EPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 532 aa, Artificial Sequence
  ABAB sequence (FZ002)
QVQLVETGGGLVQPGGSLRLSCAASGFTLDYSSIGWFRQAPGKEREGVSCISSSGDSTKYADSVKGRFTTSRDNAKNTVYLQMNSLKPDDTAVYYCAAFRATMCGVFPLSPYGKDDWGKGTLVTVSSGGGGSGGGGSGGGGSQVQLVESGGGLVQPGGSLRLSCEASGFTLDYYGIGWFRQPPGKEREAVSYISASARTILYADSVKGRFTISRDNAKNAVYLQMNSLKREDTAVYYCARRRFSASSVNRWLADDYDVWGRGTQVAVSSGGGSGGGSGGGSGGGSQLQLVETGGGLVQPGGSLRLSCAASGFTFSDYVMTWVRQAPGKGPEWIATINTDGSTMRDDSTKGRFTISRDNAKNTLYLQMTSLKPEDTALYYCARGRVISASAIRGAVRGPGTQVTVSSGGGGSGGGGSGGGGSQVQLVESGGGLVQTGGSLRLSCASSGSIAGFETVTWSRQAPGKSLQWVASMTKTNNEIYSDSVKGRFIISRDNAKNTVYLQMNSLKPEDTGVYFCKGPELRGQGIQVTVSS

SEQ ID NO: 6 - protein, 120 aa, Artificial Sequence
  TNF-alfa antibody amino acid sequence (FZ006)
EVQLLESGGGLVQPGGSLRLSCAASGRTFSTYAMGWFRQAPGKEREFVATVDNDGFTMHADSVKGRFTISRDNSKNTLYLQMNSLRAEDTAVYYCAAGQYSKFVESYAYWGQGTLVTVSS

SEQ ID NO: 7 - protein, 263 aa, Artificial Sequence
  TNF-alfa antibody amino acid sequence (FZ006m)
QVQLQDSGGGLVQAGGSLRLSCAASGGTFSSIIMAWFRQAPGKEREFVGAVSWSGGTTVYADSVLGRFEISRDSARKSVYLQMNSLKPEDTAVYYCAARPYQKYNWASASYNVWGQGTQVTVSSGGGGSGGGGSGGGGSQVQLQDSGGGLVQAGGSLRLSCAASGGTFSSIIMAWFRQAPGKEREFVGAVSWSGGTTVYADSVLGRFEISRDSARKSVYLQMNSLKPEDTAVYYCAARPYQKYNWASASYNVWGQGTQVTVSS

SEQ ID NO: 8 - protein, 267 aa, Artificial Sequence
  Bi-specific antibodies against IL-17A and TNF-alfa amino acid sequence (FZ008a)
EVQLLESGGGLVQPGGSLRLSCAASGRTFSTYAMGWFRQAPGKEREFVATVDNDGFTMHADSVKGRFTISRDNSKNTLYLQMNSLRAEDTAVYYCAAGQYSKFVESYAYWGQGTLVTVSSGQAGQGGGGSGGGGSGGGGSLQQVQLVQSGGGLVQAGGSLRLSCAASGGTFATSPMGWLRQAPGKGTEFVAAISPSGGDRIYADSVKGRFTISRDNAGYFIYLQMNSLKPEDTAVYYCAVRRRFDGTSYYTGDYDSWGQGTLVTVSS

SEQ ID NO: 9 - protein, 125 aa, Artificial Sequence
  Bi-specific antibodies against IL-17A and TNF-alfa amino acid sequence(FZ008b-VHH1)
EVQLLESGGGLVQPGGSLRLSCAASGRTFSTYAMGWFRQAPGKEREFVATVDNDGFTMHADSVKGRFTISRDNSKNTLYLQMNSLRAEDTAVYYCAAGQYSKFVESYAYWGQGTLVTVSSGQAGP

SEQ ID NO: 10 - protein, 125 aa, Artificial Sequence
  Bi-specific antibodies against IL-17A and TNF-alfa amino acid sequence (FZ008b-VHH2)
QVQLVQSGGGLVQAGGSLRLSCAASGGTFATSPMGWLRQAPGKGTEFVAAISPSGGDRIYADSVKGRFTISRDNAGYFIYLQMNSLKPEDTAVYYCAVRRRFDGTSYYTGDYDSWGQGTLVTVSS

SEQ ID NO: 11 - protein, 146 aa, Artificial Sequence
  IL-22 amino acid sequence (FZ010m)
LPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 12 - protein, 146 aa, Artificial Sequence
  IL-22 amino acid sequence (FZ010)
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 13 - protein, 124 aa, Artificial Sequence
  Anti-Rotavirus VHH amino acid sequence (FZ014)
MADVQLQASGGGFVQPGDSLSLSCAASGGTFSSYSIGWFRQGPGKEREFVATISSSDSPWYGEPAKGRFTVARVNAKNTAYLHLNRLKPEDTATYYCAAGSVQHMANENEYVYWGQGTQVTVSS

SEQ ID NO: 14 - protein, 258 aa, Artificial Sequence
  Bi-specific VHHs-Fc (FZ016a)
QVKLQESGGGLVQAGESLRLSCAASGSNFSINGVGWYRQAPGKQRELVAGITNGGYTSYADSVKGRFTISTDNAKNTVYLQMNSLRPEDTAVYYCNANFQIHRSGADYVRNYWGQGTQVTVSAGGGGSGGGGSGGGGSQVKLQQSGGGLVQPGGSLRLSCAASESTISINTLGWYRQAPGNQRELVATITTGGTTNYADSVKGRFTISRDNAKNTVYLQMNNLEPGDTAVYYCNLKRRDLQSRFGGYWGQGTQVTVSS

SEQ ID NO: 15 - protein, 258 aa, Artificial Sequence
  Bi-specific VHHs-Fc (FZ016b)
QVKLQESGGGLVQAGESLRLSCAASGSNFSINGVGWYRQAPGKQRELVAGITNGGYTSYADSVKGRFTISTDNAKNTVYLQMNSLRPEDTAVYYCNANFQIHRSGADYVRNYWGQGTQVTVSAGGGGSGGGGSGGGGSQVQLQQSGGGLVQPGGSLRLSCVASESTVSINIMGWYRQAPGKQRELVATITTGGTTNYADSVKGRFTISRDNAKNTVYLQMNSLEPEDTAVYYCNLKRRDLQARFGGYWGQGTQVTVSS

SEQ ID NO: 16 - protein, 129 aa, Artificial Sequence
  Mono-specific (FZ016c)
QVKLQQSGGGLVQPGGSLRLSCAASESTISINTLGWYRQAPGNQRELVATITTGGTTNYADSVKGRFTISRDNAKNTVYLQMNNLEPGDTAVYYCNLKRRDLQSRFGGYWGQGTQVTVSSYPYDVPDYA

SEQ ID NO: 17 - protein, 30 aa, Artificial Sequence
  GLP-1 amino acid sequence (FZ022a)
HAEGTFTSDVSSYLEGQAAKEFIAWLVKGR

SEQ ID NO: 18 - protein, 155 aa, Artificial Sequence
  Leptin amino acid sequence (FZ024)
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGCYPYDVPDYA

SEQ ID NO: 19 - protein, 451 aa, Artificial Sequence
  Anti-TNF-alfa IgG1 acid sequence (FZ026-HC)
EVQLVESGGGLVQPGRSLRLSCAASGFTFDDYAMHWVRQAPGKGLEWVSAITWNSGHIDYADSVEGRFTISRDNAKNSLYLDMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, Artificial Sequence
  Anti-TNF-alfa IgG1 acid sequence (FZ026-LC)
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 314 aa, Artificial Sequence
  VHH against cwp84 fused with Lysin domain amino acid sequence(FZ028a)
MKICITVGHSILKSGACTSADGVVNEYQYNKSLAPVLADTFRKEGHKVDVIICPEKQFKTKNEEKSYKIPRVNSGGYDLLIELHLNASNGQGKGSEVLYYSNKGLEYATRICDKLGTVFKNRGAKLDKRLYILNSSKPTAVLIESFFCDNKEDYDKAKKLGHEGIAKLIVEGVLNKNINGGGGSGGGGSGGGGSQVQLVESGGGLVQAGGSLRLSCVASTSTFPINEMGWYRQAPGKQRELVAAITRVGTTNYANSVKGRFTISRDKTSNTKNTVYLQMNSLKPEDTAVYYCNVNLFADTGNYWGQGTLVTVSS

SEQ ID NO: 22 - protein, 311 aa, Artificial Sequence
  VHH against cwp84 fused with Lysin domain amino acid sequence(FZ028b)
MKVVIIPGHTLIGKGTGAVGYINESKETRILNDLIVKWLKIGGATVYTGRVDESSNHLADQCAIANKQETDLAVQIHFNSNATTSTPVGTETIYKTNNGKTYAERVNTRLATVFKDRGAKSDVRGLYWLNHTIAPAILIEVCFVDSKADTDYYVNNKDKVAKLIAEGILNKSGSGGGSEGGGSEGGGSEGGQVQLVESGGGLVQAGGSLRLSCVASTSTFPINEMGWYRQAPGKQRELVAAITRVGTTNYANSVKGRFTISRDKTSNTKNTVYLQMNSLKPEDTAVYYCNVNLFADTGNYWGQGTLVTVSS

SEQ ID NO: 23 - protein, 31 aa, Artificial Sequence
  GLP-1 amino acid sequence (FZ022b)
HAEGTFTSDVSSYLEGQAAKEFIAWLVKGRG

SEQ ID NO: 24 - protein, 39 aa, Artificial Sequence
  GLP-1 amino acid sequences (FZ022c)
HGEGTFTSDLSKQMEEEAVRLFIEWLKNGGPSSGAPPPS

SEQ ID NO: 25 - protein, 125 aa, Artificial Sequence; identical to SEQ ID NO: 10
  Anti-IL-17A antibody amino acid sequence (FZ004)

SEQ ID NO: 26 - protein, 58 aa, Artificial Sequence [sequence omitted]

SEQ ID NO: 27 - protein, 160 aa, Artificial Sequence
  1L-10 amino acid sequence (FZ030)
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

(19) United States
(12) Patent Application Publication
(10) Pub. No.: US 2024/0271082 A1
(43) Pub. Date: Aug. 15, 2024

(54) GENETICALLY-MODIFIED SACCHAROMYCES YEAST STRAINS AS PREVENTATIVE AND THERAPEUTIC AGENTS

(71) Applicant: Fzata, Inc., Halethorpe, MD (US)

(72) Inventors: Hua Yu, Halethorpe, MD (US); Lydia Nyasae, Halethorpe, MD (US); Kyle Hoban, Halethorpe, MD (US); Zhiyong Yang, Halethorpe, MD (US)

(73) Assignee: Fzata, Inc., Halethorpe, MD (US)

(21) Appl. No.: 18/568,774

(22) PCT Filed: Jun. 10, 2022
(86) PCT No.: PCT/US2022/033037

§ 371 Date: Dec. 8, 2023

Related U.S. Application Data
(60) Provisional application No. 63/209,651, filed on Jun. 11, 2021.
(60) Provisional application No. 63/299,693, filed on Jan. 14, 2022.
(60) Provisional application No. 63/317,385, filed on Mar. 7, 2022.

(51) Int. Cl. C12N 1/18; A61K 38/00; A61K 39/00; A61P 31/04; C07K 14/54; C07K 14/575; C07K 16/12; C07K 16/24; C12N 15/81

(52) U.S. Cl. CPC C12N 1/185; A61P 31/04; C07K 14/5428; C07K 14/5759; C07K 16/1282; C07K 16/241; C07K 16/244; C12N 15/81; A61K 38/00; A61K 2039/505; C07K 2317/22; C07K 2317/31; C07K 2317/52; C07K 2317/569; C07K 2317/76; C12N 2510/00

(57) ABSTRACT

The disclosure relates to engineered strains of yeast that express a therapeutic polypeptide(s) and/or comprise a nucleic acid encoding a therapeutic polypeptide(s), as well as methods and uses for the same.

### REFERENCE TO RELATED APPLICATIONS

This application claims the benefit of the priority date of U.S. provisional application, 63/209,651, filed Jun. 11, 2021, U.S. provisional application, 63/299,693, filed Jan. 14, 2022, and U.S. provisional application 63/317,385, filed Mar. 7, 2022, the contents of each of which are incorporated herein by reference in their entirety.

### BACKGROUND

Manufacturing of conventional therapeutic polypeptide(s) at scales beyond one liter (e.g., small-scale, large-scale, or commercial scale) in bioreactors followed by downstream purification of therapeutic polypeptide(s) is a costly and time-consuming process. In the United States, therapeutic polypeptide(s) intended for administration by injection or infusion have to be in compliance with US FDA current good manufacturing practice (CGMP) regulations establishing identity, strength, purity, and other qualities. Furthermore, purified polypeptides (e.g., therapeutic polypeptide(s)) are notoriously fragile and likely to misfold, aggregate or precipitate which can result in a loss of efficacy. Thus, maintaining therapeutic polypeptide(s)' efficacy from manufacturing to time of administration can require careful formulation, gentle handling and/or cold-chain distribution of the fragile purified therapeutic polypeptide(s).

These factors, for example, make therapeutic polypeptide(s) orders of magnitude more expensive to develop, manufacture, and distribute than small molecules also intended for therapeutic use. Additionally, therapeutic polypeptide(s) are often administered by healthcare professionals using an injection or intravenous infusion, requiring patients to travel to healthcare facilities to receive treatment. This can reduce patient compliance and lead to poor patient outcomes.

Some therapeutic polypeptide(s) include, for example monoclonal antibodies. Some examples include, without limitation, adalimumab, infliximab, secukinumab, and ixekizumab, which neutralize pro-inflammatory cytokines and are extremely successful for treating many inflammatory diseases. However, these therapeutic polypeptide(s) require needle injection and their long-term use is associated with loss of effectiveness and serious side effects due to anti-drug antibody responses and general immune suppression. Additionally, there are currently limited alternatives for other routes of administration, as delivering therapeutic polypeptide(s) (e.g., proteins, polypeptides, antibodies or functional fragments thereof) via an oral route into the gastrointestinal (GI) tract must overcome several major obstacles: 1) it is expensive to manufacture large quantities of therapeutic polypeptide(s) for oral delivery; 2) therapeutic polypeptides are sensitive to the high acidity of stomach fluid and likely to lose therapeutic efficacy; and 3) therapeutic polypeptide(s) are typically sensitive to GI enzymatic digestion, losing efficacy.

Despite advances in research relating to, for example, Crohn's disease, ulcerative colitis, celiac disease, and other inflammatory conditions or inflammation-related conditions, there remains a scarcity of treatments that are potent and efficacious for chronic use. Furthermore, there is an established commercial and medical need for therapeutic polypeptide(s), that are inexpensive and can be prepared without the need for downstream polypeptide processing and purification, that are not immunogenic, that obviate cold-chain distribution and cold storage, and that are patient friendly, for example, can be self-administered in oral dosage forms.

These needs and other needs are satisfied by the present disclosure.

### SUMMARY

The present disclosure provides a platform for an oral route of delivery for therapeutic polypeptide(s) utilizing engineered Saccharomyces yeast that are capable of synthesizing such therapeutic polypeptide(s) in the gut. The present disclosure likewise provides methods of treatment utilizing the disclosed platform, as well as additional related methods and uses.

In one aspect, the present disclosure provides engineered strains of Saccharomyces yeast comprising:
at least one site-specific chromosomal insertion of a nucleic acid encoding a therapeutic polypeptide,
wherein the therapeutic polypeptide is selected from a binding protein comprising an antigen-binding domain, an immunoglobulin, an antibody, a cytokine, a hormone, and a chemokine or a combination thereof.

In some embodiments, the yeast is Saccharomyces boulardii.

In some embodiments, the yeast may further comprise a complete or partial deletion of URA3. In some embodiments, the yeast may further comprise complete or partial deletion of GAP1. In some embodiments, the yeast is ura3(−/−) and gap1(−/−).

In some embodiments, nucleic acid encoding the therapeutic polypeptide is incorporated into at least two different positions in the yeast's genome or at least one hot spot in the yeast's genome. In some embodiments, nucleic acid encoding the therapeutic polypeptide is incorporated into at least two different chromosomes. In some embodiments, the at least two different chromosomes comprise chromosomes VII and XVI.

In some embodiments, the yeast may further comprise a nucleic acid sequence encoding a dihydrofolate reductase (DHFR) incorporated into the yeast's genome, wherein the DIFR is optionally a mammalian DFHR. Additionally or alternatively, in some embodiments, the yeast may further comprise one or more exogenous nucleic acids encoding a yeast DFR1.

In some embodiments, the therapeutic polypeptide is a binding protein that comprises a structure selected from VHH, Fc-VHH, VHH-Fc, VHH-VHH, VHH-VHH-VHH-VHH, Fc-VHH-VHH, VHH-Fc-VHH, and VHH-VHH-Fc, wherein each or any of the VHH or Fc domains are attached to another VHH or Fc domain via an optional linker sequence.

In some embodiments, the therapeutic polypeptide is a binding protein that binds to TcdA, TcdB, or a combination thereof. In some embodiments, the therapeutic polypeptide comprises an amino acid sequence of SEQ ID NO: 5

In some embodiments, the therapeutic polypeptide is a binding protein that binds to TNF-α. In some embodiments, the binding protein is an IgG or comprises at least one VHH domain. In some embodiments, the therapeutic protein comprises an amino acid sequence selected from any one of SEQ ID NOs: 6, 7, 19, and 20.

In some embodiments, the therapeutic polypeptide is a binding protein that binds to IL-17A. In some embodiments, the binding protein is an IgG or comprises at least one VHH domain. In some embodiments, the therapeutic protein comprises an amino acid sequence of SEQ ID NO: 25.

In some embodiments, the therapeutic polypeptide is a bi-specific binding protein that binds to TNF-α and IL-17A. In some embodiments, the binding protein is an IgG or comprises at least two VHH domains. In some embodiments, the therapeutic protein comprises an amino acid sequence selected from any one of SEQ ID NOs: 8, 9, and 10.

In some embodiments, the therapeutic polypeptide is a binding protein that binds to a norovirus or a rotavirus. In some embodiments, the binding protein is an IgG or comprises at least one VHH domain. In some embodiments, the therapeutic protein comprises an amino acid sequence selected from any one of SEQ ID NOs: 13, 14, 15, and 16.

In some embodiments, the therapeutic polypeptide is a VHH that binds to cwp84 and is fused with a lysin domain. In some embodiments, the therapeutic protein comprises an amino acid sequence selected from any one of SEQ ID NOs: 21 and 22.

In some embodiments, the therapeutic polypeptide is a cytokine or chemokine. In some embodiments, the cytokine is IL-22 or IL-10. In some embodiments, the cytokine or chemokine is fused to an Fc domain. In some embodiments, the therapeutic protein comprises an amino acid sequence selected from any one of SEQ ID NOs: 11, 12, and 27.

In some embodiments, the therapeutic polypeptide is GLP1. In some embodiments, the therapeutic protein comprises an amino acid sequence selected from any one of SEQ ID NOs: 17, 23, and 24.

In some embodiments, the therapeutic polypeptide is leptin. In some embodiments, the therapeutic protein comprises an amino acid sequence of SEQ ID NO: 18.

In some embodiments, the yeast comprises at least 2, at least 3, at least 4, at least 5, at least 6, at least 7, at least 8, at least 9, or at least 10 or more copies of the nucleic acid encoding the therapeutic polypeptide incorporated into its genome.

In some embodiments, the yeast may further comprise at least one site-specific chromosomal insertion of a second nucleic acid encoding a second therapeutic polypeptide, wherein the second therapeutic polypeptide is selected from a binding protein comprising a VHH domain, an immunoglobulin, a cytokine, and a chemokine or a combination thereof.

In another aspect, the present disclosure provides methods of binding an antigen in vivo, comprising administering to a subject an engineered strain of Saccharomyces yeast as disclosed herein (e.g., the foregoing aspect and embodiments). In some embodiments, the antigen is selected from TcdA, TcdB, both TcdA and TcdB, TNF-α, IL-17A, both TNF-α and IL-17A, cwp84, a rotavirus protein, or a norovirus protein.

In another aspect, the present disclosure provides methods of treating or preventing a disease or condition, comprising administering to a subject in need thereof an effective amount of an engineered strain of Saccharomyces yeast of any one of claims 1-37.

In some embodiments, the disease or condition is an inflammatory condition. In some embodiments, the inflammatory condition is selected from inflammatory bowel disease (IBD), gut inflammation, Crohn's disease, and ulcerative colitis.

In some embodiments, the disease or condition is an infection. In some embodiments, the infection is a C. difficile infection, a norovirus infection, a rotavirus infection, or a combination thereof. In some embodiments, the subject additionally has IBD.

In some embodiments, the disease or condition is irritable bowel syndrome (IBS).

In some embodiments, the disease or condition is neurodegenerative disease

In some embodiments, the disease or condition is diabetes.

In some embodiments, the disease or condition is obesity.

In some embodiments, the disease or condition is fatty liver disease.

In some embodiments, the disease or condition is a metabolic disease.

In some embodiments, the disease or condition is graft-versus-host disease (GVHD).

In some embodiments, the disease or condition is an autoimmune disease.

In another aspect, the present disclosure provides methods of selecting an engineered stain of Saccharomyces yeast, wherein the Saccharomyces yeast comprises a nucleic acid sequence encoding a dihydrofolate reductase (DHFR) incorporated into the yeast's genome, one or more exogenous nucleic acids encoding a yeast DFR1, or a combination thereof, the method comprising contacting the Saccharomyces yeast with methotrexate and sulfanilamide. In some embodiments, the DHFR is a mammalian DHFR.

In some embodiments, the methotrexate is in a concentration of 1 nM to 1 mM. In some embodiments, the sulfanilamide is in a concentration of 0.1 to 10 mg/mL.

In some embodiments, the engineered stain of Saccharomyces yeast comprises: at least one site-specific chromosomal insertion of a nucleic acid encoding a therapeutic polypeptide, wherein the therapeutic polypeptide is selected from a binding protein comprising an antigen-binding domain, an immunoglobulin, an antibody, a cytokine, a hormone, and a chemokine or a combination thereof.

In some embodiments, the yeast is Saccharomyces boulardii.

In some embodiments, the yeast is ura3(−/−) and gap1(−/−).

In some embodiments, nucleic acid encoding the therapeutic polypeptide is incorporated into at least two different positions in the yeast's genome.

In some embodiments, the yeast comprises at least 2, at least 3, at least 4, at least 5, at least 6, at least 7, at least 8, at least 9, or at least 10 or more copies of the nucleic acid encoding the therapeutic polypeptide incorporated into its genome.

In some embodiments, the yeast further comprises at least one site-specific chromosomal insertion of a second nucleic acid encoding a second therapeutic polypeptide, wherein the second therapeutic polypeptide is selected from a binding protein comprising a VHH domain, an immunoglobulin, a cytokine, and a chemokine or a combination thereof.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1E show in vitro characterization of a previously-developed strain using similar techniques as disclosed herein, Sb-ABAB (S. boulardii transformed with plasmid containing ABAB transgene cassette (pURA3-AT-yABAB-cMyc)). FIG. 1A: ABAB antibody in Sb-ABAB culture supernatant detected by western blotting with antibody specific against cMyc tag. FIG. 1B: Toxin neutralizing activity of Sb-ABAB culture supernatant compared to purified Fc-ABAB. FIG. 1C: In vitro growth of Sb, Sb-EP (S. boulardii strain carrying an empty plasmid as a control) and Sb-ABAB. FIG. 1D: ABAB expression level in Sb-ABAB culture supernatants over multiple passages. FIG. 1E: Antibiotic resistance profile of wild type Sb and Sb-ABAB.

FIGS. 2A-2F show characterization of previously-developed strain Sb-ABAB in vivo. FIG. 2A: Mice pre-exposed to antibiotics were gavaged with PBS, Sb, Sb-EP and Sb-ABAB (10¹⁰ CFU) daily for 7 days. FIG. 2B: Mouse weight was monitored. FIG. 2C: Yeast shed from different groups were monitored and the CFU reached peak on day 1 post gavage but rapidly dropped to undetectable levels 3 days after the final dosing. FIG. 2D: Intestinal lavages collected on day 3 were able to neutralize TcdB (10 μg/mL) on Vero cell monolayers. FIG. 2E: ABAB expression levels of Sb-ABAB culture supernatants over multiple passages in vitro for yeast strains recovered from fecal samples, Sb-ABAB(f), versus non-passaged strains from frozen master stocks. FIG. 2F shows ABAB levels on day 3 feces determined by ELISA. All experimental data represent one of at least three separate experiments with 3 mice per group and are presented as ±s.e.m.

FIGS. 3A-3D show bivalent anti-TNF-α VHH-Fc fusion is significantly more potent than monovalent anti-TNF-α VHH for neutralizing TNF-α cytotoxicity. L929 cells (FIG. 3A) were treated with 100 ng/mL of TNF-α alone (FIG. 3B) or together with monovalent anti-TNF-α (FIG. 3C) or bivalent anti-TNF-α VHH-Fc (FIG. 3D) for either 6 h or 24 h. Cell rounding (which may be indicative of apoptosis) was observed under microscope.

FIGS. 4A-4G show oral Sb-amTNF (FZ006m) protected mice from DSS-induced colitis as well as comorbidity of DSS and CDI. FIG. 4A shows oral administration of FZ0006m had some protective trend against DSS-induced colitis, a mild colitis model. A more severe comorbidity disease model was used in FIGS. 4B-4G. DSS-colitis and CDI comorbidity mice were orally administered with either PBS or engineered yeast daily for 3 days before and 7 days after C. difficile spore challenge. (FIGS. 4B-4F) Oral Sb-amTNF protected mice from weight loss (FIG. 4B), death (FIG. 4C), up-regulation of inflammatory cytokine TNF-α (FIG. 4D) and IL-6 (FIG. 4E), length reduction of colons (FIG. 4F), and colonic tissue damages (FIG. 4G). n=10. (*) indicates P<0.05; (**) P<0.01, and (***) P<0.001.

FIGS. 5A-5C show Activities of dimeric anti-TNF-α VHHs. FIG. 5A: Neutralizing activity. The serially diluted VHHs-Fc or Humira were mixed with 10 μM human TNF-α before being applied to L929 cells. After 24h incubation, the antibody-mediated inhibitions of cytotoxicity were measured. FIG. 5B: Binding affinity. The binding of serially diluted G1 or Humira on TNF-α-coated ELISA plates. FIG. 5C: Blocking the interaction of TNF-α and TNFR1. Serially diluted G1 or Humira were mixed with 100 ng/ml of biotinylated human TNF-α before adding to TNFR1-coated wells. The ability of antibody-mediated inhibition of TNF-α binding to TNFR1 was determined by the competition ELISA.

FIGS. 6A-6C show characterization of Sb-ahTNF (FZ006) strains. FIG. 6A: Growth rates of two Sb-ahTNF strains is comparable to their parental strain. FIG. 6B: Stable production of antibody by Sb-ahTNF after extensive in vitro culture. Sb-ahTNF or Sb-EP were passed in YPD media, starting from 0.1 OD, for 8 days. Supernatants from every 24-hr culture were collected and the expression of ahTNF was measured by ELISA. FIG. 6C: Neutralizing activity of ahTNF in the supernatant from Sb-ahTNF day-7 culture in FIG. 6B. The concentration of the antibody in the supernatant was determined by ELISA and then serially diluted before mixing with 3 ng/ml of recombinant human TNF-α. After 30 min of incubation, the mixture was applied to L929 monolayers and cytotoxicity was measured with resazurin viability kit. Control groups included purified G1 and HUMIRA® in the same concentrations.

FIGS. 7A-7D show oral Sb-amTNF and Sb-ABAB protected mice from comorbidity of CDI and adoptive T-cell transfer colitis (TCC). FIG. 7A: Diagram of experimental design. Groups of T-cell transferred RAG−/− mice (n=4) were challenged with C. difficile spores and then treated with PBS or yeast. Mice were sacrificed and colonic tissues were analyzed. FIG. 7B: H&E staining of colonic tissues of mice from different groups: a) RAG−/−, b) TCC, c) TCC treated with Sb-amTNF, d) TCC+CDI, e) TCC+CDI treated with Sb-ABAB, and f) TCC+CDI treated with Sb-amTNF and Sb-ABAB. FIG. 7C: Histology scores of FIG. 7B. The scores were assessed blindly based on colonic morphology, infiltration of inflammatory cells and signs of cell deaths. Normal control RAG−/− mice score 0, no inflammation; TCC mice showed moderate inflammation; TCC+CDI mice exhibited severe inflammation. TCC mice treated with Sb-amTNF showed mild inflammation; comorbidity mice treated with Sb-ABAB showed moderate inflammation, which was reduced to mild inflammation when also treated with Sb-amTNF. P values between the groups are shown. FIG. 7D: The fold change of IFN-7 mRNA expression among the indicated groups were compared and the P values among the groups are shown.

FIG. 8 shows the concentration of ABAB antibody secreted into supernatant of parent strain, pep4 mutant clones, and negative control cultures in rich YPD medium as measured by ELISA.

FIG. 9A-9B demonstrates the kinetics of Sb-ABAB colonization and secretion of ABAB in piglets. Two gnotobiotic piglets (2 days of age) were fed with 10¹⁰ CFU of Sb-ABAB daily for 11 days (from day 1 to 11). Fecal swaps were collected daily from 1 day before oral Sb-ABAB and continued till 3 days after the final dose. Fecal Sb-ABAB CFU (FIG. 9A) and ABAB concentration (FIG. 9B) measured by ELISA were determined.

FIG. 10A-10B demonstrate prophylactic efficacy of Sb-ABAB against CDI in mice. FIG. 10A shows a Kaplan-Meier survival curve. FIG. 10B shows H&E stained colonic tissue sections. Statistical analysis was performed by comparing the indicating groups using a Log-rank (Mantel-Cox) test. N=10, * p value <0.05.

FIG. 11 shows oral Sb-ahTNF reduces the expression of TcdB induced TNF-α in human colonic tissues. The fresh human colonic tissues were obtained from normal regions of colon cancer patients (UCLA Pathology). The tissues were cut into 3×3 mm size and placed in RPMI 1640 media (1 ml). The tissues were treated with nothing (blank control) or supernatants (2.5 μl) from Sb-EP or Sb-ahTNF culture for 30 minutes, followed by the addition of PBS or toxin B. The tissues were then incubated at 37° C. for 4 hours. The TNF-α levels in the supernatants were measured by ELISA (DY210, R&D Systems).

FIG. 12A-12B demonstrates repeated doses of oral Sb-amTNF do not induce anti-drug antibody responses. FIG. 12A shows a diagram of experimental design. Groups of mice (n=5) were treated with PBS, Sb-amTNF (10⁹ CFU/dose), or purified anti-TNF-α VHH/VHH-Fc (10 mg/Kg per injection). Oral Sb-amTNF were three courses of daily treatments, with 36 doses in total. Purified anti-mouse VHH/VHH-Fc fusion polypeptide was ip injected two courses, with 6 doses in total. Fecal and serum samples were collected for ELISA. FIG. 12B shows ELISA detection of fecal IgA and serum IgG response. Fecal samples (1:1 w/v in PBS) and serum samples (1000× dilution in PBS) were added to ELISA plates coated with purified anti-TNF VII/VHH-Fc and OD of IgA in fecal and IgG in serum samples were measured.

FIG. 13 shows a summary of methods of gene insertions.

FIG. 14 shows exemplary DNA sequences of homologous arms employed for the development of engineered S. boulardii strains.

FIG. 15 shows a schematic of Delta and Sigma insertion sites.

FIG. 16 demonstrates transformation efficiency at Sigma and Delta sites.

FIG. 17 demonstrates mean yeGFP fluorescence after 24 hour-culture of clones with GOI inserted at Sigma or Delta sites.

FIG. 18 demonstrates ABAB expression level from S. boulardii in which the gene of interest was inserted at either pTEF or pTDH3 is 3-5 times higher compared to those inserted at Delta site.

FIG. 19 demonstrates stability of FZ002 clones. Different clones were passed daily for 11 days. The supernatants of 24 hour culture were used to measure ABAB expression by ELISA.

FIG. 20 shows the early production of ABAB by the clones. The different clones at the indicated cell numbers were cultured for 3 hours and ABAB in the supernatants was measured.

FIG. 21 shows expression of anti-human TNF-α-Fc comparison among different insertion locations (pTEF, pTDH3 and Delta site). FZ006h is the strain in which, the anti-human TNF-α-Fc cassette is inserted at Delta site(s), with robust grow in both YPD and SDCAA medium and highest and stable expression level among clones with the same inserted site. To detect the expression property of anti-human TNF-α-Fc from strains, of which, the gene of interest cassette inserted in promoter of TEF and TDH3, compare the expression level of anti-human TNF-α-Fc from pTEF- and pTDH3-insertion strain versus that of FZ006h. Based on the standard curve (left), anti-human TNF-α-Fc expression level from S. boulardii of which the gene of interest inserted in pTEF or pTDH3 are 2-5 times higher compared to that inserted at Delta site.

FIG. 22 demonstrates generation of uracil auxotrophic S. boulardii yeast strain. Knockout of Ura3 gene (top) was completed using wild-type S. boulardii which were chemically transformed with G418 antibiotic-resistance knockout cassette to replace Ura3 gene and its promoter via homologous recombination. Antibiotic cassette is flanked with LoxP sites. A screening strategy to isolate double allele knockouts (Ura3−/−::G418+/+) clones (bottom) was conducted by transformed S. boulardii clones with successful deletion of Ura3 gene in both alleles were isolated on minimal complete media containing G418 and counter selection agent 5′FOA which is toxic to yeast that are Ura3+/+ or Ura3+/−.

FIG. 23 shows Ura3^{-/-}::G418^{+/+} candidate clones (E37 and E38) can grow on media containing (i) 5′FOA or (ii) G418, but cannot grow on media lacking uracil.

FIG. 24 demonstrates PCR on genomic DNA of Ura3^{-/-}::G418^{+/+} candidate clones (E37 and E38) confirms loss of URA3 gene in both alleles and replaced by G418 antibiotic-resistance cassette

FIG. 25 shows knockout of GAP1 gene. Uracil auxotroph (Ura3^{-/-}::G418^{+/+}, clone E38) was chemically transformed with Phleomycin antibiotic-resistance knockout cassette to replace GAP1 gene and its promoter via homologous recombination. Antibiotic cassette is flanked with LoxP sites.

FIG. 26 demonstrates screening and isolation of knockout clones that are GAP1(−/−)::Phleo(+/+). Transformed Sb clones with successful deletion of GAP1 gene in both alleles (extremely rare event) were isolated on minimal media containing L-Proline (as a nitrogen source), and counterselection agent, D-Histidine (toxic to yeast cells that are GAP1+/+ or GAP1+/−)

FIG. 27 shows GAP1(−/−)::Phleo(+/+) candidate clones (A2-1, A2-6, A2-7) can grow on media containing (i) D-His at 0.16% or 0.5% or (ii) Phleomycin at 5 ug/mL, but cannot grow on minimal media where L-citrulline is the sole source of nitrogen.

FIG. 28 shows PCR on genomic DNA of GAP1(−/−)::Phleo(+/+) candidate clones (A2-1) confirms loss of GAP1 gene in both alleles and replaced by Phleomycin antibiotic-resistance cassette.

FIG. 29 shows excision of both antibiotic cassettes at URA3 and GAP1 loci. To remove both G418- and Phleomycin-antibiotic resistance cassettes in Sb clone A2-7, cells were chemically transformed with plasmid carrying Cre-recombinase enzyme (pPL5071-TEF1-Cre-URA3). Successful transformants were isolated on media lacking uracil. Random clones were selected and underwent 3 rounds of re-streaking (passaging) on various selection media so as to (i) lose the pPL5071 plasmid, while confirming loss of both antibiotic cassettes.

FIG. 30 demonstrates simultaneous screening for clones that have successfully excised both antibiotic cassettes and lost the Cre-recombinase plasmid (i) Candidate clones grow well on rich media. No growth on (ii) YPD+G418, (iii) YPD+Phleomycin, suggests loss of both G418- and Phleomycin-antibiotic-resistance cassettes. Likewise, GAP(−/−) and URA3(−/−) status are confirmed by failure to grow on (iv) MC L-citrulline media and (v) SD-URA, respectively. (vi) Growth on SC+5′FOA confirms that the candidate clones do not carry any URA3 gene and have thus lost the pPL5071-TEF1-Cre-URA3 plasmid.

FIG. 31 demonstrates confirmation of loss of both antibiotic cassettes at URA3 and GAP1 loci by PCR.

FIG. 32 demonstrates verification of absence of antibiotic cassettes anywhere in genome.

FIG. 33 shows mIL-22 average expression level correlates to MTX concentration in DHFR selection.

FIG. 34 demonstrates screening of anti-hIL-17A-Fc expressing clones with URA3 or DHFR selection marker, of which the gene of interest (GOI) cassettes were inserted at Delta sites. After transformation on FZMYA06-16 (URA3−/−, GAP1−/−) strain with DNA fragment containing GOI cassettes together with URA3 or DHFR selection marker cassette which flanked by Delta insertion homologues arm. Positive clones were picked up from corresponding plate: SDCAA+URA3 plate for URA3 selection and YPD+MTX for DHFR selection. 184 positive clones each were sub-cultured at 600 μL of YPD for 24 hours shaking at 250 rpm at 37° C. Culture supernatant were diluted 10× and detected by ELISA. Data readout at OD450 nm.

FIG. 35 demonstrates screening of anti-hTNF-α-Fc-expressing clones with URA3 or DHFR selection marker, of which the GOI cassettes was inserted pTDH3 sites. After transformation on FZMYA06-16 (URA−/−, GAP1−/− strain with DNA fragment comprising GOI cassettes together with URA3 or DHFR selection marker cassette which flanked by pTDH3 homologues arm. Positive clones were picked up from corresponding plate: SDCAA without uracil for URA3 selection and YPD+MTX for DHFR selection. 84 positive clones each were subcultured at 600 μL of YPD for 24 hours shaking at 250 rpm at 37° C. Culture supernatants were diluted 20× and detected by ELISA. Data readout at OD450 nm.

FIG. 36 demonstrates Pep4 deletion enhances secreted polypeptide stability. The concentration of ABAB antibody in supernatant of pep4 mutant clones and parent and WT strain cultures in minimal medium was determined by ELISA and adjusted to account for differences in the optical density of cultures measured at a 600-nm wavelength, Expression/OD600 (E/O).

FIG. 37 shows potential Thr1 and Thr4 null clones (numbered sectors) were grown on rich YPD and minimal SD (without threonine) media. Parent (+) and S. cerevisiae thr1 (−) strain controls included.

FIG. 38 demonstrates Thr1 and Thr4 null clones were grown on rich YPD and minimal SD media either without threonine (SD) or with standard 76 ug/mL threonine (SD+threonine). Parent (+) and S. cerevisiae thr1 (−) strains included.

FIG. 39 demonstrates cultures were inoculated with equal numbers of a given yeast strain and GFP+ cells in minimal medium. GFP fluorescence relative to culture density at 16-hour time point shown here. Fresh medium was inoculated with competitive culture every day for four days. Values were normalized to WT. GFP+ is a noncompetitive culture of GFP-expressing cells only.

FIG. 40 shows S. boulardii (Sb.) expressing anti-TNF-α VHH3. Transformed Sb. expressed anti-TNF-α VHH3-HA (VHH format) with different selection marker (grey and black, URA3 auxtrophic selection; colored, FZE1 selection). 100× diluted supernatant harvested after 48h culture were reactive to human TNF-α, and HA tag was detected by HRP-anti-HA tag. OD450 nm was measured for specific binding activity.

FIG. 41 shows S. boulardii (Sb.) expressing anti-TNF-α VHH3. Transformed Sb. expressed anti-TNF-α VHH3-HA (VHH format, orange line) and anti-TNF-α VHH3s-Fc (black lines). Serially diluted supernatant harvested after 48h culture were mixed with human TNF-α (3 ng/ml) before applied to L929 cells. After 24h incubation, the antibody-mediated inhibitions of cytotoxicity were presented as inhibition %. Sb. expressed anti-TNF-α VHH3-Fc supernatant is more potency than that produced anti-TNF-α VHH3 only.

FIG. 42 demonstrates neutralizing activity of anti-TNF-α VHH3 expression yeast supernatant. Transformed S. boulardii expressed anti-TNF-α VHH3 (VHH format). 40× diluted supernatant harvested after 72 hours induced culture were mixed with human TNF-α (6.25 ng/ml) before applied to L929 cells. After 24 hours incubation, the antibody-mediated inhibitions of cytotoxicity were measured as Proliferation FLI. Red line shows the “Proliferation FLI” of non-transformed S. boulardii culture supernatant.

FIG. 43 shows the expression of functional Sc-FV-Fc antibodies by Expi293 cells. The expression of Sc-FV-Fc by Expi293 was measured by SDS gel (top) as well as ELISAs (bottom). 5 μl of the following supernatant was loaded to SDS gel for quantitation. Lanel: AVA LV-GS-HV-Fc; Lane 2: CAN LV-GS-HV-Fc; Lane 3,4: GIM LV-GS-HV-Fc; Lane 5:1 μg BSA; Lane 6,7: HUM LV-GS-HV-Fc; Lane 8,9: MAV-LV-GS-HV-Fc; Lane 10,11: SILT LV-GS-HV-Fc. Quantitative ELISA was performed with 50× dilution of supernatant reacting with Protein A coated ELISA wells. HRP-conjugated goat anti human IgG (gamma) was used for detection antibody binding at OD450 nm. Anti-TNF-α VHH3/VHH3-Fc used as standard. To detect specific antigen binding activity, the serially diluted supernatant was added to corresponding antigen-coated ELISA wells and detected by RP-conjugated goat anti human IgG (gamma) at OD450 nm. Data were summarized in Table (bottom). Y, yes.

FIG. 44 shows the expression of Sc-Fv-Fc antibodies by S. boulardii (Sb.). The expression of Sc-FV-Fc by Sb. was measured by ELISAs. Sc-FV-Fc-expressing Sb. Supernatant were harvest after 48 hours culture in YPD. 2× diluted supernatant were added to Protein A coated ELISA wells. RP-conjugated goat anti human IgG (gamma) was used for detection antibody binding at OD450 nm. Anti-TNF-α VHH3/VHH3-Fc used as standard. To detect specific antigen binding activity, 2× diluted supernatant was added to corresponding antigen-coated ELISA wells and detected by HRP-conjugated goat anti human IgG (gamma) at OD450 nm. ND, not determined.

FIG. 45 shows expression of functional VHHs (monomer, dimer), VHHs-Fc, VHH-Sc-FV, VHH-Fc-VHH antibodies by S. boulardii (Sb.). The expression of Sc-FV-Fc by Sb. and antigen specific binding activity was measured by ELISAs. Sc-FV-Fc-expressing Sb. Supernatant were harvest after 48 hours culture in YPD. The serially diluted supernatant were added to Protein A coated ELISA wells. HA or Fc tag was detected by HRP-anti-HA tag and HRP-conjugated goat anti human IgG (gamma) correspondingly. OD450 nm was measured. Bioactivity was detected by neutralizing assays. ND, not determined; NA, not available

FIG. 46 shows an exemplary mouse db/db obesity model experimental design.

FIG. 47 shows FZO10m reduces blood glucose in db/db mice. 9 weeks old male db/db mice (n=4/group) were gavaged daily with either 10⁹ CFU FZO10m (Engineered S. boulardii (Sb) constitutively expressing functional mouse IL22 fused with Fc fragment) or Sb-control for 3 weeks. Body weight as well as food and water consumption were measured on Day 0, 5, 10, 15 & 21). On Day 22, EchoMRI and fasting blood glucose level was measured. No significant difference in body weight between the Sb control and FZO10m group was observed. Treatment with FZO10m significantly reduced the food and water consumption in mice and significantly lowered fasting blood glucose level in the db/db mice.

FIG. 48 demonstrates an exemplary mouse high fat diet obesity model.

FIG. 49 demonstrates FZO10m reduces blood glucose in mice with high fat diet. 8 week old male c57BL6/J mice (n=4) were fed with HFD for 11 days before they were gavaged daily with either 10⁹ CFU FZO10m or Sb-control along with HFD. Body weight was measured on Day 1, 7, 13, 19 and 22. EchoMRI and fasting blood glucose level was measured on Day 22. Compared with mice in both the HFD or the sb-control group, mice treated with 12 days of FZO10m had significantly lower body weight and fasting blood glucose level.

FIG. 50 shows a diagram of exemplary generation of VHH1-Fc-VHH2 transgene constructs for genomic insertion. Steps for generating the four constructs of exemplary final integration cassettes (FIC1, FIC2, FIC3, FIC4) for yeast chromosomal integration. Anti-IL-17A gene was synthesized in a pUC plasmid, pUC-aIL17A. The pUC-aIL17A plasmid, along with a vector that harbored an anti-TNF-α-Fc gene, were digested with restriction enzymes, BamH1 and NheI, to obtain four different vectors and one insert. The insert was then ligated into the vectors for transformation into competent E. coli cells. Four colonies were picked from each construct and plasmids were extracted and validated via enzyme digestion. The correct plasmids were used to transform yeast.

FIG. 51 shows a diagram of exemplary generation of VHH1-VHH2-Fc transgene constructs for genomic insertion. Gibson assembly was used due to a lack of suitable restriction enzyme sites. Exemplary anti-IL-17A VHH2 genes were amplified by PCR with primers containing homologous overhangs and mixed with four individual linearized vectors containing an anti-TNF-α VHH2-Fc with different desired promoters and selection markers for Gibson assembly. Following assembly, E. coli transformations were performed and four clones from each of the four vectors were picked for plasmid extraction.

FIG. 52 shows final insertion cassette (FIC) plasmids containing VHH1-Fc-VHH2 and VHH1-VHH2-Fc gene were diagnosed with restriction enzymes (KpnI/NheI and DraI/NcoI) and (NcoI/DraI) respectively.

FIG. 53A-53B shows the expression level of bi-specific antibody with different final insertion cassettes.

FIG. 54 shows a top clone was selected with cell-based neutralization assays. FIC5 clones with the highest expression of VHH1-VHH2-Fc were selected for validating neutralization activities against human recombinant TNFα (left) and IL-17A (right). Clone G7-E1 (FZ008) showed consistent high expression of the fusion polypeptide and neutralizing activities against TNFα and IL-17A.

FIG. 55 shows exemplary plasmids for FZMYA06-16 (ura3(−/−), gap1(−/−), Cir+) development.

FIG. 56 shows exemplary strategies for FZMYA06-16 development.

FIG. 57 shows exemplary platform for auxotrophic S. boulardii strain development from parental MYA796 to FZMYA06-16.

FIG. 58 shows exemplary final characterization of FZMYA06-16 (growth phenotype).

FIG. 59 shows exemplary final characterization of FZMYA06-16 (PCR confirmation the site-specific deletion of both URA3 gene and replaced antibiotic cassette).

FIG. 60 shows exemplary final characterization of FZMYA06-16 (PCR confirmation the site-specific deletion of GAP1 gene and replaced antibiotics cassette).

FIG. 61 shows exemplary final characterization of FZMYA06-16 (PCR confirmation no antibiotics gene G418 or Phleo-R in genome).

FIG. 62 shows exemplary final characterization of FZMYA06-16 (sequencing LoxP-scar at URA3 and GAP1 loci in genome).

FIG. 63 shows exemplary final characterization of FZMYA06-16 (growth curve at different pH conditions).

FIG. 64 shows exemplary gene of interest (GOI) cassette containing plasmid platform.

FIG. 65 shows a schematic insertion of GOI cassette in promoter region using ends-in homologous recombination method.

FIG. 66 shows exemplary generation of an engineered strain of Saccharomyces yeast.

FIG. 67 shows an exemplary flow chart for development strategy of engineered strains of Saccharomyces yeast expressing different formats of therapeutic polypeptides using platforms described herein.

FIG. 68 demonstrates an exemplary FZ002 development flow chart.

FIG. 69 shows exemplary PCR of pTEF-ABAB-URA3/DHFR cassettes.

FIG. 70 shows a schematic of exemplary first electroporation of FZMYA06-16.

FIG. 71 shows a schematic of exemplary screening processing.

FIG. 72 shows functional activity (neutralizing) screening.

FIG. 73 shows exemplary ELISA screening.

FIG. 74 shows exemplary ELISA for clone uniformity screening.

FIG. 75 demonstrates site-specific insertion of expression cassette.

FIG. 76 demonstrates exemplary second electroporation of FZMYA06-16. Sb pTEF-ABAB-DHFR clone B2 and B10 were selected for competent cell preparation for the 2nd Electroporation. Sb pTEF-ABAB-URA3 clone C11 and D10 were selected for competent cell preparation for the 2nd Electroporation

FIG. 77 shows expression cassette PCR from plasmid for second electroporation of pTEF-ABAB+ cells.

FIG. 78 shows second electroporation positive clone screening.

FIG. 79 shows second electroporation positive clone screening.

FIG. 80 shows second electroporation positive clone screening.

FIG. 81 shows exemplary top clones chosen from each group.

FIG. 82 shows exemplary selected top ABAB-expression clone uniformity.

FIG. 83 shows exemplary selected top ABAB-expression clone uniformity.

FIG. 84 shows exemplary ABAB expressing S. boulardii (FZ002 clones) characterization.

FIG. 85 shows exemplary FZ002 characterization (growth curve).

FIG. 86 shows exemplary FZ002 characterization (growth curve).

FIG. 87 shows exemplary FZ002 characterization (growth phenotype; passage one).

FIG. 88 shows exemplary FZ002 characterization (growth phenotype; passage eleven).

FIG. 89 shows exemplary FZ002 characterization (stability). Different FZ002 clones (2E9, 6G1, delta+HS #1) and negative control (wile-type Sb) were continuously passaged every 24 hours for 11 passages (around of 100 generations), seeding OD600 at 0.1, and cultured at 37° C. shaking 250 rpm. Crude culture supernatants from each passage were collected and assayed by ELISA with TcdB capture and expression of ABAB detected by HRP-conjugated anti-Llama IgG (H+L).

FIG. 90 shows exemplary Sb-ABAB characterization (genotype confirmation). S. Boulardii vs. S. cerevisiae Fungal ITS Library: 0.0% difference in sequence alignment with S. cerevisiae boulardii; 0.71% difference with S. cerevisiae. Results were reported as S. cerevisiae because they do not report out subspecies.

FIG. 91 shows exemplary FZ002 characterization (genomic insertion).

FIG. 92 shows exemplary FZ002 characterization (genomic insertion).

FIG. 93 shows exemplary FZ002 characterization (genomic insertion).

FIG. 94 shows exemplary FZ002 characterization (genomic insertion).

FIG. 95 shows exemplary FZ002 characterization (neutralizing activity against TcdB).

FIG. 96 shows exemplary FZ002 characterization (anti-fungal sensitivity).

FIG. 97 shows exemplary Sb-ABAB characterization (antibiotics sensitivity). Sb-ABAB was similar to wild type, not sensitive to the following antibiotics at indicated concentrations used for C. difficile infection treatment: 42 μg/mL Colistin, 35 μg/ml Gentamicin, 400 μg/ml Kanamycin, 215 μg/ml Metronidazole, 45 μg/ml Vancomycin, 19.8 μg/mL Clindamycin.

FIG. 98 shows exemplary Sb-ABAB characterization (anti-fungi activity). Sb-ABAB was sensitive to G418, Clotrimazole, Ketoconazole, Itraconazole at indicated concentrations. The antifungal sensitivity of FZ002 sensitivity is similar as wild type. Neither WT nor FZ002 cells were sensitive to cefmetazole or ceftazidime, even at 10 mM.

FIG. 99 shows exemplary Sb-ABAB characterization (GI environmental resistance). Sb-ABAB and wild type were treated with the indicated GI-environment media condition for 1 hour before culture at 37° C. CFU counting after 24 hour culture showed Sb-ABAB was similar as wild type, resistant to the most testing condition, but sensitive to HCL buffer 1.2 (mimic the gastric environment) and 0.01 bile salts.

FIG. 100 shows exemplary FZ002 characterization (PK (in vivo); GOI expression in fecal/GI track (in vivo). C57BL/6 male mice were dosed with FZ002 1×10⁹/doses/day from DO to D3 (total 4 doses) after fecal sample collected. Fecal sample was collected on DO, and one day after the first, second, third dosing or one and 4 days after the final dosing. Each line indicates the kinetic change of ABAB level in fecal sample of each mouse.

FIG. 101 shows exemplary FZ002 characterization (colony forming unit (CFU)). Timepoint 1 CFU (left): 1 mL sample was taken from 100 mL SDC Medium inoculated with 1 mL FZ002-6G1 (lot 2021-AUG-11, 10\hat{ }8 cells/ml) and incubated at 37° C. and 250 rpm for 0 hr Sample name: AW-PRO-0123-FOS-SAM10.1.4.2-09DEC21 Results: ˜8.83E+04 CFU. Timepoint 4 CFU (right): 1 mL sample was taken from 100 mL SDC Medium inoculated with 1 mL FZ002-6G1 (lot AW21024-016-FOS-RCB-10dec.2021, 10\hat{ }8 cells/ml) and incubated at 37° C. and 250 rpm for 0 hr Sample name: AW-PRO-0123-FOS-SAM10.6.2-14DEC21 Results: ˜1.82E+05 CFU

FIG. 102 shows exemplary ABAB expression from random picked colonies. 14 colonies showed >20 ng/mL per 1 OD600 of cells. 3 colonies showed low expression (between 13 ng/ml and 20 ng/ml per 1 OD600 of cells of ABAB). 2 colonies showed no detectable expression of ABAB.

FIG. 103 shows exemplary FZ006 development flow chart.

FIG. 104 shows exemplary cassettes-containing plasmid construction and cassettes preparation.

FIG. 105 shows exemplary the first electoporation and colony formation after the electroporation of FZMYA06-16.

FIG. 106 shows exemplary screening processing.

FIG. 107 shows an exemplary ELISA screening.

FIG. 108 shows exemplary ELISA for clone uniformity screening.

FIG. 109 shows exemplary site-specific insertion of expression cassette.

FIG. 110 shows the second electroporation of pTEF-ahTNF-α+ cells. Single colony from the 2 top clones transformed with pTEF-aTNF-α-Fc-URA3 (clone B10, C2) for transformation crossover with pTDH3-URA3 cassette. Single colony from the 2 top clones transformed with pTEF-aTNF-α-Fc-DHFR (clone A2, A4) for transformation crossover with pTDH3 cassette. FZMYA06-16 for single transformation of pTDH3 cassette or Co-transformation of pTEF and pTDH3 cassette.

FIG. 111 shows cassettes preparation for second electroporation of pTEF-ahTNF-α+ cells.

FIG. 112 shows second electroporation with pTEF/pTDH3-ahTNF-α-DFR1 cassettes in FZMAY06-16.

FIG. 113 shows exemplary second electroporation positive clone screening.

FIG. 114 shows exemplary second electroporation positive clone screening.

FIG. 115 shows exemplary second electroporation positive clone screening.

FIG. 116 shows exemplary top clones from each group re-streaked on corresponding selection plate.

FIG. 117 shows exemplary top clones chosen from each group.

FIG. 118 shows exemplary FZ006 characterization.

FIG. 119 shows exemplary FZ006 characterization (acute expression level).

FIG. 120 shows exemplary FZ006 characterization (24 hour expression level of cells cultured at passage 1 and passage 11).

FIG. 121 shows exemplary FZ006 characterization (2 hour acute expression of top clones and clone uniformity).

FIG. 122 shows exemplary FZ006 characterization (growth phenotype).

FIG. 123 shows exemplary FZ006 characterization (growth phenotype).

FIG. 124 shows exemplary FZ006 characterization (growth curve).

FIG. 125 shows exemplary FZ006 characterization (growth curve).

FIG. 126 shows exemplary FZ006 characterization (expression stability).

FIG. 127 shows exemplary FZ006 characterization (expression stability).

FIG. 128 shows exemplary characterization of site-specific insertion of expression cassette.

FIG. 129 shows exemplary characterization of site-specific insertion of expression cassette.

FIG. 130 shows an exemplary FZ006m development flow chart.

FIG. 131 shows an exemplary cassette-containing plasmid and cassette preparation for FZ006m development.

FIG. 132 shows exemplary electroporation of FZMYA06-16.

FIG. 133 shows exemplary screening processing of FZ006m.

FIG. 134 shows exemplary ELISA screening of FZ006m.

FIG. 135 demonstrates ELISA for clone uniformity screening of FZ006m.

FIG. 136 shows exemplary construction of pTDH3-aTNF-α-Fc-aIL17A-DHFR.

FIG. 137 shows exemplary construction of pTDH3-aTNF-α-Fc-aIL17A-DFR1.

FIG. 138 shows exemplary construction of pTEF-aTNF-α-Fc-aIL17A-DHFR.

FIG. 139 shows exemplary construction of pTEF-aTNF-α-Fc-aIL17A-DFR1.

FIG. 140 shows exemplary construction of pTEF-aTNF-α-aIL17A-Fc DFR1.

FIG. 141 shows exemplary FZ008 development flow chart.

FIG. 142 shows exemplary cassettes-containing plasmid construction and cassettes preparation.

FIG. 143 shows exemplary cassettes-containing plasmid construction and cassettes preparation.

FIG. 144 shows exemplary electroporation of FZMYA06-16.

FIG. 145 shows an exemplary screening process of FZ008 clones (VHH-Fc-VHH).

FIG. 146 shows an ELISA screening of FZ008_ahTNF-α-Fc-ahIL17A.

FIG. 147 shows an ELISA screening of FZ008_ahTNF-α-ahIL17A-Fc.

FIG. 148 shows exemplary Sb-aTNF-α-aIL17A characterization.

FIG. 149 shows exemplary characterization (specific binding activity). Both formats binding to hTNF-α (left) and hIL-17A (right), but FZ008_ahTNF-α-ahIL17A-Fc showed higher expression level compared to FZ008_aTNF-α-Fc-aIL17A.

FIG. 150 shows exemplary characterization (neutralizing activity).

FIG. 151 shows exemplary characterization (growth phenotype).

FIG. 152 shows exemplary characterization (site-specific insertion of expression cassette).

FIG. 153 shows exemplary FZO10m development flow chart.

FIG. 154 shows exemplary cassettes-containing plasmid construction and cassettes preparation.

FIG. 155 shows exemplary electroporation of FZMYA06-16.

FIG. 156 shows an exemplary screening process of FZ010m clones.

FIG. 157 shows ELISA screening of clones with pTEF/pTDH3-mIL22-Fc-URA3 cassettes.

FIG. 158 shows ELISA screening of FZ010m_pTEF-mIL22-Fc-DHFR.

FIG. 159 shows ELISA screening of FZ010m_pTDH3-mIL22-Fc-URA3/DHFR.

FIG. 160 shows ELISA screening of Co-transformation of FZ010m_pTEF-URA3+pTDH3-DHFR.

FIG. 161 exemplary characterization of uniformity.

FIG. 162 shows selection of top expression clone uniformity.

FIG. 163 shows food and water consumption.

FIG. 164 shows body weight and fasting blood glucose levels.

FIG. 165 shows an exemplary FZ014 development flow chart.

FIG. 166 shows exemplary cassettes preparation directly from golden gate reaction.

FIG. 167 shows pTDH3-2KD1-Fc-Aprotinin-DFR1 cassette preparation.

FIG. 168 shows positive clones after electroporation with different cassettes in FZMYA06-16.

FIG. 169 shows an exemplary screening process of FZ014 clones.

FIG. 170 shows ELISA screening of FZ014_2KD1-Fc.

FIG. 171 shows ELISA screening of FZ014_Aprotinin-2KD1-Fc.

FIG. 172 shows ELISA screening of FZ014_2KD1-Fc-Aprotinin.

FIG. 173 shows expression comparison of the three formats of 2KD1-Fc.

FIG. 174 shows exemplary characterization of FZ024 (growth curve).

FIG. 175 shows exemplary characterization of FZ014 (growth curve).

FIG. 176 shows exemplary characterization of FZ014 (site-specific insertion of expression cassette).

FIG. 177 shows exemplary characterization of FZ014 (ELISA to detect 2KD1 binding to RVA antigen).

FIG. 178 shows exemplary characterization of FZ014 (virus neutralizing assay). Virus=Wa att HRV Strain, working dilution 1/500 >1/250 dil 1-2; MA104 passage 16 from Friday to Monday; Assay Development >Infected cells were stained with Nanobody 2KD1 labeled with Alexa 488, working dilution: 1/500 in PBS-Evan Blue, 45 min 37° C.

FIG. 179 shows an exemplary FZ016 development flow chart.

FIG. 180 shows exemplary cassettes preparation directly from golden gate reaction.

FIG. 181 shows exemplary electroporation of FZMYA06-16.

FIG. 182 shows exemplary screening processing.

FIG. 183 shows exemplary ELISA screening of FZ016_M6-M4-Fc.

FIG. 184 shows exemplary ELISA screening of FZ016_M6-M5-Fc.

FIG. 185 shows expression comparison of FZ016_M6-M4-Fc vs FZ016_M6-M5-Fc.

FIG. 186 shows an exemplary ELISA blocking assay to detect the blocking effect of 2KD1 on interruption the adherence of NoVr VLP GII.4/1974 VLP Native Antigen to pig gastric mucin.

FIG. 187 shows an exemplary ELISA blocking assay to detect the blocking effect of 2KD1 on interruption the adherence of NoVr VLP GII.4/1974 VLP Native Antigen to H type 3 carbohydrate.

FIG. 188 shows a schematic structure of FZO20 development.

FIG. 189 shows an exemplary overview of FZO20 development.

FIG. 190 shows an exemplary FZO20 development flow chart.

FIG. 191 shows exemplary cassettes containing plasmid construction.

FIG. 192 shows exemplary cassettes preparation for FZO20 development.

FIG. 193 shows exemplary first electroporation of FZMYA06-16.

FIG. 194 shows an exemplary screening processing for FZ002_ABAB (pTDH3-DHFR).

FIG. 195 shows ELISA screening of FZ002_ABAB expression (pTDH3-DHFR).

FIG. 196 shows a top Sb-ABAB (pTDH3-DHFR) clone uniformity check.

FIG. 197 shows exemplary FZ002_ABAB-2E9 (pTDH3-ABAB-DHFR) characterization (growth phenotype).

FIG. 198 shows exemplary FZ002_ABAB-2E9 (pTDH3-ABAB-DHFR) characterization (growth curves).

FIG. 199 shows exemplary FZ002_ABAB-2E9 (pTDH3-ABAB-DHFR) characterization (stability).

FIG. 200 shows exemplary ABAB-2E9 (pTDH3-ABAB-DHFR) characterization (acute expression).

FIG. 201 shows exemplary ABAB-2E9 (pTDH3-ABAB-DHFR) characterization (genome specific insertion site).

FIG. 202 shows exemplary generation of S. boulardii stain of FZ002-2E9B2 and characterization.

FIG. 203 shows a schematic of FZE1 amplification copy number.

FIG. 204 shows generation of S. boulardii strain of FZ002-2E9-B2 by FZE1 amplifying the copy number. Growth recovery defined as: during FZE1 treatment, overnight culture, the OD600 is at least half of that of cells not treated by FZE1, which is considered the growth recovery. Once the cells are recovered, plating cells on YPD plate for expression level test or move on to the next level treatment (FZE1 higher concentration).

FIG. 205 shows generation of S. boulardii strain of FZ002-2E9B2 by FZE1 amplifying the expression level.

FIG. 206 shows generation S. boulardii strain of FZ002-2E9-B2 by FZE1 amplifying the expression level.

FIG. 207 shows exemplary schematic for generation of S. boulardii strain of FZ020 and characterization.

FIG. 208 shows TNF-α-Fc expression cassette transformation and transformants screening.

FIG. 209 shows ELISA screening of ahTNF-α+ABAB.

FIG. 210 shows exemplary FZO20 characterization (growth phenotype).

FIG. 211 shows exemplary FZO20 characterization (genome specific insertion site).

FIG. 212 shows exemplary FZO20 characterization (genome specific insertion site).

FIG. 213 shows exemplary FZO20 characterization (binding activity to both TcdB and human TNFa).

FIG. 214 shows exemplary FZO20 characterization (neutralizing activity against TcdB and human TNFa).

FIG. 215 shows an exemplary FZ024 development flow chart.

FIG. 216 shows PCR of Leptin-HA-DFR1/Leptin-Fc-DFR1 cassettes for FZ024 development.

FIG. 217 shows electroporation of FZMYA06-16.

FIG. 218 shows an exemplary screening processing of FZ024 clones.

FIG. 219 shows an exemplary ELISA screening of FZ024 clones.

FIG. 220 shows exemplary generation of “Leptin-HA” strain for mouse studies. Two frozen vials were used (each ˜1×10\hat{ }8 cells in 1 mL) from a cell bank of FZY14-E6 to inoculate 2×50 mL flasks of YPD.

FIG. 221 shows an exemplary FZ028 development flow chart.

FIG. 222 shows exemplary cassettes preparation directly from golden gate reaction for FZ028 development.

FIG. 223 shows electroporation of FZMYA06-16.

FIG. 224 shows exemplary screening processing of FZ028 clones.

FIG. 225 shows exemplary ELISA screening of FZ028 clones.

FIG. 226 shows exemplary FZO10 development flow chart.

FIG. 227 shows exemplary cassettes-containing plasmid construction and cassettes preparation for FZO10 development.

FIG. 228 shows exemplary electroporation of FZMYA06-16.

FIG. 229 shows an exemplary screening process.

FIG. 230 shows a first 2 hour acute ELISA screening of FZ010_pTDH3-hIL22 fused with yFc(N297Q) at N- or C-terminal.

FIG. 231 shows a first 2 hour acute ELISA screening of FZ010_pTDH3-hIL22-DFR1.

FIG. 232 shows exemplary assessment of uniformity.

FIG. 233 shows a second 2 hour acute ELISA screening of FZ010_pTDH3-hIL22 fused with yFc(N297Q) at N- or C-terminal.

FIG. 234 shows a second 2 hour acute ELISA screening of FZ010_pTDH3-hIL22-DFR1.

FIG. 235 shows an exemplary flowchart for development of FZE1 (combination of methotrexate and sulfanilamide) selection for S. boulardii (Sb) (test FZE1 effective dose in Sb).

FIG. 236 demonstrates testing of methotrexate (MTX) effective dose in S. boulardii (Sb). pCEV-G4-Km-DHFR and pCEV-G4-Km transformed FZMYA06-16, transformed single clone was pickup for characterization of growth with MTX. Cells were seeded at OD=0.2, 24 hour reading. MTX alone was not enough to cause an effect on growth inhibition even at 500 nM.

FIG. 237 demonstrates testing of sulfanilamide effective dose in S. boulardii (Sb). Sulfanilamide was prepared in DMSO. Carrier DMSO did not show toxicity to Sb. Sulfanilamide can be used lower than 10 mg/mL.

FIG. 238 demonstrates testing of sulfanilamide effective dose in S. boulardii (Sb). Sulfanilamide (<5 mg/ml) can be used for combination with MTX.

FIG. 239 demonstrates testing of FZE1 (combination of MTX and sulfanilamide (sulfa)) effective dose in S. bouldardii (Sb). For combination, MTX>10-100 μM, Sulfa can be used at 1 mg/ml.

FIG. 240 demonstrates testing of FZE1 (combination of MTX and sulfanilamide (sulfa)) effective dose in S. boulardii (Sb). For combination, MTX can be used at 50-250 μM, Sulfa can be used at 1 mg/ml.

FIG. 241 demonstrates confirmation of FZE1 dose for selection in Sb. DHFR selected transformants can be selected form MTX at 50-300 μM plus Sulfa at 1 mg/ml. In some instances, an optimized dose for DHFR selection is MTX at 250 μM plus Sulfa at 1 mg/ml due to clear background and relative more transformants for screening.

FIG. 242 shows amino acid sequence similarity of mouse DHFR and yeast DFR1.

FIG. 243 shows a schematic of development of S. boulardii (Sb) DFR1 as a selection marker.

FIG. 244 demonstrates S. boulardii (Sb) anti-TNF-α-Fc-DFR1 inserted at different locations showed FZE1 (Sulfa 1 mg/mL+50 μM MTX) selected clone exhibit highest-expression of GOI.

FIG. 245 demonstrates screening of high-expression clones by DHFR. Screening of anti-hIL-17A-Fc-expressing clones with URA3 or DHFR selection marker, of which the GOI cassettes was inserted at Delta sites. After transformation into FZMYA06-16 (URA3−/−, GAP1−/−) strain with DNA fragment with GOI cassettes with URA3 or DHFR selection marker which flanked by Delta insertion homologues arm. Positive clones were picked up from corresponding plate: SDCAA-URA3 plate for URA3 selection and YPD+FZE1 for DHFR selection. 184 positive clones each were sub-cultured at 600 μl of YPD for 24 hour shaking at 250 rpm at 37° C. Culture supernatants were diluted 10× and detected by ELISA. Data readout at OD450 nm.

FIG. 246 demonstrates screening of high-expression clones by DHFR. Screening of anti-hTNF-α-Fc-expressing clones with positive URA3 or DHFR selection marker, of which the GOI cassettes was inserted pTDH3 sites. After transformation on FZMYA06-16 (URA3−/−, GAP1−/−) strain with DNA fragment with GOI cassettes with URA3 or DHFR selection marker which flanked by pTDH3 homologues arm. Positive clones were picked up from corresponding plate: SDCAA+URA3 plate for URA3 selection and YPD+FZE1 for DIFR selection. 84 positive clones each were sub-cultured at 600 μl of YPD for 24 hour shaking at 250 rpm at 37° C. Culture supernatant were diluted 20× and detected by ELISA. Data readout at OD450 nm.

FIG. 247 shows schematic of amplification of copy number and increase of gene expression by FZE1.

FIG. 248 shows amplification of copy numbers of GFP expression cassettes by culturing engineered yeast carrying dihydrofolate reductase in of the media containing FZE1.

FIG. 249 shows increase of the GFP expression by FZE1. Culture both low and high-expression clones in FZE1 medium for adaption elevates the expression level.

FIG. 250 demonstrates the increase of the expression of ABAB by FZ002 after culturing the yeast in the media containing FZE1. FZ002 clones 2E9 and 6G1 were continued to be cultured in FZE1 culture medium until adaption (Left panel). Single clones were pick up to measure the ABAB expression by ELISA, OD 450 were shown (Right panel).

FIG. 251 demonstrates the increase of the expression of ABAB by FZ002 after culturing the yeast in the media containing FZE1.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present disclosure provides technologies related to engineered strains of Saccharomyces yeast that express a therapeutic polypeptide(s) and/or comprise a nucleic acid encoding a therapeutic polypeptide(s). Such engineered strains of Saccharomyces yeast can provide stable and constitutive expression of therapeutic polypeptide(s), in some embodiments, without the need for conventional in vitro polypeptide expression systems (e.g., CHO or E. coli cells) that utilize bioreactors or bacterial fermenters followed by downstream polypeptide processing and purification prior to use as a therapeutic.

Engineered strains of Saccharomyces yeast of the present disclosure can be delivered, for example, orally or rectally, allowing for self-administration by a subject as opposed to a health care professional. Such self-administration can lead to improved therapeutic compliance and ultimately favorable treatment and/or prevention of a disease and/or disorder.

Many modifications and other embodiments disclosed herein will be readily apparent to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an engineered strain of Saccharomyces yeast”, “a genetically-modified yeast,” “a carrier,” or “an inflammatory bowel disease,” includes, but is not limited to, mixtures or co-occurrence of two or more such engineered strains of Saccharomyces yeast, genetically-modified yeasts, carriers, or inflammatory bowel diseases, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, ‘less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, ‘greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

“Diabetes” refers to a chronic disease characterized by hyperglycemia. Traditionally, a majority of diabetes cases fall into two broad pathogenic categories, Type 1 diabetes (T1D) and Type 2 diabetes (T2D). However, in some subjects, this classification is not applicable as other genetic, immunological, or neuroendrocrinological pathways are involved in pathogenesis.

“Inflammatory bowel disease” (IBD) refers to a group of gastrointestinal disorders characterized by a chronic nonspecific inflammation of portions of the gastrointestinal tract. Ulcerative colitis (UC) and Crohn's Disease (CD) are the most prominent examples of IBD in humans. However, IBD is also known to occur in animals, particularly dogs, cats and horses. IBD is associated with many symptoms and complications, including growth retardation in children, rectal prolapse, blood in stools (e.g. melena and/or hematochezia), wasting, iron deficiency, and anemia, (e.g. iron deficiency anemia and anemia of chronic disease or of chronic inflammation). The etiology or etiologies of IBD are unclear.

“Ulcerative colitis” (UC) refers to a chronic, non-specific, inflammatory, and ulcerative disease having manifestations primarily in the colonic mucosa. It is frequently characterized by bloody diarrhea, abdominal cramps, blood and mucus in the stools, malaise, fever, anemia, anorexia, weight loss, leukocytosis, hypoalbuminemia, and an elevated erythrocyte sedimentation rate (ESR). Complications of UC can include hemorrhage, toxic colitis, toxic megacolon, occasional rectovaginal fistulas, and an increased risk for the development of colon cancer. Ulcerative colitis is also associated with complications distant from the colon, such as arthritis, ankylosing spondylitis, sacroiliitis, posterior uveitis, erythema nodosum, pyoderma gangrenosum, and episcleritis. Treatment varies considerably with the severity and duration of the disease. For instance, fluid therapy to prevent dehydration and electrolyte imbalance is frequently indicated in a severe attack. Additionally, special dietary measures are sometimes useful. Medications include various corticosteroids, sulfasalazine and some of its derivatives, and possibly immunosuppressive drugs.

“Crohn's disease” (CD) shares many features in common with ulcerative colitis. Crohn's disease is distinguishable in that lesions tend to be sharply demarcated from adjacent normal bowel, in contrast to the lesions of ulcerative colitis which are fairly diffuse. Additionally, Crohn's disease predominantly afflicts the ileum (ileitis) or the ileum and colon (ileocolitis). In some cases, the colon alone is diseased (granulomatous colitis) and sometimes the entire small bowel is involved (jejunoileitis). In rare cases, the stomach, duodenum, or esophagus are involved. Lesions include a sarcoid-type epithelioid granuloma in roughly half of clinical cases. Lesions of Crohn's disease can be transmural, including deep ulceration, edema, and fibrosis, which can lead to obstruction and fistula formation as well as abscess formation. This contrasts with ulcerative colitis which usually yields much shallower lesions, although occasionally the complications of fibrosis, obstruction, fistula formation, and abscesses are seen in ulcerative colitis as well.

As used herein, “administering” can refer to an administration that is oral and/or rectal and can be continuous or intermittent. In various aspects, the disclosed organisms, compositions, and/or preparations can be administered therapeutically; that is, administered to treat an existing disease or condition. In further various aspects, the disclosed organisms, compositions, and/or preparations can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including macromolecules such as proteins, peptides, hormones, nucleic acids, gene constructs, as well as genetically modified microorganisms, and the like. The agent may be a biologically active agent used in medical, including veterinary, applications. The term therapeutic agent also includes without limitation, medicaments; vitamins; mineral supplements; substances used for the treatment, prevention, diagnosis, cure or mitigation of disease or illness; or substances which affect the structure or function of the body such a probiotic micro-organisms, pre-biotics; or pro-drugs, which become biologically active or more active after they have been placed in a predetermined physiological environment.

As used herein, “therapeutic polypeptide” refers to a protein or peptide that possesses a biological activity that is implicated or involved in the treatment or prevention of a disease or condition.

As used interchangeably herein, “subject,” “individual,” or “patient” can refer to a vertebrate organism, such as a mammal (e.g., human, dog, cat, horse, pig, chicken, turkey, goats, sheep, cattle, rabbit). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. Exemplary conditions that can be treated include, but are not limited to, Crohn's disease, ulcerative colitis, and other inflammatory bowel diseases and/or an inflammation-related condition, as well as diabetes, obesity, and certain infections. The effect can be therapeutic in terms of a partial or complete cure of a disease, condition, symptom or adverse effect attributed to the disease, disorder, or condition. The term “treatment” as used herein can include (a) inducing remission of the disease or condition being treated; (b) inhibiting the disease, i.e., arresting its development; and (c) relieving the disease, i.e., mitigating or ameliorating the disease and/or its symptoms. Those in need of treatment (e.g., subjects in need thereof) can include those already with the disorder, disease, or condition and/or those suspected of having the disease, disorder, or condition. Treating the disease, disorder, or condition can include ameliorating at least one symptom of the particular disease, disorder, or condition, even if the underlying pathophysiology is not affected.

As used herein, the terms “preventing,” “preventing,” or “prophylactic” refer to halting, precluding, averting, obviating, forestalling, stopping, or hindering the development of a disease or condition that a subject may be predisposed to developing prior to the development of the disease or condition. In some embodiment, “preventing” or “prophylactic” may simply refer to decreasing the likelihood that a subject (e.g., a subject with a predisposition to develop a disease or condition) develops a given disease or condition (e.g., Crohn's disease, diabetes, ulcerative colitis, etc.), as it is understood that most prophylactic agents are not 100% effective in preventing a disease or condition in every subject that receives it. In some embodiments, “preventing” or “prophylactic” may refer the prevention of a disease flair if the subject is currently in remission while receiving the treatment (i.e., “preventing” can, in certain contexts, be synonymous with maintaining remission).

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, or condition, or to decreasing in the rate of advancement of a disease, disorder, or condition.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disease, disorder, and/or condition being treated and the severity of the disease, disorder, and/or condition; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease, disorder and/or condition, in some instances, the desired response can be inhibiting the progression of the disease, disorder and/or condition. This may involve only slowing the progression of the disease, disorder and/or condition temporarily. However, in other instances, it may be desirable to halt the progression of the disease, disorder, and/or condition permanently. This can be monitored by routine diagnostic methods known to one of ordinary skill in the art for any particular disease, disorder and/or condition.

As used herein, the term “prophylactically effective amount” refers to an amount effective for preventing onset or initiation of a disease, disorder, and/or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons, or for virtually any other reasons.

A response to a therapeutically effective dose of a disclosed compound and/or pharmaceutical composition, for example, can be measured by determining the physiological effects of the treatment or medication, such as the decrease or lack of disease, disorder, and/or condition symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the dosage timing and so on. Dosage can vary, and can be administered in one or more dose administrations daily, for one or several days.

As used herein, the term “probiotic microorganism” or “probiotic bacterium” or “probiotic” refers to a microorganism or bacterium (e.g., Saccharomyces yeast, engineered strains of Saccharomyces yeast, including, for example, engineered strains of S. boulardii), which, when administered in an effective amount, confers a health or wellness benefit on a host.

As used herein, the term “promoter” is a transcription regulatory sequence at least sufficient to promote the transcription of a nucleotide sequence in DNA into an RNA transcript. A transcript transcribed from a promoter typically includes sequences from the promoter downstream of the transcription start site, as well as downstream sequences that, in the case of mRNA, encode an amino acid sequence. Promoters are the best-characterized transcriptional regulatory sequences because of their predictable location immediately upstream of transcription start sites. Promoters include sequences that modulate the recognition, binding and transcription initiation activity of the RNA polymerase. These sequences can be cis acting or can be responsive to trans acting factors. Promoters, depending upon the nature of the regulation, can be constitutive or regulated. They are often described as having two separate segments: core and extended promoter regions. The core promoter includes sequences that are sufficient for RNA polymerase recognition, binding and transcription initiation. The core promoter includes the transcriptional start site, an RNA polymerase binding site, and other general transcription binding sites and is where the pre-initiation complex forms and the general transcription machinery assembles. The pre-initiation complex is generally within 50 nucleotides (nt) of the transcription start site (TSS). The core promoter also includes a sequence for a ribosome binding site, necessary for translation of an mRNA into a polypeptide. The extended promoter region includes the so-called proximal promoter, which extends to about 250 nucleotides upstream of the transcriptional start site (i.e., −250 nt). It includes primary regulatory elements such as specific transcription factor binding sites. It has been found that many genes have transcription regulatory elements located further up-stream. In particular, a fragment that includes most of the transcription regulatory elements of a gene can extend up to 700 nt or more up-stream of the transcription start site. (See, e.g., U.S. 2007-0161031.) In certain genes, transcription regulatory sequences have been found thousands of nucleotides upstream of the transcriptional start site.

As used herein, a nucleotide sequence is “operatively linked” or “operably linked” with a transcription regulatory sequence when the transcription regulatory sequence functions in a cell to regulate transcription of the nucleotide sequence. This includes promoting transcription of the nucleotide sequence through an interaction between a polymerase and a promoter.

As used herein, the term “mutation” refers to an alteration in a nucleotide sequence or amino acid sequence. A mutation can include a substitution of one or more nucleotides (a single nucleotide substitution is referred to as an “SNV” or “point mutation”), one or more nucleotide additions or one or more nucleotide deletions, as well as the changes in amino acid sequence, if any, resulting from these nucleotide alterations.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural, and intrasternal injection and/or infusion.

As used herein, a “vector” is a replicable nucleic acid from which one or more heterologous polypeptides can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes. Selection and use of such vehicles are well known to those of skill in the art. A vector also includes “virus vectors” or “viral vectors.” Viral vectors are engineered viruses that are operably linked to exogenous genes to transfer (as vehicles or shuttles) the exogenous genes into cells. As used herein, an “expression vector” includes vectors capable of expressing DNA that is operably linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

The term “pharmaceutically acceptable” describes a material that is not biologically or otherwise undesirable, i.e., without causing an unacceptable level of undesirable biological effects or interacting in a deleterious manner.

The term “contacting” as used herein refers to bringing a disclosed compound or pharmaceutical composition in proximity to a cell, a target protein or polypeptide, or other biological entity together in such a manner that the disclosed compound or pharmaceutical composition can affect the activity of the a cell, target protein or polypeptide, or other biological entity, either directly; i.e., by interacting with the cell, target protein or polypeptide, or other biological entity itself, or indirectly; i.e., by interacting with another molecule, co-factor, factor, or protein or polypeptide on which the activity of the cell, target protein or polypeptide, or other biological entity itself is dependent.

### Engineered Strains of Saccharomyces Yeast

The present disclosure provides, among other things, engineered strains of Saccharomyces yeast. In some embodiments, engineered strains of Saccharomyces yeast express one or more therapeutic polypeptide(s) as described herein. In some embodiments, engineered strains of Saccharomyces yeast comprise a nucleic acid encoding one or more therapeutic polypeptide(s) described herein.

Also disclosed are technologies for development of engineered strains of Saccharomyces yeast. In one aspect, the methods result in stable, high expression of heterologous genes in the engineered strains of Saccharomyces yeast. In another aspect, the engineered strains of Saccharomyces yeast constitutively secrete therapeutic polypeptide(s) (e.g., in the gastrointestinal tract of a subject). In still another aspect, the methods allow for multiple gene insertions, the expression of full-length antibodies, the expression of multiple therapeutic polypeptide(s), and/or the expression of multiple enzymes for the generation of specific metabolites.

In one aspect, the present disclosure relates to an engineered strain of Saccharomyces yeast that produces at least one (e.g., 1, 2, 3, 4 or more) therapeutic polypeptide(s) capable of binding with one or more specific targets (e.g., disease targets) in a subject, wherein the therapeutic polypeptide(s) is produced in vivo in the subject. In another aspect, the at least one (e.g., 1, 2, 3, 4 or more) therapeutic polypeptide(s) comprises a mammalian (e.g., a human, dog, cat, horse, pig, chicken, turkey, goats, sheep, cattle) cytokine or chemokine. Exemplary, non-limiting cytokines include, IL-1, IL-2, IL-4, TNF-α, IL-17A, IL-6, IL-8, IL-10, IL-12, GM-CSF, IL-13, IL-18, IL-20, IL-22, IL-23, IL-25, IL-27, IL-35, IL-39, TGF-β, or any combination thereof. In one aspect, inhibition of an inflammatory cytokine is superior to that of an appropriate reference standard (e.g., inhibition of an inflammatory cytokine produced by existing biologics). In another aspect, the at least one therapeutic polypeptide(s) comprises an antibody or a functional fragment thereof. In another aspect, the therapeutic polypeptide(s) comprises one or more of soluble receptors or binding proteins, one or more cytokines or chemokines, or any combination thereof. In another aspect, the therapeutic polypeptide(s) comprises for example, a hormone, enzyme, antimicrobial peptide, microbial peptide, or a group of therapeutic enzymes for the synthesis of therapeutic metabolites.

In a further aspect, a nucleic acid encoding a therapeutic polypeptide(s) can be inserted in a site-specific manner in a “hot spot”, in one or multiple copies, can produce a therapeutic polypeptide(s) stably and/or at a high level, or any combination of these features. For the purposes of the present disclosure, a “hot spot” is a high expression genomic locus or loci. In some embodiments, site-specific insertions insert one or more nucleic acids encoding a therapeutic polypeptide(s) in multiple sites of a genome (e.g., in 2, 3, 4, 5, or 6 or more sites). In some embodiments, site-specific insertion does not disrupt and/or replace any genes endogenously present in the genome. Exemplary hot spots include, without limitation, tef1, tdh3, tdh2, tef2, eno2, rpla1, rpla2, rpla3, rpla10e, pdc1, adh1, adh2, gpm1, fba1, rp147a, pgk1, rpla4, ssm1a, ssm1b, rpl5a, rpl5b, upf1, rp116a, rp116b, cup1a, cup1b, rps31a, rp12a, rp12b, rps28a, rp135b, pyk1, rp19a, rp19b, rp127a, rps21, rp143a, nab1a, nab1b, urp1a, and rps18eb.

In some embodiments, site-specific insertion is conducted using transposon-directed insertion (e.g., at Ty, Delta, Sigma, pTEF, and/or pTDH3 sites). In some embodiments, site-specific insertion is disruptive site-specific insertion (e.g., a nucleic acid to be inserted disrupts the originally present nucleic acid). In some embodiments, site-specific insertion is non-disruptive site-specific insertion (e.g., a nucleic acid to be inserted replaces the originally present nucleic acid). In some embodiments, site-specific insertion is ends-in-site-specific insertion (e.g., in a hot spot). In some embodiments, site-specific insertion is ends-out-site-specific insertion (e.g., in a hot spot). In some embodiments, site-specific insertion methods are highly-efficient relative to an appropriate reference standard (e.g., a different site-specific insertion method).

In a further aspect, a nucleic acid encoding a therapeutic polypeptide(s) can be inserted in a non-site-specific manner, in one or multiple copies, can produce a therapeutic polypeptide(s) stably and/or at a high level, or any combination of these features.

In some embodiments, engineered strains of Saccharomyces yeast comprise modifications (e.g., insertions, deletions, mutations) to increase therapeutic polypeptide(s) expression, secretion, and/or stability.

In some embodiments, engineered strains of Saccharomyces yeast comprise modifications (e.g., insertions, deletions, mutations, etc.) to increase safety (e.g., biocontainment) of such live biotherapeutic products.

### Saccharomyces Strains

In some embodiments, engineered strains of Saccharomyces yeast of the present disclosure is a species of Saccharomyces genus. For example, and without limitation, Saccharomyces genus include Candida genus, Schizosaccharomyces genus, Kluyveromyces genus, Pichia genus, Issachenkia genus, Yarrowia genus, or Hansenula genus. A species classified as Saccharomyces genus may be, for example, S. cerevisiae, S. bayanus, S. boulardii, S. bulderi, S. cariocanus, S. cariocus, S. chevalieri, S. dairenensis, S. elhpsoideus, S. eubayanus, S. exiguus, S. florentinus, S. kluyveri, S. martiniae, S. monacensis, S. norbensis, S. paradoxus, S. pastorianus, S. spencerorum, S. turicensis, S unisporus, S. uvarum, or S. zonatus. A species classified as Candida genus may be, for example, C. albicans, C. ascalaphidarum, C. amphixiae, C. antarctica, C. arjgentea, C. atlantica, C. atmosphaerica, C. blattae, C. bromeliacearum, C. carpophila, C. carvajals, C. cerambvcidarum, C. chauliodes, C. corydah, C. dosseyi, C. dubliniensis, C. ergatensis, C. fructus, C. glabrata, C. fermentati, C. guilliermondii, C. haemulonii, C. insectamens, C. insectorun, C. intermedia, C. jeffresii, C. kefy r, C. krusei, C. husitaniae, C. lyxosophila, C. maltosa, C. marina, C. membranifaciens, C. milleri, C. oleophila, C. oregonensis, C. parapsilosis, C. quercitrusa, C. rugosa, C. sake, C. shehatea, C. temnochilae, C. tenuis, C. theae, C. tolerans, C. tropicalis, C. tsuchiyae, C. sinolaborantium, C. sojae, C. subhashii, C. viswanathii, C. utilis, or C. ubatubensis. A species classified as SchizosaccharomYces genus may be, for example, S. pombe, S. japonicus, S. octosporus, or S. cryophilus. A species classified as Kluyveromyces genus may be, for example, K. aestuarii, K. africanus, K. bacillisporus, K. blattae, K. dobzhanskii, K. hubeiensis, K. lactis, K. lodderae, K. marxianus, K. nonfermentans, K. piceae, K. sinensis, K. thermotolerans, K. waltii, K. wickerhamii, or K. yarrowii. A species classified as Pichia genus may be, for example, P. anomala, P. heedii, P. guilliermondii, P. kluyveri, P. membranifaciens, P. norvegensis, P. ohneri, P. pastoris, P. methanolica, or P. subpellculosa. A species classified as Issachenkia genus may be, for example, I. orientalis. A species classified as Yarrowia genus may be, for example, Y. lipolytica. A species classified as Hansenula genus may be, for example, N. subpelliculosa, N. anomala, H. polvmorpha, H. holstii Wick, or H. capsulata Wick.

In one aspect, the Saccharomyces yeast can be S. boulardii, which is an organism that is generally recognized as safe (GRAS) for probiotic use and that is, in unmodified form, a well-tolerated over-the-counter (OTC) probiotic for promoting intestinal health and amelioration of diarrhea. In one aspect, S. boulardii grows well at 37° C. and is more resistant to acidic environmental conditions than other strains of Saccharomyces yeast. However, in another aspect, molecular genetic tools for S. boulardii are not as well developed as for, for example, S. cerevisiae.

In some embodiments, engineered strains of Saccharomyces yeast do not comprise a selectable marker (e.g., an antibiotic selectable marker). In a further aspect, this complies with US Food and Drug Administration regulations regarding antibiotic resistance genes and, in a still further aspect, eliminates the possibility for microscale fungal evolution under antibiotic pressure.

In some embodiments, engineered strains of Saccharomyces yeast comprise a modification (e.g., mutation, deletion, insertion, etc.) that allows for selection of a particular engineered strain of Saccharomyces yeast (e.g., a selectable marker). In some embodiments, the modification that allows for selection comprises a partial deletion of a gene utilized as a selectable marker. In some embodiments, the modification that allows for selection comprises a complete deletion of a gene utilized as a selectable marker.

In some embodiments, a selectable marker allows for positive and/or negative selection. In some embodiments, a selectable marker comprises a prototrophic marker, an auxotrophic marker, markers conferring drug resistance, autoselection markers, and/or counterselectable markers. Non-limiting examples of genes utilized as selectable markers include ura3, gap1, leu2, his3, and trp1 (see, e.g., IMADEARTIKA, HAYATI Journal of Biosciences, Volume 16, Issue 1, 2009, Pages 40-42, ISSN 1978-3019). A plurality of selectable markers are known in the art. One of ordinary skill in the art would readily recognize and understand how to select and use such markers in accordance with technologies of the present disclosure.

In some embodiments, a selectable marker comprises a modification or deletion of an URA3 gene. URA3 encodes Orotidine 5′-phosphate decarboxylase (ODCase), an essential enzyme that catalyzes one reaction in the synthesis of pyrimidines. Without wishing to be bound by any one theory, loss of ODCase activity leads to a lack of cell growth unless uracil or uridine is added to the media. In contrast, if 5-FOA (5-Fluoroorotic acid) is added to the media, the active ODCase will convert 5-FOA into the toxic compound, 5-fluorouracil, causing cell death. Thus, engineered strains of Saccharomyces yeast comprising a deletion or inactivation of URA3, cannot survive without uracil or uridine. In some embodiments, an engineered strain of Saccharomyces yeast may have URA3 deleted [denoted as ura3^{-/-} or ura3(−/−)], while in other strains, URA3 may be mutated to be non-functional (ura3). In some embodiments, an engineered strain of Saccharomyces yeast comprises an insertion of URA3.

In some embodiments, a selectable marker comprises a modification (e.g., deletion) of a GAP1 gene. GAP1 encodes a general amino-acid permease involved in the uptake of all naturally occurring L-amino acids, related compounds, such as ornithine and citrulline, and some D-amino acids, toxic amino acid analogs such as azetidine-2-carboxylate, and the polyamines putrescine and spermidine. GAP1 is also involved in invasive growth of Saccharomyces strains. Accordingly, engineered strains of Saccharomyces yeast comprising a GAP1 modification (e.g., deletion) demonstrate reduced uptake of, for example, methionine, glycine, glutamine and regulated nitrogen sources. In addition, loss of GAP1 diminishes a Saccharomyces yeast strain's ability to grow invasively. Without wishing to be bound by any one theory, strains in which GAP1 is modified (e.g., deleted) can be selected for using minimal media where L-proline is the sole nitrogen source in combination with toxic D-His. Strains that are GAP1(−/−) can survive in media containing L-proline as the sole nitrogen source, with toxic amino acid D-histidine as the couterselection as D-His is taken up by GAP1 and is toxic to yeast cells, resulting in selection of GAP1(−/−) strains. Strains that do not comprise an inactivating modification in GAP1 (e.g., GAP1(++) strains) can be selected for on minimal media where L-citrulline is the sole nitrogen source. In some embodiments, an engineered strain of Saccharomyces yeast comprises a mutation in GAP1. In some embodiments, an engineered strain of Saccharomyces yeast comprises a deletion in GAP1. In some embodiments, an engineered strain of Saccharomyces yeast comprises an insertion of GAP1.

In some embodiments, a selectable marker comprises modification of a dihydrofolate reductase (DHFR) gene (see, e.g., MacDonald C et al., Yeast. 2015; 32(5):423-438.). DIFR is an enzyme that reduces dihydrofolic acid to tetrahydrofolic acid, using NADPH as an electron donor, which can be converted into tetrahydrofolate cofactors used in 1-carbon chemistry transfer. DI-FR confers resistance to methotrexate (MTX) and exhibits dose-effect selection. Thus, without wishing to be bound by any one theory, DI-FR can be used as a selectable marker with methotrexate, in some embodiments, in combination with Sulfanilamide, to screen for high-expression strains. In some such embodiments, methotrexate is administered for selection at about 1 nM, 5 nM, 10 nM, 20 nM, 50 nM, 75 nM, 100 nM, 150 nM, 200 nM, 250 nM, 500 nM, 750 nm, 1 μM, 5 μM, 10 μM, 20 μM, 50 μM, 75 μM, 100 μM, 150 μM, 200 μM, 250 μM, 500 μM, 750 μM, or 1 mM or any concentration in between the foregoing concentrations. In some such embodiments, methotrexate is administered for selection in combination with sulfanilamide. In some such embodiments, sulfanilamide is administered for selection at about 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 2 mg/mL, 3, mg/mL, 4 mg/mL, 5 mg/mL, 6 mg/mL, 7 mg/mL, 8 mg/mL, 9 mg/mL, or 10 mg/mL or any concentration in between the foregoing concentrations. In some such embodiments, about 0.8 nM-20 nM methotrexate or 50-250 μM methotrexate and about 0.1-10 mg/mL, 1-10 mg/mL, or 1-5 mg/mL sulfanilamide are administered. In some embodiments, a selectable marker comprises yeast homolog of DHFR, DFR1, for the selection of chromosomal insertion of transgenes. In some embodiments, DHFR has a comparable sensitivity to MTX as DFR1. DIFR is found in all organisms and is functionally conserved. Despite relatively low sequence identity between DHFR, DFR1, and other homologs, high functional similarity can, in some embodiments, permit use of a plurality of DIFR and DFR1 homologs in accordance with the presently disclosed technologies. Exemplary amino acid sequences of DI-FR and DFR1 are shown in Table 1. Given the sequence homology of mammalian DFHRs and yeast DFR1, the present technology is not limited to the use of mouse DHFR, but rather any homologue is expected to function as a selective marker as well. In some embodiments, the disclosed engineered yeast may comprise a nucleic acid sequence encoding a mammalian DHFR. In some embodiments, the disclosed yeast may comprise one or more exogenous copies of a nucleic acid sequence encoding DFR1. Given the sequence homology of mammalian DFHRs and yeast DFR1, the present technology is not limited to the use of mouse DHFR, but rather homologue from any organisms is expected to function as a selective marker as well. In some embodiments, the disclosed engineered yeast may comprise a nucleic acid sequence encoding a mammalian DI-FR. In some embodiments, the disclosed yeast may comprise one or more exogenous copies of a nucleic acid sequence encoding DFR1.

TABLE 1
Exemplary DHFR and DFR1 amino acid sequences.

| SEQ | | |
|---|---|---|
| ID | Descrip- | |
| NO: | tion | Amino acid sequence |
| 1 | sp\|P00375\|2- | VRPLNCIVAVSQNMGIGKNGDLPWPPLRNE |
| | 187 (mouse | FKYFQRMTTTSSVEGKQNLVIMGRKTWFSI |
| | DHFR) | PEKNRPLKDRINIVLSRELKEPPRGAHFLA |
| | | KSLDDALRLIEQPELASKVDMVWIVGGSSV |
| | | YQEAMNQPGHLRLFVTRIMQEFESDTFFPE |
| | | IDLGKYKLLPEYPGVLSEVQEEKGIKYKFE |
| | | VYEKKD |
| 2 | >sp\|P07807\| | MAGGKIPIVGIVACLQPEMGIGFRGGLPWR |
| | DYR_YEAST | LPSEMKYFRQVTSLTKDPNKKNALIMGRKT |
| | Dihydro- | WESIPPKFRPLPNRMNVIISRSFKDDFVHD |
| | folate | KERSIVQSNSLANAIMNLESNFKEHLERIY |
| | reductase | VIGGGEVYSQIFSITDHWLITKINPLDKNA |
| | OS = | TPAMDTFLDAKKLEEVFSEQDPAQLKEFLP |
| | Saccharomyces | PKVELPETDCDQRYSLEEKGYCFEFTLYNR |
| | cerevisiae | K |
| | (strain ATCC | |
| | 204508/ | |
| | S288c) | |
| | OX = 559292 | |
| | GN = DFR1 | |
| | PE = 1 SV = 2 | |

In some embodiments, an engineered strain of Saccharomyces yeast is auxotrophic. In one aspect, auxotrophic engineered strains of Saccharomyces yeast are less likely to survive in the environment relative to an appropriate reference standard (e.g., a parental strain of Saccharomyces yeast).

In some embodiments, engineered strains of Saccharomyces yeast have a similar growth curve compared to that of an appropriate reference standard (e.g., a parental strain of Saccharomyces yeast).

In some embodiments, extracts and/or supernatants from culture of engineered strains of Saccharomyces yeast retain at least some activity against a therapeutic target, an immune response, and/or alters at least one component of a metabolic pathway in a subject. In some such embodiments, at least some activity includes at least 50%, 55%, 60%, 65%, 70%, 75%, 80%, 85%, 90%, 95%, 96%, 97%, 98%, 99%, or 100% relative to an appropriate reference standard.

### Enhancing Stable Therapeutic Polypeptide(s) Expression

In some embodiments, engineered strains of Saccharomyces yeast comprise one or more modifications (e.g., insertions, deletions, mutations, etc.) to increase therapeutic polypeptide(s) expression, secretion, and/or stability.

In some embodiments, engineered strains of Saccharomyces yeast with increased therapeutic polypeptide(s) expression, secretion, and/or stability comprise modifications (e.g., insertions, deletions, mutations, etc.) in protease-encoding genes. In some such embodiments, a protease encoding gene is or comprises pep4, a yeast proteinase A. Pep4 is a vacuolar aspartyl hydrolase that not only degrades polypeptides in the vacuole, but also activates additional vacuolar proteases, such as Prc1 (carboxypeptidase Y), Prb1 (proteinase B), and Lap4 (aminopeptidase I). In some such embodiments, a protease encoding gene is or comprises, for example, PRC1, PRB1, and LAP4. Without wishing to be bound by any one theory, deletion and/or inactivation of proteases results in decreased degradation of a therapeutic polypeptide(s) produced by an engineered strain of Saccharomyces yeast and thus, leads to increased therapeutic polypeptide(s) expression, secretion, and/or stability.

In some embodiments, the effect of a modification to increase therapeutic polypeptide(s) expression, secretion, and/or stability is assessed using an enzyme-linked immunosorbent assay (ELISA). In some embodiments, such assessment further comprises comparison of the therapeutic polypeptide(s) expression, secretion, and/or stability of an engineered strain of Saccharomyces yeast relative to an appropriate reference standard (e.g., therapeutic polypeptide(s) expression, secretion, and/or stability in a corresponding parental strain of Saccharomyces yeast).

A plurality of methods are known in the art to assess polypeptide (e.g., therapeutic polypeptide(s)) expression, secretion, and/or stability). One of ordinary skill in the art would readily recognize and understand how to select and use such methods in accordance with the present disclosure.

### Enhancing Biologic Safety

Oral administration of S. boulardii probiotics have led to fungemia (e.g., the presence of fungi or yeast in the blood) in immunocompromised populations. Accordingly, technologies to improve safety of probiotics, including for example, engineered strains of Saccharomyces yeast described herein, are needed. In some embodiments, engineered strains of Saccharomyces yeast comprise modifications (e.g., insertions, deletions, mutations, etc.) to increase safety of such strains, and in particular for use as live biotherapeutic products (e.g., probiotics). In some embodiments, such modifications result in the build-up of toxic intermediates and lead to control (e.g., a reduction) of cellular proliferation (e.g., in eukaryotic cells).

In some embodiments, engineered strains of Saccharomyces yeast with improved biosafety comprise a modification (e.g., insertions, deletions, mutations, etc.) in genes related to metabolic pathways (e.g., for producing amino acids and/or components of nucleic acids). A plurality of genes related to metabolic pathways are known in the art and one of ordinary skill would readily appreciate and understand how to select such a gene for modification in accordance with the present disclosure. In some embodiments, such modifications to engineered strains of Saccharomyces yeast decrease immunogenicity of the engineered strains of Saccharomyces yeast and/or increase expression of the therapeutic polypeptide(s).

In some embodiments, a modification in a gene related to a metabolic pathway comprises a modification to a threonine biosynthesis gene. In some such embodiments, the threonine biosynthesis gene is thr1. Modification of thr1, an upstream enzyme in the threonine biosynthesis pathway, can result in serum-sensitivity when immunocompromised individuals are exposed to Saccharomyces yeast. Without wishing to be bound by any one theory, it is understood that this sensitivity is due to accumulation of a toxic intermediate molecule, homoserine, when the cells are exposed to low-threonine environments, such as serum, resulting in control (e.g., a reduction) of cell proliferation and thus, in production of safer biologic treatments relative to an appropriate reference standard (e.g., Saccharomyces yeast without an modified thr1). In some embodiments, an engineered strain of Saccharomyces yeast comprises a mutation in thr1. In some embodiments, an engineered strain of Saccharomyces yeast comprises a deletion in thr1. In some embodiments, an engineered strain of Saccharomyces yeast comprises an insertion in thr1.

In some embodiments, a threonine biosynthesis gene is thr4. The Thr4 enzyme is directly downstream of Thr1 in the threonine biosynthesis pathway and phosphorylates homoserine. thr4 null strains of Saccharomyces yeast exhibit similar phenotypes to thr1 null cells. Without wishing to be bound by any one theory, it is understood that the similar phenotype is a result of toxic intermediate, phosphohomoserine. In some embodiments, an engineered strain of Saccharomyces yeast comprises a mutation in thr4. In some embodiments, an engineered strain of Saccharomyces yeast comprises a deletion in thr4. In some embodiments, an engineered strain of Saccharomyces yeast comprises an insertion in thr4.

In some embodiments, modification in a gene related to a metabolic pathway comprises modification of ura3 gene. As above, ura3 encodes Orotidine 5′-phosphate decarboxylase (ODCase) which is an enzyme that catalyzes one reaction in the synthesis of pyrimidine ribonucleotides (a component of RNA). Without wishing to be bound by any one theory, loss of ODCase activity leads to a lack of cell growth unless uracil or uridine is added to the media. In some embodiments, modification of an ura3 gene results in control (e.g., a reduction) of cell proliferation and in production of safer biologic treatments relative to an appropriate reference standard (e.g., Saccharomyces yeast without a modified ura3 gene). In some embodiments, an engineered strain of Saccharomyces yeast comprises a mutation in ura3. In some embodiments, an engineered strain of Saccharomyces yeast comprises a deletion in ura3. In some embodiments, an engineered strain of Saccharomyces yeast comprises an insertion in ura3.

In some embodiments, modification in a gene related to a metabolic pathway comprises modification of a gap1 gene. As above, gap1 encodes a general amino-acid permease involved in the uptake of all naturally occurring L-amino acids, related compounds, such as ornithine and citrulline, and some D-amino acids, toxic amino acid analogs such as azetidine-2-carboxylate, and the polyamines putrescine and spermidine. Without wishing to be bound by any one theory, reduction in uptake can lead to a lack of cell growth. In some embodiments, modification of a gap1 gene results in control (e.g., a reduction) of cell proliferation and in production of safer biologic treatments relative to an appropriate reference standard (e.g., Saccharomyces yeast without a modified gap1). In some embodiments, an engineered strain of Saccharomyces yeast comprises a mutation in gap1. In some embodiments, an engineered strain of Saccharomyces yeast comprises a deletion in gap1. In some embodiments, an engineered strain of Saccharomyces yeast comprises an insertion in gap1.

### Development of Yeast Strains Expression Systems

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology, the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual, Freshney (2005) Culture of Animal Cells: A Manual of Basic Technique, 5th edition; Gait ed. (1984) Oligonucleotide Synthesis, U.S. Pat. No. 4,683,195; Hames and Higgins eds. (1984) Nucleic Acid Hybridization, Anderson (1999) Nucleic Acid Hybridization, Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

In some embodiments, the present disclosure provides technologies to develop and/or generate engineered strains of Saccharomyces yeast (e.g., engineered strains of Saccharomyces yeast produced in accordance with the present disclosure). In some such embodiments, use of PCR to amplify sequences (e.g., sequences encoding ORFs for GOI and selectable marker) can result in high-copy insertion. In some embodiments, development and/or generation of engineered strains of Saccharomyces yeast comprises yeast codon optimization of a gene of interest (GOI). In some such embodiments, a GOI is produced using, for example, synthesis and/or Polymerase Chain Reaction (PCR). In some embodiments, E. coli is transformed with a GOI and transformants are assessed by, for example, diagnostic digestion and/or sequencing. In some embodiments, an expression cassette comprising a GOI (e.g., a plasmid) is assessed by PCR and gel electrophoresis (e.g., DNA gel) to confirm the expression cassette by size. In some embodiments, purified expression cassette is electroporated into Saccharomyces yeast (e.g., FZMAY06-16) and plated for selection. In some embodiments, an expression cassette is inserted into the genome of the Saccharomyces yeast (e.g., to improve stability). In some embodiments, supernatants from positive transformants are assessed, for example by ELISA, for acute expression of the therapeutic polypeptide(s) encoded by a GOI in the supernatant. In some embodiments, one or more (e.g., two, three, four) rounds of clone screening is completed to validate the desired expression levels and purity of clones. In some embodiments, cell banks (CB) from desired clones are generated and further assessed for CB characterization.

In some embodiments, the present disclosure provides technologies for assessment of an engineered strain of Saccharomyces yeast (e.g., engineered strains of Saccharomyces yeast produced in accordance with the present disclosure). In some embodiments, an engineered strain of Saccharomyces yeast is assessed for one or more of expression level (e.g., by ELISA, Western Blot), growth phenotype, growth curve, stability (e.g., by ELISA), genotype confirmation, genome site-specific insertion (e.g., by Polymerase Chain Reaction, PCR), genome inserted gene of interest (GOI) cassette (e.g., by PCR), copy number of insertions, functional activity, antibiotic and/or antifungal sensitivity, gastrointestinal environmental survival, efficacy (e.g., in an animal model), pharmacokinetics (e.g., in vivo), GOI expression in fecal/gastrointestinal track (e.g., in vivo), and/or potency (e.g., C.F.U. identity, % of positive expression).

A plurality of methods related to Saccharomyces yeast modification, expression systems, and/or growth technologies are known in the art. One of ordinary skill in the art would readily recognize and understand how to select and use such methods in accordance with the present disclosure.

The following Table provides details of certain embodiments of yeast strains that may be utilized in various ways for the purposes of the disclosed technology.

| | S. boulardii (Sb) | | |
|---|---|---|---|
| | yeast strain | Description | Genotype |
| 1 | wildtype | Wildtype strain, diploid yeast | wildtype |
| 2 | Sb-EP | Sb yeast transformed with “Empty Plasmid” | ura3(−/−), cir⁰, |
| | | (pURA3-AT-cMyc) which does not contain the | Plasmid: URA3+ |
| | | ABAB transgene. | |
| 3 | Sb-ABAB | Sb transformed with plasmid containing ABAB | ura3(−/−), cir⁰, |
| | | transgene cassette (pURA3-AT-yABAB-cMyc). | Plasmid: URA3+, |
| | | ABAB is a tetra-specific anti-toxin VHH fusion | ABAB+ |
| | | protein targeting Clostridioides difficile TcdA and | |
| | | TcdB (AH3/5D/AA6/E3) | |
| 4 | FZMYA06-16 | Background strain. | ura3(−/−), gap1(−/−), |
| | | URA3 and GAP1 genes were knocked out from Sb | Cir⁺ |
| | | wildtype. | |
| | | Suitable for genomic integration of transgene | |
| | | cassettes. | |
| 5 | FZ002 | Expresses anti-toxin ABAB recombinant protein. | URA3+, mDHFR+, |
| | | Contains yeast-codon-optimized ABAB transgene | ABAB+, gap1(−/−), |
| | | cassettes: | Cir⁺ |
| | | yABAB-URA3 and | |
| | | yABAB-mDHFR | |
| | | which are genomically integrated into background | |
| | | strain FZMYA06-16 at specific sites on | |
| | | Chromosome VII and Chromosome XVI, | |
| | | respectively | |

Cir⁺: Sb strain contains endogenous episomal 2 um-plasmid native to S. boulardii and other Saccharomyces species (Douradinha et al., 2014)
Cir⁰: Sb strain lacking endogenous episomal 2 um-plasmid.
The 2 um-plasmid was removed in Sb-EP and Sb-ABAB so that yeast replication machinery favors retention of the exogenous plasmids (empty plasmid or pURA3-AT-yABAB-cMyc) used in these two strains
The platform strain FZMYA06-16 is auxotrophic for uracil due to its ura3(−/−) status. It is also gap1(−/−) which may limit its nitrogen uptake since GAP1 gene is a General Amino Acid permease. In the case of FZ002, one of the ABAB cassettes contains URA3, hence it is no longer auxotrophic for uracil. However, it is still gap1(−/−) which may hamper its ability to grow.

### Therapeutic Polypeptide(s)

The present disclosure provides therapeutic polypeptide(s) and engineered strains of Saccharomyces yeast that express such therapeutic polypeptide(s). In some embodiments, engineered strains of Saccharomyces yeast comprise one or more nucleic acids encoding one or more therapeutic polypeptide(s). In some embodiments, therapeutic polypeptide(s) are synthesized (e.g., expressed from engineered strains of Saccharomyces yeast comprising nucleic acids encoding one or more therapeutic polypeptide(s)) in the gastrointestinal tract of a subject. The engineered yeast of the present disclosure may have incorporated into their genome more than one (e.g., 2, 3, 4, 5, 6, 7, 8, 9, or 10 or more) copies of a gene of interest that encodes a therapeutic polypeptide.

In some embodiments, a therapeutic polypeptide(s) is or comprises a polymeric chain of amino acids that elicits therapeutic effect (e.g., a consequence of a medical treatment, the results of which are judged to be desirable and/or beneficial).

In some embodiments, a therapeutic polypeptide(s) comprises an amino acid sequence that occurs in nature. In some embodiments, a therapeutic polypeptide(s) comprises an amino acid sequence that does not occur in nature. In some embodiments, a therapeutic polypeptide(s) comprises an amino acid sequence that is engineered in that it is designed and/or produced through action of the hand of man. In some embodiments, a therapeutic polypeptide(s) may comprise or consist of natural amino acids, non-natural amino acids, or both. In some embodiments, a therapeutic polypeptide(s) may comprise or consist of only natural amino acids or only non-natural amino acids. In some embodiments, a therapeutic polypeptide(s) may comprise D-amino acids, L-amino acids, or both. In some embodiments, a therapeutic polypeptide(s) may comprise only D-amino acids. In some embodiments, a therapeutic polypeptide may comprise only L-amino acids. In some embodiments, a therapeutic polypeptide(s) may include one or more pendant groups or other modifications, e.g., modifying or attached to one or more amino acid side chains, at the therapeutic polypeptide(s)'s N-terminus, at the therapeutic polypeptide(s)'s C-terminus, or any combination thereof. In some embodiments, such pendant groups or modifications may be selected from the group consisting of acetylation, amidation, lipidation, methylation, pegylation, etc., including combinations thereof. In some embodiments, a therapeutic polypeptide(s) may be cyclic, and/or may comprise a cyclic portion. In some embodiments, a therapeutic polypeptide(s) is not cyclic and/or does not comprise any cyclic portion. In some embodiments, a therapeutic polypeptide(s) is linear. In some embodiments, a therapeutic polypeptide(s) may be or comprise a stapled therapeutic polypeptide(s). In some embodiments, the term “polypeptide(s)” may be appended to a name of a reference polypeptide, activity, or structure; in such instances it is used herein to refer to polypeptides that share the relevant activity or structure and thus can be considered to be members of the same class or family of polypeptides.

In some embodiments, a therapeutic polypeptide(s) is a monomer. In some embodiments, a therapeutic polypeptide(s) is a dimer. In some embodiments, a therapeutic polypeptide(s) is a multimer. In some embodiments, a therapeutic polypeptide(s) is a fusion polypeptide.

In some embodiments, a therapeutic polypeptide(s) is or comprises receptors, cytokines, chemokines, hormones, enzymes, antimicrobial peptides, and/or any non-natural functional proteins (such as DARPin, etc.).

In some embodiments, a therapeutic polypeptide(s) is or comprises an antibody or a functional fragment thereof. In some embodiments, an antibody is a monoclonal antibody (e.g., IgA, IgG, IgE, or IgM antibodies). In some embodiments, an antibody is or comprises a bi-specific antibody. In some embodiments, an antibody is or comprises a multi-specific anybody. In some embodiments, an antibody is a human antibody, humanized antibodies, chimeric antibodies, reverse chimeric antibodies, antibodies with light chain variable gene segments on heavy chain, antibodies with heavy chain variable gene segments on light chain, as well as, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g, anti-Id antibodies to antigen-specific TCR), or epitope-binding fragments of any of the aforementioned. Thus, “antigen binding fragment” and “antigen-binding portion” and “epitope-binding fragment” of an antigen binding molecule are also encompassed herein, and refer to fragments that retain the ability to bind to an antigen. The term “antigen-binding protein” also includes, for example, single domain antibodies (e.g., a VHH antibody or a “camelid-like” antibody), heavy chain only antibodies, covalent diabodies such as those disclosed in, for example, U.S. Pat. Appl. Pub. 20070004909, incorporated herein by reference in its entirety, and Ig-DARTS such as those disclosed in, for example, U.S. Pat. Appl. Pub. 20090060910, incorporated herein by reference in its entirety. In some certain embodiments, an antibody is a canonical antibody that includes at least two heavy (H) chains and two light (L) chains (e.g, inter-connected by disulfide bonds).

In some embodiments, an antibody comprises digestion fragments, specified portions, derivatives and/or variants thereof, including, for example, antibody mimetics or comprising portions of antibodies that mimic the structure and/or function of an antibody or specified fragment or portion thereof, including single chain antibodies and fragments thereof. There are five main heavy chain classes (or isotypes) which determine the functional activity of an antibody molecule: IgM, IgD, IgG, IgA and IgE. Each heavy and light chain contains a constant region and a variable region (also known as “domains”). In combination, the heavy and the light chain variable regions, also called the “Fab region,” specifically bind to a given antigen. Light and heavy chain variable regions contain a “framework” region interrupted by three hypervariable regions, also called “complementarity-determining regions” or “CDRs.” The extent of the framework region and CDRs has been defined (see, e.g., Kabat et al., Sequences of Proteins of Immunological Interest, U.S. Department of Health and Human Services, 1991). The Kabat database is now maintained online. The sequences of the framework regions of different light or heavy chains are relatively conserved within a species, and framework regions act to form a scaffold that provides for positioning the CDRs in correct orientation by inter-chain, non-covalent interactions.

Antibody CDRs are primarily responsible for binding to an epitope on an antigen. The CDRs of each chain are typically referred to as CDR1, CDR2, and CDR3, numbered sequentially starting from the N-terminus, and are also typically identified by the chain in which the particular CDR is located. Thus, a HCDR3 is located in the variable domain of the heavy chain of the antibody in which it is found, whereas a LCDR1 is the CDR1 from the variable domain of the light chain of the antibody in which it is found. An antibody that binds IL-3 IRA will have a specific VH region and the VL region sequence, and thus specific CDR sequences. Antibodies with different specificities generally have different CDRs. Although it is the CDRs that vary from antibody to antibody, only a limited number of amino acid positions within the CDRs are directly involved in antigen binding. These positions within the CDRs are called specificity determining residues (SDRs).

An antibody Fc fragment region (Fc) plays a role in modulating immune cell activity. The Fc region functions to guarantee that each antibody generates an appropriate immune response for a given antigen, by binding to a specific class of proteins or polypeptides found on certain cells, such as B lymphocytes, follicular dendritic cells, natural killer cells, macrophages, neutrophils, etc. and are called “Fc receptors.” Because the constant domains of the heavy chains make up the Fc region of an antibody, the classes of heavy chain in antibodies determine their class effects. The heavy chains in antibodies include alpha, gamma, delta, epsilon, and mu, and correlate to the antibody's isotypes IgA, IgG, IgD, IgE, and IgM, respectively. Thus, different isotypes of antibodies have different class effects due to their different Fc regions binding and activating different types of receptors. Exemplary Fc sequences are shown in Table 2.

TABLE 2
Exemplary Fc amino acid sequences

| SEQ | | |
|---|---|---|
| ID | Descrip- | |
| NO: | tion | Amino acid sequence |
| 3 | wFc | EPKSCDKTHTCPPCPAPELLGGPSVFLFPPKPK |
| | (wild | DTLMISRTPEVTCVVVDVSHEDPEVKFNWYVDG |
| | type | VEVHNAKTKPREEQYNSTYRVVSVLTVLHQDWL |
| | human | NGKEYKCKVSNKALPAPIEKTISKAKGQPREPQ |
| | Fc) | VYTLPPSREEMTKNQVSLTCLVKGFYPSDIAVE |
| | | WESNGQPENNYKTTPPVLDSDGSFFLYSKLTVD |
| | | KSRWQQGNVFSCSVMHEALHNHYTQKSLSLSPG |
| | | K |
| 4 | mFc | EPKSCDKTHTCPPCPAPELLGGPSVFLFPPKPK |
| | (mutant | DTLMISRTPEVTCVVVDVSHEDPEVKFNWYVDG |
| | human | VEVHNAKTKPREEQYQSTYRVVSVLTVLHQDWL |
| | Fc) | NGKEYKCKVSNKALPAPIEKTISKAKGQPREPQ |
| | | VYTLPPSREEMTKNQVSLTCLVKGFYPSDIAVE |
| | | WESNGQPENNYKTTPPVLDSDGSFFLYSKLTVD |
| | | KSRWQQGNVFSCSVMHEALHNHYTQKSLSLSPG |
| | | K |

South American camelids (e.g. alpaca and llama) produce two isotypes of immunoglobulins, IgG2 and IgG3 that lack light chains and are thus termed heavy chain antibodies or HCAbs. The VH domain of HCAbs, called VHH, is single-domain and the smallest known antigen-binding agent (˜15 kDa). VHHs are easy to express using microorganisms, including yeast, and are generally more stable than conventional antibody fragments. Because of the many favorable properties of VHHs, they have become widely used in research and show clear commercial potential. In some embodiments, an antibody of the present disclosure is or comprises VHH-Fc antibodies (e.g., one or more VHH derived from camelid heavy-chain only antibodies fused with an Fc domain of IgG, IgA, etc.). In some such embodiments, a VHH-Fc antibody is or comprises a single VHH fused to an Fc region. In some such embodiments, a VHH-Fc antibody is or comprises two VHHs fused to an Fc region (e.g., VHH-VHH-Fc, Fc-VHH-VHH, VHH-Fc-VHH). In some embodiments, two VHH may be fused together without an Fc region. In some embodiments, two VHH may be fused together with other VHHs to form trimer or tetramer. In some such embodiments, the two VHHs are the same. In some such embodiments, the two VHHs are different. In some embodiments, bioactivities between the formats of VHH-VHH-Fc, Fc-VHH-VHH, and VHH-Fc-VHH are similar. In some embodiments, bioactivities between the formats of VHH-VHH-Fc, Fc-VHH-VHH, and VHH-Fc-VHH are different. In some embodiments, a particular form yields a higher expression. In some embodiments, selection markers do not affect the overall screening nor the expression of the antibody. In sum, the present disclosure provides binding protein/polypeptide formats that include, but are not limited to VHH, Fc-VHH, VHH-Fc, VHH-VHH, Fc-VHH-VHH, VHH-Fc-VHH, and VHH-VHH-Fc, wherein each or any of the domains (i.e., VHH or Fc) may be attached to another domain via an optional linker sequence.

In some embodiments, a therapeutic polypeptide(s) of the present disclosure further comprises a tag (e.g., an HA tag, a His tag, a FLAG-tag, etc.). A plurality of tags are known in the art and one of ordinary skill in the art would readily appreciate and understand how to utilize such tags in accordance with the present disclosure.

In some embodiments, a therapeutic polypeptide(s) of the present disclosure further comprises a linker. In some such embodiments, a linker is a polypeptide linker. A plurality of linkers are known in the art and one of ordinary skill in the art would readily appreciate and understand how to use such linkers in accordance with the present disclosure.

In some embodiments, a therapeutic polypeptide(s) of the present disclosure further comprises an aprotinin polypeptide. Exemplary aprotinin sequences are shown in Table 3.

TABLE 3
Exemplary aprotinin amino acid sequences.

| SEQ | | |
|---|---|---|
| ID | Descrip- | |
| NO: | tion | Amino acid sequence |
| 26 | Aprotinin | RPDFCLEPPYTGPCKARIIRYFYNAKAGLC |
| | | QTFVYGGCRAKRNNFKSAEDCMRTCGGA |

In some embodiments, a therapeutic polypeptide(s) of the present disclosure is expressed from a recombinant expression vector. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome (e.g., engineered strains of Saccharomyces yeast) (see, for example, Chen et al., Sci Transl. Med 2020).

In some embodiments, an expression vector is one into which a nucleic acid comprising a desired DNA sequence (e.g., a DNA sequence (GOI) that encodes a therapeutic polypeptide(s)) may be inserted by restriction and ligation such that it is operatively linked to regulatory sequences and may be expressed as an RNA transcript. In some embodiments, vectors may further comprise one or more marker sequences, (e.g., as described elsewhere herein) suitable for use in the identification of cells which have or have not been transformed or transfected with the vector. Additional exemplary markers include, for example, genes encoding proteins or polypeptides which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein).

As used herein, a coding sequence and regulatory sequences are said to be “operatively” linked when they are covalently linked in such a way as to place the expression or transcription of the coding sequence under the influence or control of the regulatory sequences. In some embodiments, if it is desired that the coding sequences be translated into a functional polypeptide (e.g., therapeutic polypeptide(s)), two DNA sequences are said to be operatively linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a polypeptide (e.g., therapeutic polypeptide(s)). Thus, a promoter region would be operatively linked to a coding sequence if the promoter region were capable of effecting transcription of that DNA sequence such that the resulting transcript can be translated into the desired protein or polypeptide (e.g., therapeutic polypeptide(s)).

In some embodiments, when a nucleic acid encoding a therapeutic polypeptide of the present disclosure is expressed in a cell (e.g., Saccharomyces yeast), a variety of transcription control sequences (e.g., promoter, enhancer sequences) can be used to direct its expression. The promoter can be a native promoter, i.e., the promoter of the gene in its endogenous context, which provides normal regulation of expression of the gene. In some embodiments the promoter can be constitutive, including, for example, pADH1, pADH2, pHXT7, pHXT4, pHXT2, pPKG1, pPYK1, pTPL1, pSED1a, pSED1b, pJEN1, the promoter is unregulated allowing for continual transcription of its associated nucleic acid (e.g., a nucleic acid encoding a therapeutic polypeptide(s)). In some embodiments the promoter can be inducible, including, for example pICL1, pMAL62, pGUT1, pFBP1, pSUC2, pCUP1, pHGT9, pHGT10, pHGT12, pHGT17, pPCK1. In some embodiments, an inducible promoter is active upon the presence or absence of chemicals or under some conditions. Non-limiting examples of promoters include pTDH3 and pTEF. A variety of conditional promoters also can be used, such as promoters controlled by the presence or absence of a molecule. In some embodiments, a nucleic acid encoding a therapeutic polypeptide(s) is inserted at an endogenous promoter region of a host cell (e.g., engineered strain of Saccharomyces yeast). In some such embodiments, insertion at an endogenous promoter region results in better expression and/or less stress for the host relative to an appropriate reference standard (e.g., an engineered strain of Saccharomyces yeast wherein an exogenous promoter is included in the nucleic acid encoding a therapeutic polypeptide(s)).

In some embodiments, regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operatively linked nucleic acid.

In some embodiments, regulatory sequences comprise enhancer sequences or upstream activator sequences as desired. The choice and design of an appropriate vector is understood in the art. One of ordinary skill in the art would readily recognize and understand how to select and use such vectors in accordance with technologies of the present disclosure.

Expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. In some embodiments, cells are engineered by the introduction into the cells of heterologous DNA (RNA). That heterologous DNA (RNA) is placed under operative control of transcriptional elements to permit the expression of the heterologous DNA in the host cell (e.g., engineered strains of Saccharomyces yeast). As one of ordinary skill in the art would readily appreciate, therapeutic polypeptide(s) described herein can also be expressed in other cell types.

A nucleic acid molecule that encodes therapeutic polypeptide(s) of the present disclosure can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding a therapeutic polypeptide(s) of the present disclosure also may be accomplished by integrating the nucleic acid molecule into the genome. The incorporation of nucleic acids (e.g., encoding a therapeutic polypeptide(s) described herein) can be accomplished either by incorporation of the new nucleic acid into the genome of a yeast cell (e.g., engineered strains of Saccharomyces yeast), or by transient or stable maintenance of the new nucleic acid as an episomal element. In some embodiments, in eukaryotic cells, a permanent, inheritable genetic change is generally achieved by introduction of a nucleic acid into the genome of the cell.

### Antitoxin ABAB

In some embodiments, a therapeutic polypeptide(s) of the present disclosure is or comprises an antitoxin to Toxin A (also referred to as TcdA) and/or Toxin B (also referred to as TcdB) (Antitoxin ABAB) produced by C. difficile.

In some embodiments, an antitoxin ABAB is or comprises an antibody or functional fragment thereof against TcdA, TcdB, or a combination thereof.

In some embodiments, an antitoxin to C. difficile Toxin A and/or Toxin B is or comprises a tetra-specific VHH fusion polypeptide. In some such embodiments, a tetra-specific VHH fusion polypeptide comprises four distinctive toxin-neutralizing VHHs, two against Toxin A and two against Toxin B. In some such embodiments, a tetra-specific VHH fusion polypeptide is fused to an Fc fragment (e.g., a human IgG1 Fc fragment).

In some embodiments, engineered strains of Saccharomyces yeast express an antitoxin ABAB.

Exemplary antitoxin ABAB sequences are summarized in Table 4.

TABLE 4
Exemplary antitoxin ABAB amino acid
sequences.

| SEQ | | |
|---|---|---|
| ID | Descrip- | |
| NO: | tion | Amino acid sequence |
| 5 | FZ002 | QVQLVETGGGLVQPGGSLRLSCAASGFTLDY |
| | | SSIGWFRQAPGKEREGVSCISSSGDSTKYAD |
| | | SVKGRFTTSRDNAKNTVYLQMNSLKPDDTAV |
| | | YYCAAFRATMCGVFPLSPYGKDDWGKGTLVT |
| | | VSSGGGGSGGGGSGGGGSQVQLVESGGGLVQ |
| | | PGGSLRLSCEASGFTLDYYGIGWFRQPPGKE |
| | | REAVSYISASARTILYADSVKGRFTISRDNA |
| | | KNAVYLQMNSLKREDTAVYYCARRRFSASSV |
| | | NRWLADDYDVWGRGTQVAVSSGGGSGGGSGG |
| | | GSGGGSQLQLVETGGGLVQPGGSLRLSCAAS |
| | | GFTFSDYVMTWVRQAPGKGPEWIATINTDGS |
| | | TMRDDSTKGRFTISRDNAKNTLYLQMTSLKP |
| | | EDTALYYCARGRVISASAIRGAVRGPGTQVT |
| | | VSSGGGGSGGGGSGGGGSQVQLVESGGGLVQ |
| | | TGGSLRLSCASSGSIAGFETVTWSRQAPGKS |
| | | LQWVASMTKTNNEIYSDSVKGRFIISRDNAK |
| | | NTVYLQMNSLKPEDTGVYFCKGPELRGQGIQ |
| | | VTVSS |

### Anti-TNF-α

In some embodiments, a therapeutic polypeptide(s) of the present disclosure is or comprises an antibody against TNF-α. Exemplary sequences of antibodies against TNF-α are summarized in Table 5.

In some embodiments, engineered strains of Saccharomyces yeast express antibodies against TNF-α (e.g., FZ006). In some embodiments, engineered strains of Saccharomyces yeast express antibodies against TNF-α and antitoxin ABAB (e.g., FZ020).

TABLE 5
Exemplary anti-TNF-α antibody amino acid
sequences.

| SEQ | | |
|---|---|---|
| ID | Descrip- | |
| NO: | tion | Amino acid sequence |
| 6 | FZ006 | EVQLLESGGGLVQPGGSLRLSCAASGRTFSTYA |
| | (Sb- | MGWFRQAPGKEREFVATVDNDGFTMHADSVKGR |
| | ahTNF) | FTISRDNSKNTLYLQMNSLRAEDTAVYYCAAGQ |
| | | YSKFVESYAYWGQGTLVTVSS |
| 7 | FZ006m | QVQLQDSGGGLVQAGGSLRLSCAASGGTFSSII |
| | (Sb- | MAWFRQAPGKEREFVGAVSWSGGTTVYADSVLG |
| | amTNF) | RFEISRDSARKSVYLQMNSLKPEDTAVYYCAAR |
| | | PYQKYNWASASYNVWGQGTQVTVSSGGGGSGGG |
| | | GSGGGGSQVQLQDSGGGLVQAGGSLRLSCAASG |
| | | GTFSSIIMAWFRQAPGKEREFVGAVSWSGGTTV |
| | | YADSVLGRFEISRDSARKSVYLQMNSLKPEDTA |
| | | VYYCAARPYQKYNWASASYNVWGQGTQVTVSS |

In some embodiments, a therapeutic polypeptide(s) of the present disclosure is or comprises an antibody against IL-17A. Exemplary Sequences of antibodies against IL-17A are summarized in Table 6.

In some embodiments, engineered strains of Saccharomyces yeast express antibodies against IL-17A (e.g., FZ004).

TABLE 6
Exemplary anti-IL-17A antibody amino acid
sequences.

| SEQ | | |
|---|---|---|
| ID | Descrip- | |
| NO: | tion | Amino acid sequence |
| 25 | FZ004 | QVQLVQSGGGLVQAGGSLRLSCAASGGTFATSP |
| | | MGWLRQAPGKGTEFVAAISPSGGDRIYADSVKG |
| | | RFTISRDNAGYFIYLQMNSLKPEDTAVYYCAVR |
| | | RRFDGTSYYTGDYDSWGQGTLVTVSS |

Bi-Specific Antibodies Against hIL-17A and TNF-α

In some embodiments, a therapeutic polypeptide(s) of the present disclosure is or comprises a bi-specific antibody. In some embodiments, a strain of engineered strains of Saccharomyces yeast efficiently secretes a functional bi-specific neutralizing antibody. In some such embodiments, the format of the antibody comprises two fused anti-cytokine single-domain antibodies, or VHHs, either at both N-terminus and C-terminus of human IgG1 Fc or at the N-terminus only (VHH-Fc-VHH and VHH-VHH-Fc). In some embodiments, a functional bi-specific antibody is or comprises a bi-specific antibody against IL-17 and TNF-α. In some embodiments, different promoters drive a different expression of the anti-TNFa/IL-17Abi-specific antibody gene.

In some embodiments, engineered strains of Saccharomyces yeast express a bi-specific antibody against IL-17A and TNF-α (e.g., FZ008).

Exemplary sequences of bi-specific antibodies against IL-17A and TNF-α are summarized in Table 7.

TABLE 7
Exemplary bi-specific antibodies against
IL-17A and TNF-α amino acid sequences.

| SEQ | | |
|---|---|---|
| ID | Descrip- | |
| NO: | tion | Amino acid sequence |
| 8 | FZ008a | EVQLLESGGGLVQPGGSLRLSCAASGRTFSTYAMG |
| | | WFRQAPGKEREFVATVDNDGFTMHADSVKGRFTIS |
| | | RDNSKNTLYLQMNSLRAEDTAVYYCAAGQYSKFVE |
| | | SYAYWGQGTLVTVSSGQAGQGGGGSGGGGSGGGGS |
| | | LQQVQLVQSGGGLVQAGGSLRLSCAASGGTFATSP |
| | | MGWLRQAPGKGTEFVAAISPSGGDRIYADSVKGRF |
| | | TISRDNAGYFIYLQMNSLKPEDTAVYYCAVRRRFD |
| | | GTSYYTGDYDSWGQGTLVTVSS |
| 9 | FZ008b- | EVQLLESGGGLVQPGGSLRLSCAASGRTFSTYAMG |
| | VHH1 | WFRQAPGKEREFVATVDNDGFTMHADSVKGRFTIS |
| | (anti- | RDNSKNTLYLQMNSLRAEDTAVYYCAAGQYSKFAV |
| | TNF-α) | ESYYWGQGTLVTVSSGQAGP |
| 10 | FZ008b- | QVQLVQSGGGLVQAGGSLRLSCAASGGTFATSPMG |
| | VHH2 | WLRQAPGKGTEFVAAISPSGGDRIYADSVKGRFTI |
| | (anti- | SRDNAGYFIYLQMNSLKPEDTAVYYCAVRRRFDGT |
| | IL- | SYYTGDYDSWGQGTLVTVSS |
| | 17A) | |

### IL-22

In some embodiments, a therapeutic polypeptide(s) of the present disclosure is or comprises an IL-22 polypeptide (e.g., a functional IL-22 polypeptide). Exemplary sequences of IL-22 polypeptides are summarized in Table 8.

In some embodiments, an engineered strain of Saccharomyces yeast disclosed herein expresses IL-22 polypeptide (e.g., FZO10). In some such embodiments, an IL-22 polypeptide is linked to an Fc domain. In some embodiments, an engineered strain of Saccharomyces yeast disclosed herein expresses IL-22 polypeptide and a bi-specific antibody against IL-17A and TNF-α (e.g., FZ012).

TABLE 8
Exemplary IL-22 amino acid sequences expressed
by engineered yeast strains.

| SEQ | | |
|---|---|---|
| ID | Descrip- | |
| NO: | tion | Amino acid sequence |
| 11 | FZ010m | LPVNTRCKLEVSNFQQPYIVNRTFMLAKEASLADNN |
| | | TDVRLIGEKLFRGVSAKDQCYLMKQVLNFTLEDVLL |
| | | PQSDRFQPYMQEVVPFLTKLSNQLSSCHISGDDQNI |
| | | QKNVRRLKETVKKLGESGEIKAIGELDLLFMSLRNA |
| | | CV |
| 12 | FZ010 | APISSHCRLDKSNFQQPYITNRTFMLAKEASLADNN |
| | | TDVRLIGEKLFHGVSMSERCYLMKQVLNFTLEEVLF |
| | | PQSDRFQPYMQEVVPFLARLSNRLSTCHIEGDDLHI |
| | | QRNVQKLKDTVKKLGESGEIKAIGELDLLFMSLRNA |
| | | CI |

### Anti-Rotavirus VHH

In some embodiments, a therapeutic polypeptide(s) of the present disclosure is or comprises an anti-Rotavirus VHH (e.g., anti-rotavirus VHH, 2KD1). In some embodiments, an anti-Rotavirus VHH is fused with aprotinin. Aprotinin is a single chain polypeptide isolated from bovine lung with antifibrinolytic and anti-inflammatory activities. As a broad-spectrum serine protease inhibitor, aprotinin bovine competitively and reversibly inhibits the activity of a number of different esterases and proteases, including trypsin, chymotrypsin, kallikrein, plasmin, tissue plasminogen activator, and tissue and leukocytic proteinases, resulting in attenuation of the systemic inflammatory response (SIR), fibrinolysis, and thrombin generation. This agent also inhibits pro-inflammatory cytokine release and maintains glycoprotein homeostasis. Without wishing to be bound by any one theory, fusion of Aprotinin with an anti-Rotavirus VHH (e.g., 2KD1) may reduce or preclude digestion of anti-Rotavirus VHH (e.g., anti-rotavirus VHH, 2KD1) in the gastrointestinal tract and/or reduce inflammation.

In some embodiments, an engineered strain of Saccharomyces yeast disclosed herein expresses an anti-Rotavirus VHH (e.g., FZ014).

Exemplary sequences of anti-Rotavirus VHH polypeptides are summarized in Table 9.

TABLE 9
Exemplary anti-Rotavirus VHH amino acid
sequences.

| SEQ | | |
|---|---|---|
| ID | Descrip- | |
| NO: | tion | Amino acid sequence |
| 13 | FZ014 | MADVQLQASGGGFVQPGDSLSLSCAASGGTFSSYS |
| | | IGWFRQGPGKEREFVATISSSDSPWYGEPAKGRFT |
| | | VARVNAKNTAYLHLNRLKPEDTATYYCAAGSVQHM |
| | | ANENEYVYWGQGTQVTVSS |

### Mono- and Bi-Specific VHHs-Fc Against Norovirus

In some embodiments, a therapeutic polypeptide(s) of the present disclosure is or comprises mono-specific or bi-specific VHHs-Fc against norovirus (e.g., M6M5-Fc and M6M4-Fc). In some such embodiments, VHHs against norovirus further comprises a tag (e.g., an HA tag).

In some embodiments, engineered strains of Saccharomyces yeast disclosed herein express bi-specific VHHs-Fc against norovirus (e.g., M6M5-Fc and M6M4-Fc) (e.g., FZ016). In some embodiments, engineered strains of Saccharomyces yeast disclosed herein express a mono-specific VHHs-Fc against norovirus (e.g., FZ016c). In some embodiments, engineered strains of Saccharomyces yeast disclosed herein express bi-specific VHHs-Fc against norovirus and antitoxin ABAB (e.g., FZ018).

Exemplary sequences of bi-specific or mono-specific VHHs-Fc against norovirus are summarized in Table 10.

TABLE 10
Exemplary bi-specific VHHs-Fc (FZ016a,
FZ016b) and mono-specific (FZ016c)
amino acid sequences against norovirus.
Bolded and underlined sequences denote
HA tag sequence.

| SEQ | | |
|---|---|---|
| ID | Descrip- | |
| NO: | tion | Amino acid sequence |
| 14 | FZ016a | QVKLQESGGGLVQAGESLRLSCAASGSNFSINGV |
| | | GWYRQAPGKQRELVAGITNGGYTSYADSVKGRFT |
| | | ISTDNAKNTVYLQMNSLRPEDTAVYYCNANFQIH |
| | | RSGADYVRNYWGQGTQVTVSAGGGGSGGGGSGGG |
| | | GSQVKLQQSGGGLVQPGGSLRLSCAASESTISIN |
| | | TLGWYRQAPGNQRELVATITTGGTTNYADSVKGR |
| | | FTISRDNAKNTVYLQMNNLEPGDTAVYYCNLKRR |
| | | DLQSRFGGYWGQGTQVTVSS |
| 15 | FZ016b | QVKLQESGGGLVQAGESLRLSCAASGSNFSINGV |
| | | GWYRQAPGKQRELVAGITNGGYTSYADSVKGRFT |
| | | ISTDNAKNTVYLQMNSLRPEDTAVYYCNANFQIH |
| | | RSGADYVRNYWGQGTQVTVSAGGGGSGGGGSGGG |
| | | GSQVQLQQSGGGLVQPGGSLRLSCVASESTVSIN |
| | | IMGWYRQAPGKQRELVATITTGGTTNYADSVKGR |
| | | FTISRDNAKNTVYLQMNSLEPEDTAVYYCNLKRR |
| | | DLQARFGGYWGQGTQVTVSS |
| 16 | FZ016c | QVKLQQSGGGLVQPGGSLRLSCAASESTISINTL |
| | | GWYRQAPGNQRELVATITTGGTTNYADSVKGRFT |
| | | ISRDNAKNTVYLQMNNLEPGDTAVYYCNLKRRDL |
| | | QSRFGGYWGQGTQVTVSSYPYDVPDYA |

### GLP-1

In some embodiments, a therapeutic polypeptide(s) of the present disclosure is or comprises a GLP-1 polypeptide (e.g., a functional GLP-1 polypeptide). Exemplary sequences of GLP-1 polypeptides are summarized in Table 11.

In some embodiments, engineered strains of Saccharomyces yeast disclosed herein express a GLP-1 polypeptide (e.g., a functional GLP-1 polypeptide, a human functional GLP-1 polypeptide) (e.g., FZ022).

TABLE 11
Exemplary GLP-1 amino acid sequences.

| SEQ | | |
|---|---|---|
| ID | Descrip- | |
| NO: | tion | Amino acid sequence |
| 17 | FZ022a | HAEGTFTSDVSSYLEGQAAKEFIAWLVKGR |
| 23 | FZ022b | HAEGTFTSDVSSYLEGQAAKEFIAWLVKGRG |
| 24 | FZ022c | HGEGTFTSDLSKQMEEEAVRLFIEWLKNGGP |
| | | SSGAPPPS |

### Leptin

In some embodiments, a therapeutic polypeptide(s) of the present disclosure is or comprises a leptin polypeptide. (e.g., a functional leptin polypeptide). Exemplary sequences of leptin polypeptides are summarized in Table 12. In some such embodiments, a leptin polypeptide further comprises a tag (e.g., an HA tag).

In some embodiments, engineered strains of Saccharomyces yeast disclosed herein express a leptin polypeptide (e.g., a functional leptin polypeptide, a human functional leptin polypeptide) (e.g., FZ024).

TABLE 12
Exemplary leptin amino acid sequences.
Bolded and underlined sequences denote
HA tag sequence.

| SEQ | | |
|---|---|---|
| ID | Descrip- | |
| NO: | tion | Amino acid sequence |
| 18 | FZ024 | VPIQKVQDDTKTLIKTIVTRINDISHTQSVSSKQ |
| | | KVTGLDFIPGLHPILTLSKMDQTLAVYQQILTSM |
| | | PSRNVIQISNDLENLRDLLHVLAFSKSCHLPWAS |
| | | GLETLDSLGGVLEASGYSTEVVALSRLQGSLQDM |
| | | LWQLDLSPGCYPYDVPDYA |

### Anti-Human TNF-α IgG1

In some embodiments, a therapeutic polypeptide(s) of the present disclosure is or comprises an anti-TNF-α IgG1. In some such embodiments, an anti-TNF-α IgG1 comprises humira. In some such embodiments, an anti-TNF-α IgG1 comprises adalimumab. Exemplary sequences of anti-TNF-α IgG1 are summarized in Table 13.

In some embodiments, engineered strains of Saccharomyces yeast disclosed herein express an anti-TNF-α IgG1 (e.g., FZ026).

TABLE 13
Exemplary anti-TNF-α IgG1 acid sequences.

| SEQ | | |
|---|---|---|
| ID | Descrip- | |
| NO: | tion | Amino acid sequence |
| 19 | FZ026- | EVQLVESGGGLVQPGRSLRLSCAASGFTFDDYAMHW |
| | HC | VRQAPGKGLEWVSAITWNSGHIDYADSVEGRFTISR |
| | | DNAKNSLYLDMNSLRAEDTAVYYCAKVSYLSTASSL |
| | | DYWGQGTLVTVSSASTKGPSVFPLAPSSKSTSGGTA |
| | | ALGCLVKDYFPEPVTVSWNSGALTSGVHTFPAVLQS |
| | | SGLYSLSSVVTVPSSSLGTQTYICNVNHKPSNTKVD |
| | | KRVEPKSCDKTHTCPPCPAPELLGGPSVFLFPPKPK |
| | | DTLMISRTPEVTCVVVDVSHEDPEVKFNWYVDGVEV |
| | | HNAKTKPREEQYNSTYRVVSVLTVLHQDWLNGKEYK |
| | | CKVSNKALPAPIEKTISKAKGQPREPQVYTLPPSRE |
| | | EMTKNQVSLTCLVKGFYPSDIAVEWESNGQPENNYK |
| | | TTPPVLDSDGSFFLYSKLTVDKSRWQQGNVFSCSVM |
| | | HEALHNHYTQKSLSLSPGK |
| 20 | FZ026- | DIQMTQSPSSLSASVGDRVTITCRASQGIRNYLAWY |
| | LC | QQKPGKAPKLLIYAASTLQSGVPSRFSGSGSGTDFT |
| | | LTISSLQPEDVATYYCQRYNRAPYTFGQGTKVEIKR |
| | | TVAAPSVFIFPPSDEQLKSGTASVVCLLNNFYPREA |
| | | KVQWKVDNALQSGNSQESVTEQDSKDSTYSLSSTLT |
| | | LSKADYEKHKVYACEVTHQGLSSPVTKSFNRGEC |

VHH Against Cwp84 Fused with Lysin Domain

In some embodiments, a therapeutic polypeptide(s) of the present disclosure is or comprises a VHH against cwp84 fused with Lysin domain. Exemplary sequences of VHH against cwp84 fused with Lysin domain are summarized in Table 14.

In some embodiments, engineered strains of Saccharomyces yeast disclosed herein express a VHH against cwp84 fused with Lysin domain (e.g., FZ028).

TABLE 14
Exemplary VHH against cwp84 fused with Lysin
domain amino acid sequences.

| SEQ | | |
|---|---|---|
| ID | Descrip- | |
| NO: | tion | Amino acid sequence |
| 21 | FZ028a | MKICITVGHSILKSGACTSADGVVNEYQYNKSLAPVL |
| | | ADTFRKEGHKVDVIICPEKQFKTKNEEKSYKIPRVNS |
| | | GGYDLLIELHLNASNGQGKGSEVLYYSNKGLEYATRI |
| | | CDKLGTVFKNRGAKLDKRLYILNSSKPTAVLIESFFC |
| | | DNKEDYDKAKKLGHEGIAKLIVEGVLNKNINGGGGSG |
| | | GGGSGGGGSQVQLVESGGGLVQAGGSLRLSCVASTS |
| | | TFPINEMGWYRQAPGKQRELVAAITRVGTTNYANSV |
| | | KGRFTISRDKTSNTKNTVYLQMNSLKPEDTAVYYCN |
| | | VNLFADTGNYWGQGTLVTVSS |
| 22 | FZ028b | MKVVIIPGHTLIGKGTGAVGYINESKETRILNDLIVK |
| | | WLKIGGATVYTGRVDESSNHLADQCAIANKQETDLAV |
| | | QIHFNSNATTSTPVGTETIYKTNNGKTYAERVNTRLA |
| | | TVFKDRGAKSDVRGLYWLNHTIAPAILIEVCFVDSKA |
| | | DTDYYVNNKDKVAKLIAEGILNKSGSGGGSEGGGSE |
| | | GGGSEGGQVQLVESGGGLVQAGGSLRLSCVASTSTFP |
| | | INEMGWYRQAPGKQRELVAAITRVGTTNYANSVKGR |
| | | FTISRDKTSNTKNTVYLQMNSLKPEDTAVYYCNVNLF |
| | | ADTGNYWGQGTLVTVSS |

### IL-10

In some embodiments, a therapeutic polypeptide(s) of the present disclosure is or comprises an IL-10 polypeptide. (e.g., a functional IL-10 polypeptide). Exemplary sequences of IL-10 polypeptides are summarized in Table 15.

In some embodiments, engineered strains of Saccharomyces yeast disclosed herein express an IL-10 polypeptide (e.g., a functional IL-10 polypeptide, a human functional leptin polypeptide) (e.g., FZ030).

TABLE 15
Exemplary 1L-10 amino acid sequences.

| SEQ | | |
|---|---|---|
| ID | Descrip- | |
| NO: | tion | Amino acid sequence |
| 27 | FZ030 | SPGQGTQSENSCTHFPGNLPNMLRDLRDAFSRVKTFF |
| | | QMKDQLDNLLLKESLLEDFKGYLGCQALSEMIQFYL |
| | | EEVMPQAENQDPDIKAHVNSLGENLKTLRLRLRRCH |
| | | RFLPCENKSKAVEQVKNAFNKLQEKGIYKAMSEFDIF |
| | | INYIEAYMTMKIRN |

Table 16 summarizes exemplary therapeutic polypeptide(s) structure and sequences of the present disclosure.

TABLE 16
Exemplary therapeutic polypeptide(s) structures and amino acid sequences.

| | | | Format | | | | | | | |
|---|---|---|---|---|---|---|---|---|---|---|
| | | | | Fc-fusion | | | Aprotinin (Ap)-fusion | | | |
| | | Therapeutic | Non- | Fc- | Fc | Fc | Ap- | Ap | | |
| Bio Leads | Structure | peptide | fusion | fusion | type | position | fusion | position | [symbol] gG | Tag |
| FZ002 | Tetramer | S1_FZ002 | Yes | | | | | | | No |
| FZ004 | Dimer | S2_FZ004 | | Yes | S23_wFc | C-terminal | | | | No |
| FZ006 | Dimer | S3_FZ006 | | Yes | S23_wFc | C-terminal | | | | No |
| FZ006m | Tetramer | S4_FZ00 [symbol] m | | Yes | S23_wFc | C-terminal | | | | No |
| FZ00 [symbol] a | Tetramer | S5_FZ00 [symbol] | | Yes | S23_wFc | C-terminal | | | | No |
| FZ008b | Tetramer | S3_FZ00 [symbol] (VHH1) | | Yes | S23_wFc | Between 2 VHHs | | | | |
| | | S2_FZ004(VHH2) | | | | | | | | |
| FZ010m | Dimer | S6_FZ010m | | Yes | S23_wFc | C-terminal | | | | No |
| FZ010a | monomer | S7_FZ010 | Yes | | | | | | | |
| FZ010b | Dimer | S7_FZ010 | | Yes | S24_mFc(N297Q) | N-terminal | | | | No |
| FZ010c | Dimer | S7_FZ010 | | Yes | S24_mFc(N297Q) | C-terminal | | | | No |
| FZ014a | Dimer | S8_FZ014 | | Yes | S23_wFc | C-terminal | | | | No |
| FZ014b | Dimer | S8_FZ014 | | Yes | S23_wFc | C-terminal | Yes | N-terminal | | |
| | | | | | | | S25_Ap | @S8_FZ0 [symbol] 4 | | No |
| FZ014c | Dimer | S8_FZ014 | | Yes | S23_wFc | C-terminal | Yes | C-terminal | | No |
| | | | | | | | S25_Ap | @wFc | | |
| FZ016a | Tetramer | S9_FZ016a | | Yes | S23_wFc | C-terminal | | | | No |
| FZ016b | Tetramer | S10_FZ016b | | Yes | S23_wFc | C-terminal | | | | No |
| FZ016c | Monomer | S11_FZ016c | Yes | | | | | | | HA |
| FZ020 | Mu [symbol] imer | S1_FZ002 + | | | | | | | | No |
| | | S3_FZ006 | | | | | | | | |
| FZ022a | | S12_FZ022a | | | | | | | | No |
| FZ022b | | S13_FZ022b | | | | | | | | No |
| FZ022c | | S14_FZ022c | | | | | | | | No |
| FZ024a | Monomer | S15_FZ024 | Yes | | | C-terminal | | | | No |
| FZ024b | Dimer | S15_F2024 | | Yes | S23_wFc | C-terminal | | | | No |
| FZ026 | | S16_FZ026-HC | | | | | | | Yes | No |
| | | S17_FZ026-LC | | | | | | | | |
| FZ02 [symbol] a | Tetramer | S18_FZ028a(LCD1- | | Yes | S23_wFc | C-terminal | | | | No |
| | | VHH) | | | | | | | | |
| FZ02 [symbol] b | Tetramer | S19_FZ028b (LCD2- | | Yes | S23_wFc | C-terminal | | | | No |
| | | VHH) | | | | | | | | |
| FZ0 [symbol] 0a | Dimer | S20_FZ030 | Yes | | | | | | | |
| FZ030b | Dimer | S20_FZ030 | | Yes | S24_mFc(N297Q) | C-terminal | | | | No |
| FZ030c | Dimer | S20_FZ030 | | Yes | S24_mFc(N297Q) | C-terminal | Yes | N-terminal | | No |
| | | | | | | | S25_Ap | @S8_FZ030 | | |

M, mouse version; wFc, wild-type human Fc; mFc; mutant human Fc; HC, heavy chain; LC, light chain. Bold/underlined, HA tag; [symbol]
indicates data missing or illegible when filed

### Uses

The present disclosure provides, among other things, engineered strains of Saccharomyces yeast that have therapeutic or clinical utility, compositions comprising the same, and methods of treatment of various diseases and conditions using the same. Also described herein are methods of administering the engineered strains of Saccharomyces yeast and/or compositions thereof to a subject in need thereof. In some aspects, the subject can have an inflammatory bowel disease such as, for example, Crohn's disease, ulcerative colitis, or another inflammatory bowel disease, or can have an immune-related condition, or liver diseases, graft verse host disease, diabetes, obesity, neurodegenerative diseases, pain, stroke, cardiovascular diseases, infectious diseases, autoimmune diseases, colon cancer and other GI malignancies, or dysbiosis affecting the skin, mouth, gastrointestinal tract, vagina, or another organ. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Also disclosed herein are methods of treating (e.g., including treating during remission to, for example, minimize symptoms) or preventing (e.g., including prevention of relapse, maintenance treatment) a disease, disorder, and/or condition associated with inflammation in a subject. In some embodiments, the methods comprise administering to the subject a therapeutically-effective amount of engineered strains of Saccharomyces yeast described herein and/or the disclosed pharmaceutical compositions as described herein to a subject in need thereof. In some embodiments, a subject can be a vertebrate such as, for example, a human, dog, cat, horse, cow, pig, sheep, goat, rabbit, chicken, or turkey. In some embodiments, the mammal is a human. In some embodiments, the disease is associated with inflammation. In some embodiments, the disease can be, for example, ulcerative colitis, Crohn's disease, another inflammatory bowel disease, colon cancer, diabetes, obesity, eczema, bacterial vaginosis, vaginal yeast infection, Alzheimer's disease, stress, depression, anxiety, bipolar disorder, neurodegenerative diseases, pain, stroke, cardiovascular diseases, infectious diseases, autoimmune diseases, or any combination thereof.

In some aspects, the subject has the disease associated with inflammation and treatment with engineered strain of Saccharomyces yeast described herein is considered therapeutic. In other aspects, the subject is at risk of a disease associated with inflammation (e.g., an immune-related condition) and treatment with an engineered strain of Saccharomyces yeast is considered prophylactic. In some embodiments, the engineered strain of Saccharomyces yeast has further probiotic effects such as, for example, those associated with wild type (not engineered or parental) strains of Saccharomyces yeast, including, for example, S. boulardii. In some aspects, the treatment reduces at least one symptom of a disease, disorder and/or condition associated with inflammation in the subject relative to the symptom in the subject prior to treatment. In another aspect, the symptom can be, for example, one or more of diarrhea, fever, fatigue, weight loss, blood in the stool, abdominal cramping, abdominal pain, reduced appetite, intestinal damage, rash, itching, mouth sores, or any combination thereof.

Also disclosed herein are methods of treating or preventing a disease associated with inflammation in a subject, the methods comprising administering to the subject a therapeutically-effective amount of an engineered strain of Saccharomyces yeast described herein and/or a disclosed pharmaceutical compositions to the subject. In another aspect, the subject can be a mammal and the disease can be associated with inflammation. In one aspect, the disease can be ulcerative colitis, Crohn's disease, celiac disease, irritable bowel syndrome (IBS), colitis induced by C. difficile or another bacterium or virus or by T-cell transfer or by dextran sulfate sodium (DSS), another inflammatory bowel disease, an infectious gastrointestinal disease including, but not limited to, rotavirus, norovirus, cytomegalovirus, a herpesvirus, an enteric virus, an adenovirus, human papillomavirus, acute self-limited colitis, bacterial enterocolitis, a clostridial disease of the gut, a mycobacterial infection of the GI tract, a spirochetal infection of the GI tract, a fungal infection of the GI tract, a protozoal infection or helminthic infection of the GI tract, gut inflammation (e.g., chronic gut inflammation), a cardiovascular disease including, but not limited to, coronary artery disease, peripheral arterial disease, cerebrovascular disease, renal artery stenosis, aortic aneurysm, cardiomyopathy, hypertensive heart disease, heart failure, pulmonary heart disease, a cardiac dysrhythmia, endocarditis, inflammatory cardiomegaly, myocarditis, valvular heart disease, congenital heart disease, rheumatic heart disease, a cancer such as, for example, esophageal cancer, stomach cancer, rectal cancer, small intestinal cancer, gastrointestinal stromal tumors, nasopharyngeal cancer, colon cancer, or another gastrointestinal cancer, diabetes (type 1 or type 2), hypoglycemia, hypercholesterolemia, or another metabolic disease, obesity, fatty liver disease, steatohepatitis, cirrhosis, liver cancer, eczema, psoriasis, hidradenitis suppurativa, skin ulcers, bacterial vaginosis, vaginal yeast infection, rotavirus, norovirus HIV-associated inflammation, rheumatoid arthritis, psoriatic arthritis, ankylosing spondylitis, amyotrophic lateral sclerosis, systemic lupus erythematosus, food allergies, malabsorption, diarrhea, acid reflux (e.g. GERD), dysbiosis, diverticulitis, sepsis, solid tumors including, but not limited to, breast cancer, bladder cancer, head and neck squamous cell carcinomas, melanoma, neuroblastoma, lung cancer, ovarian cancer, non-small cell lung cancer, liquid tumors including, but not limited to, lymphoma, large B-cell lymphoma, diffuse large B-cell lymphoma, acute myelogenous leukemia, aging associated with inflammation, neurological diseases including, but not limited to, Alzheimer's disease, depression, stress, anxiety, bipolar disorder, schizophrenia, multiple sclerosis, Parkinson's disease, stroke, or any combination thereof. In some aspects, the subject has the disease associated with inflammation. In other aspects, the subject is at risk of the disease associated with inflammation and autoimmune diseases. In some aspects, the treatment reduces at least one symptom of a disease associated with inflammation in the subject relative to the symptom in the subject prior to treatment. In another aspect, the symptom can be diarrhea, fever, fatigue, weight loss, blood in the stool, abdominal cramping, abdominal pain, reduced appetite, intestinal damage, rash, itching, mouth sores, or any combination thereof.

In some embodiments, a method of treating and/or preventing a disease associated with inflammation in a subject comprises administering to the subject a therapeutically-effective amount of an engineered strain of Saccharomyces yeast described herein or a composition (e.g., a pharmaceutical composition) comprising the same.

### Clostridium Difficile Infection (CDI)

Clostridium difficile, or Clostridioides difficile (C. difficile), is a Gram-positive, spore-forming, anaerobic bacillus, which is widely distributed in the intestinal tract of humans and animals and in the environment. Spores of C. difficile are transmitted by the fecal-oral route, and the pathogen is widely present in the environment. Potential reservoirs for C. difficile include asymptomatic carriers, infected patients, the contaminated environment and animal intestinal tract (canine, feline, porcine, avian). Approximately 5% of adults and 15-70% of infants are colonized by C. difficile, and the colonization prevalence is several times higher in hospitalized patients or nursing home residents (Czepiel J et al. Eur J Clin Microbiol Infect Dis. 2019; 38(7):1211-1221).

Infection with C. difficile mostly occurs as a result of spore transmission. Spores are resistant to heat, acid, and antibiotics. The main protective barrier against C. difficile infection (CDI) is the normal intestinal microflora. After reaching the intestine, bile acids play an important role in the induction of C. difficile spore germination. In vitro, primary bile acids (e.g., cholic acid and chenodeoxycholic acid) generally stimulate germination of C. difficile spores; the secondary bile acids (e.g., deoxycholic acid and lithocholic acid) inhibit this process.

When the balance of gut microorganisms is disrupted, C. difficile starts to dominate and colonize the large intestine which, without wishing to be bound by any one theory, may be the first step of infection. The pathogen is not invasive, and virulence is understood as mostly due to two major toxins, which damage the epithelial cell cytoskeleton, leading to disruption of tight junctions, fluid secretion, neutrophil adhesion, and local inflammation, resulting in a breakdown of gut barrier integrity and loss of functionality. Of most importance to disease pathogenesis of C. difficile are toxins, A and B, which are both enterotoxic and cytotoxic; however, traditionally, toxin A is named “enterotoxin A” (TcdA) and toxin B, “cytotoxin B” (TcdB). C. difficile transferase (CDT; or binary toxin) is a third toxin produced by some C. difficile strains, including the epidemic PCR ribotypes 027. Toxins are transported to the cell cytoplasm, where they inactivate the Rho family of GTPases. The Rho protein takes part in actin polymerization, and therefore stabilizes the cell cytoskeleton. As a result of Rho protein inactivation, the inflammatory process intensifies. In more severe cases, microulcerations covered with pseudomembranes (composed of destroyed intestinal cells, neutrophils, and fibrin) start to occur on the intestinal mucosal surface.

In some embodiments, engineered strains of Saccharomyces yeast as described herein are useful to treat and/or prevent CDI. In some such embodiments, engineered strains of Saccharomyces yeast useful in the treatment and/or prevention of CDI express antitoxin ABAB as described herein. In some such embodiments, engineered strains of Saccharomyces yeast useful in the treatment and/or prevention of CDI comprise S. boulardii expressing an antitoxin ABAB (e.g., FZ002) as described herein.

In some embodiments, engineered strains of Saccharomyces yeast as described herein are useful to treat and/or prevent CDI associated with inflammatory bowel disease (IBD). In some cases, CDI is associated with worse outcomes in subjects diagnosed with or suffering from IBD. In some such embodiments, engineered strains of Saccharomyces yeast useful in the treatment and/or prevention of CDI associated with IBD express anti-TNFα and antitoxin ABAB (e.g., FZ020) as described herein.

### Inflammatory Bowel Disease (IBD)

Inflammatory Bowel Disease (IBD) is characterized by chronic inflammation of the gastrointestinal track and understood to result from the interaction between genetic and environmental factors which influence the immune response. Ulcerative colitis (UC) and Crohn's disease (CD) are major forms of IBD and are chronic and frequently relapsing illnesses characterized by bloody diarrhea and abdominal cramps that require long-term medical therapy, frequent hospitalizations, and even surgery. The diseases are characterized by dysregulation of the mucosal immune system and dysbiosis of gut microbiota. According to the Centers for Disease Control and Prevention (CDC), approximately 3.1 million US adults (or 1.3%) are suffering from IBD with a direct treatment cost of more than seven (7) billion dollars annually. Despite significant efforts to develop therapeutics, the hospitalization rate for these diseases in the US has been steadily increasing in the past several decades, and a significant increase in hospitalization rates from 44.2 to 59.7 people per 100,000 occurred during the 10-year period from 2003 to 2013. Moreover, total hospitalization costs also continue rising, leading to substantial financial losses for patients, insurance companies, and employers. The severe complications of IBD can be debilitating, and eventually may lead to death.

The cause of inflammatory bowel diseases is unknown. The pathogenesis of CD and UC most likely involves interaction between genetic and environmental factors, such as bacterial agents, although no definite etiological agent has been identified thus far. The main theory is that abnormal immune response, possibly driven by intestinal microflora, occurs in IBD. However, it is understood that T-cells play an important role in the pathogenesis. Activated T-cells can produce both anti-inflammatory and pro-inflammatory cytokines. One existing therapy involves systemic treatment with anti-TNF monoclonal antibodies. Single intravenous doses, ranging from 5 to 20 mg/kg, of the cA2 infliximab antibody resulted in a drastic clinical improvement in active Crohn's disease. The use of systemically administered recombinant IL-10 in a 7 day treatment regime using doses ranging from 0.5 to 25 μg/kg showed reduced Crohn's disease activity scores and increased remission. These strategies, however, require systemic administration via injection of the purified protein or polypeptide therapeutic, which is both expensive and not convenient for the patient. Existing strategies for UC management also involve using anti-inflammatory and immunosuppressive drugs, such as corticosteroids, which are often associated with generalized immunosuppression. However, up to 40% of patients do not respond to initial treatment. Among initial responders, 13%-46% patients relapse within the subsequent year. Therefore, current treatment options for UC are not optimal and novel treatments are needed.

TNF-α plays critical roles in the pathogenesis of UC in both animal models and humans and has been widely accepted as a therapeutic target for inflammatory disorders such as IBD. US Food and Drug Administration (FDA)-approved anti-TNF-α biologics including Infliximab, Adalimumab, Golimumab, and Certolizumab pegol, have revolutionized therapy for a variety of chronic inflammatory disorders, including rheumatoid arthritis, psoriatic arthritis, ankylosing spondylitis, CD, and UC. All of these antibody therapeutics must be administered parenterally. Although systemically delivered anti-TNF-α biologics are highly efficacious, their long-term use is often associated with loss of effectiveness due to anti-drug antibody responses and immunosuppressive side effects. IL-17A is the most widely studied member of the IL-17 family. IL-17A plays a critical role in host defense against various microbial pathogens as well as tissue inflammation. IL-17A producing CD4+ T helper cells, also called Th17 cells, have been studied extensively in the past decade and have been shown to be potent inducers of tissue inflammation and have been associated with the pathogenesis of many experimental autoimmune diseases and human inflammatory conditions. Substantial evidence suggests that IL-17A producing cells including Th17 cells are involved in human psoriasis, rheumatoid arthritis, multiple sclerosis, inflammatory bowel diseases, and asthma. Anti-IL-17A is FDA approved for the treatment of psoriasis and this pathway has also been studied in asthma, rheumatoid arthritis, multiple sclerosis, transplant rejection, and inflammatory bowel disease.

Interleukin-22 (IL-22) is often described as a cytokine that is expressed by immune cells but that exclusively acts on non-immune cells. Its role is best understood at so called barrier surfaces such as the skin, lungs, and gut where the effects of IL-22 ligation typically involve proliferation, regeneration, or activation of innate immune mechanisms. IL-22 can act synergistically with IL-17 and/or TNFa. In the intestine, IL-22 signaling promotes important functions including host defense against pathogens and wound healing. The application of intestinal recombinant IL-22 may have a beneficial impact on liver and pancreas damage, ulcerative colitis, graft-versus-host disease.

In some embodiments, engineered strains of Saccharomyces yeast as described herein are useful to treat and/or prevent IBD (e.g., UC and CD). In some such embodiments, engineered strains of Saccharomyces yeast useful in the treatment and/or prevention of IBD express anti-TNFα as described herein. In some such embodiments, engineered strains of Saccharomyces yeast useful in the treatment and/or prevention of IBD comprise S. boulardii expressing an anti-TNFα antibody (e.g., FZ006) as described herein. In some such embodiments, engineered strains of Saccharomyces yeast useful in the treatment and/or prevention of IBD express a bi-specific antibody against IL-17A and TNF-α described herein. In some such embodiments, engineered strains of Saccharomyces yeast useful in the treatment and/or prevention of IBD comprise S. boulardii expressing a bi-specific antibody against IL-17A and TNF-α (e.g., FZ008). In some such embodiments, engineered strains of Saccharomyces yeast useful in the treatment and/or prevention of IBD express an IL-22 polypeptide described herein. In some such embodiments, engineered strains of Saccharomyces yeast useful in the treatment and/or prevention of IBD comprise S. boulardii expressing IL-22 (e.g., FZO10). In some such embodiments, engineered strains of Saccharomyces yeast useful in the treatment and/or prevention of IBD express IL-22 and a bi-specific antibody against IL-17A and TNF-α described herein. In some such embodiments, engineered strains of Saccharomyces yeast useful in the treatment and/or prevention of IBD comprise S. boulardii expressing IL-22 and a bi-specific antibody against IL-17A and TNF-α (e.g., FZ012). In some such embodiments, engineered strains of Saccharomyces yeast useful in the treatment and/or prevention of IBD express an anti-human TNF-α IgG1 as described herein. In some such embodiments, engineered strains of Saccharomyces yeast useful in the treatment and/or prevention of IBD comprise S. boulardii expressing an anti-human TNF-α IgG1 (e.g., FZ026).

### Gut Inflammation & Neurological Disease

Gut inflammation is a contributing factor to many serious diseases and disorders, such as metabolic disorders and neurological diseases that significantly affect the life quality of human beings, resulting in substantial functional loss, morbidity, and even mortality. Recent studies have demonstrated that the chronic intestinal inflammation is a potential key factor for the initiation and development of neurodegenerative disease, including, for example, depression, Parkinson's disease, and Alzheimer's disease.

Neurodegnerative diseases occur when neurons in the central nervous system (e.g., the brain and spinal cord) or the peripheral nervous system lose function over time and die. The risk of contracting a neurodegenerative disease increases dramatically with age. The average lifespan of a human is increasing every year, meaning more people will be affected with neurodegenerative diseases in the coming decades. One potential culprit of neurodegenerative diseases that has become increasingly recognized is gut inflammation. The association between the brain and the gut via the Gut-Brain axis is becoming increasingly apparent. One such connection between the brain and the gut begins with the Enteric Nervous System (ENS). The ENS comprises two layers of hundreds of millions of nerve cells that line the gastrointestinal tract from the esophagus to the rectum and can trigger emotional shifts. There is evidence that irritation from the gastrointestinal (GI) system can send signals via the ENS to the central nervous system (CNS). Thus, gut inflammation can trigger mood changes via the gut-brain axis. Interestingly, psychological disorders such as anxiety and depression is widespread in patients with Irritable Bowel Syndrome (IBS). Accordingly, treating and/or preventing gut inflammation may result in the treatment and/or prevention of neurodegenerative and/or neurological disease.

Proinflammatory cytokines, such as, for example, TNF-α and Interleukin 17A (IL-17A) are key immune mediators that contribute to the pathogenesis of many inflammatory disease as well as chronic gut inflammation and immune-related conditions. Without wishing to be bound by any one theory, use of neutralizing antibodies that specifically bind to cytokines and block their interaction with receptors on immune cells, therefore inhibiting downstream inflammatory pathways, may provide a means to control cytokine-mediated inflammation. Accordingly, in some embodiments, engineered strains of Saccharomyces yeast useful in the treatment and/or prevention of gut inflammation and immune-related disease (e.g., neurological disease) express an antibody against proinflammatory cytokines, including for example, a bi-specific antibody against IL-17A and TNF-α and/or an anti-TNF-α IgG1 described herein. In some such embodiments, engineered strains of Saccharomyces yeast useful in the treatment and/or prevention of gut inflammation and/or immune-related conditions comprise S. boulardii expressing a bi-specific antibody against IL-17A and TNF-α (e.g., FZ008). In some such embodiments, engineered strains of Saccharomyces yeast useful in the treatment and/or prevention of gut inflammation comprises S. boulardii expressing an anti-TNF-α VHH (e.g., FZ006). In some such embodiments, engineered strains of Saccharomyces yeast useful in the treatment and/or prevention of gut inflammation comprises S. boulardii expressing an anti-TNF-α IgG1 (e.g., FZ026). In some embodiments, engineered strains of Saccharomyces yeast useful in the treatment and/or prevention of gut inflammation comprise S. boulardii expressing an anti-IL-17A antibody (e.g., FZ004) as described herein. In some embodiments, engineered strains of Saccharomyces yeast useful in the treatment and/or prevention of gut inflammation comprise S. boulardii expressing an IL-10 (e.g., FZ030) as described herein.

### Diabetes

Diabetes is a chronic disease characterized by hyperglycemia. Impaired glucose tolerance and hyperglycemia are the main clinical and diagnostic features of diabetes and are understood to be the result of an absolute or relative insulin deficiency or resistance to its action. Chronic hyperglycemia associated with diabetes can result in end organ dysfunction and failure which can involve, for example, the retina, kidneys, nerves, and blood vessels.

Traditionally, a majority of diabetes cases fall into two broad pathogenic categories, Type 1 diabetes (T1D) and Type 2 diabetes (T2D). However, in some subjects, this classification is not applicable as other genetic, immunological, or neuroendrocrinological pathways are involved in pathogenesis. T1D is related to a lack of insulin due, at least in part, to immune-mediated destruction of pancreatic beta-cells. T2D is the most common form of diabetes and is understood to result, at least in part, from insulin resistance. The emerging role of inflammation in both T1D and T2D pathophysiology and associated metabolic disorders, has generated increasing interest in targeting inflammation to improve prevention and improve control of both T1D and T2D.

In some embodiments, engineered strains of Saccharomyces yeast useful in the treatment and/or prevention of diabetes express a GLP-1 therapeutic polypeptide(s) described herein. In some such embodiments, engineered strains of Saccharomyces yeast useful in the treatment and/or prevention of diabetes comprise S. boulardii expressing GLP-1 polypeptide (e.g., a human active GLP-1 therapeutic polypeptide(s)) described herein (e.g., FZ022).

In some embodiments, engineered strains of Saccharomyces yeast useful in the treatment and/or prevention of diabetes express a leptin therapeutic polypeptide(s) described herein. In some such embodiments, engineered strains of Saccharomyces yeast useful in the treatment and/or prevention of diabetes comprise S. boulardii expressing a leptin polypeptide (e.g., a human active leptin therapeutic polypeptide(s) described herein) (e.g., FZ024).

In some embodiments, engineered strains of Saccharomyces yeast useful in the treatment and/or prevention of diabetes express an IL-22 therapeutic polypeptide(s) described herein. In some such embodiments, engineered strains of Saccharomyces yeast useful in the treatment and/or prevention of diabetes comprise S. boulardii expressing an IL-22 therapeutic polypeptide(s) described herein (e.g., FZO10).

In some embodiments, engineered strains of Saccharomyces yeast as described herein are useful to treat and/or prevent low-grade chronic gut inflammation associated with diabetes. In some embodiments, engineered strains of Saccharomyces yeast useful in the treatment and/or prevention of gut inflammation associated with diabetes express anti-TNF-α as described herein. In some embodiments, engineered strains of Saccharomyces yeast useful in the treatment and/or prevention of gut inflammation associated with diabetes comprise S. boulardii expressing an anti-TNF-α antibody (e.g., FZ006) as described herein. In some embodiments, engineered strains of Saccharomyces yeast useful in the treatment and/or prevention of gut inflammation associated with diabetes comprise S. boulardii expressing an anti-IL-17A antibody (e.g., FZ004) as described herein. In some embodiments, engineered strains of Saccharomyces yeast useful in the treatment and/or prevention of gut inflammation associated with diabetes comprise S. boulardii expressing an IL-10 (e.g., FZ030) as described herein. In some embodiments, engineered strains of Saccharomyces yeast useful in the treatment and/or prevention of gut inflammation associated with diabetes express a bi-specific antibody against IL-17A and TNF-α described herein. In some embodiments, engineered strains of Saccharomyces yeast useful in the treatment and/or prevention of gut inflammation associated with diabetes comprise S. boulardii expressing a bi-specific antibody against IL-17A and TNF-α (e.g., FZ008). In some embodiments, engineered strains of Saccharomyces yeast useful in the treatment and/or prevention of gut inflammation associated with diabetes express IL-22 polypeptide or IL-22-Fc fusion described herein. In some embodiments, engineered strains of Saccharomyces yeast useful in the treatment and/or prevention of gut inflammation associated with diabetes comprise S. boulardii expressing IL-22 (e.g., FZO10). In some embodiments, engineered strains of Saccharomyces yeast useful in the treatment and/or prevention of gut inflammation associated with diabetes express IL-22 and a bi-specific antibody against IL-17A and TNF-α described herein. In some embodiments, engineered strains of Saccharomyces yeast useful in the treatment and/or prevention of gut inflammation associated with diabetes comprise S. boulardii expressing IL-22 and a bi-specific antibody against IL-17A and TNF-α (e.g., FZ012). In some embodiments, engineered strains of Saccharomyces yeast useful in the treatment and/or prevention of gut inflammation associated with diabetes express an anti-human TNF-α IgG1 as described herein. In some such embodiments, engineered strains of Saccharomyces yeast useful in the treatment and/or prevention of gut inflammation associated with diabetes comprise S. boulardii expressing an anti-human TNF-α IgG1 (e.g., FZ026).

### Obesity

Obesity, as defined in adults by a body mass index (BMI) of greater than or equal to 30, is a growing public health problem worldwide. The world health organization has reported that 13% of adults over the age of 18 are clinically obese, totaling more than 600 million people. The health risks from obesity arise from its association with the increased risk of several diseases including hypertension, Type 2 diabetes, cardiovascular disease, osteoarthritis, kidney failure, liver disease and several types of cancer. Interestingly, chronic inflammation, a phenotype associated with obesity, has been known to be major factor that contributes to the disease progression of the above chronic conditions.

Obesity-associated inflammation is first triggered by excess nutrients and is primarily localized in specialized metabolic tissues such as white adipose tissue, which acts as a major source of energy and is primarily composed of adipocytes. Adipocytes are endocrine cells that secrete a large range of cytokines, hormones and growth factors, referred to as adipokines, and specialize in the storage of energy as triglycerides in cytoplasmic lipid droplets. Excess nutrients leads to activation of metabolic signaling pathways including c-Jun N-terminal kinase (JNK), nuclear factor κ B (NFκB), and protein kinase R. Without wishing to be bound by any one theory, it is understood that activation of these pathways leads to an induction of a low-level of inflammatory cytokines resulting in a low-grade inflammatory response. Excess nutrients and obesity also lead to the hyperplasia and hypertrophy of white adipose tissue adipocytes, as well as the extensive tissue remodeling and an increase in free fatty acids resulting in changes in adipokine production and a low-grade inflammatory response. Obesity also leads to increased endoplasmic reticulum stress resulting in activation of the unfolded protein response, which leads to activation of NFκB, JNK and increased oxidative stress, and in turn the upregulation of inflammatory cytokines. These pathways all contribute to the initiation of obesity associated inflammation. While obesity associated inflammation is primarily localized in white adipose tissue, other tissues have been shown to have increased inflammation under obesity, including the liver, pancreas, and brain.

In some embodiments, engineered strains of Saccharomyces yeast useful in the treatment and/or prevention of obesity express a GLP-1 therapeutic polypeptide(s) described herein. In some such embodiments, engineered strains of Saccharomyces yeast useful in the treatment and/or prevention of obesity comprise S. boulardii expressing a human active GLP-1 therapeutic polypeptide(s) described herein (e.g., FZ022).

In some embodiments, engineered strains of Saccharomyces yeast useful in the treatment and/or prevention of obesity express a leptin therapeutic polypeptide(s) described herein. In some such embodiments, engineered strains of Saccharomyces yeast useful in the treatment and/or prevention of obesity comprise S. boulardii expressing a human active leptin therapeutic polypeptide(s) described herein (e.g., FZ024).

In some embodiments, engineered strains of Saccharomyces yeast useful in the treatment and/or prevention of obesity express an IL-22 therapeutic polypeptide(s) described herein. In some such embodiments, engineered strains of Saccharomyces yeast useful in the treatment and/or prevention of obesity comprise S. boulardii expressing an IL-22 therapeutic polypeptide(s) described herein (e.g., FZO10).

In some embodiments, engineered strains of Saccharomyces yeast as described herein are useful to treat and/or prevent chronic gut inflammation associated with obesity. In some embodiments, engineered strains of Saccharomyces yeast useful in the treatment and/or prevention of gut inflammation associated with obesity express anti-TNF-α as described herein. In some such embodiments, engineered strains of Saccharomyces yeast useful in the treatment and/or prevention of gut inflammation associated with obesity comprise S. boulardii expressing an anti-TNFα antibody (e.g., FZ006) as described herein. In some embodiments, engineered strains of Saccharomyces yeast useful in the treatment and/or prevention of gut inflammation associated with obesity express a bi-specific antibody against IL-17A and TNF-α described herein. In some such embodiments, engineered strains of Saccharomyces yeast useful in the treatment and/or prevention of gut inflammation associated with obesity comprise S. boulardii expressing a bi-specific antibody against IL-17A and TNF-α (e.g., FZ008). In some embodiments, engineered strains of Saccharomyces yeast useful in the treatment and/or prevention of gut inflammation associated with obesity express a IL-22 alone or IL-22-Fc fusion described herein. In some such embodiments, engineered strains of Saccharomyces yeast useful in the treatment and/or prevention of gut inflammation associated with obesity comprise S. boulardii expressing IL-22 (e.g., FZO10). In some embodiments, engineered strains of Saccharomyces yeast useful in the treatment and/or prevention of gut inflammation associated with obesity express IL-22 and a bi-specific antibody against IL-17A and TNF-α described herein. In some such embodiments, engineered strains of Saccharomyces yeast useful in the treatment and/or prevention of gut inflammation associated with obesity comprise S. boulardii expressing IL-22 and a bi-specific antibody against IL-17A and TNF-α (e.g., FZ012). In some embodiments, engineered strains of Saccharomyces yeast useful in the treatment and/or prevention of gut inflammation associated with obesity express an anti-human TNF-α IgG1 as described herein. In some such embodiments, engineered strains of Saccharomyces yeast useful in the treatment and/or prevention of gut inflammation associated with obesity comprise S. boulardii expressing an anti-human TNF-α IgG1 (e.g., FZ026). In some embodiments, engineered strains of Saccharomyces yeast useful in the treatment and/or prevention of gut inflammation associated with obesity comprise S. boulardii expressing an anti-IL-17A antibody (e.g., FZ004) as described herein. In some embodiments, engineered strains of Saccharomyces yeast useful in the treatment and/or prevention of gut inflammation associated with obesity comprise S. boulardii expressing an IL-10 (e.g., FZ030) as described herein.

### Fatty Liver Disease (FLD) and Other Liver Diseases

Fatty Liver Disease (FLD, or “Fatty Liver”) corresponds to the presence of macrovesicular changes without inflammation (steatosis) and lobular inflammation in the absence of significant alcohol use. It can be divided into two subgroups: NAFL (Non-Alcoholic Fatty Liver) or simply Steatosis and NASH (Non-Alcoholic Steatohepatitis). NAFL is defined as the presence of hepatic steatosis with no evidence of hepatocellular injury in the form of ballooning of the hepatocytes. NASH is defined as the presence of hepatic steatosis and inflammation with hepatocyte injury (ballooning), Malloryhyaline, and mixed lymphocytic and neutrophilic inflammatory infiltrate in perivenular areas with or without fibrosis.

Interestingly, in both nonalcoholic and alcoholic liver disease models, interleukin-20 (IL-20) family of cytokines reduces liver injury and inflammation. For example, interleukin-22 (IL-22), a member of the IL-20 subfamily, controls lipid metabolism in the liver via activation of the STAT3 signaling pathway. Accordingly, in some embodiments, engineered strains of Saccharomyces yeast useful in the treatment and/or prevention of FLD express an IL-22 therapeutic polypeptide(s) described herein. In some such embodiments, an engineered strain of Saccharomyces yeast useful in the treatment and/or prevention of FLD comprise S. boulardii expressing an IL-22 therapeutic polypeptide(s) described herein (e.g., FZO10).

The gut-liver axis describes the physiological interplay between the gut and the liver and has important implications for the maintenance of health. Disruptions of this equilibrium are an important factor in the evolution and progression of many liver diseases. Gut inflammation leads to impaired gut barrier function, allowing translocation of microbes and microbial products named microbial or pathogen-associated molecular patterns (MAMPs/PAMPs) to liver causing liver inflammation. Accordingly, in some embodiments, engineered strains of Saccharomyces yeast useful in the treatment and/or prevention of FLD express anti-inflammatory therapeutic polypeptide(s), such as anti-TNFa, anti-IL-17A described herein. In some such embodiments, an engineered strain of Saccharomyces yeast useful in the treatment and/or prevention of FLD comprise S. boulardii expressing anti-TNFa, anti-IL-17A therapeutic polypeptide(s) described herein (e.g., FZ006, FZ008, FZ026).

### Graft-Versus-Host Disease (GVHD)

Graft-versus-host disease (GVHD) is a systemic disorder that occurs when the graft's immune cells recognize the host as foreign and attack the recipient's body cells. “Graft” refers to transplanted, or donated tissue, and “host” refers to the tissues of the recipient. GVHD is a significant cause of morbidity among subjects receiving treatments such as allogeneic cell therapies or transplants. Immune cell recognition of the host can induce a “cytokine storm”, a related pro-inflammatory reaction caused by cytokines.

Accordingly, in some embodiments, engineered strains of Saccharomyces yeast useful in the treatment and/or prevention of GVHD express an IL-22 therapeutic polypeptide(s) and/or mono-specific or bi-specific antibody against IL-17A and/or TNF-α described herein. In some such embodiments, an engineered strain of Saccharomyces yeast useful in the treatment and/or prevention of GVHD comprise S. boulardii expressing an IL-10 or IL-22 therapeutic polypeptide(s) and/or mono-specific or bi-specific antibody against IL-17A and/or TNF-α described herein (e.g., FZ004, FZ006, FZ008, FZO10, FZ012, FZ030, and FZ026). S. boulardii expressing an IL-10 or IL-22 therapeutic polypeptide(s) and/or mono-specific or bi-specific antibody against IL-17A and TNF-α described herein (e.g., FZ004, FZ006, FZ008, FZO10, FZ012, FZ030, and FZ026) can also be used in the treatment of metabolic diseases.

### Gastrointestinal Viruses (e.g., Rotavirus, Norovirus)

Viruses are the agents of acute infectious gastroenteritis which results in inflammation of the inside lining of the gastrointestinal tract, producing a syndrome of vomiting, watery diarrhea, or both that begins abruptly in otherwise healthy persons. Two distinct viruses account for the majority of cases. Rotaviruses are the principal agent of sporadic, severe gastroenteritis in young children and are responsible for the death of approximately 1600 children daily worldwide, mainly in developing countries. Noroviruses are the principal agent of epidemic infectious gastroenteritis in both infants and adults. For example, outbreaks of gastroenteritis in closed settings, such as cruise ships and nursing homes, are a typical manifestation of norovirus infections. However, noroviruses are also a common cause of sporadic, severe gastroenteritis in young children (see, e.g., Franco MA. et al., Goldman's Cecil Medicine. 2012; 2144-2147).

Rotaviruses, which belong to the family Reoviridae, are large, icosahedral, nonenveloped viruses with a segmented, double-stranded RNA genome and a triple-layered protein coat. Rotaviruses are classified into groups A through G on the basis of the presence of cross-reactive antigenic epitopes and their overall genetic relatedness. Group A rotaviruses are the principal enteric pathogen of humans and many other species. Group B viruses have been identified sporadically in outbreaks of adult diarrheal illness in China and more recently in studies of children with sporadic gastroenteritis, principally in India. Group C rotaviruses are relatively infrequently associated with diarrheal disease in humans and animals around the world. Groups D through G rotaviruses have been isolated only from animals, primarily avian species. Rotaviruses are 100-nm particles that have three concentric layers of proteins: the core is composed of VP1, VP2, and VP3 and the segmented, double-stranded RNA genome; the intermediate layer is formed by VP6, the most abundant and antigenic structural viral protein; and the external layer is composed of VP7 and VP4. The genome is composed of 11 segments of double-stranded RNA that together are approximately 18 kilobases and encode six structural and six nonstructural proteins. As is the case among virtually all other RNA viruses, the rotavirus RNA polymerase is error prone and, along with selective pressure such as the evolution of immunity, drives viral diversity. For rotaviruses, gene reassortment, which is the mixing of gene segments from different parental viruses in cells coinfected by two or more strains, and rearrangement of the viral genome also contribute to genetic diversity. Reassortment of gene segments between animal and human rotavirus strains also occurs in natural settings, especially in less developed countries.

Noroviruses, which are one of the five genera of the Caliciviridae family, are nonenveloped, icosahedral viruses with a relatively small, positive-sense, single-stranded RNA genome. The norovirus genus is further classified into five genogroups (GI to GV), only three of which (GI, GII, and GIV) are known to infect humans. GIII and GV viruses infect bovines and mice, respectively, and to date these animal viruses have not been shown to infect humans. Viruses in each genogroup are further divided into genotypes (more than 25 have been described) and subgroups. Norwalk virus is a prototype genogroup I genotype 1 (GI.1) virus. The norovirus genome is approximately 7.7 kilobases in size and consists of three open reading frames, the first of which encodes the nonstructural proteins that are essential for virus replication. The second open reading frame encodes the major capsid protein, viral protein 1 (VP1). When it is expressed as a recombinant protein, 180 molecules of VP1 auto assemble into virus-like particles that are critical to the study of noroviral epidemiology and immunity.

Accordingly, in some embodiments, engineered strains of Saccharomyces yeast useful in the treatment and/or prevention of Rotavirus express an anti-Rotavirus VHH described herein. In some such embodiments, an engineered strain of Saccharomyces yeast useful in the treatment and/or prevention of Rotavirus comprises S. boulardii expressing anti-Rotavirus VHH described herein (e.g., FZ014).

In some embodiments, engineered strains of Saccharomyces yeast useful in the treatment and/or prevention of norovirus express bi-specific VHHs-Fc against norovirus (e.g., M6M5-Fc and M6M4-Fc) described herein. In some such embodiments, an engineered strain of Saccharomyces yeast useful in the treatment and/or prevention of norovirus comprises S. boulardii expressing bi-specific VHHs-Fc against norovirus (e.g., M5, M6M5-Fc and M6M4-Fc) described herein (e.g., FZ016).

In some embodiments, a subject is infected with both norovirus and C. difficile. Accordingly, in some embodiments, engineered strains of Saccharomyces yeast useful in the treatment and/or prevention of co-infection with norovirus and C. difficile express bi-specific VHHs-Fc against norovirus (e.g., M6M5-Fc and M6M4-Fc) and an antitoxin ABAB described herein. In some such embodiments, an engineered strain of Saccharomyces yeast useful in the treatment and/or prevention of co-infection with norovirus and C. difficile comprises S. boulardii expressing bi-specific VHHs-Fc against norovirus (e.g., M6M5-Fc and M6M4-Fc) and an antitoxin ABAB described herein (e.g., FZ018).

### Gastrointestinal Infections

IL-22 is an important cytokine for maintaining homeostasis at various mucosal barriers, including the gastrointestinal tract. During infection with enteropathogens, IL-22 is highly upregulated, leading to induction of multiple antimicrobial factors and wound healing and restoration of barrier function. In the intestine, IL-22 signaling promotes important functions including host defense against pathogens and wound healing. The application of intestinal recombinant IL-22 may have a beneficial impact in liver and pancreas damage, ulcerative colitis, graft-versus-host disease.

In some embodiments, engineered strains of Saccharomyces yeast useful in the treatment and/or prevention of enteric pathogen colonization and infections express IL-22 polypeptides described herein. In some such embodiments, an engineered strain of Saccharomyces yeast useful in the treatment and/or prevention of enteric pathogen colonization and infections comprises S. boulardii expressing IL-22 polypeptides described herein (e.g., FZ010).

### Cardiovascular Diseases

Intestinal inflammation leads to the reduced integrity of the gut barrier, which in turn increases circulating levels of bacterial structural components and microbial metabolites, including trimethylamine-N-oxide and short-chain fatty acids, which may facilitate the development of cardiovascular diseases (CVD). In some embodiments, engineered strains of Saccharomyces yeast as described herein are useful to treat and/or prevent gut inflammation associated with CVD. In some embodiments, engineered strains of Saccharomyces yeast useful in the treatment and/or prevention of gut inflammation associated with CVD express anti-TNF-α as described herein. In some such embodiments, engineered strains of Saccharomyces yeast useful in the treatment and/or prevention of gut inflammation associated with CVD comprise S. boulardii expressing an anti-IL17A or anti-TNF-α antibody (e.g., FZ004 or FZ006) as described herein. In some embodiments, engineered strains of Saccharomyces yeast useful in the treatment and/or prevention of gut inflammation associated with CVD express a bi-specific antibody against IL-17A and TNF-α described herein. In some such embodiments, engineered strains of Saccharomyces yeast useful in the treatment and/or prevention of gut inflammation associated with CVD comprise S. boulardii expressing a bi-specific antibody against IL-17A and TNF-α (e.g., FZ008). In some embodiments, engineered strains of Saccharomyces yeast useful in the treatment and/or prevention of gut inflammation associated with CVD express a IL-22 alone or IL-22-Fc fusion described herein. In some such embodiments, engineered strains of Saccharomyces yeast useful in the treatment and/or prevention of gut inflammation associated with CVD comprise S. boulardii expressing IL-22 (e.g., FZO10). In some embodiments, engineered strains of Saccharomyces yeast useful in the treatment and/or prevention of gut inflammation associated with CVD express IL-22 and a bi-specific antibody against IL-17A and TNF-α described herein. In some such embodiments, engineered strains of Saccharomyces yeast useful in the treatment and/or prevention of gut inflammation associated with CVD comprise S. boulardii expressing IL-22 and a bi-specific antibody against IL-17A and TNF-α (e.g., FZ012). In some embodiments, engineered strains of Saccharomyces yeast useful in the treatment and/or prevention of gut inflammation associated with CVD express an anti-human TNF-α IgG1 as described herein. In some such embodiments, engineered strains of Saccharomyces yeast useful in the treatment and/or prevention of gut inflammation associated with CVD comprise S. boulardii expressing an anti-human TNF-α IgG1 (e.g., FZ026).

### Irritable Bowel Syndrome (IBS)

Irritable bowel syndrome (IBS) is a gastrointestinal-related disorder that manifests as persistent abdominal pain or discomfort, which is commonly correlated with altered bowel habits as well as defecation frequency and form. Although IBS was once assumed to primarily affect the Western population, it is becoming increasingly prevalent in developing Asian countries. IBS is subclassified as constipationpredominant IBS (IBS-C), diarrhea-predominant IBS (IBS-D), alternating or mixed IBS (A/M-IBS), and postinfectious IBS (PI-IBS). Patients with IBS are increasingly presenting with a wide range of neuropsychiatric symptoms, such as deterioration in gastroenteric physiology, including visceral hypersensitivity, altered intestinal membrane permeability, and gastrointestinal motor dysfunction. Gut-brain axis links the interrelations between the enteric and central events in IBS-related gastrointestinal, neurological, and psychiatric pathologies. The pathophysiology of IBS is involved in altered signaling by the gut-brain axis, dysbiosis, abnormal visceral pain signaling and intestinal immune activation. The immune activation plays roles in the pathogenesis of IBS through the bidirectional communication between the nervous system and the immune system. Gut microbiota is associated with IBS and the changes in the composition, temporal stability, and metabolic activity of the gut microbiota have been described in patients with IBS.

In some embodiments, engineered strains of Saccharomyces yeast as described herein are useful to treat and/or prevent IBS through affecting gut inflammation, barrier function, and gut microbiota, such as FZ004, FZ006, FZ008, FZO10, FZ012, FZ026, and FZ030.

### Compositions (e.g., Pharmaceutical Compositions)

In another aspect, disclosed herein is a pharmaceutical composition including one or more engineered strains of Saccharomyces yeast and at least one pharmaceutically-acceptable carrier or diluent. In some aspects, the pharmaceutical composition can be formulated as an oral dosage form, a rectal dosage form, a vaginal dosage form, or a topical dosage form. In some aspects, the oral dosage form comprises an enteric coating. In still another aspect, the pharmaceutical compositions disclosed herein are less expensive to produce than monoclonal or polyclonal antibodies. In any of these aspects, administration of the pharmaceutical composition does not induce an anti-drug response.

In various aspects, the present disclosure relates to pharmaceutical compositions comprising a therapeutically effective amount of engineered strains of Saccharomyces yeast described herein. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed engineered strain of S. boulardii organism, a pharmaceutically acceptable carrier, optionally one or more other therapeutic agent, and optionally one or more adjuvant. The disclosed pharmaceutical compositions include those suitable for oral administration.

In various aspects, the present disclosure also relates to a pharmaceutical composition comprising a pharmaceutically acceptable carrier or diluent and, as active ingredient, a therapeutically effective amount of one or more engineered strains of Saccharomyces yeast. In one aspect, the therapeutically-effective amount of the pharmaceutical composition includes from about 1 billion to about 10 billion colony forming unit (CFU) of an engineered strain of Saccharomyces yeast, or about 1, 2, 3, 4, 5, 6, 7, 8, 9, or about 10 billion CFU of the engineered strain of Saccharomyces yeast, or a combination of any of the foregoing values, or a range encompassing any of the foregoing values.

In practice, the organisms of the present disclosure (e.g., engineered strains of Saccharomyces yeast) can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient, or for vaginal administration including bioadhesive delivery systems, phase change poloxamers, tablets, suppositories, creams, gels, vaginal rings, and the like, for rectal administration (i.e. suppositories and/or enemas), or for topical administration (e.g., solutions, lotions, creams, ointments, gels, pastes, aerosol foams, aerosol sprays, powder, solids, transdermal patches, and the like). Further, the compositions can be presented as a powder, as granules, as a solution, as a suspension in an aqueous liquid, as a non-aqueous liquid, as an oil-in-water emulsion or as a water-in-oil liquid emulsion. In some embodiments, compositions are presented in combination with food (e.g., feed provided to animals such as piglets or poultry, medical food for humans). In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; powder packets; wafers; and segregated multiples thereof. This list of unit dosage forms is not intended to be limiting in any way, but merely to represent typical examples of unit dosage forms.

The pharmaceutical compositions disclosed herein comprise one or more engineered strains of Saccharomyces yeast of the present disclosure as an active ingredient, a pharmaceutically acceptable carrier, and optionally one or more additional therapeutic agents. In various aspects, the disclosed pharmaceutical compositions can include a pharmaceutically acceptable carrier and an engineered strains of Saccharomyces yeast described herein. In a further aspect, an engineered strains of Saccharomyces yeast described herein can also be included in a pharmaceutical composition in combination with one or more other therapeutically active compounds. The instant compositions include compositions suitable for oral administration. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In some embodiments, disclosed compounds (e.g., engineered strains of Saccharomyces yeast described herein) are encapsulated. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, sucrose, trehalose, inulin, ACPs, alginate, sodium ascorbate, magnesium sulfate, pectin, starches (for example corn starch or amylose), dextran, avicel (microcrystalline cellulose), maltodextrin, magnesium stearate, silicone dioxide, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, ascorbic acid, sodium ascorbate, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with 01-012-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate, magnesium sulfate, and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example Eudragit® RS), copolymerizates of acrylic and methacrylic acid esters and trimethyl ammonium methacrylate (for example Eudragit® RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid or a liquid. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, avicel (microcrystalline cellulose), maltodextrin, magnesium stearate, silicone dioxide, and stearic acid. Examples of liquid carriers are sugar syrup, peanut oil, olive oil, and water.

In various aspects, a binder can include, for example, starch, gelatin, natural sugars such as glucose or beta-lactose, corn sweeteners, natural and synthetic gums such as acacia, tragacanth, alginate, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed microorganism in contact with one or more biodegradable polymers. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed microorganism can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In another aspect, an engineered strain of Saccharomyces yeast can be lyophilized in the pharmaceutical composition. In some embodiments, an engineered strain of Saccharomyces yeast can be mixed with cryoprotectants to enhance viability during the freeze drying. In some aspects, the pharmaceutical composition can be formulated as an oral dosage form. In one aspect, the oral dosage form can be a capsule, a tablet, a caplet, a gelcap, a powder, a liquid solution, a suspension, or any combination thereof. In some aspects, the oral dosage form comprises an enteric coating.

In an aspect, an oral dosage form is preferred to other methods of administering therapy targeting inflammatory cytokines. In some aspects, standard therapy in, for example, Crohn's disease, ulcerative colitis, and the like, may involve administration of biologics and/or other therapeutic agents by injection or infusion. In some aspects, such drug administration reduces patient compliance as travel to treatment sites must be arranged and medical personnel need to be trained to administer treatments by injection or infusion. In other aspects, therapeutic agents administered by injection or infusion require sterility and/or may provoke immune responses in the subjects. Thus, in one aspect, the disclosed pharmaceutical compositions and oral dosage forms represent a significant improvement over current therapies since they can be self-administered by patients at home and do not provoke an immune response. In still another aspect, the pharmaceutical compositions disclosed herein are less expensive to produce than monoclonal or polyclonal antibodies.

In various aspects, a solid oral dosage form, such as a tablet or a capsule, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethylcellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances (e.g., phosphate buffered saline, PBS), flavor correcting agents, sweeteners, colorants, antioxidants, complex formers, and the like. Complex formers which may be for example be considered are: chelate formers such as ethylene diamine tetraacetic acid, nitrilotriacetic acid, diethylene triamine pentaacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed microorganism in a disclosed liquid dosage form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed organism or mixture of organisms; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment of conditions such as, for example, Crohn's disease, ulcerative colitis, or other forms of inflammatory bowel diseases, an appropriate dosage level will generally be about from about 1 billion to about 10 billion CFU per unit dosage and can be administered in single or multiple doses. Within this range the dosage can be about 1, 5, 10, 15, 20, 25, or about 30 billion CFU of genetically modified S. boulardii per day. The compound can be administered on a regimen of 1 to 4 times per day, preferably once or twice per day. In an exemplary aspect, an oral dosage form can include 5 billion CFU in a 250 mg capsule and two capsules can be administered twice per day. This dosing regimen can be adjusted to provide the optimal therapeutic response.

In one aspect, in the pharmaceutical composition, the oral dosage form comprises from about 1 billion colony forming units (CFUs) to about 10 billion CFUs of the engineered strain of Saccharomyces yeast per unit dose, or about 1, 5, 6, 7, 8, 9, or about 10 billion CFUs per unit dose, or a combination of any of the foregoing values, or a range encompassing any of the foregoing values. In some aspects, the pharmaceutical composition is acid resistant.

In another aspect, the method includes administering the pharmaceutical composition for a period of from about 3 days to about 4 weeks, a plurality of months, or a plurality of years, or about 3, 4, 5, 6, or 7 days, or 2, 3, or 4 weeks, or about 2, 3, 4, 5, 6, 7, 8, 9, 10, or 11 months, or about 1, 2, 5, 10, 15, 20, 25, 30, 35, 40, 45, 50, 55, 60, 65, 70, 75, 80 or more years, or a combination of any of the foregoing values, or a range encompassing any of the foregoing values. In another aspect, the pharmaceutical composition is administered from once per day to four times per day, or 1, 2, 3, or 4 times per day.

Such unit doses as described hereinabove and hereinafter can be administered more than once a day, for example, 2, 3, 4, 5 or 6 times a day. In various aspects, such therapy can extend for a number of weeks or months, and in some cases, years or as long as patient symptoms persist. In one aspect, in the case of an inflammatory bowel disease, the oral dosage form can be administered as long as symptoms persist. In one aspect, in an example of an inflammatory bowel disease, the oral dosage form can be administered to maintain disease remission for years or lifetime. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be a 1 mg to about 100 mg tablet or 1 mg to about 300 mg tablet taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The disclosed pharmaceutical compositions can further comprise other therapeutically active compounds, which are usually applied in the treatment of the above mentioned pathological or clinical conditions.

### Administration

Administration of technologies of the present disclosure (e.g., engineered strains of Saccharomyces yeast and pharmaceutical compositions comprising the same) can be effected in one dose, continuously or intermittently. Methods of determining the most effective means and dosage of administration are known to those of ordinary skill in the art and will vary with the composition used, the purposes of the therapy, etc. Single or multiple administrations can be carried out with the dose level and pattern being selected by the treating physician. In some embodiments, technologies of the present disclosure are administered once daily for a period of time. In some embodiments, technologies of the present disclosure administered twice daily for a period of time. In some such embodiments, a period of time includes, for example, days (e.g., 1, 2, 3, 4, 5, 6, 7, 8, 9, 10, 14, 21, or 30 days), months (e.g., 1, 2, 3, 4, 5, 6, 7, 8, 9, 10, 11, or 12 months), or years (e.g., 1, 2, 5, 10, 15, 20, 25, 30, 35, 40, 45, 50, 55, 60, 65, 70, 75, 80, or more years).

Suitable dosage formulations and methods of administering the technologies of the present disclosure are known in the art. Route of administration can also be determined and method of determining the most effective route of administration are known to those of skill in the art and will vary with the composition used for treatment, the purpose of the treatment, the health condition or disease stage of the subject being treated and target cell or tissue. Non-limiting examples of route of administration include oral administration, vaginal, nasal administration, injection, topical application and by suppository.

In any of these aspects, the engineered strain of Saccharomyces yeast occupies the intestine of the subject from about 3 days to about 5 days after oral delivery of the pharmaceutical composition. In one aspect, due to this transient occupation of the intestine, dosage of the pharmaceutical compositions disclosed herein, as well as duration of treatment, can be carefully controlled using the disclosed oral delivery mechanisms.

### Combination Therapies

Also provided herein are methods for treating and/or preventing a disease, disorder, and/or condition in a subject in need thereof comprising administering to the subject an effective amount of an engineered strain of Saccharomyces yeast, as described herein, or a pharmaceutical composition comprising the same and a therapeutically-effective amount of an antibiotic. In some embodiments, the antibiotic is administered at the same time as the engineered strain of Saccharomyces yeast as described herein, or a pharmaceutical composition comprising the same. In some embodiments, the antibiotic is administered before the engineered strain of Saccharomyces yeast as described herein, or a pharmaceutical composition comprising the same. In some embodiments, the antibiotic is administered after the engineered strain of Saccharomyces yeast, as described herein, or a pharmaceutical composition comprising the same.

In some embodiments, the antibiotic can be selected from, for example, metronidazole, ciprofloxacin, rifaximin, ampicillin, tetracycline, amoxicillin, doxycycline, levofloxacin, clavulanate potassium, vancomycin, sulfamethosazol, trimethoprim, clindamycin, tinidazole, tylosin, or any combination thereof. In one aspect, the pharmaceutical compositions disclosed herein are superior to probiotic compositions including only bacteria due to the ability to co-administer antibiotics if required. Further in this aspect, unlike bacterial probiotics, the engineered strains of Saccharomyces yeast are not susceptible to antibiotics.

In some embodiments, the efficacy of parentally used drug can be boosted by modulating gut function. It is well known that gut microbes may affect cancer treatment. Without wishing to be bound by any one theory, gut inflammation can affect the abundance and composition of gut microbes which in turn affects anti-cancer treatment, thus, in some embodiments, engineered strains of Saccharomyces yeast can be co-administrated with anti-cancer agents to enhance treatment efficacy.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: The Platform of Sb-mSdAb (BioPYM™) Technology

The work described herein builds upon a novel platform technology, against, for example, enteric pathogens by engineering a probiotic yeast, Saccharomyces boulardii, to secrete, for example, multi-specific single-domain (VHH) antibodies (Sb-mSdAb), directly neutralizing major intestinal disease biomarkers. This platform is also referred to as Bioengineered Probiotic Yeast Medicines (BioPYM™)

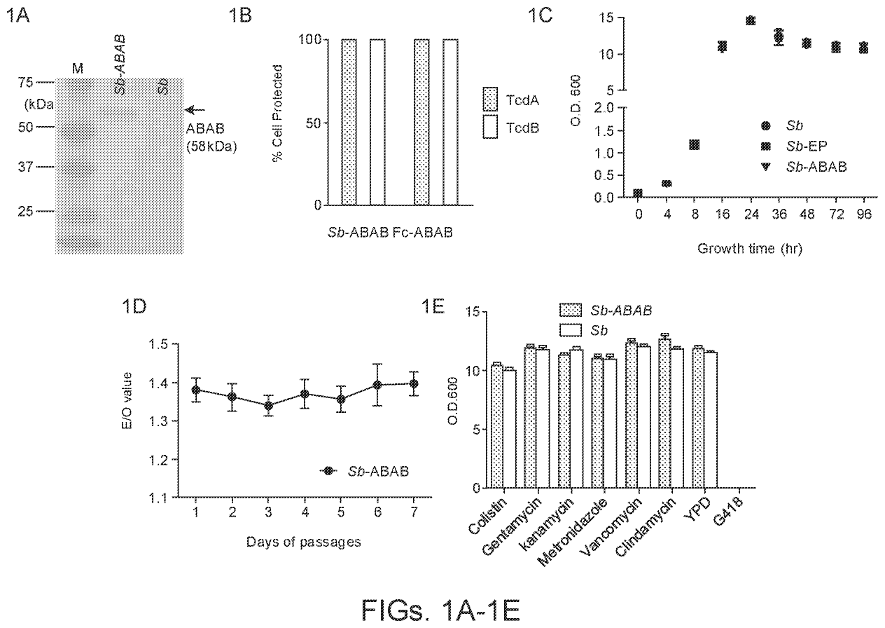

Using this innovative technology, we have generated an exemplary therapeutic lead of Sb-ABAB, designated FZ002, that constitutively secretes a tetra-specific, single-domain antibody ABAB as a mSdAb fusion polypeptide consisting of 4 non-overlapping VHHs that potently and broadly neutralizes the two major C. difficile enterotoxins, TcdA and TcdB. The secretion of functional ABAB was verified both in vitro and in vivo. For in vitro characterization, secretion of ABAB was verified by western immunoblotting (FIG. 1A), and the expected neutralizing activity of secreted ABAB against both TcdA and TcdB similar to purified Fc-ABAB fusion polypeptide was also confirmed (FIG. 1B). The final Sb-ABAB clone had growth comparable to the wild type Sb and the Sb transformed with an empty plasmid (Sb-EP) controls (FIG. 1C) and to stably secrete ABAB over 7 days of passages (FIG. 1D). Sb-ABAB was similarly resistant to a panel of antibiotics compared to wild type, but not to antifungal G418, which is toxic to eukaryotic cells (FIG. 1E).

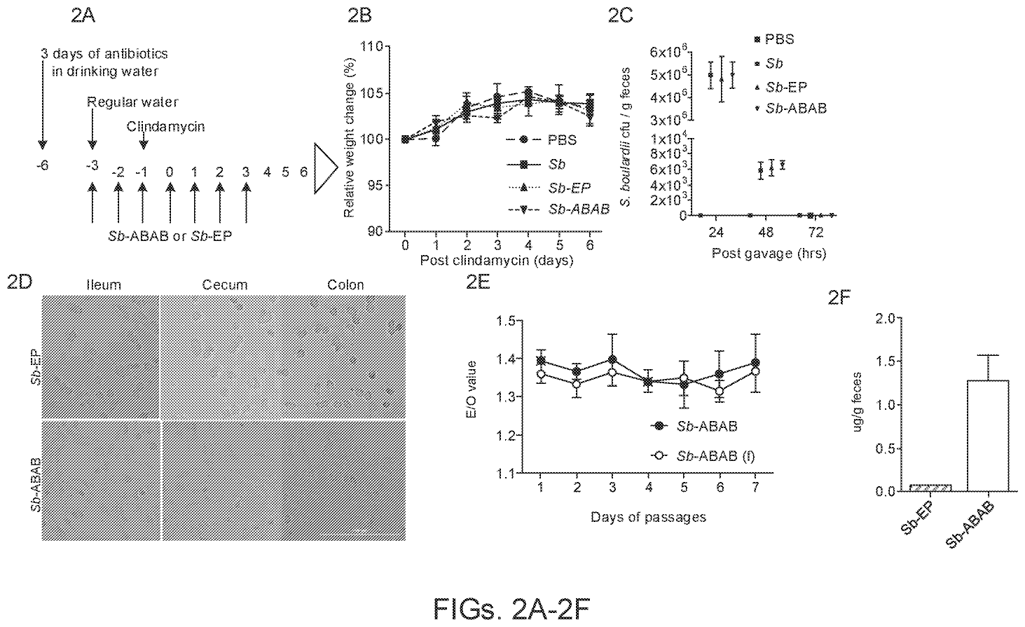

To examine in vivo properties of Sb-ABAB and mouse tolerability of the highest achievable doses, we orally gavaged mice with 10¹⁰ colony forming units (CFU) of the engineered yeast (FIG. 2A). Mice that were gavaged with S. boulardii (Sb), Sb-EP (Sb yeast transformed with “Empty Plasmid” (pURA3-AT-cMyc) which does not contain the ABAB transgene), or Sb-ABAB gained similar weights as placebo (PBS) (FIG. 2B), and these strains shared comparable persistence patterns in these mice (FIG. 2C). Moreover, Sb-ABAB, but not Sb-EP, secreted functional ABAB into the mouse ileum, cecum, and colon after oral gavage (FIG. 2D). Finally, Sb-ABAB recovered from mouse feces continued to stably secrete ABAB (FIG. 2E). ABAB levels in day 3 feces were determined by ELISA (FIG. 2F). Moreover, we demonstrated that oral FZ002 provided significant protections against both primary and recurrent C. difficile infection in mice.

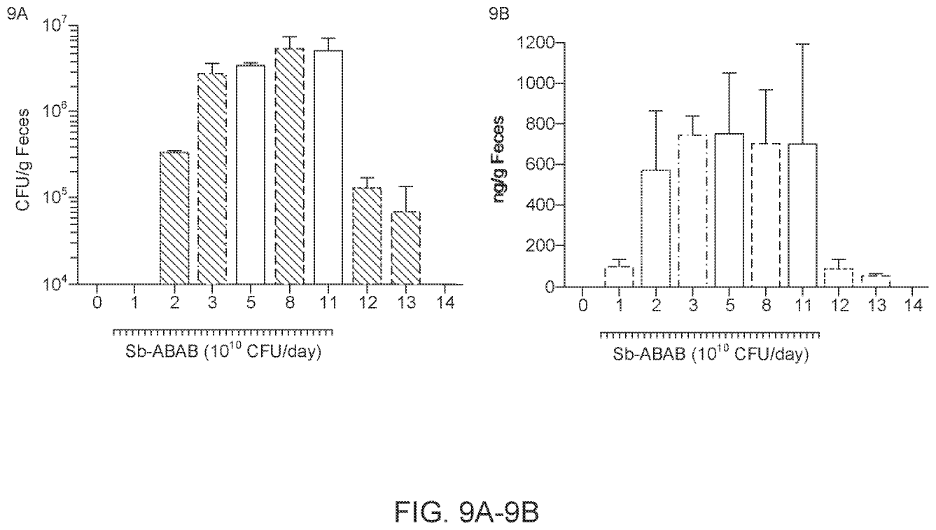

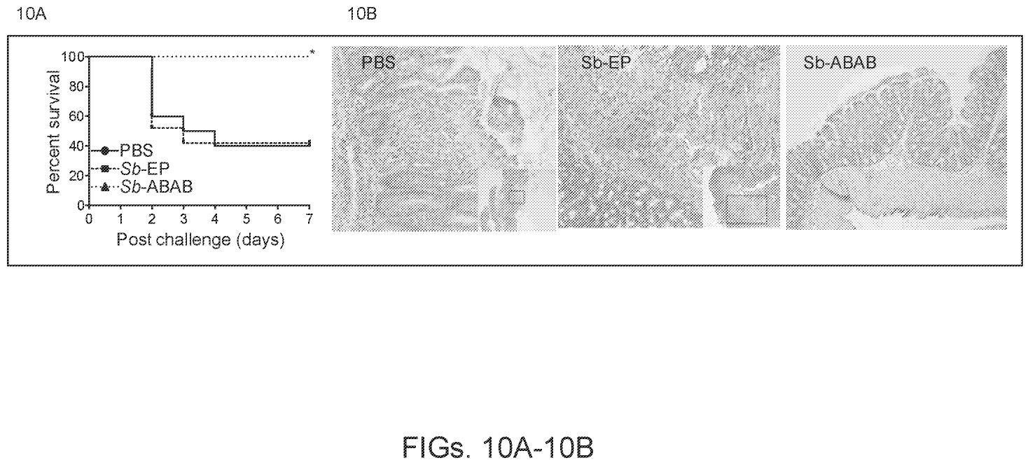

FIG. 9A-9B shows that piglets administered with Sb-ABAB shed live yeast and ABAB in their feces. FIG. 10A-10B shows that mice gavaged with Sb-ABAB are protected against C. difficile induced death and intestinal inflammations. Mice were gavaged with PBS, Sb-EP, or Sb-ABAB (109 CFU/dose/day) for three days before bacterial challenge and continued for another three days after bacterial challenge. Mice were challenged with 104 CFU of C. difficile spores. Mouse survival and intestinal histology are shown.

### Example 2: Construction of Sb-amTNF

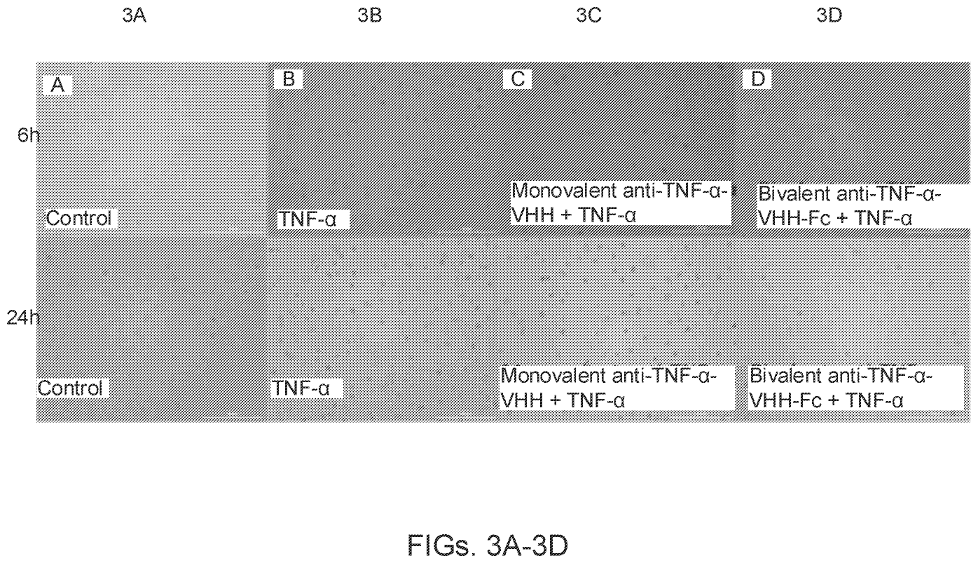

We constructed a prototype yeast Sb-amTNF (FZ006m) secreting an anti-mouse TNF-α VHH, which allows us to evaluate the therapeutic potential of the engineered yeast against intestinal inflammatory diseases in conventional mice. We demonstrated that the homodimer of anti-mouse TNF-α VHH, when fused with Fc, displayed the highest neutralizing activity (FIGS. 3A-3D). We therefore engineered the Sb-amTNF strain by inserting VHH/VHH-Fc cassettes into the pTEF or pTDH loci of the auxotrophic Sb strain. Sb-amTNF stably secreted functional anti-mouse TNF-α VHH/VHH-Fc as detected by ELISA and a cell culture assay (FIGS. 3A-3D).

### Example 3: Oral Sb-amTNF Reduces Severity of Intestinal Inflammatory Diseases

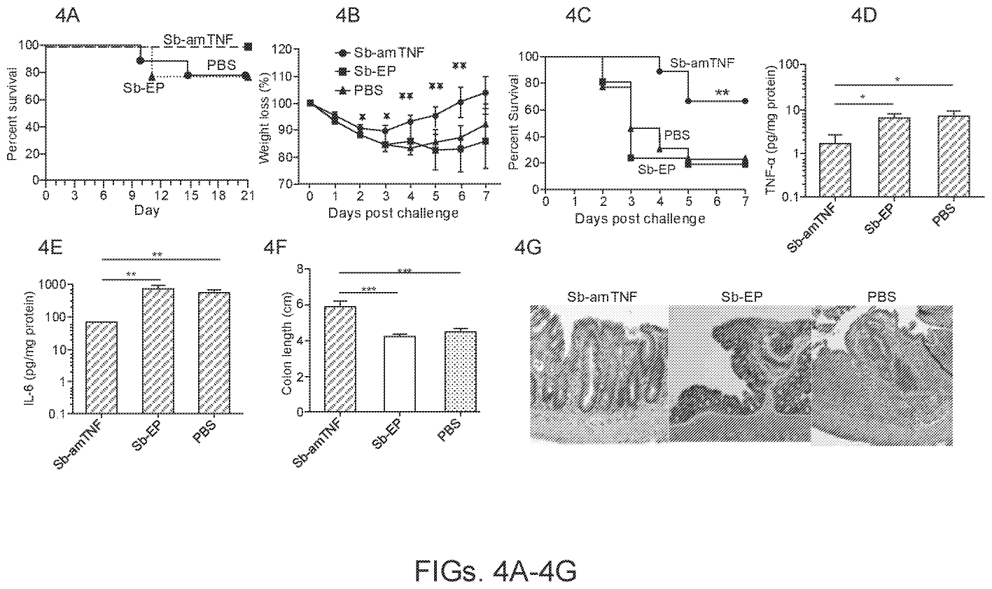

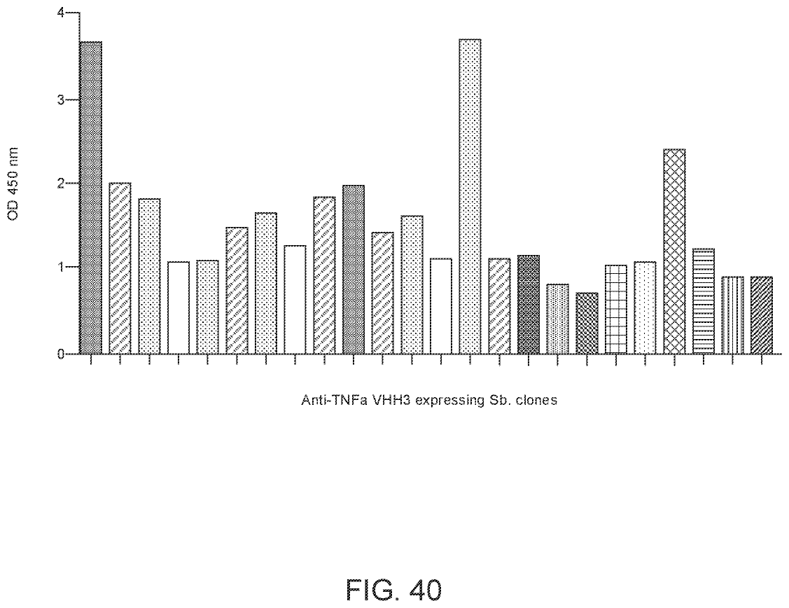

We first tested whether intestinal delivery of a potent TNF-α-neutralizing antibody ameliorates symptoms of dextran sulfate sodium (DSS)-colitis. Mice were on drinking water with 2% DSS for 8 days as described previously. Starting on day 3, mice were gavaged daily with 10 CFU of Sb-amTNF for 11 days. Control groups were gavaged with the same amount of Sb-EP control or PBS. FIG. 4A shows that none of those mice treated with oral Sb-amTNF died whereas 20% of mice succumbed in control groups. As DSS-colitis alone did not induce a significant death outcome in mice, we utilized a significantly more severe intestinal inflammation model of DSS-colitis comorbid with C. difficile infection. In this comorbidity model, mice developed severe intestinal inflammation, tissue damages, and 80% of the mice died. Since TNF-α was significantly upregulated, we investigated whether anti-TNF-α antibody intestinally delivered by engineered S. boulardii would ameliorate disease severity and reduce intestinal inflammation. Groups of comorbid mice were orally administered with PBS, Sb-EP, or Sb-amTNF at 10⁹ CFU/dose daily from day −3 to day 7 (11 doses total). Significant weight loss was evident and about 80% of mice had succumbed to the disease in the PBS and Sb-EP control groups, whereas oral Sb-amTNF conferred substantial protection with significantly lower weight loss (FIG. 4B) and 40% of mice succumbed (FIG. 4C). The upregulation of colonic proinflammatory cytokines TNF-α (FIG. 4D) and IL-6 (FIG. 4E) was also significantly reduced. Subsequently, the colon length reductions and tissue damages were also significantly alleviated in mice treated with oral Sb-amTNF (FIGS. 4F-4G). In summary, our proof-of-concept data demonstrated that intestinal delivery of anti-TNF-α neutralizing antibody via engineered S. boulardii (oral Sb-amTNF) significantly ameliorates disease severity by reducing intestinal inflammation and tissue damages.

### Example 4: Construction of a Yeast Strain Secreting Anti-Human TNF-α Neutralizing Antibody Sb-ahTNF (FZ006)

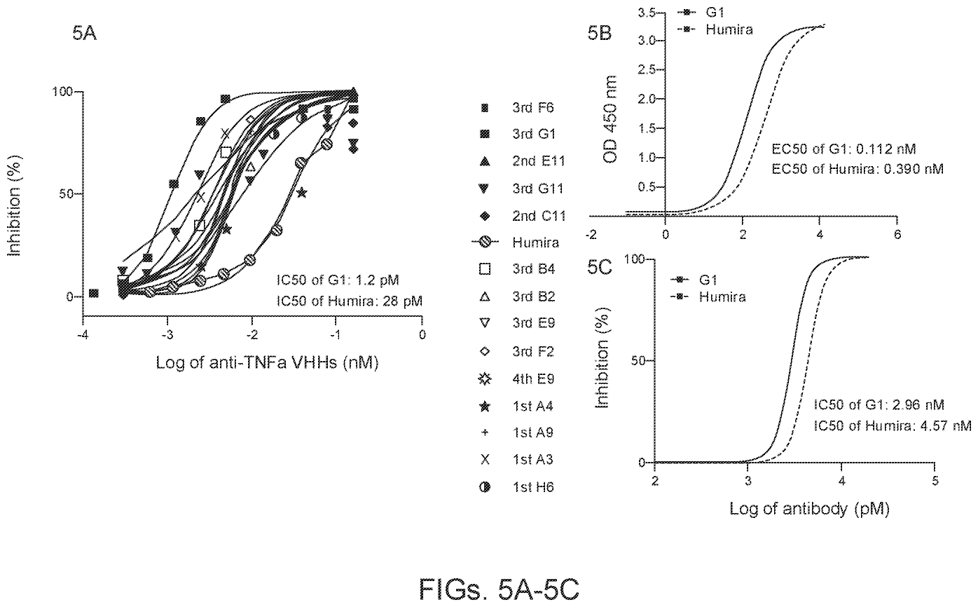

From an immune VHH yeast display library, 8 VHHs that bind to human TNF-α protein or polypeptide and neutralize its cytotoxic activity on L929 cells have been identified. We then randomly paired these VHHs to form monomer VHH-Fc and dimeric VEHVHH-Fc and identified 14 candidates exhibiting the best neutralizing activities (FIG. 5A). Comparing to Adalimumab (brand name as HUMIRA®), a heterodimer G1 was 24-fold more potent in neutralizing human TNF-α-mediated cytotoxicity of L929 cells (FIG. 5A). G1 had a higher binding affinity to TNF-α than Humira in an ELISA (FIG. 5B) and was more potent in blocking the binding of TNF-α to TNFR1 as determined by a competition ELISA (FIG. 5C).

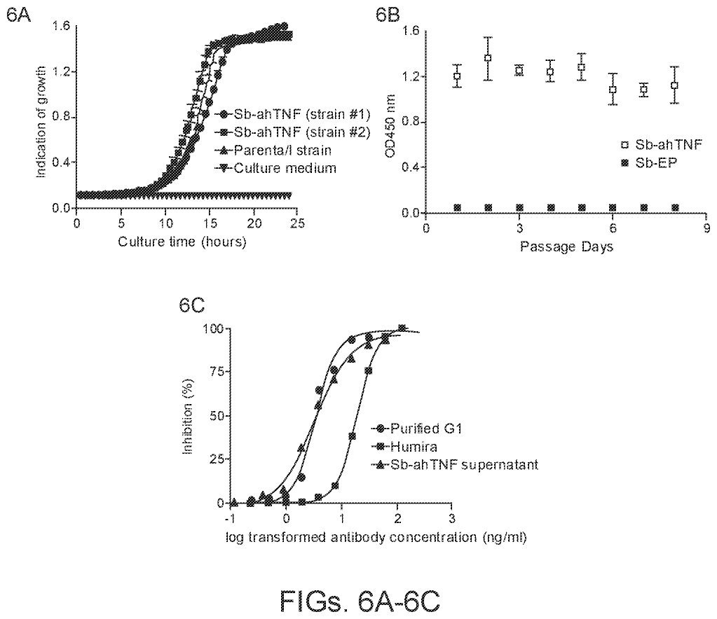

To construct engineered yeast secreting anti-human TNF-α antibody, the expression cassette of ahTNF (VHH-Fc fusion) was optimized and inserted into the yeast genome using the same strategy by which we generated other therapeutic leads. The resultant yeast Sb-ahTNF strains (designated as FZ006) had the same growth rate as the parental strain (FIG. 6A) and stably expressed ahTNF over nearly 100-generations (8 days) of culture (FIG. 6B). A representative 24-hr culture supernatant collected on the day 7 passages had 1.2 ug/ml of ahTNF. The ahTNF in the yeast supernatant had a similar neutralizing activity as the purified G1 antibody from HEK293 culture, which is significantly more potent than Humira in neutralizing cytotoxicity of human TNF-α on cultured L929 cells (FIG. 6C). These data demonstrated that FZ006 stably expresses a fully functional anti-human TNF-α antibody.

### Example 5: Experimental Methods

### Sequencing

Whole-genome sequencing can be carried out using PacBio P6C4 chemistry and Illumina HiSeq-1000 high-throughput sequencing technology followed by standard assembly and annotation procedures, with particular attention to loci where cassettes, for example ahTNF cassettes, were inserted or sequencing of site-specific insertion can be completed. Growth of Sb Strains

Growth kinetics of FZ006 and related strains (e.g., engineered strains of Saccharomyces yeast described herein), as well as stability, homogeneity, and temperature resistance can be characterized. Knowledge of these factors can be used for upstream scale-up processes for making commercial preparations of the disclosed products. Media was chemically defined and free from animal-derived content. Synthetic yeast nitrogen base (YNB) with glucose and a yeast synthetic drop-out medium supplement was used. Both Sb-amTNF and Sb-ahTNF, for example, grow well in this media as wild type yeast.

Daily passage of engineered strains of Saccharomyces yeast described herein, including, for example, FZ006 in YNB media was performed for 10 days, or about 100-120 generations of engineered strains of Saccharomyces yeast described herein, including, for example, FZ006, and growth was monitored by measuring optical density of the culture and viability of the yeast. Temperature resistance experiments were conducted by culturing engineered strains of Saccharomyces yeast described herein, including, for example, FZ006, and a parental strain for 4 hours at different temperatures (30, 37, 40, and 45° C.). After culture conditions are optimized, high-density culturing in a BioFlo 320 will be conducted, allowing scaling of process development as needed. Engineered strains of Saccharomyces yeast described herein, including, for example, FZ006 will be sampled at different culture densities during fermentation and monitored for stability and homogeneity by measuring growth and secretion (e.g., ahTNF secretion) of randomly-picked clones.

### Gastric, Intestinal, and Bile Salts Stress Conditions

Stress resistance against the intestinal environment was evaluated following previously described protocols for some leads, such as FZ002 and FZ006. Cultures of yeast cells, either engineered yeast or wild type, (OD=0.8-1.2) were harvested by centrifugation and washed with distilled water before incubation at 37° C. for 1 hour in: 1) a simulated gastric environment constituted by an aqueous solution containing 3 g/L pepsin (3200-4500 U/mg) and 5 g/L NaCl, pH 2.0; 2) a simulated intestinal environment aqueous solution containing 1 g/L pancreatin (903 U/mg) and 5 g/L NaCl, pH 8.0; and 3) YPD (Yeast extract, Peptone, Dextrose) liquid medium supplemented with 0.1% of a mixture of bile salts (50% sodium cholate and 50% of sodium deoxycholate). For experiments on solid medium, cells grown exponentially in liquid YPD (4%) were diluted to an OD of 1.0 and 5 μL of this dilution will be used to inoculate solid YPD medium (4%) supplemented with different concentrations of bile salts. Colonies were visualized after 48 hours at 37° C. The parameters generated here will be useful for validating the growth of the engineered lead yeast strains in future CMC development.

### Antimicrobial Drug Sensitivity

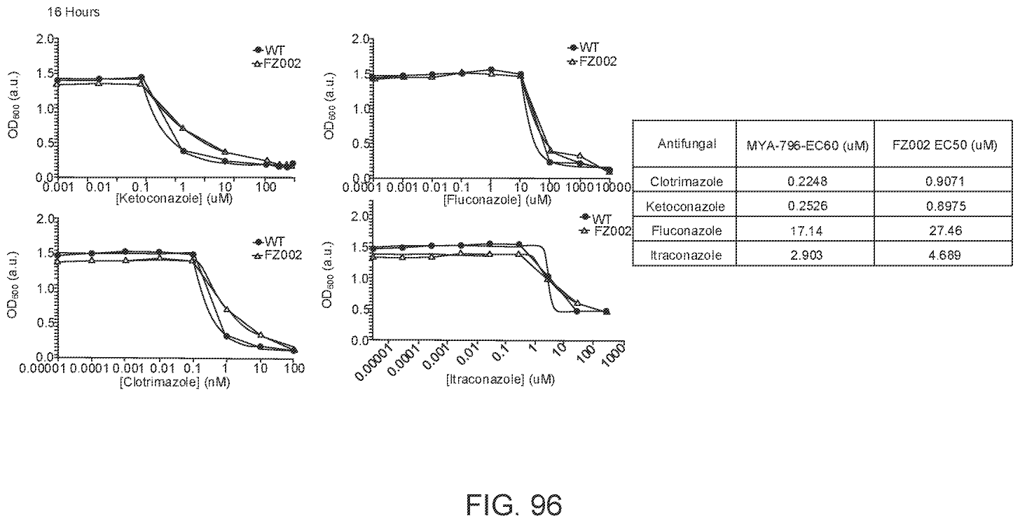

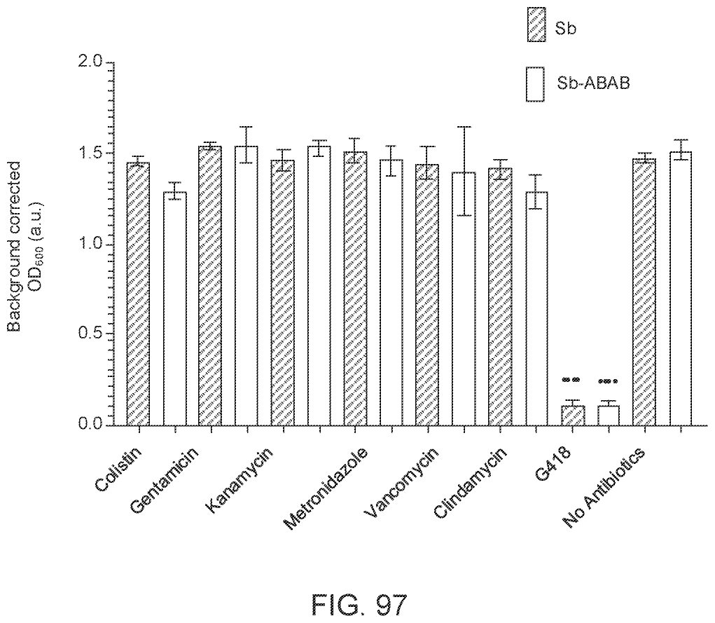

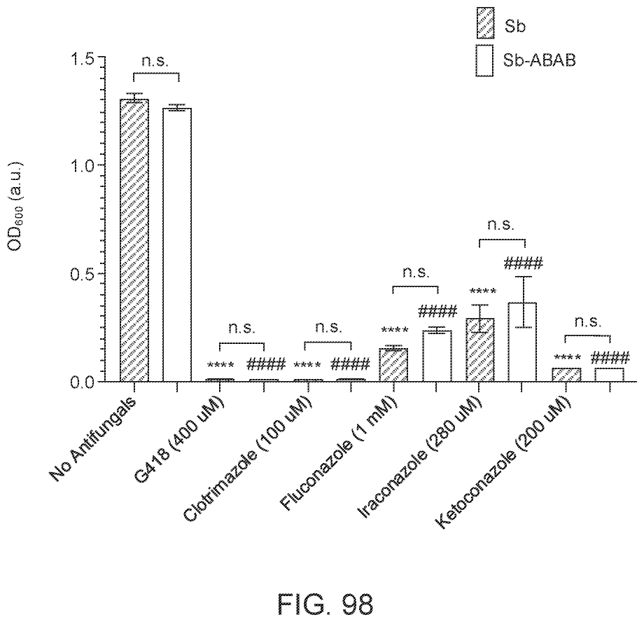

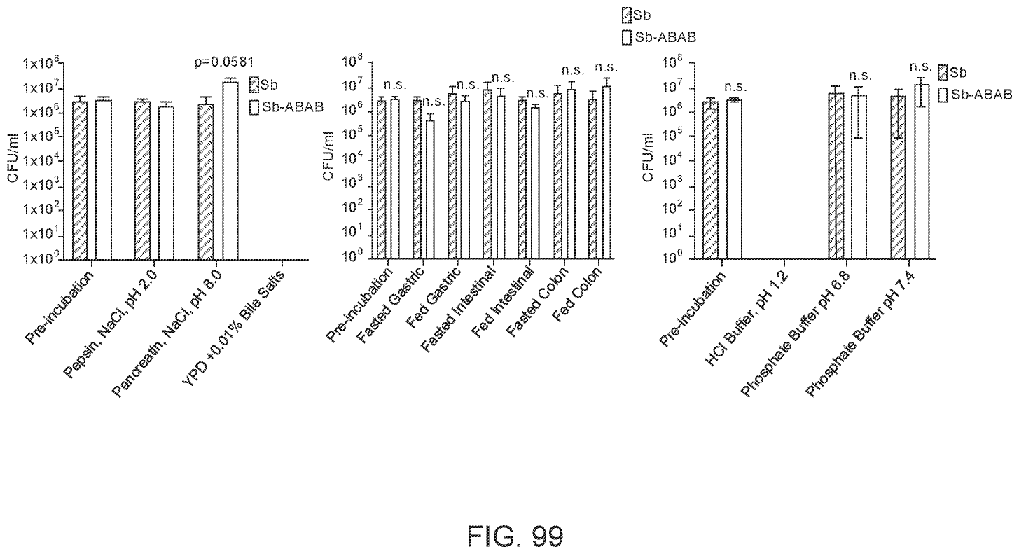

Exemplary Sb-ABAB and FZ002 were tested for sensitivity against panels of antifungal and antibacterial agents (FIG. 96-99) as described previously. This important QC assay will not only be useful for confirming the identity of engineered yeast strains, including, for example, FZ006 and FZ002, but also help to determine potential bacterial contamination. Four antifungal agents (clotrimazole, fluconazole, itraconazole, and ketoconazole) were tested (FIG. 96). A panel of anti-bacterial agents have been tested for another exemplary therapeutic lead FZ002 and Sb-ABAB against C. difficile (FIG. 1E and FIG. 97). Additional agents including amoxicillin, ampicillin, bacitracin, ceftriaxone, chloramphenicol, cloxacillin, enrofloxacin, erythromycin, methicillin, penicillin G, streptomycin, and tetracycline from Sigma Aldrich can be assessed. The parental strain was tested in parallel and no changes in the antibiotic resistance profile was identified as reported previously (FIG. 1E).

### Example 6: Development of Analytical Methods

### Identity of FZ006 and Additional Exemplary Engineered Saccharomyces Yeast Strains

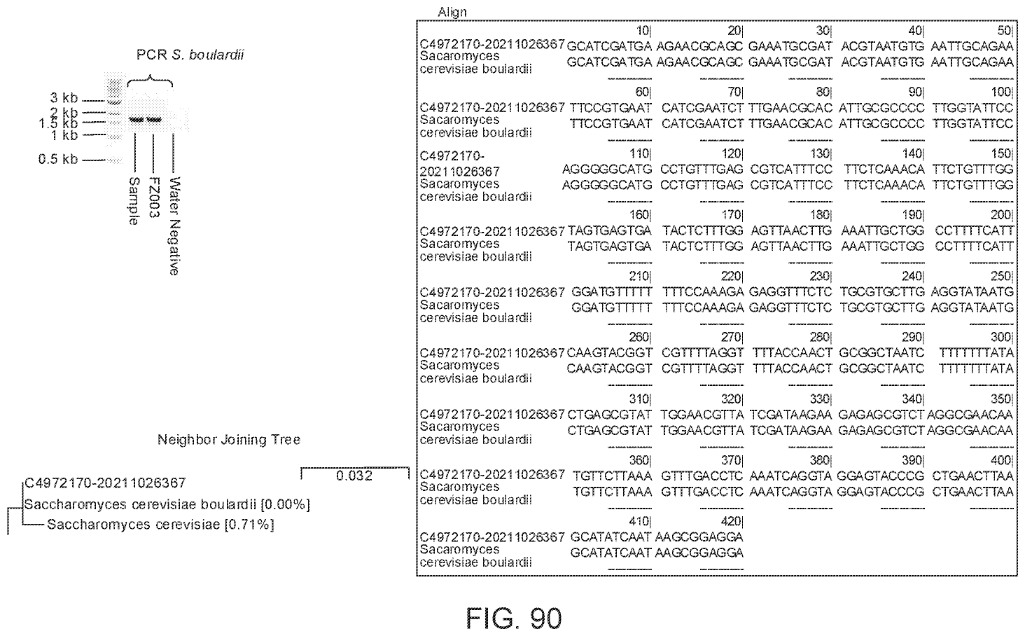

The whole genome sequence of FZ006 and other exemplary engineered strains of Saccharomyces yeast described herein sets the foundation of its identity. In addition, the biochemical/phenotypical characteristics, can be used to validate the identity of FZ006 and other exemplary engineered strains of Saccharomyces yeast described herein. To develop analytic methods to assure the identity of the engineered Saccharomyces yeast strains described herein (including, e.g., FZ006) during process development and manufacturing, the primers used to amplify the rDNA internal transcribed spacer region (ITS) was used (FIG. 90). In addition, primers for amplifying 18S and 26S ribosomal RNA genes (on ATCC product sheet), and the ahTNF cassette and flanking sequences can be also synthesized. These primers can be used to amplify the corresponding genes for sequencing. Besides sequence information, morphology and growth kinetics, the stress resistance pattern and antibiotic/antifungal resistance profiles described above can be used to determine the identity of the engineered Saccharomyces yeast strains, including, for example, FZ006.

### Strength of FZ006 and Additional Exemplary Engineered Saccharomyces Yeast Strains

The strength of exemplary engineered Saccharomyces yeast strains described herein, including, for example, FZ006 are determined by, for example, two factors, the number of viable yeast and their production of GOI (e.g., a nucleic acid encoding, for example, ahTNF). The viability of the engineered Saccharomyces yeast strains described herein, including for example, FZ006, are determined by staining with vital dyes under an automated cell counter and counting colony-forming units (CFU) on agar plates. The production of therapeutic polypeptide(s) (e.g., ahTNF) is measured by the amount of antibody secretion in picograms by one yeast in 24 hours (picogram per cell per day, PCD) under a given culture condition. Synthetic YNB culture media will be used, transferring 0.1 OD of an overnight culture to 50 mL of fresh media to culture the exemplary engineered Saccharomyces yeast strains described herein, including, for example, FZ006, until a later log phase of OD10. The culture supernatants can be collected every 3 hours over a 24-hour period, and the amount of therapeutic polypeptide(s) production, including, for example ahTNF, can be determined by ELISA.

### Quality and Purity

Quality and purity can be determined primarily during the manufacturing process. The parameters that can be used include, for example, sterility, physical appearance, pH of the cultures, endotoxin levels, bioburden, viability, purity, and identity. To ensure the quality and purity of exemplary engineered Saccharomyces yeast strains described herein, including, for example, FZ006, all chemicals used for culture can be United States Pharmacopoeia (USP) grade or food grade to generate a cell bank. Cell banks can be developed following, for example, CVD standard protocol. Exemplary engineered Saccharomyces yeast strains described herein, including, for example, FZ006, can be grown on agar plates with non-selective chemically defined media. Cultures can be examined to ensure uniformity and morphology of colony type before harvest and resuspension in media supplemented with 20% of glycerol. Frozen vials (100) can be prepared and stored in a dedicated −80° C. freezer. Three vials can be tested for purity based on the tests described in the USP. Should any microbial contamination be identified, the bank can be discarded and a new cell bank can be generated.

### Example 7: Pharmacokinetic and Safety Profiles of Exemplary Engineered Saccharomyces Yeast Strains, Including, for Example, FZ006 and FZ002

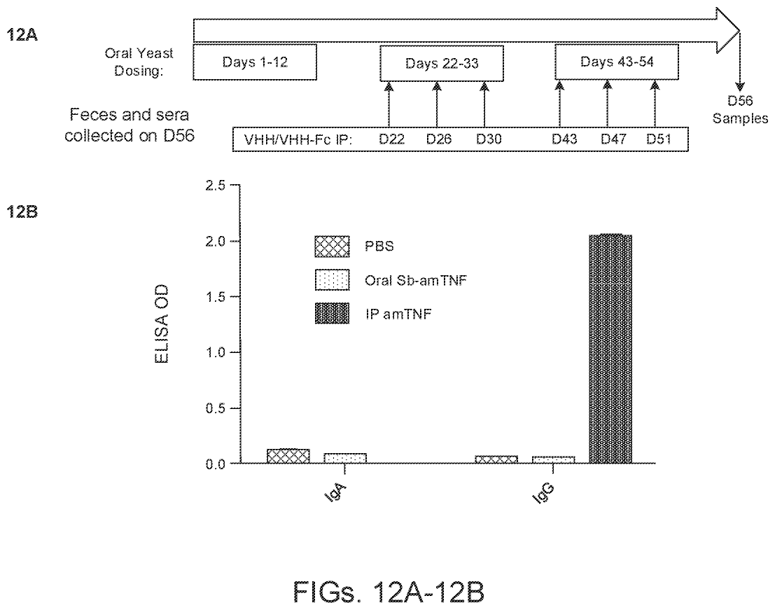

Oral Sb-amTNF prevented mice from intestinal tissue damage and inflammation associated DSS-colitis, suggesting that live Sb-amTNF was distributed to the colons and produced a therapeutic level of amTNF. Pharmacokinetic (PK) properties of FZ006, Sb-ABAB, and FZ002 were evaluated by determining the yeast gastrointestinal distribution, attachment or excretion, secretion of ahTNF or ABAB as well as yeast and antibody or ABAB shedding (FIG. 2A-2F, Chen et al, Sci. Transl. Med 2020). Potential anti-drug responses was determined by evaluating host anti-amTNF IgAs and IgGs after oral long-lasting FZ006m administration (FIG. 12A-12B). Furthermore, the ability of, for example, intestinal FZ006, to translocate to circulation can be determined in immunosuppressed mice.

### Pharmacokinetics of Exemplary Engineered Saccharomyces Yeast Strains, Including, for Example, FZ006 and FZ002

A preliminary PK study indicated that oral gavage of FZ002 led to mouse shedding live yeast and secretion of ABAB in mouse intestines (FIGS. 2A-2E). Oral Sb-amTNF also led to a significant reduction of colitis symptoms by ameliorating intestinal inflammation (FIGS. 4A-4G). These data demonstrated that live yeast reached to the mouse lower intestines after oral administration and secreted a therapeutic dose of neutralizing antibodies. Yeast intestinal distribution can be systematically investigated alongside production of antibody and excretion. Mice can be administered with 10⁸, 10⁹, and 10¹⁰ CFU/dose/day for 7 days. Including the dose of 10¹⁰ CFU allows us to assess mouse tolerability of oral FZ006 at the highest dose possibly achieved through gavage needle. Feces can be collected from each mouse before oral yeast administration (for background) and then daily until 7 days after withdrawal of yeast treatment). In each group, 6 mice (3 males and 3 females) can be sacrificed on day 0, 1, 3, 6, 9, and 14, and the following tissues/organs can be collected: small and large intestine (different segments and lavages from ileum, cecum, and colon), mesenteric lymph nodes (MLN), liver, spleen, and blood samples. All samples can be analyzed based on assays previously described. Mouse feces and intestinal lavages can be diluted in PBS and plated on sabouraud agar plates that can be selected for growth of Sb to determine CFU among the groups, as previously described. Sb colonies can be randomly picked up and put into liquid culture to measure the secretion (e.g., secretion of ahTNF) for validating the stable antibody production. To assess absorption and potential systemic dissemination of live yeast, MLN, spleen, and liver tissues can be weighted, homogenized and filtered. Then, along with blood samples, these tissues can be plated on sabouraud agar to identify colonies; if any colonies are observed, the yeast can be analyzed with PCR for ahTNF genes as described previously. For assessing the absorption, distribution, and excretion of ahTNF, feces, intestinal contents, segments of intestinal tissue, MLN, liver tissue lysates, and blood samples can be diluted in protease-containing PBS, and the amounts of, for example, ahTNF, can be quantified via standard ELISA.

Host Anti-ahTNF Responses after Exemplary Oral FZ006 Administration

Whether oral FZ006 induces mucosal IgA and systemic IgG against secreted ahTNF during the multiple courses of FZ006 treatments can be determined. A dose of oral FZ006 that shows the highest intestinal secretion of ahTNF in PK studies and administrate for total 21 doses is chosen, so that whether long-lasting oral FZ006 induces any potential anti-drug response that may reduce FZ006 therapeutic potential can be assessed. Groups of mice (n=10, 5 males and 5 females) can be orally administered with FZ006 daily for 7 days (considered as one oral FZ006 treatment cycle) followed by a 14-day resting period. This can repeat 3 times with 3 oral FZ006 cycles (21 doses of FZ006 total). Control groups can be administered with the same amount of Sb-EP. A control of systemically injected purified ahTNF (i.v. injection of ahTNF at 10 mg/kg) can also be included. Mouse feces and blood samples can be collected on day 0 before the first oral dose of yeast and then on the day of 7 days after each oral FZ006 cycle. Ten days after the final FZ006 dosing, mice can be sacrificed, and intestinal lavages can be collected. Specific IgA from feces and intestinal lavages, as well as the serum IgG against ahTNF, can be measured by ELISA using plates coated with purified anti-human TNF antibody. Should anti-ahTNF antibody responses be detected, neutralization of ahTNF activity by host anti-ahTNF antibodies was determined using cell-based bioassay (FIG. 5A).

Determination of Sb Systemic Translocation in Immunosuppressed Individuals after Exemplary Oral FZ006 Administration

Sb has been used as a probiotic since the 1950s and investigated extensively in clinical trials with an excellent safety profile. However, rare cases of fungemia were identified, mostly in those individuals with severe comorbidities and have central venous catheters in intensive care units. In animals, 10¹⁰ CFU, the highest possible dose achieved via gavage needles, of engineered yeast have been administered without observing any adverse effects. Whether, for example, the oral FZ006 leaks into systemic circulation in immunosuppressed mice can be determined. Mice (n=10, 5 males and 5 females) can be injected ip with 100 mg/kg cyclophosphamide on alternate days (0, 2, 4, 6, and 8) for a total of five injections to induce immunosuppression. On day 3, mice are gavaged with 10¹⁰ CFU of FZ006 per day for 7 consecutive days. Control groups of mice can be given with PBS or Sb-EP. Mice are sacrificed on day 10; blood, MLN, spleen, and liver tissues can be collected; and the presence of live yeast is determined. In addition, the samples are plated on BHI agar plates to determine bacterial counts, which allow us to determine whether, for example, FZ006 or Sb-EP treatment affects bacterial translocation. Previous studies found that probiotic Sb yeast helped maintaining gut barrier function and reduced bacterial translocation from the intestinal lumen to blood circulation. Oral FZ006 and other exemplary engineered strains of Saccharomyces yeast described herein may exhibit additional beneficial effects on gut barrier function by reducing intestinal inflammation through secretion of therapeutic polypeptide(s) (e.g., of anti-human TNF-α) when administrated in humans.

### Further Experiments and Expected Results

Characterization of other products (e.g., engineered strains of Saccharomyces yeast described herein, for example FZ006) can be conducted. When analyzing the PK results, the following can be monitored: 1) when mice start to shed, for example, FZ006 after oral dosing and how long mice continue shedding the yeasts after the final dosing; 2) when the yeast shedding becomes stable; 3) the intestinal location of live yeast; 4) the relationship between oral doses and the intestinal therapeutic polypeptide(s) (e.g., ahTNF) production; and 5) the intestinal distribution of therapeutic polypeptide(s), (e.g., ahTNF) and their systemic dissemination. These studies can lead to useful information regarding the kinetics of intestinal engineered strains of Saccharomyces yeast described herein, for example, FZ006, and the secretion and distribution of therapeutic polypeptide(s) (e.g., ahTNF). It is expected that there can be undetectable systemic dissemination of live yeast and absorption of therapeutic polypeptide(s) (e.g., ahTNF) in normal mice. Without wishing to be bound by any one theory, due to the complicated intestinal environment and constitutive production of therapeutic polypeptide(s) (e.g., ahTNF) by engineered strains of Saccharomyces yeast (e.g., FZ006), the intestinal metabolism of therapeutic polypeptide(s) (e.g., ahTNF) can be difficult to measure precisely. However, the PK study can allow assessment of the steady intestinal levels of therapeutic polypeptide(s) (e.g., ahTNF) during oral yeast administration. Although the intestinal therapeutic polypeptide(s) (e.g., ahTNF) levels may fluctuate at different pathophysiological conditions, the ADME/PK studies and animal efficacy studies can provide knowledge regarding the oral doses of engineered strains of Saccharomyces yeast (e.g., FZ006) that lead to therapeutic levels of intestinal therapeutic polypeptide(s) (e.g., anti-TNF antibody). This information can be important for determining future doses and schedules for oral engineered strains of Saccharomyces yeast (e.g., FZ006) treatment in clinical trials.

VHHs are generally non-immunogenic, even with systemic injections. Exemplary therapeutic polypeptide(s), ahTNF, delivered to intestines via probiotic yeasts should be at even lower risk of inducing an anti-drug response. Should a detectable level of IgA or IgG against, for example, ahTNF be induced with 21 doses of, for example, FZ006 over an 8-week period, it can be determined whether the magnitude of anti-VHHs responses is dose- and time-dependent, so that the degree of anti-drug response may be minimized by optimizing the dosing and schedule of engineered strains of Saccharomyces yeast, including, FZ006. Sb showed protective effects in immunosuppressed mice and prevented bacterium and pathogen translocation to circulation. It is possible that low counts of Sb are detected systemically in immunosuppressed mice when high doses of Sb are administered; in this case, mice may experience adverse effects, which will be monitored closely during the administration of engineered strains of Saccharomyces yeast, including, FZ006 or Sb-EP. Any adverse effects can be correlated with the number of Sb in circulation; if such a correlation is established and significant unfavorable effects occur, experiments can be repeated to evaluate whether systemic fluconazole can efficiently eliminate yeast from the bloodstream and reduce the symptoms. In such a case, a serum-sensitive yeast can be engineered for therapeutic strains so that the engineered yeast rapidly dies upon disseminating into the bloodstream.

### Example 8: Preclinical Efficacy Evaluation of FZ006 in Human TNF-α Transgenic Mice

Since FZ006 secretes ahTNF (VHH-Fc fusion) that only neutralizes human TNF-α, its therapeutic efficacy can be validated using human TNF-α transgenic mice. IBD is a complicated disease and no single animal disease model can recapitulate the disease pathogenesis in humans. Therefore, both DSS- and adoptive T cell transfer-induced colitis can be used as models, since these are the two most widely used animal colitis models to mimic some aspects of human UC pathogenesis.

The DSS-induced colitis model is one of the most widely used model types in preclinical studies due to its relatively straightforward protocol. The colitis results from the damaging effect that DSS, a negatively charged sulfated polysaccharide, has on epithelial cells. Inflammation, which is limited to the colon, is induced mainly by proinflammatory cytokines, i.e., TNF-α, produced by innate immune cells, and is largely characterized by ulcers and granulocyte infiltration. The adoptive T cell transfer model is used to induce chronic colonic inflammation, which resembles some key aspects of human IBD. The model is developed after the adoptive transfer of naive CD4⁺ T cells (i.e., CD4+CD45RBhi) from donor mice into syngeneic immunodeficient recipients, which causes colonic inflammation predominantly around ten weeks after cell transfer. This inflammation has been attributed to the lack of Tregs cells in the naive T cell population, and it is consistent with observations in IBD patients that show intestinal tissue with an influx of CD4+CD45RB^{hi} T cells that produce less IL-10, IL-4 and more proinflammatory cytokines, such as TNF-α, than CD4⁺CD45RB^{low} T cells compared to control patients. Both DSS and adoptive T cell transfer models have been widely used to evaluate the therapeutic efficacy of anti-TNF-α agents against UC because the inflammation is predominantly localized to the colon and TNF-α plays a major role in the immunopathologies of the colitis. Therefore, these two models can be used to assess the therapeutic efficacy of FZ006.

### Efficacy of Oral FZ006 Against DSS-Induced Colitis in Human TNF-α Transgenic Mice

Since FZ006 secretes anti-human TNF-α VHH-Fc that only neutralizes human TNF-α, we can validate its therapeutic efficacy using human TNF-α transgenic Tg1006 mice. The Tg1006 mTNFKO mice (mTNFα: −/−, hTNFα: +/−; from Taconic) expressing only human TNF-α can spontaneously develop severe arthritis in both forepaws and hind paws in around 20 weeks of age due to overexpression of human TNF-α. A previously published protocol can be followed to induce both DSS acute and chronic colitis in Tg1006 mice. In an acute colitis model, mice are fed with 3% (wt/vol) DSS in drinking water for 7 days. Long-lasting chronic colitis can be induced by three cycles of 2% DSS (8 days of DSS induction, 14 days of water). Since these Tg mice overexpress human TNF-α, we expect they can develop more severe colitis symptoms than normal conventional mice in both acute and chronic models.

To evaluate the therapeutic efficacy of FZ006 against acute colitis, mice can be orally administered with FZ006 (10⁷, 10⁸ or 10⁹ CFU) daily for 11 days, starting on day 3 of DSS treatment. Control mice can be gavaged with water or 10⁹ CFU of Sb-EP. The clinical symptoms, such as body weight, diarrhea, body temperature, and survival can be monitored. In addition to clinical symptoms, experiments can be designed to assess the protection of oral FZ006 against mouse intestinal tissue damage and inflammation. The same day after the final yeast dosing and 7 days after final dosing, 4 mice (2 males and 2 females) are sacrificed, and colon tissues from these mice are collected to assess tissue damages and inflammation via histology. In addition, cytokine levels in the blood and colonic tissues can be assessed using multiplex ELISA as described previously. Besides monitoring disease symptoms, fecal samples and intestinal lavages can be collected to monitor intestinal distribution and shedding of live yeast, as well as the secretion of ahTNF.

For evaluating the therapeutic efficacy of FZ006 against chronic colitis, mice can be gavaged with water, Sb-EP, or FZ006 (10⁷, 10⁸ or 10⁹ CFU) daily from day 3 to 14; day 24 to 35; and day 45 to 56; ranges that cover some DSS treatment and post-treatment periods. Mice can be monitored for clinical symptoms, such as body weight, diarrhea, body temperature, and mouse survival. In addition to clinical symptoms, experiments can be designed to assess the protection of oral FZ006 against mouse intestinal tissue damages and inflammation. The same dosing schedules can be performed on all except 4 mice (2 males and 2 females) from each group, which can be sacrificed on the day of the last oral yeast dose of each cycle. Colon tissues from these mice can be collected for length measurement and gross examination. Intestinal epithelial damages and inflammation can be examined via histology. Proinflammatory cytokines and chemokines in intestinal tissues and blood circulations can be assessed using multiplex ELISA as we described previously. Besides monitoring disease symptoms, fecal samples and intestinal lavages can be collected to monitor intestinal distribution and shedding of live yeast, as well as the secretion of ahTNF. It can be examined whether anti-ahTNF IgGs and IgAs are induced by assessing fecal and blood samples for anti-drug responses as proposed previously. These results can be compared with data generated from previous experiments in order to understand whether host intestinal inflammatory disease alters the PK of FZ006 and anti-drug response.

### Therapeutic Efficacy of FZ006 in an Immune T-Cell Transfer Model Using Rag Mice

In this model, we can first prepare CD4⁺CD45Rb^{hi} T-cells from 8-week-old Tg1006 mice using published protocols. In short, the donor splenocytes are isolated from gently minced spleens through a 70 μm cell strainer. The T-cells are separated from other cells using T-cell EasySep kit (STEMCELL Technologies). CD4⁺CD45Rb^{hi} T-cells are isolated using cell sorter and transferred to sex-matched RAG^{-/-} recipient mice (Jackson Laboratories). Chronic colitis can be fully developed in the transplanted recipient mice 10 weeks later. Control groups are RAG-mice without T cell transfer that do not develop colitis.

Based on previous observations, the inflammatory disease occurs predominantly at around ten weeks after T cell transfer. When diarrhea occurs at approximately 10 weeks after T-cell injection, mice can be orally gavaged daily with FZ006 for 14 days. The dose of FZ006 that exhibits the best protection in the DSS colitis model as determined previously can be used. Control groups can be gavaged with PBS or the same dose of Sb-EP. Disease symptoms are closely monitored during and after oral yeast treatment. Should the diarrhea occur again after the cessation of FZ006 treatment, another course of oral FZ006 treatment can be started. Up to three episodes of treatment courses can be performed for chronic DSS-colitis in order to evaluate whether oral FZ006 can reduce the severity as well as duration of colitis in T cell-transferred mice when compared to control treatments. In addition to monitoring disease symptoms, 4 mice from each group can be sacrificed at the end of first episode of FZ006 treatment and intestinal tissues can be collected to assess mucosal protection as described previously.

### Expected Results

Sb-amTNF protected conventional mice from DSS- and C. difficile-induced severe colitis, suggesting that the yeast strain expressed a therapeutic dose of anti-mouse TNF-α in the intestines. It is expected that oral FZ006 can exhibit a similar PK property as Sb-amTNF and express anti-human TNF-α in mouse intestine; thus, the human-specific FZ006 should significantly reduce DSS-colitis in human TNF-expressing Tg1006 transgenic mice. It is also expected that oral FZ006 can protect mice from colitis in the adoptive T cell transfer model. Since the transgenic mice only express human TNF-α, the TNF-α overexpression may drive significantly more severe colitis than conventional mice. In such a case, significant protection was observed because oral Sb-amTNF provided significant protection against severe colitis induced by DSS together with C. difficile (FIGS. 4A-4G). Given high affinity and potent neutralizing activity of G1, it may even be the case that significant efficacy of oral FZ006 at lower doses of 10⁷ or 10⁸ CFU is observed against the intestinal inflammation, reducing clinical symptoms, intestinal tissue damages, and inflammation, when compared to control groups. One potential problem is that the RAG^{-/-} mice adoptively transferred with CD4⁺CD45Rb^{hi} T-cells overexpressing human TNF-α may experience onset of intestinal inflammation and disease symptoms much earlier than usually observed. Mouse disease symptoms can be monitored as early as 6 weeks after T cell transfer. Typically, however, the adoptive T cell transfer model is highly reproducible, and the time window of disease onset should be easily identified. Based on these experiments, an oral yeast dosing schedule for testing FZ006 therapeutic efficacy can be designed.

In testing therapeutic efficacy, an oral dose of yeast at up to 109 CFU per day can be used. In human studies, up to 2 g of dried Sb per day has been administered in capsules (4×10¹⁰) per day. Given the body size difference between mouse and human, 10⁹ CFU of oral yeast daily in mice is a significantly higher dose. As shown in FIG. 2C, mice only shed around 10⁶ to 10⁷ CFU of live yeast/g feces after daily oral gavage of yeast at 10¹⁰ CFU, suggesting that the majority of gavaged yeast in solution did not survive and were presumably killed by stomach acidity. Therefore, although a high oral dose in mice is used in these experiment, the amount of live yeast reaching the colon is relatively low. This problem can be mitigated by future formulation development in which the live yeast in dry powder can be encapsulated with enteric-coated capsules, protecting live yeast from stomach acidity and substantially improving therapeutic potential.

### Statistical Analysis

Differences in Sb shedding and anti-TNF-α antibody levels can be assessed using the Fisher's exact test. The Kaplan Meier method and Wilcoxon and log-rank tests can be used to compare survival times and rates. Comparisons of clinical symptoms weight loss and diarrhea can be made using analysis of variance and co-variance and the Duncan multiple comparison method. Data are presented as mean±SEM or SD. The protective effect of FZ006 can be analyzed with either a two-tailed Mann-Whitney test or unpaired t test. All statistical analyses can be performed using the GraphPad Prism v.6, and data can be considered significant at a P value of less than 0.05.

### Selection of Animals

The impact of sex on the response to engineered yeast in colitis is unknown. To reduce biological variables, both male and female animals can be used. For mice, 6- to 10-week-old C57BL/6J with a weight of 16-22 g can be used. The Tg1006 breeders with C57BL background will be from Taconic. Each group can have 10-24 animals to minimize experimental errors from differences among the mice. A description of animal usage, procedure, and power and statistical analysis is provided above.

### Example 9: Additional Comorbidity Model of CDI with Adoptive T-Cell Transfer Colitis

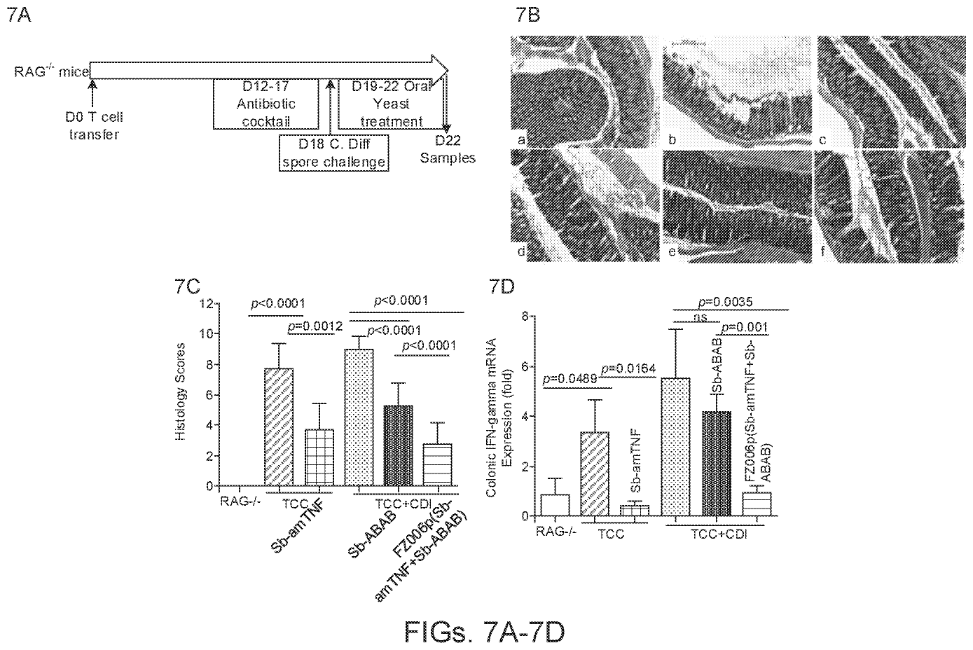

The method of T-cell transfer colitis (TCC) was described previously. CD4⁺CD45Rb^{hi} T-cells from 8-week-old female C57BL/6 donor mice were prepared and injected into the female RAG/recipient mice on day 0 (FIG. 7A). Moderate colitis developed at three weeks after T-cell injection (TCC alone group, FIGS. 7B-7D). CDI was induced by antibiotic cocktail treatment followed with oral gavage of C. difficile spores (VP 110463, 10⁴/mouse, FIG. 7A), and comorbid mice developed severe colitis (TCC+CDI group, FIGS. 7B-7D). For yeast treatment, groups of mice were orally administrated with Sb-amTNF (109 CFU/day/mouse), Sb-ABAB (109 CFU/day/mouse), or FZ006p (Sb-amTNF+Sb-ABAB, 5×10⁸ CFU each/day/mouse) from day 19 to 22 (FIG. 7A). The mice were sacrificed on day 22, and colonic tissues were collected for histology and quantitative real-time RT-PCR analysis of interferon gamma (IFN-7) mRNA expression. The sections were H&E stained, and microphotographs were recorded at multiple locations and scored blindly by two investigators.

These new preliminary data show that oral Sb-amTNF reduced colonic tissue damage (FIGS. 7B-7C) and inflammation (FIG. 7A) in TCC mice. Although oral Sb-ABAB alone did not significantly reduce the expression of IFN-γ mRNA in colonic tissues (FIG. 7D), its combination with Sb-amTNF substantially reduced colonic inflammation and tissue damage in comorbid mice (FIGS. 7B-7D), indicating the synergistic effects of the combinational therapy. These data are consistent with those presented in FIGS. 6A-6C that oral FZ006p significantly protected mice against comorbidity of CDI and DSS colitis.

### Example 10: Development of Yeast Strains and Applications

The present example demonstrates exemplary methods for the development of yeast strains. ° MYA-796 is a parental diploid strain from ATCC, and is officially designated as Saccharomyces cerevisiae Meyen ex. E. C. Hansen (also known as Saccharomyces boulardii or Saccharomyces cerevisiae var. boulardii), a whole genome sequencing strain. Genomic information can be extracted from GenBank JRHY00000000 Saccharomyces sp. ‘boulardii’ strain ATCC MYA-796, whole genome shotgun sequencing project.

^{d}MYA-796 (ura3−/−) is an uracil auxotrophic strain generated on ° MYA-796 parental strain due to the absence of functional ura3 genes. Ura3 is a gene on chromosome V in MYA-796. Ura3 encodes Orotidine 5′-phosphate decarboxylase (ODCase), which is an enzyme that catalyzes one reaction in the synthesis of pyrimidine ribonucleotides (a component of RNA). Ura3 allows for both positive and negative selection, making the dMYA-796 (ura3−/−) a powerful tool for genome manipulation. This strain was genetically modified using homologous recombination and combined with loxp-loop out strategy. Double-knock out of Ura3 genes can be generated by one or two steps of homologous recombination with two different antibiotic cassettes which are flanked with 5-HAL-ura3 (40 bp) and 6-HAL-ura3 (40 bp) on the outside and LoxP sites on inner side to replace URA3 genes. Double positive strains with both antibiotics resistances were screened, then further Ura3 counter selection (negative selection) with 5′-FOA for URA3 double knockout phenotype was completed. PCR-based site-specific deletion of genomic Ura3 genes and insertion of antibiotic marker cassettes was completed using Cre-LoxP to delete both antibiotic cassettes. LoxP (locus of X(cross)-over in P1) sites are 34-base pair long recognition sequences (ATAACTTCGTATAGCATACATTATACGAAGTTAT; SEQ ID NO: 23) consisting of two 13-bp long palindromic repeats separated by an 8-bp long asymmetric core spacer sequence. After successfully transforming a Cre-plasmid in, the loop out of the antibiotics marker cassettes flanked with LoxP sites was induced. Cre-plasmid positive clones were selected, then naturally lose Cre-plasmid by culturing the clones without selection-stress. Screening the clones losing the original antibiotics resistance, losing Cre-plasmid selection marker, but positive for 5′-FOA counter selection was conducted. PCR and sequencing was conducted to confirm the deletion of antibiotics cassette and LoxP scars. CRISPR/Cas9 was also used to indicative genes and create desirable variants. Exemplary variants include ^{e}MYA-796 (ura3−/−, gap1−/−), ^{f}MYA-796 (ura3−/−, gap1−/−, pep4−/−), ^{g}MYA-796 (ura3−/−, gap1−/−, prb1−/−), ^{h}MYA-796 (ura3−/−, gap1−/−, thr1−/−), ¹MYA-796 (ura3−/−, gap1−/−, thr4−/−).

### Mutations in Protease Genes for Enhanced, Stable Protein or Polypeptide(s) Expression and Secretion

In addition to maximizing expression of therapeutic polypeptide(s) by integrating at highly expressed regions of the yeast genome with cassettes that utilize the cellular machinery of these loci, protein or polypeptide (e.g., therapeutic polypeptide(s)) expression, secretion, and stability was also increased by deleting protease-encoding genes. Pep4, the yeast proteinase A Pep4 is a vacuolar aspartyl hydrolase that not only degrades proteins or polypeptides in the vacuole but also activates additional vacuolar proteases, including Prc1 (carboxypeptidase Y), Prb1 (proteinase B), and Lap4 (aminopeptidase I), was targeted. Without wishing to be bound by any one theory, by deleting pep4, it was hypothesized to increase the secretion of proteins or polypeptides of interest (e.g., therapeutic polypeptide(s)) by removing or preventing the maturation of all of these degradative enzymes.

To test the efficacy of this approach, pep4 was deleted from an S. boulardii strain that secretes a neutralizing antibody for C. difficile toxins A and B (Sb-ABAB). Using a chemical transformation protocol, Sb-ABAB cells were transformed with PCR-amplified cassettes that conferred resistance to either geneticin or phleomycin. The homology sequences for these cassettes were designed to completely remove the pep4 open reading frame and a short sequence of the gene promoter near the start codon. Each resistance gene was surrounded by two LoxP sites to allow for later removal by transformation of a Cre recombinase plasmid; a Cre recombinase plasmid was designed that contained the selectable and counter-selectable gene, gap1, since an exemplified engineered strain of Saccharomyces yeast is a gap1 mutant. Cells with successful pep4-targeting integrations were selected on rich medium containing geneticin and phleomycin, and pep4 mutant clones were confirmed by colony PCR. Sb-ABAB pep4 Clones 10 and 6b were confirmed to be pep4 mutants.

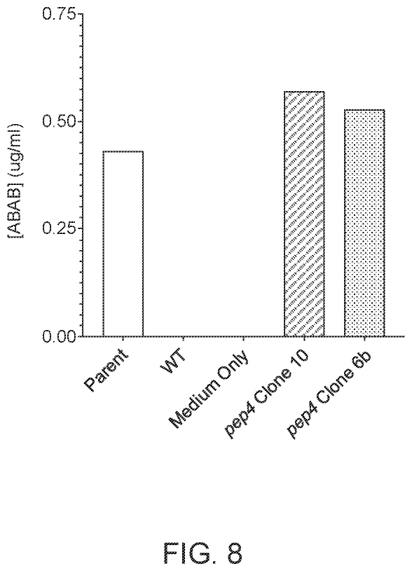

These protease null clones were cultured in rich YPD medium for 24 hours alongside the parent Sb-ABAB cells, as well as wild-type (WT) yeast that do not secrete ABAB antibodies and un-inoculated medium as negative controls. Supernatants were collected from each culture and the concentration of ABAB in the samples was determined using a designed ELISA protocol, followed by comparison to concentration reference standards included on the ELISA plate. The designed ELISA involves coating plates with C. difficile toxin B, blocking with milk, incubating the plate with milk-diluted supernatants, and treatment with HRP conjugated antibodies targeting the therapeutic polypeptide(s). The results showed that deletion of pep4 led to an increase in the concentration of ABAB in the supernatant by approximately 42% (FIG. 8).

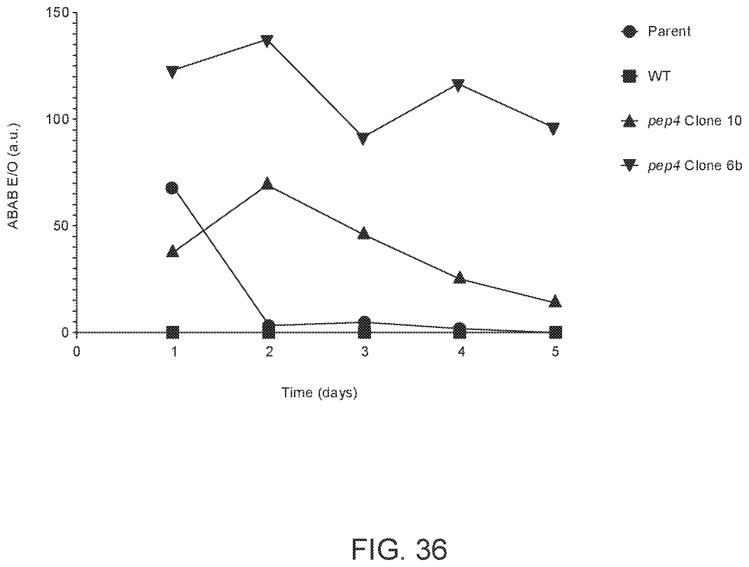

As intracellular proteases can degrade proteins or polypeptides (e.g., therapeutic polypeptide(s)) in the extracellular environment due either to secretion or release after cell death, the loss of pep4 and the inhibition of enzymes it activates was tested to determine if increases in the stability of protein or polypeptides of interest over longer time periods was observed. Sb-ABAB pep4 null clones were cultured alongside control strains in unbuffered, minimal medium that becomes acidified during yeast growth; previously, it was observed that protein or polypeptide stability beyond 24 hours is low in unbuffered medium. Supernatant samples were collected from each culture every 24 hours for 5 days and determined the concentration of ABAB antibodies using the ELISA protocol outlined above. The data showed that the concentration of ABAB was higher and more stable in samples from pep4 mutants (FIG. 36) In addition to minimizing the degradation of therapeutic polypeptide(s) released into the gut during treatment, this approach enables use of S. boulardii cells for high, stable expression and purification of secreted proteins or polypeptides of interest. Due to the success of this approach, introduction of pep4 mutations into other biotherapeutic yeast strains (e.g., engineered strains of Saccharomyces yeast) by such a pipeline and similar strategies to target other proteases, both individually and in combination with other protease deletions will be pursued. These include the direct targeting of, for example, Prc1, Prb1, and Lap4 that are inhibited in the absence of Pep4, as well as the plasma membrane-localized protease, Yps1.

### Mutations in Key Genes for Safer Biologics

The present example demonstrates, among other things, use of mutations in key genes to generate safer biologics. Cases in which orally administered S. boulardii probiotics have led to fungemia in immunocompromised populations have been documented. To create safer live biotherapeutic products, mutations were introduced in threonine biosynthesis genes. Mutation of thr1, an upstream enzyme in the threonine synthesis pathway, can cause serum-sensitivity in S. cerevisiae (Kingsbury & Mccusker, 2010). Without wishing to be bound by any one theory, this sensitivity is understood to be due to the accumulation of a toxic intermediate molecule, homoserine, when the cells are exposed to low-threonine environments, such as serum. The Thr4 enzyme is directly downstream of Thr1 in this pathway and phosphorylates homoserine. thr4 null strains exhibit a very similar phenotypes to thr1 deletion cells, since phosphohomoserine is also a toxic intermediate.

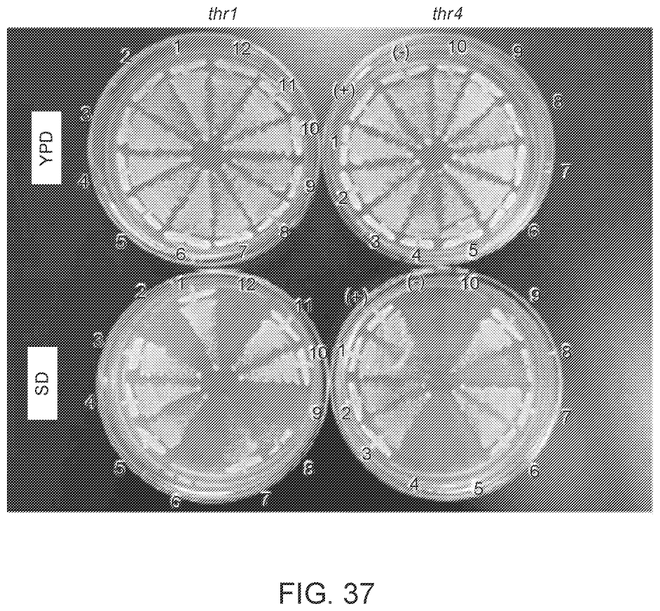

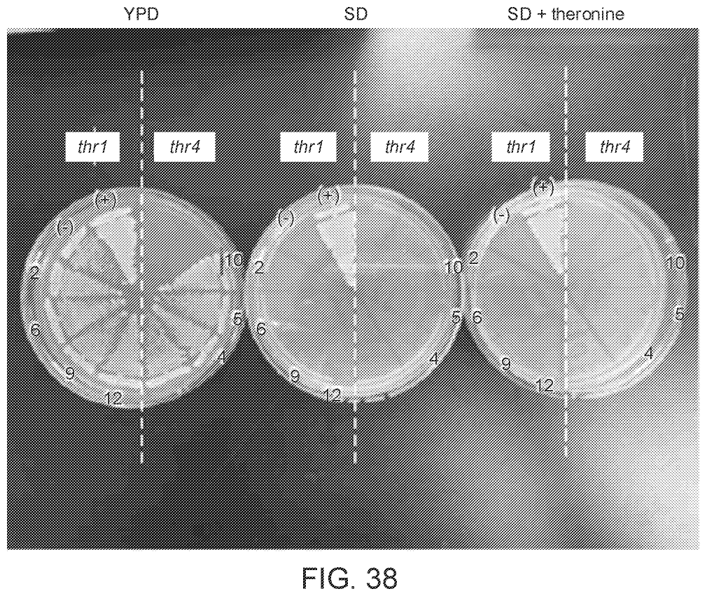

Using the same strategies described for mutating protease genes, above, either thr1 or thr4 were deleted in exemplary biotherapeutic yeast to construct safer yeast therapeutics. To screen for threonine auxotroph clones, cells were grown from clones on both rich medium (YPD) and minimal medium lacking threonine (SD). Multiple clones were identified that grew on rich medium, but did not grow on medium without threonine, indicating that threonine synthesis genes were successfully deleted (FIG. 37). These strains were also unable to grow on minimal medium with amino acids supplemented at standard concentrations, including threonine at 76 ug/ml (FIG. 38). The concentration of threonine in human plasma after analysis by SPE-LC-MS/MS was reported to be approximately 16.7 μg/ml after (Calderon-Santiago et al., 2012), far lower than the 76 μg/ml that is insufficient for growth of our therapeutic S. boulardii cells. Therefore, the threonine auxotrophic mutants are less likely to cause fungemia and make for a safer live biologic.

In addition to increasing the biosafety of therapeutics (e.g., engineered strains of Saccharomyces yeast as described herein) for patients, auxotrophic mutations, such as those in thr1, thr4, and the deletion of ura3 in yeast platform, are also a strategy for enhanced biocontainment. Once excreted from the gut of a patient, the need for therapeutic yeast (e.g., engineered strains of Saccharomyces yeast as described herein) to acquire specific amino acids from the environment for growth makes them environmentally safer (FIG. 37). Even in cases where the nutrients necessary for growth are present, engineered yeast described herein are less competitively fit compared to the wild-type (WT) parent strain, MYA-796.

To demonstrate this decrease in fitness, a competitive assay was developed in which therapeutic cells are cultured and passaged in competition with a GFP-expressing strain, which has an otherwise WT background, in a 24-well plate. After accounting for differences in density, the amount of GFP fluorescence emitted from the culture after excitation was measured using a plate reader. A culture of more fit cells will emit less fluorescence over time as the proportion of GFP+ cells decreases; in contrast, cultures of less fit yeast will emit more fluorescence over time as the percentage of GFP-expressing cells increases. Samples of the co-cultures were then used to inoculate fresh medium each day for four days.

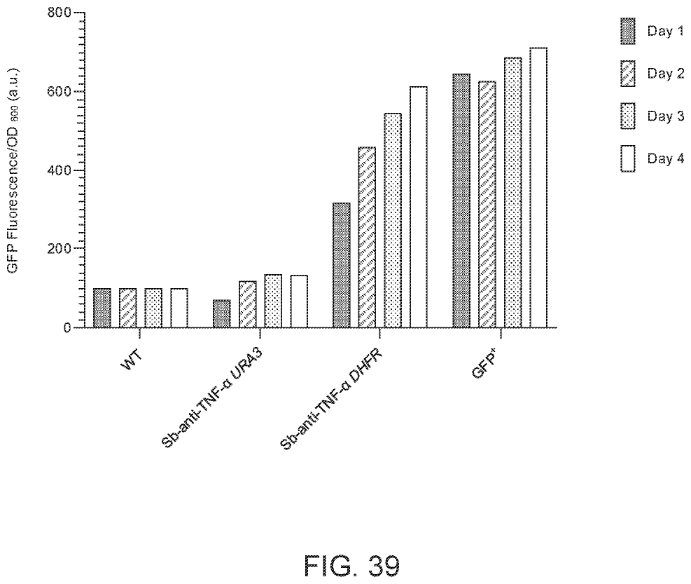

Two yeast strains were co-cultured that both secrete anti-TNF-alpha antibodies (Sb-anti-TNF-alpha) with the GFP+ yeast. Both Sb-anti-TNF-alpha strains were also ura3 mutants. In one strain, integration of the antibody cassette was selected for using ura3 as a marker, while the DHFR gene was used for selection in the second. The fluorescence from the uracil-prototroph strain remained similar to the control co-culture of WT and GFP+ cells over the course of the experiment (GFP+; FIG. 39). However, the fluorescence from the DHFR competition was higher than the WT control on Day 1 and increased to the level of the GFP+ control culture that contained only GFP-expressing cells. Though whether the presence of the DHFR gene contributes to the observed decrease in fitness is under investigation, these data suggest that ura3 mutants are less competitively fit than uracil prototrophs.

The auxotrophic mutations introduced, including ura3, thr1, and thr4, appear to be an effective biocontainment strategy that enhances the safety of our therapeutic strains for the environment. It is envisaged to test other auxotrophic genes as well, both in the uracil and threonine biosynthetic pathways and others. Commonly utilized yeast gene markers will be targeted, such as trp1 (tryptophan), lys2 (lysine), his3 (histidine), leu2 (leucine), and other genes encoding enzymes involved in the synthesis of these amino acids.

### Example 11: DSS and Bacterial Colitis

The present example demonstrates that intestinal delivery of anti-TNF-α neutralizing antibody via engineered S. boulardii (oral Sb-amTNF or FZ006m) significantly ameliorates disease severity by reducing intestinal inflammation and tissue damages. Engineered Sb expressing an anti-mouse TNF-α is effective in protecting mice from DSS colitis or colitis caused by DSS and Clostridioides difficile infection. First, whether intestinal delivery of a TNF-α-neutralizing antibody ameliorates symptoms of dextran sulfate sodium (DSS)-colitis was tested. Mice were on drinking water with 2% DSS for 8 days as described previously (see, e.g., Wirtz, S., et al. Nat Protoc 12, 1295-1309 (2017)). Starting on day 3, mice were gavaged daily with 10⁹ CFU of Sb-amTNF for 11 days. Control groups were gavaged with the same amount of Sb-EP control or PBS. FIG. 4A shows that none of those mice treated with oral Sb-amTNF died whereas 20% of mice succumbed in control groups. As DSS-colitis alone did not induce a significant death outcome in mice, we utilized a significantly more severe intestinal inflammation model of DSS colitis comorbid with C. difficile infection (see, e.g., Zhou, F., et al. Inflamm Bowel Dis 24, 573-582 (2018)). In this comorbidity model, mice developed severe intestinal inflammation, tissue damages, and 80% of the mice died. Since TNF-α was significantly upregulated, whether antiTNF-α antibody intestinally delivered by engineered S. boulardii would ameliorate disease severity and reduce intestinal inflammation was tested. Groups of comorbid mice were orally administered with PBS, Sb-EP, or Sb-amTNF at 10⁹ CFU/dose daily from day −3 to day 7 (11 doses total). Significant weight loss was evident and about 80% of mice had succumbed to the disease in the PBS and Sb-EP control groups, whereas oral Sb-amTNF conferred substantial protection with significantly lower weight loss (FIG. 4B) and 40% of mice succumbed (FIG. 4C). The upregulation of colonic proinflammatory cytokines TNF-α (FIG. 4D) and IL-6 (FIG. 4E) was also significantly reduced. Subsequently, the colon length reductions and tissue damages were also significantly alleviated in mice treated with oral Sb-amTNF (FIGS. 4F and 4G).

### Example 13: Human Colonic Tissues

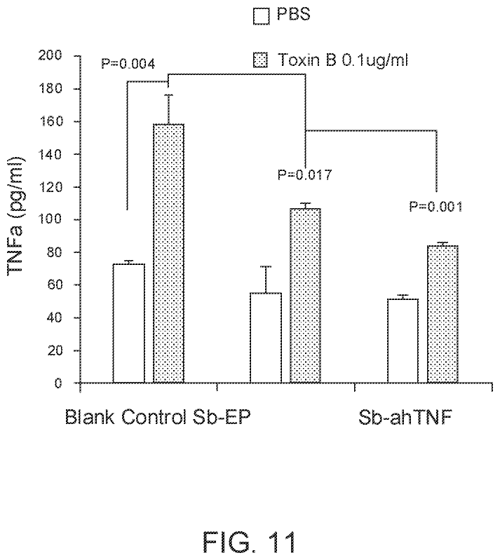

C. difficile toxins are proinflammatory and induce colitis in both animals and humans (see, e.g., Yu, H., et al. Clin Vaccine Immunol 24(2017), Zhang, Y., et al. Cell Mol Gastroenterol Hepatol 5, 611-625 (2018)). Oral Sb-amTNF diminishes colonic inflammation in mice (FIG. 4A-4G and FIG. 7A-7D). Since Sb-ahTNF (FZ006) expresses an antibody against human TNF-α, whether Sb-ahTNF reduced C. difficile toxin-induced expression of proinflammatory cytokines in human colonic tissues was evaluated. Results in FIG. 11 showed that the supernatant from control Sb-EP culture significantly reduced TcdB-induced upregulation of TNF-α in human colonic tissues, which is consistent with results presented herein and other reports of anti-inflammatory properties of this probiotic yeast (see, e.g., Sougioultzis, S., et al. Biochem Biophys Res Commun 343, 69-76 (2006), Castagliuolo, I. et al. Infect Immun 64, 5225-5232 (1996), Pothoulakis, C. Aliment Pharmacol Ther 30, 826-833 (2009)). The supernatant from Sb-ahTNF culture further reduced TNF-α upregulation (FIG. 11, Sb-EP vs Sb-ahTNF, P=0.001), demonstrating that the secretion of neutralizing antibodies against TNF-α by Sb diminished proinflammatory activity of TcdB in human colonic tissues.

Oral Sb-amTNF administration does not induce anti-drug response. SbamTNF constitutively expresses a bi-valent VHH fused with human Fe. Experiments were conducted to determine whether long-term, repeated oral dosing Sb-amTNF would induce an anti-antibody response that could potentially reduce drug activity. After 36 oral doses (109 CFU/dose) of Sb-amTNF over 8-week period, mice failed to produce any detectable anti-antibody IgA in feces or IgG in sera, whereas those mice injected with purified VHH/VH-Fc induced potent anti-antibody IgG responses (FIG. 12A-12B).

### Example 14: Exemplary Genes of Interest (GOI)

The present example demonstrates a summary of exemplary nucleic acids (genes of interest (GOIs)) encoding therapeutic polypeptide(s). Table 14.1 summarizes exemplary GOIs.

TABLE 14.1
Exemplary GOIs

| GOI | Summary |
|---|---|
| ABAB | ABAB is a tetra-specific antibody that potently and broadly neutralizes |
| | Toxin A and Toxin B of Clostridioides difficile (C. difficile). ABAB is a |
| | fusion of 4 single-domain antibodies (VHHs), two binding to two |
| | different epitopes of Toxin A and two binding to two different epitopes |
| | of Toxin B. ABAB is designed to treat C. difficile infection (CDI) and |
| | comorbidity with CDI. |
| Anti-TNF-α (anti- | Anti-TNF-α is an antibody that neutralizes an important inflammatory |
| tumor necrosis | cytokine TNF-α. TNF-α is a multifunctional proinflammatory cytokine |
| factor α) | that belongs to the tumor necrosis factor (TNF) superfamily. This |
| | cytokine is mainly secreted by macrophage and exhibits its function via |
| | binding to its receptors TNFRSF1A/TNFR1 and TNFRSF1 B/TNFBR. |
| | Dysregulation of TNF-alpha expression is thought to be a critical |
| | pathogenic mechanism in numerous autoimmune diseases, including |
| | inflammatory bowel disease (IBD), rheumatoid arthritis (RA), and |
| | ankylosing spondylitis (AS). Anti-TNF-α antibodies are designed to |
| | treat GI inflammation such as IBD. |
| Anti-mTNF-α | Anti-mouse TNF-α (Anti-mTNF-α), a homo-dimer of VHH fused with |
| | Fc fragment, which is specific binding to mouse TNF-α, and blockade |
| | of mouse TNF-α-induced cytotoxicity on L929 cell. |
| Anti-human TNF-α | A humanized-VHH monomer, which is specific to hTNF-α, and |
| VHH (Anti-hTNF- | neutralizes hTNF-α-induced cytotoxicity on L929 cell. |
| α) | |
| Anti-human TNF- | A hetero-dimer VHH antibody, which is highly specific to hTNF-α, |
| α-VHHs, G1 (Anti- | while very weak binding to mTNF-α at high concentration. Specific |
| hTNF-α) | binding EC50 is 0.112 nM, more than 3 times less than that of Humira |
| | (EC50 of humira is 0.390 nM). IC50 of anti-human TNF-αVHHs is |
| | 1.2 pM, 20 times more powerful to block hTNF-α-induced cytotoxicity |
| | versus that of Humira (IC50: 28 pM). It inhibits hTNF-α binding to its |
| | both receptors TNFR1 and TNFR2. Kinetics parameters are as KD at |
| | 9.725E−10 (M), Ka at 3.110E+5 (1/ms), Kd at 3.107E−4 (1/ms). |
| Anti-human TNF- | A Fc-fusion humanized VHH monomer of Anti-human TNF-α-VHH, |
| α-VHH-Fc, A3 | which is highly specific to hTNF-α, barely binding to mTNF-α. Specific |
| (Anti-hTNF-α-Fc) | binding EC50 is 0.141 nM, around 3 times less than that of Humira |
| | (EC50 of humira is 0.390 nM). Anti-human TNF-α-VHH is 4 times |
| | more potency in neutralizing activity compared with Humira (IC50: |
| | 28 pM), with IC50 of 7 pM. It inhibits hTNF-α binding to its both |
| | receptors TNFR1 and TNFR2. Kinetics parameters are as KD at |
| | 2.999E−10 (M), Ka at 6.257E+5 (1/ms), Kd at 1.877E−4 (1/ms). |
| Mouse-IL-22 (mIL- | IL-22 is an IL-10 family cytokine, produced by several populations of |
| 22) | immune cells at a site of inflammation. It takes effect on non- |
| | hematopoietic cells - mainly stromal and epithelial cells. It is important |
| | for the modulation of tissue responses during inflammation. Through |
| | activation of Stat3-signaling cascades, the cytokine induces proliferative |
| | and anti-apoptotic pathways, as well as anti-microbial molecules, that |
| | help maintain epithelial integrity, prevent tissue damage and aid in its |
| | repair. |
| anti-human IL- | Anti-hIL-17A-Fc is a Fc-fusion VHH monomer against hIL-17. This |
| 17A-Fc (anti-hIL- | antibody specifically binds to hIL-17 and it mediates inhibitions of IL- |
| 17A-Fc) | 17 A induced cell proliferation of human IL-17 reporter HEK293 cells, |
| | which were stably express receptors of hIL-17 A Its target, hIL-17 is a |
| | member of IL-17 family. IL-17 A promotes inflammation by inducing |
| | various proinflammatory cytokines and chemokines, recruiting |
| | neutrophils, enhancing antibody production, and activating T cells. IL- |
| | 17A expression is also augmented in autoimmune diseases such as |
| | multiple sclerosis and rheumatoid arthritis. |
| Canakinumab (anti- | yCAN-sc-FV-Fc is a sc-FV formed antibody fused with Fc fragment. |
| IL1beta) (yCAN- | sc-FV part are composed of heavy chain and light chain of |
| sc-FV-Fc) | Canakinumab. Its target hIL-1 beta. By neutralizing IL-1β, preliminary |
| | evidence suggests that canakinumab inhibits pro-tumor inflammation |
| | (PTI) to 1) enhance anti-tumor immune response; 2) reduce tumor cell |
| | proliferation, survival and invasiveness; and 3) impair angiogenesis. |
| | Pro-tumor inflammation enables tumor development by driving cancer- |
| | causing processes and by suppressing anti-tumor immune responses. |
| Gimsilumab (anti- | yGIM-sc-FV-Fc is a sc-FV format antibody fused with Fc fragment. sc- |
| GM-CSF) (yGIM- | FV part are composed of heavy chain and light chain of Gimsilumab. Its |
| sc-FV-Fc) | target human granulocyte-macrophage colony-stimulating factor (GM- |
| | CSF), also known as colony-stimulating factor 2 (CSF2), which is a |
| | myeloid cell growth factor and proinflammatory cytokine. At the acute |
| | and severe stage marked by a “cytokine storm” and the overactivation of |
| | inflammatory myeloid cells that infiltrate and damage tissue, Inhibition |
| | of GM-CSF may be able to reverse this pathology. GMCSF has been |
| | identified as a key mediator of chronic inflammation in models of |
| | colitis. GM-CSF-deficient mice developed more severe colitis in |
| | dextran sodium sulfate (DSS)-induced colitis reported that. GM-CSF |
| | modulates the macrophage phenotype to prevent intestinal fibrosis. In |
| | line with a potential beneficial role of GM-CSF in IBD, GM-CSF |
| | administration can improve IBD in some patients. High levels of |
| | circulating anti-GM-CSF autoantibodies have been found to correlate |
| | with worse Crohn's disease (CD) prognosis. |
| Humira (anti-TNF- | yHUM-sc-FV-Fc is a sc-FV format antibody fused with Fc fragment. |
| alpha) (yHUM-sc- | sc-FV part are composed of heavy chain and light chain of Humira. |
| FV-Fc) | HUMIRA is a TNF blocker. HUM IRA is to reduce the signs and |
| | symptoms of moderate to severe rheumatoid arthritis (RA) in adults, |
| | psoriatic arthritis (PsA) in adults moderate to severe polyarticular |
| | juvenile idiopathic arthritis (JIA) in children 2 years of age and older, |
| | ankylosing spondylitis (AS) in adults, Moderate to severe hidradenitis |
| | suppurativa (HS) in people 12 years and older. Humira havs been shown |
| | not only to reduce the symptoms of IBD, but also result in healing of the |
| | inflamed intestine. Humira is also a prescription medicine used to treat |
| | moderate to severe ulcerative colitis in adults and children 5 years of |
| | age and older. It also used to treat moderate to severe Crohn's disease |
| | (CD) in adults and children 6 years of age and older. |
| Avastin (Anti- | yAVA-sc-FV-Fc is a sc-FV format antibody fused with Fc fragment. sc- |
| VEGF) (yAVA-sc- | FV part are composed of heavy chain and light chain of Avastin. |
| FV-Fc) | Avastin is vascular endothelial growth factor (VEGF) inhibitor to |
| | indicated prevent the growth of new blood vessels. It is approved to |
| | combine with other anti-cancer durg or cytokine to treat different |
| | persistence, recurrent, or metastatic cancers including colon cancer. |
| Mavrilimumab | yMAV-sc-FV-Fc is a sc-FV format antibody fused with Fc fragment. |
| (anti-GM-CSF | sc-FV part are composed of heavy chain and light chain of |
| Receptor) (yMAV- | Mavrilimumab. Mavrilimumab is an investigational fully-human |
| sc-FV-Fc) | monoclonal antibody that targets granulocyte macrophage colony |
| | stimulating factor receptor alpha (GM-CSFRa). Currently, released |
| | report from Lancet of the evaluation that whether mavrilimumab, a |
| | monoclonal antibody to the GM-CSF receptor, would improve |
| | outcomes in patients with COVID-19 pneumonia and systemic |
| | hyperinflammation. Unfortunately, there was no significant difference |
| | in the proportion of patients alive and off oxygen therapy at day 14, |
| | although benefit or harm of mavrilimumab therapy in this patient |
| | population remains possible given the wide confidence intervals, and |
| | larger trials should be completed. |
| Siltuximab (anti- | ySIL T-sc-FV-Fc is a sc-FV format antibody fused with Fc fragment. |
| IL6) (ySIL T-sc- | sc-FV part are composed of heavy chain and light chain of Siltuximab. |
| FV-Fc) | Siltuximab is a chimeric (human-mouse) monoclonal immunoglobulin |
| | G1-kappa antibody. Siltuximab prevents the binding of IL-6 to soluble |
| | and membrane-bound IL-6 receptors by forming high affinity |
| | complexes with human interleukin-6 (IL-6). Its use is indicated for the |
| | treatment of adult patients with multicentric Castleman's disease (MCD) |
| | who are human immunodeficiency virus (HIV) negative and human |
| | herpesvirus-8 (HHV-8) negative. MCD is a rare blood disorder caused |
| | by dysregulated IL-6 production, proliferation of lymphocytes, and |
| | subsequent enlargement of the lymph nodes. |
| yeGFP | yeFGP (Yeast-enhanced green fluorescent protein) yeFGP, a |
| | quantitative reporter of gene expression in yeast, a fast tool to observe |
| | the expression level. |

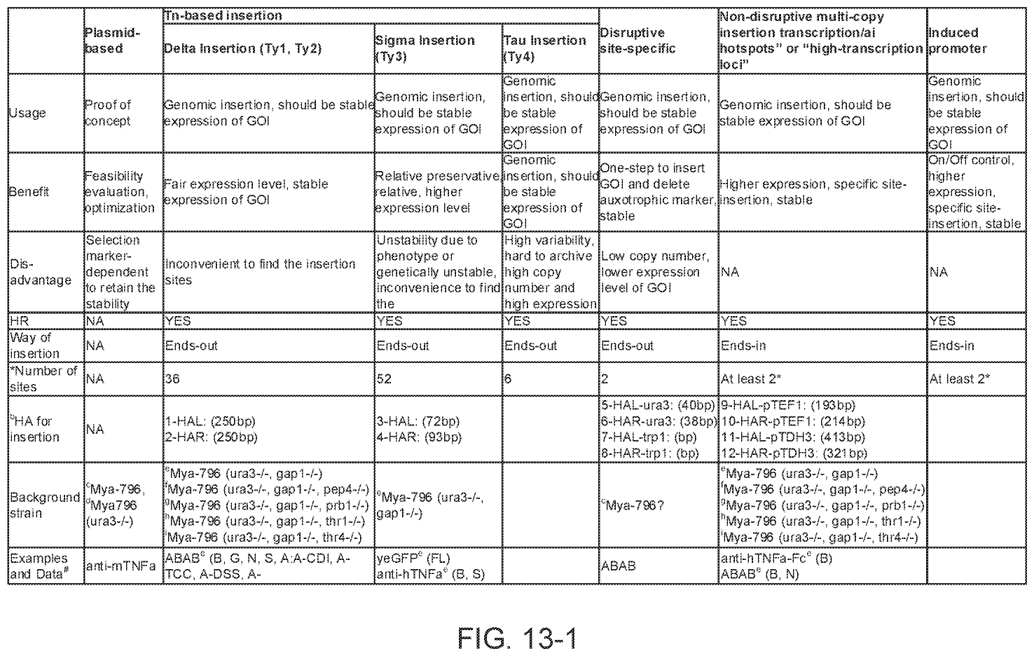

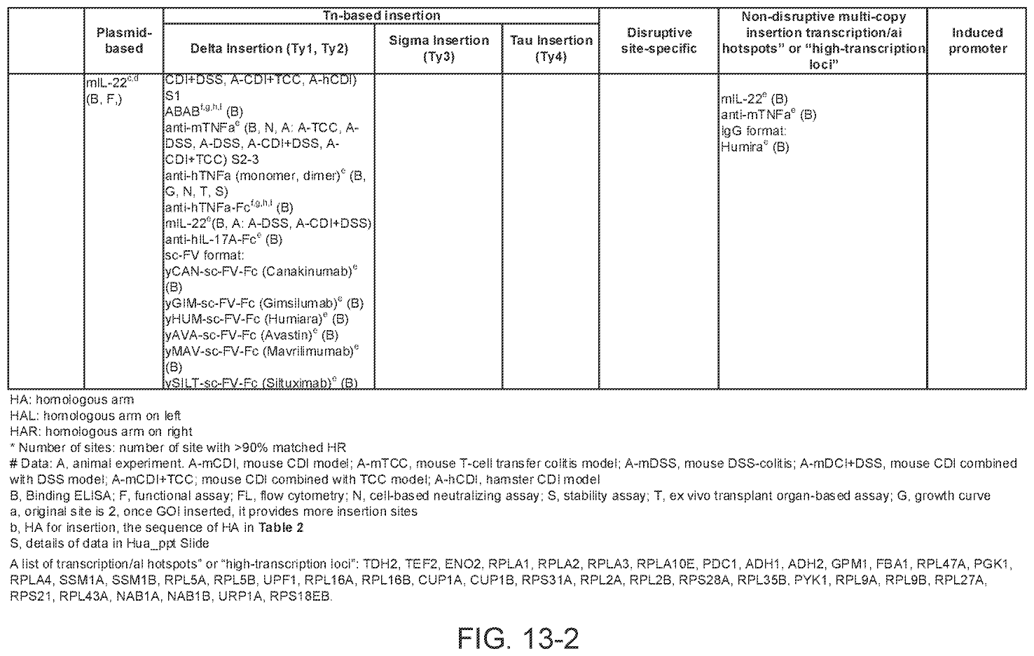

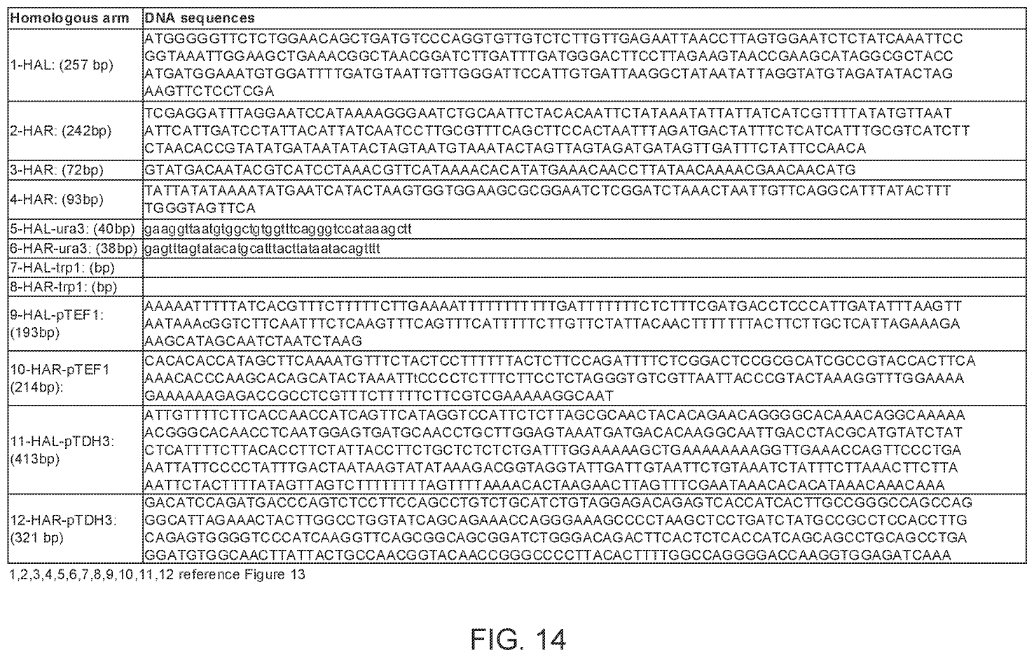

A summary of methods of gene insertions is shown in FIG. 13 and exemplary DNA sequences of homologous arms employed for the development of engineered S. boulardii strains are shown in FIG. 14.

### Example 15: Disruptive Site-Specific and Tn-Based Insertion

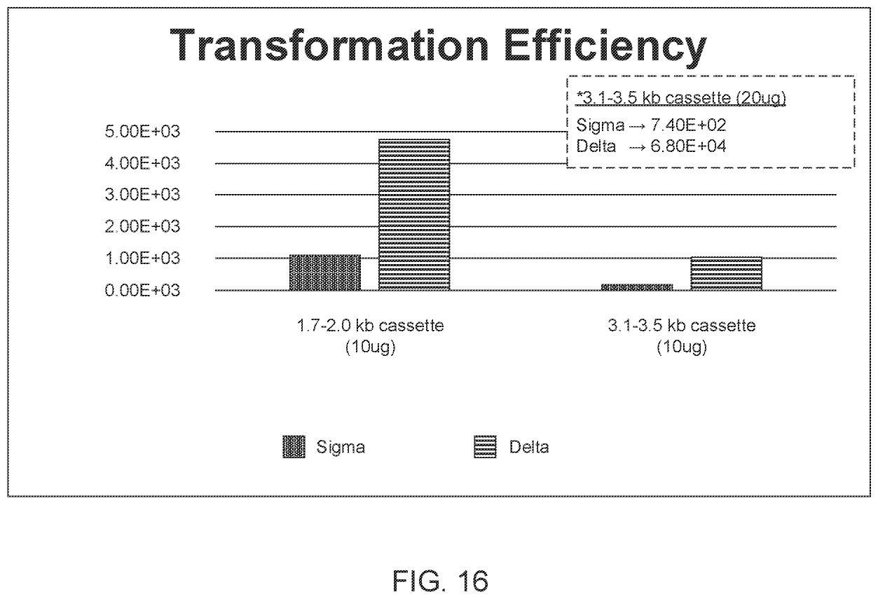

The present example demonstrates disruptive site-specific and Tn-based insertion. Sb strain (ura3^{-/-} and gap^{-/-}) was electroporated with 10 μg of linear DNA of different sizes for integration into the genome via conventional (ends-out) homologous recombination. Transformation efficiency at Delta sites was 4.3-fold and 5.9-fold higher than Sigma sites for the ˜2 kb-cassettes and ˜3 kb-cassettes, respectively (FIG. 16). Interestingly, use of CRISPR for integration resulted in a 3-fold decrease (˜2 kbcassette) and ≥100-fold decrease (˜3 kb-cassettes) in transformation efficiency at the Delta sites, but did not improve nor decrease transformation efficiency for the Sigma sites. For the Sigma sites, increasing the amount of linear DNA to 20 μg for the ˜3 kb-cassette resulted in a 4.1-fold increase in transformation efficiency, while the Delta sites had a dramatic 64.5-fold increase in the same.

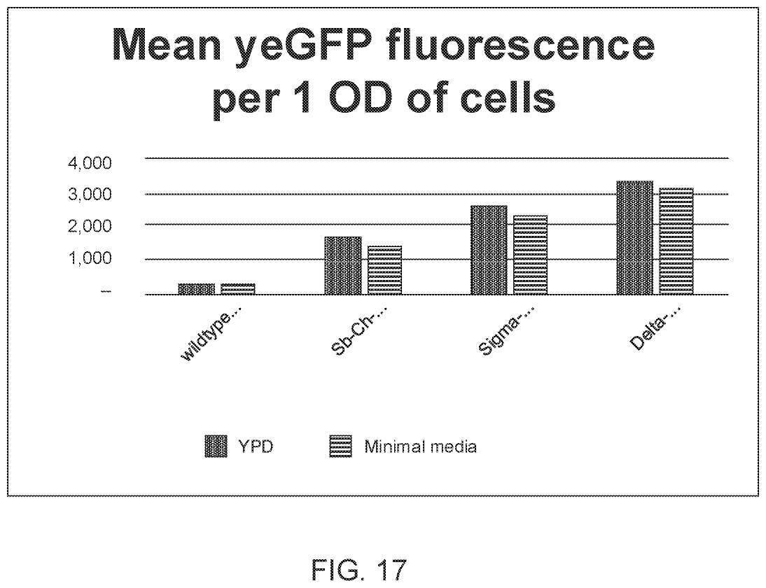

Sb strains (ura3^{-/-} and gap1^{-/-}) were electroporated with yeGFP-expression cassette targeted for integration at Sigma or Delta sites. Single colonies were cultured for 24 hours in rich media (YPD) or minimal media followed by FACS analysis to measure yeGFP-fluorescence. In general, clones with yeGFP at Delta sites had 28%-36% higher fluorescence than Sigma clones, implying higher yeGFP-expression in Delta clones. A representative clone from Sigma and Delta groups is shown. For comparison, Sb-Ch-yeGFP which has yeGFP cassette at ura3-locus showed 36%-56% less yeGFP fluorescence intensity than the Sigma or Delta clones, suggesting overall lower yeGFP expression from the ura-locus (FIG. 17).

### Example 16: Generation of Exemplary Final Constructs (FICs) and Assessment of Same

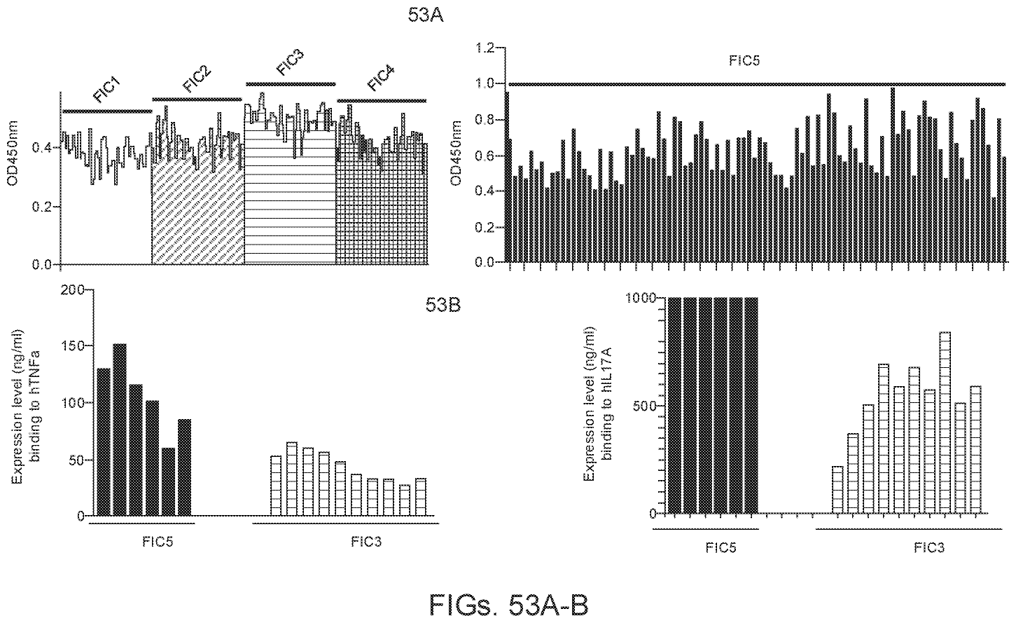

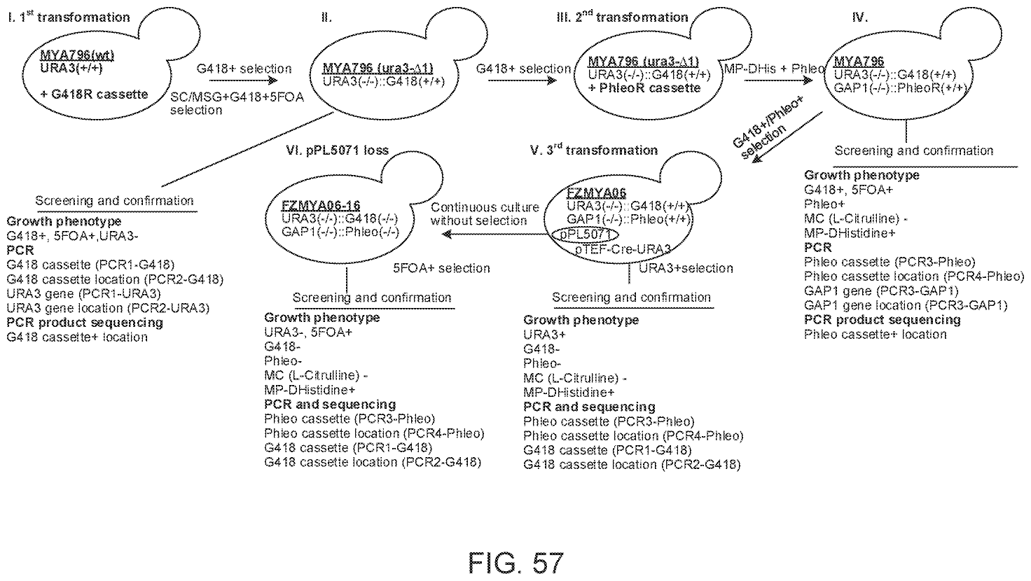

The present example demonstrates generation of exemplary final constructs (FICs) with different combination of antibody gene formats, promoters, and selection markers for probiotic yeast transformation (e.g., generation of engineered strains of Saccharomyces yeast). Resultant plasmids were first diagnosed with restriction digestions (FIG. 57) and DNA sequencing. Transgene cassettes, along with selection markers, were electroporated into competent S. boulardii and the yeast was plated on agar plates. Single colonies from the selection plates were collected and cultured in synthetic media containing specific chemicals for selection of those clones with correction genomic insertion of the transgene cassettes. Two rounds of screening were performed. For the first round, a total of approximately 200 clones from each FIC construct were evaluated for their expression of a bi-specific antibody by ELISA (FIG. 53A). Specifically, the Fc fusion polypeptides in the supernatants were captured by coated streptavidin in a 96-well plate and detected using a HRP conjugated anti-human IgG1 heavy chain antibody. This assay allowed assessment of the total Fc fusion polypeptide among the different clones to identify high expression strains (FIG. 53A). In the subsequent screening round, approximately 6-10 clones with the highest protein or polypeptide (e.g., therapeutic polypeptide(s)) expression were further evaluated by ELISA against recombinant TNF-α (FIG. 53B, left) and recombinant IL-17A (FIG. 53B, right). These data showed that cassette with VHH1-VHH2-Fc format displayed the highest expression levels by ELISA against both TNF-α and IL-17A (FIG. 53B) and therefore these clones from FIC number 5 (FIC5) were chosen for functional assay.

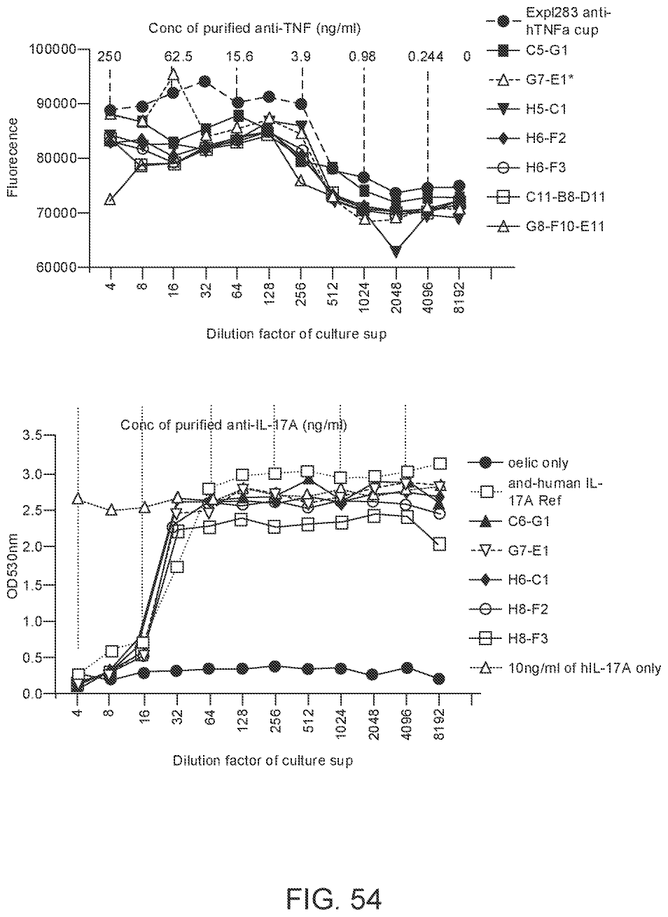

Cell-based neutralization assays were used to further assess FIC5 clones. To measure anti-TNF-α neutralizing activity of the bi-specific antibody secreted by the yeast clones, serially diluted supernatants were mixed with human TNF-α (10 μM) before applied to L929 cells. After 24 hours of incubation, the antibody-mediated inhibitions of TNF-α mediated cytotoxicity were measured (FIG. 54, left). To evaluate anti-IL-17A neutralizing activity of the bi-specific antibody secreted by the yeast clones, serially diluted supernatants were mixed with human recombinant IL-17A (10 ng/mL) before applied to HEK293 reporter cells. The bioactivity of human IL-17A can be measured by the reporter HEK293 cells, thus the antibody-mediated inhibitions of IL-17A bioactivity were measured (FIG. 54, right). The supernatant from Clone G7-E1 (also referred to as FZ008) showed the high neutralizing activities against both TNF-α and IL-17A.

Thus, it was determined that promoter P2 drove a higher secretion of exemplary anti-TNF-α/IL-17A bispecific antibody. The bioactivities between two exemplary formats, VHH-VHH-Fc and VHH-Fc-VHH were comparable, but the former format yielded a higher expression in the probiotic yeast. Selection markers did not particularly affect the overall screening or expression of the antibody.

### Example 17: Exemplary Generation and Characterization of Engineered Strains of Saccharomyces Yeast

The present example demonstrates exemplary generation and characterization of engineered strains of Saccharomyces yeast described herein.

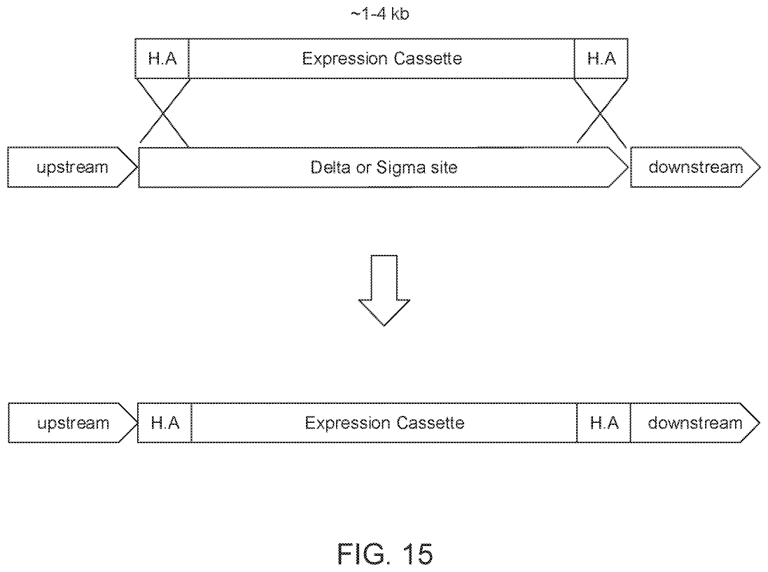

FIGS. 15-17 demonstrate disruptive site-specific and Tn-based insertion.

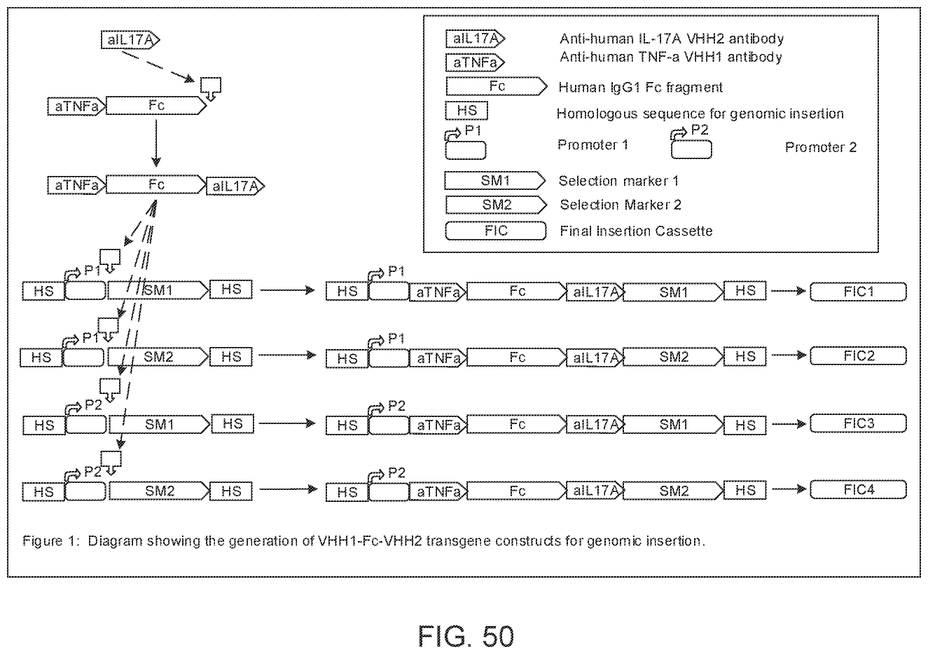

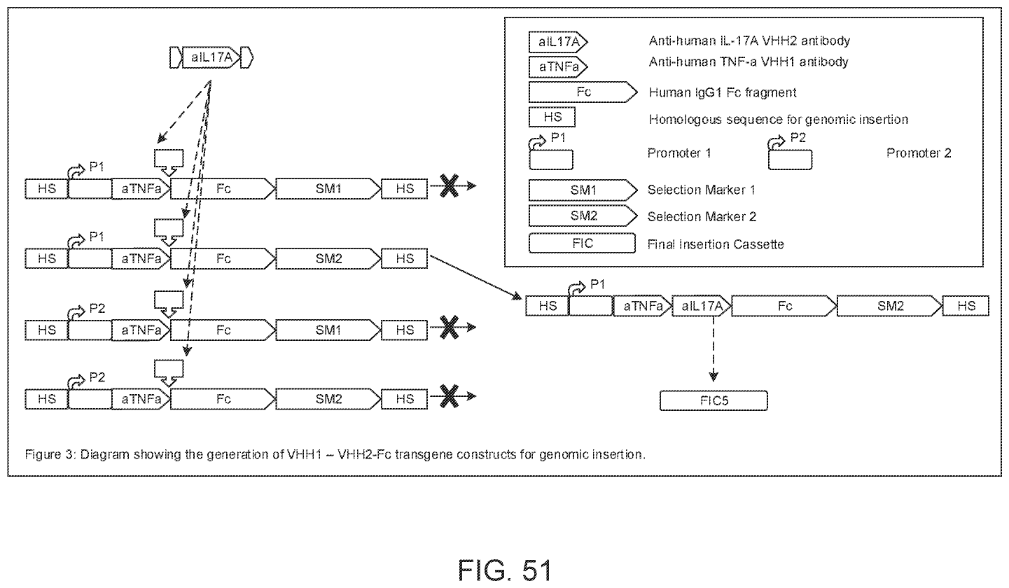

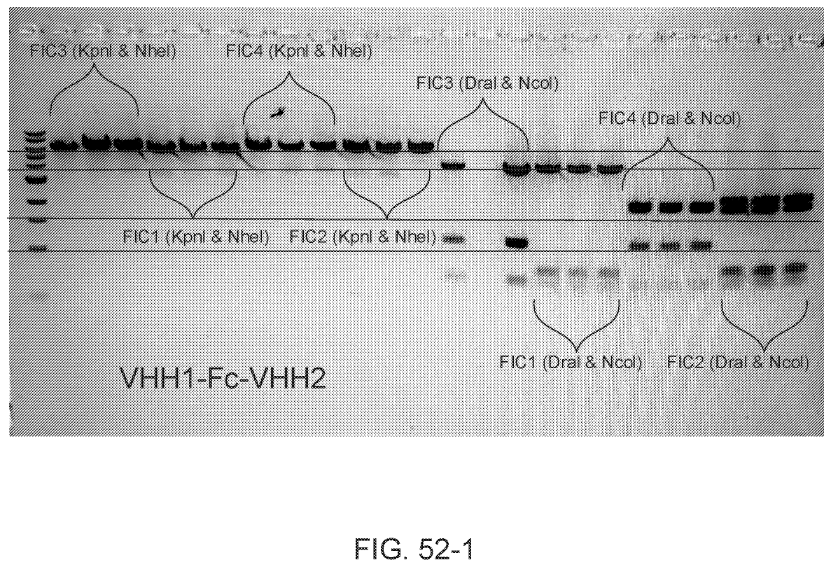

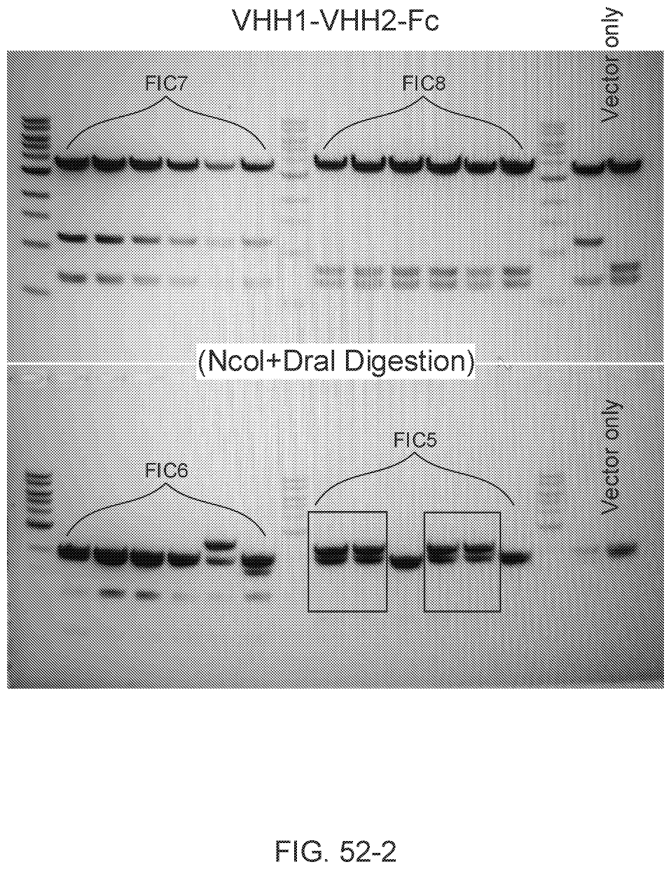

FIGS. 50-52 demonstrate steps for generating final integration cassettes for yeast chromosomal integration and diagnostic digestion of same.

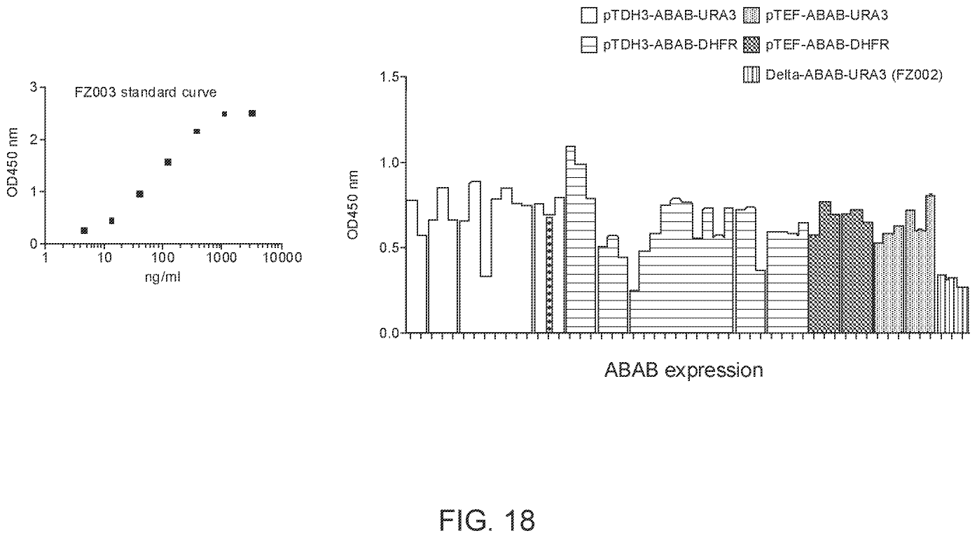

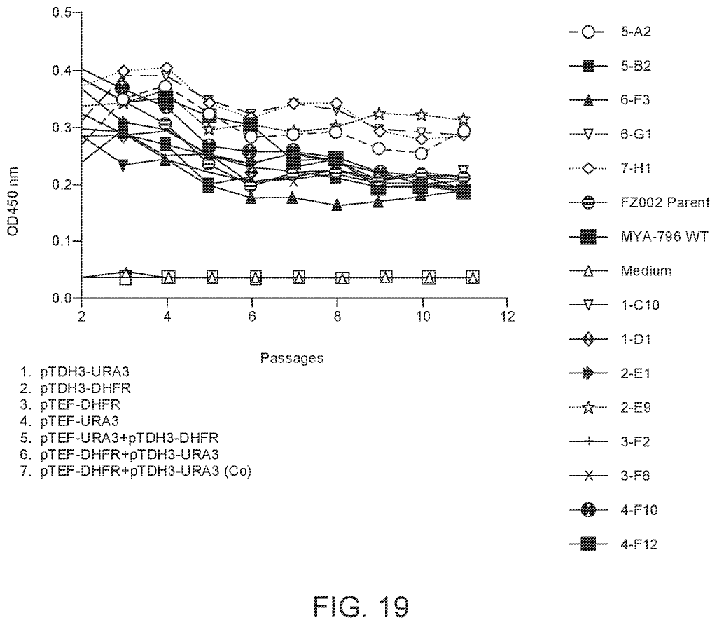

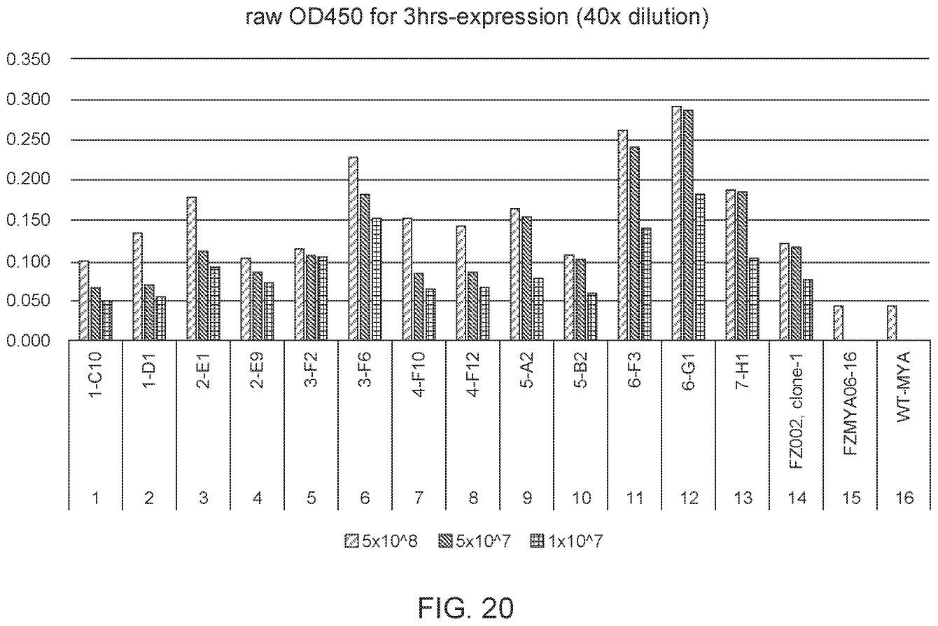

FIGS. 18-20 demonstrates expression of antitoxin ABAB and stability of the clones among different inserted locations (e.g., pTEF, pTDH3, and Delta site).

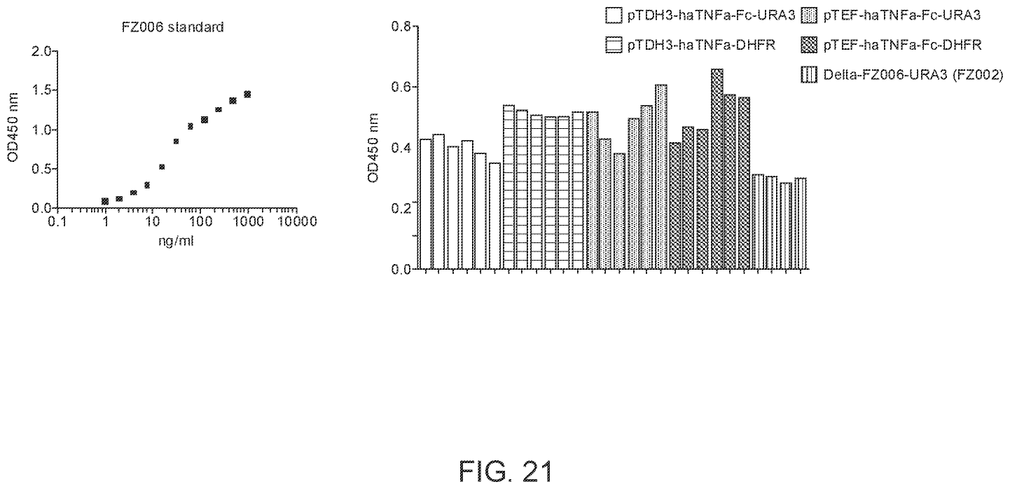

FIG. 21 demonstrate expression of hTNFα-Fc comparison among different inserted locations (e.g., pTEF, PTDH3, and Delta site).

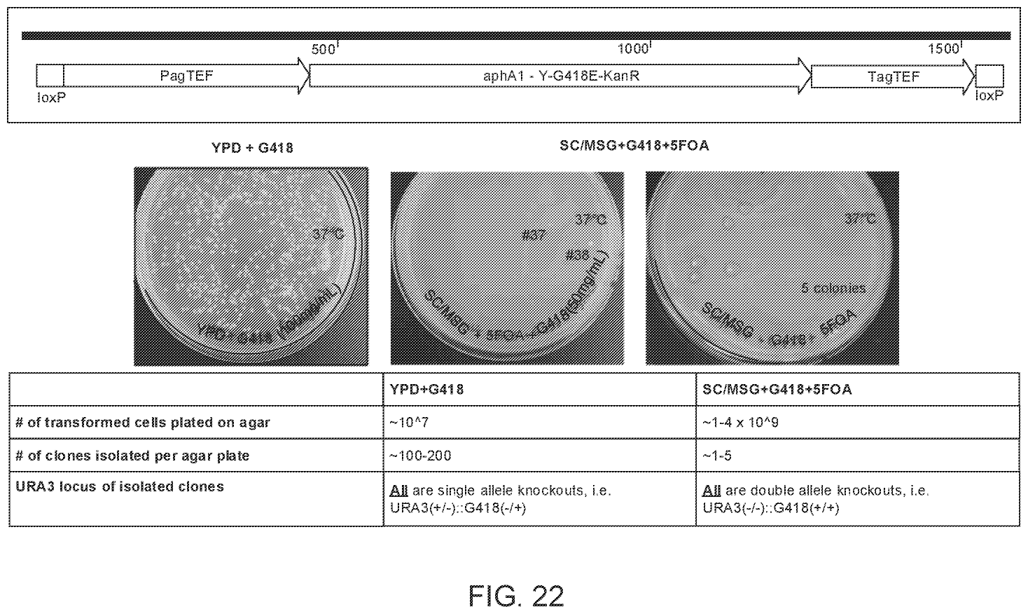

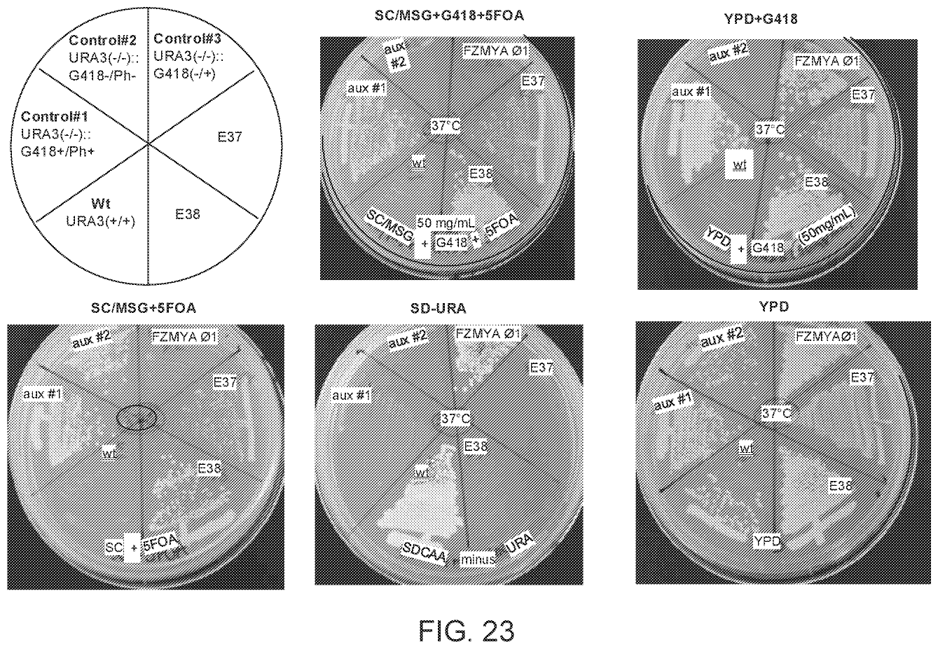

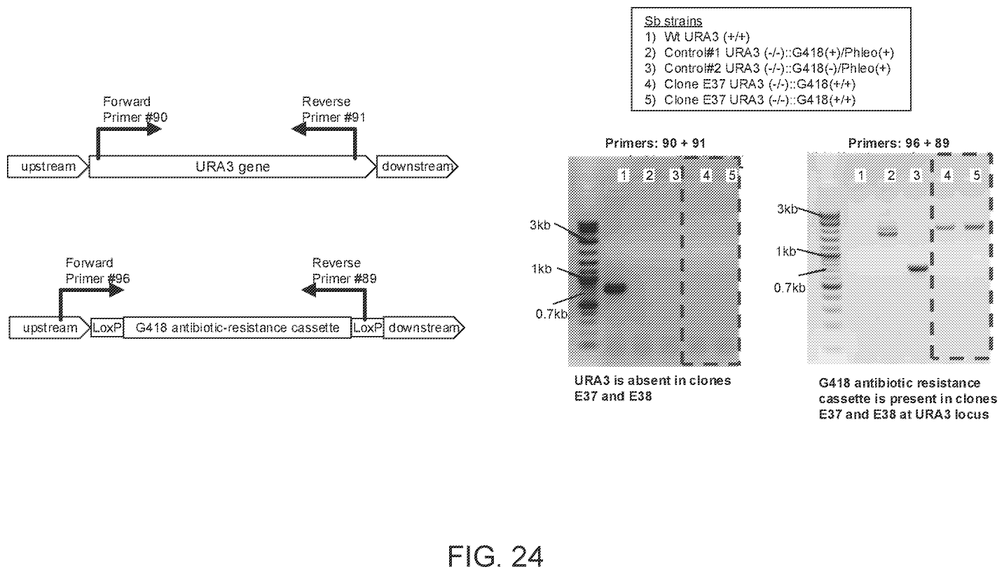

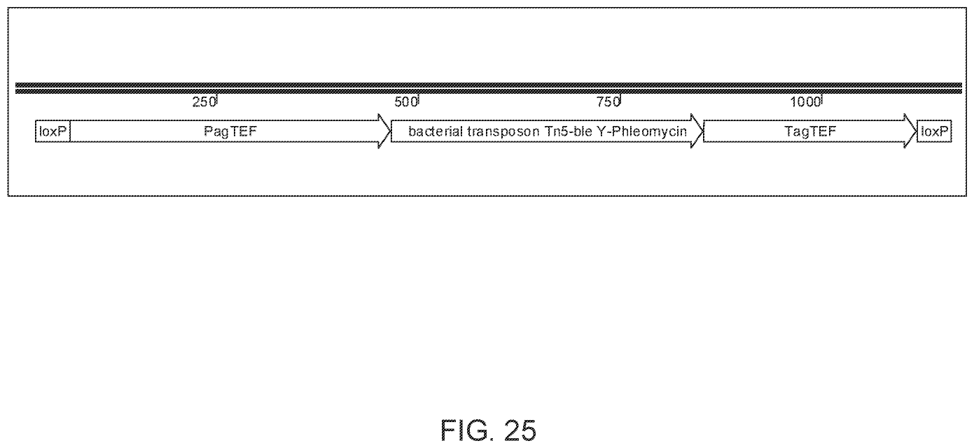

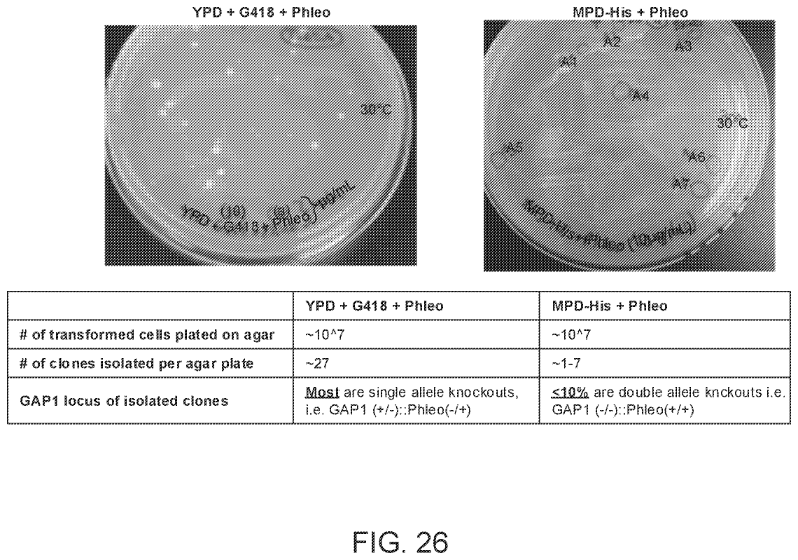

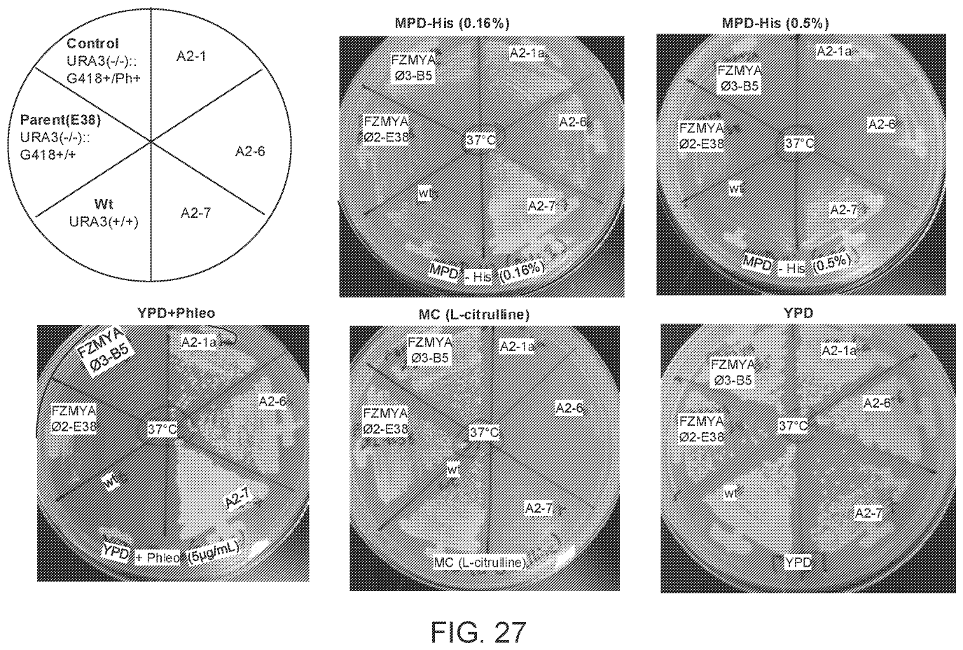

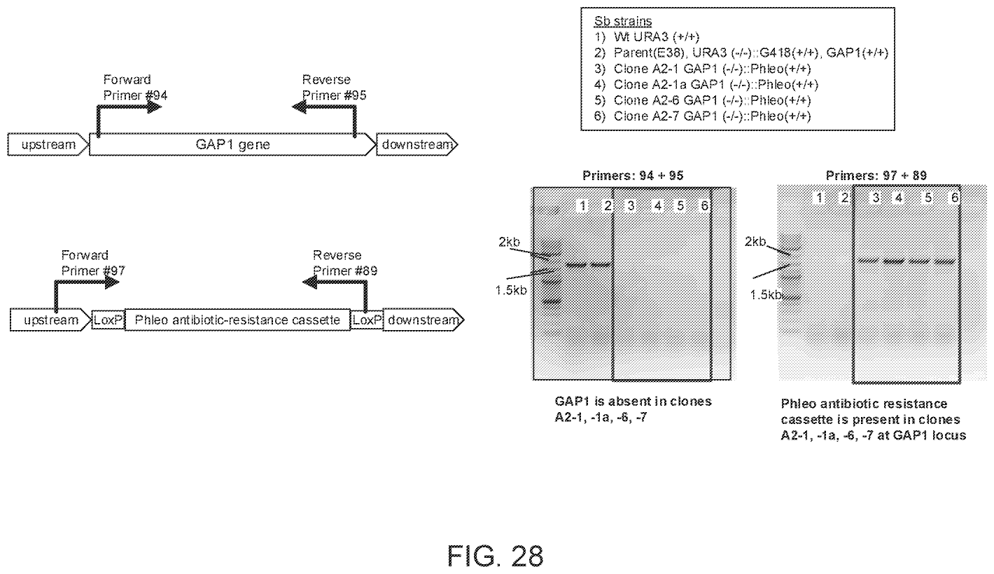

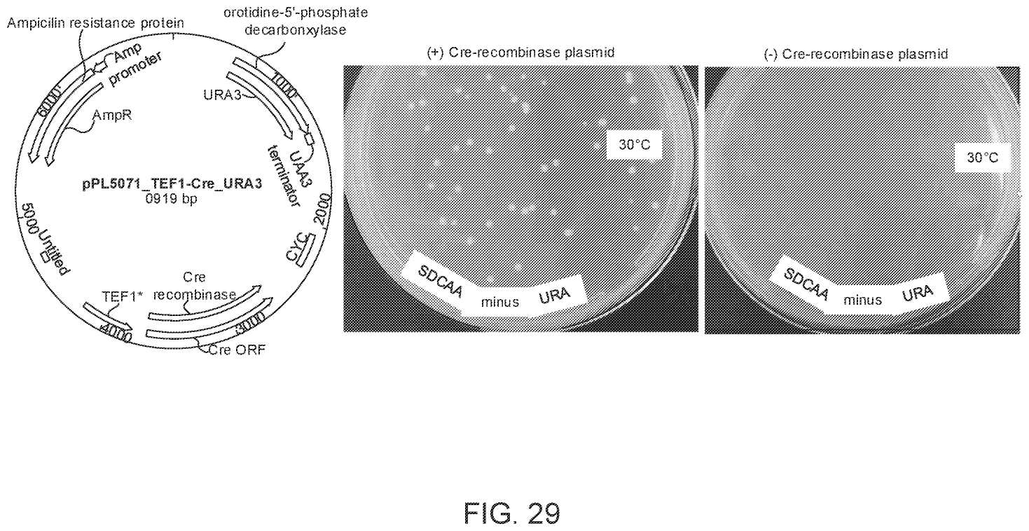

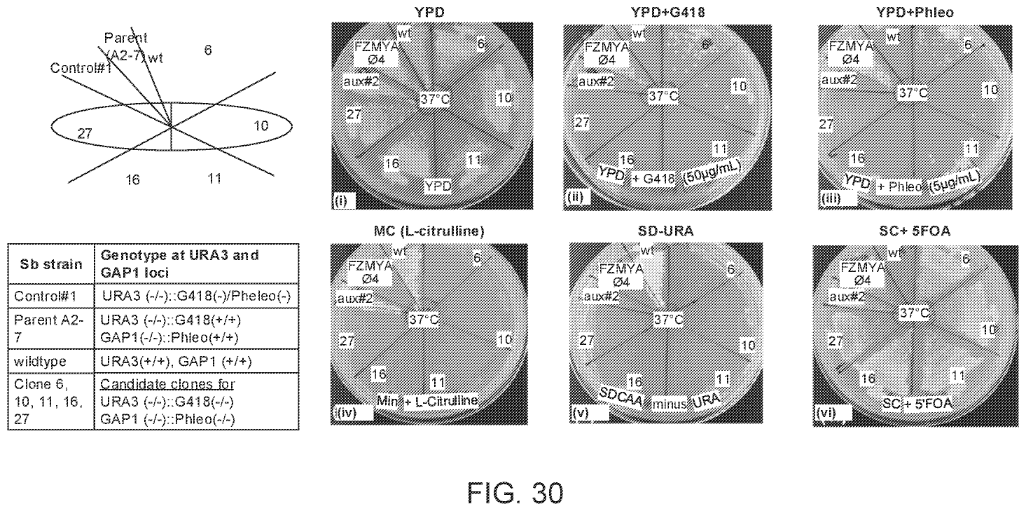

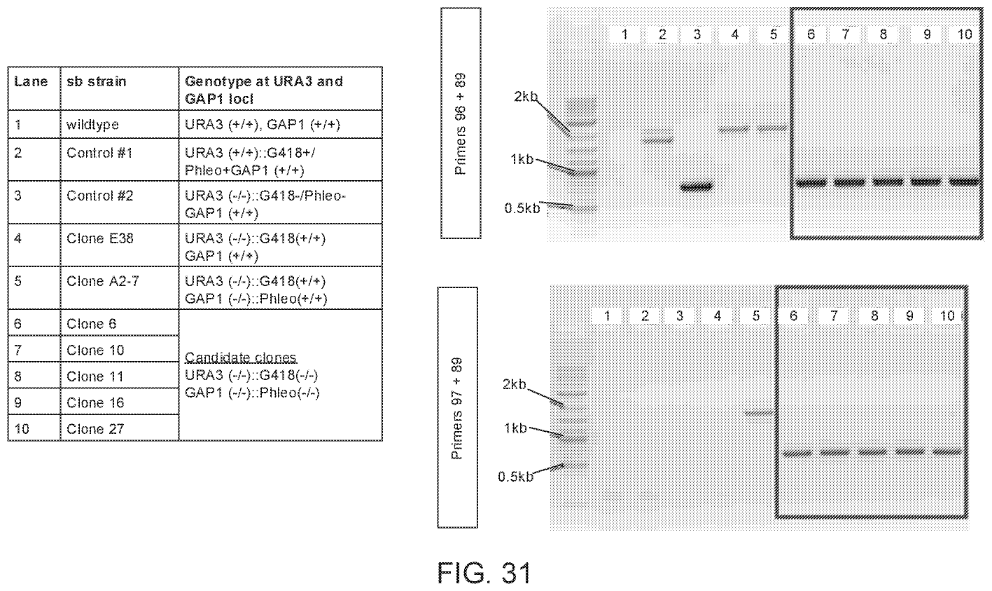

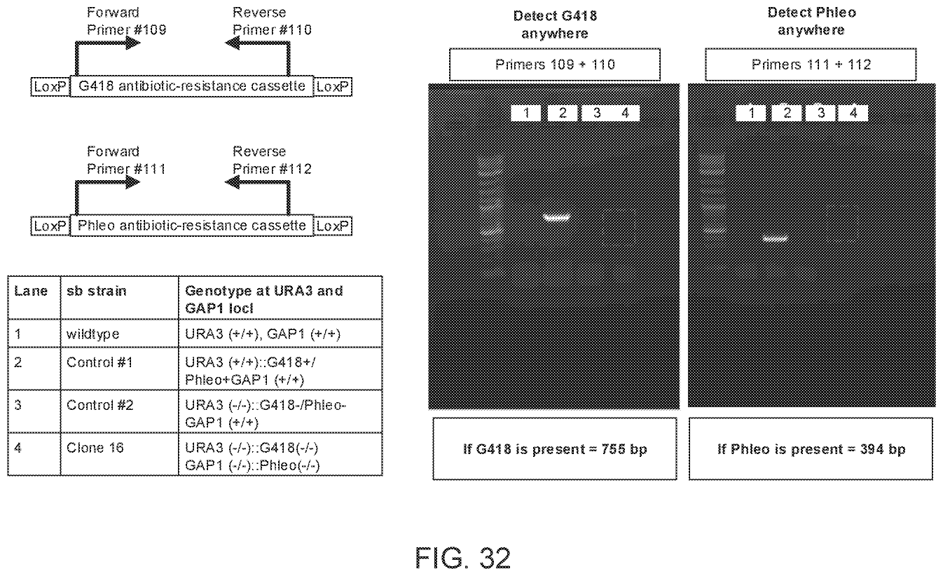

FIGS. 22-32 show exemplary generation and validation of auxotrophic S. boulardii yeast strains.

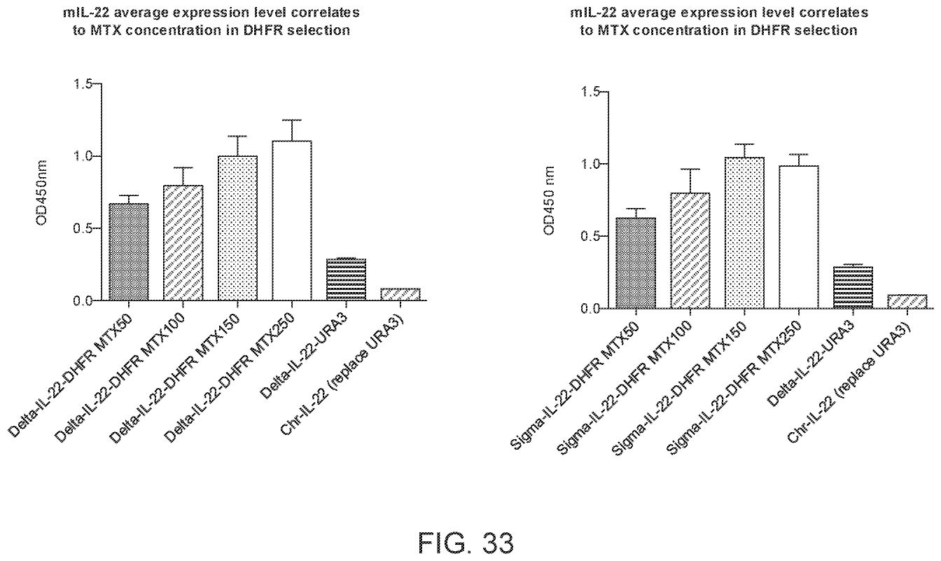

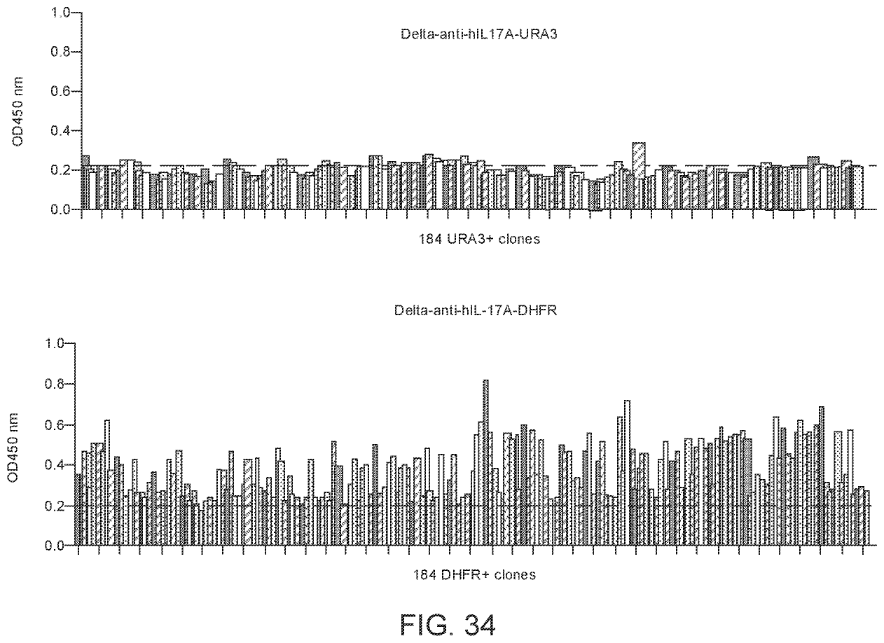

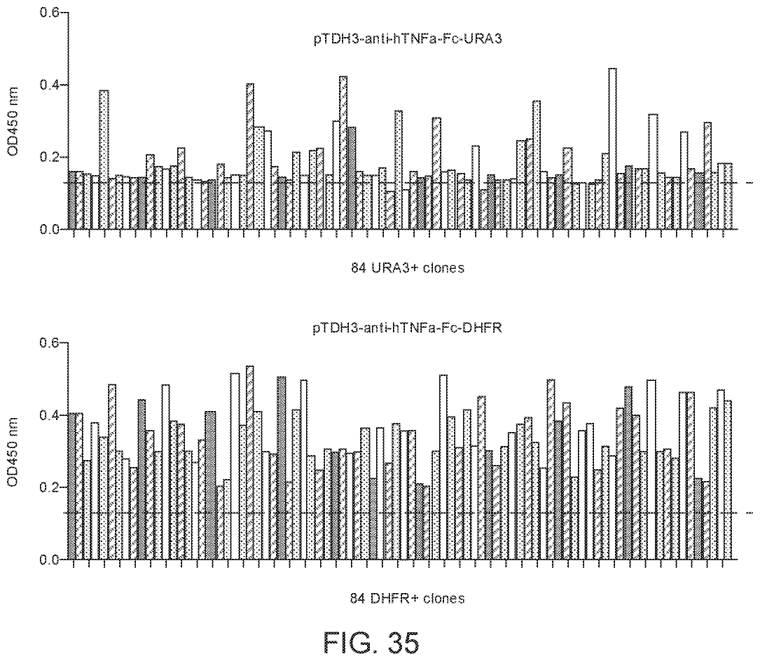

FIGS. 33-35 demonstrates DHFR/DFR1 systems.

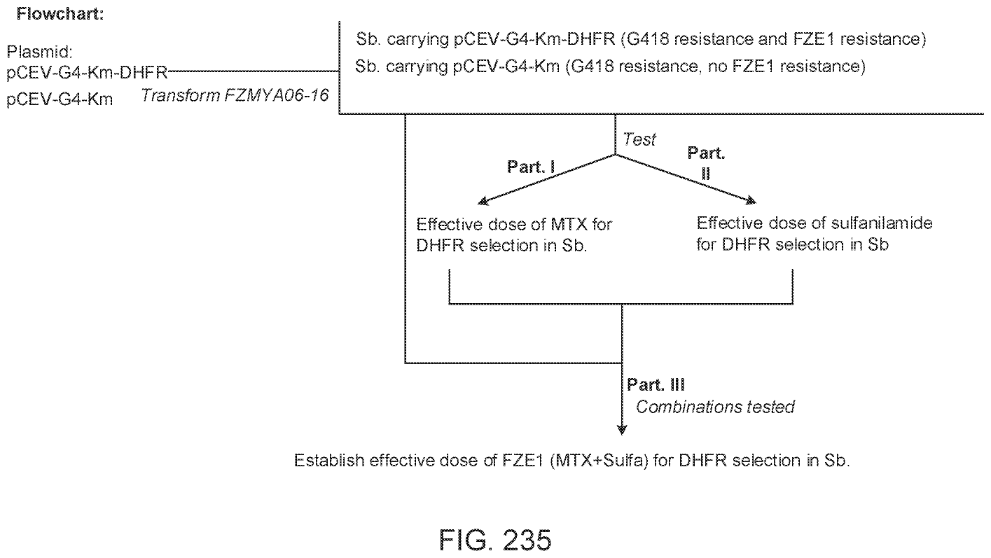

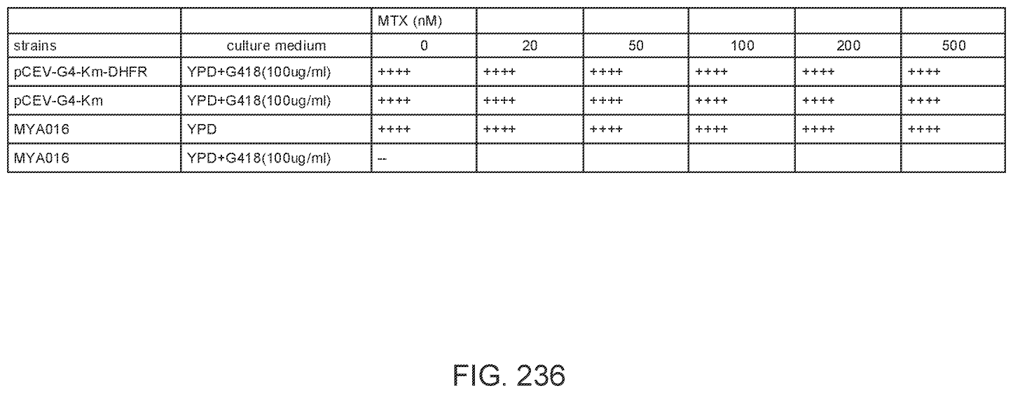

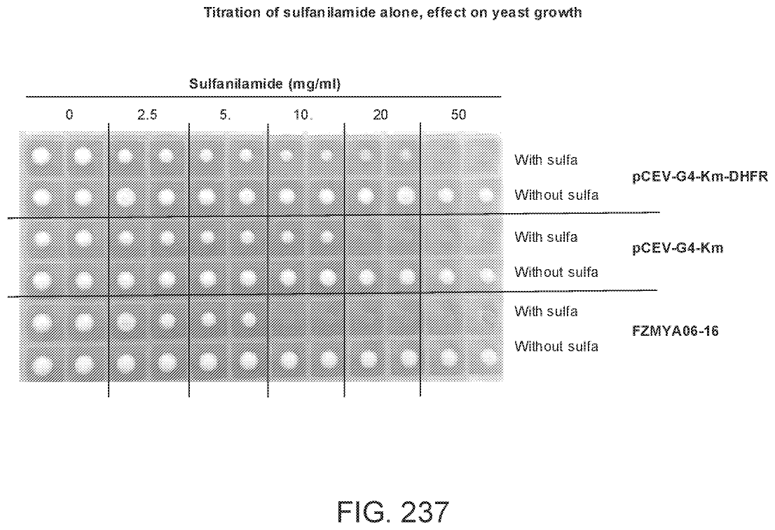

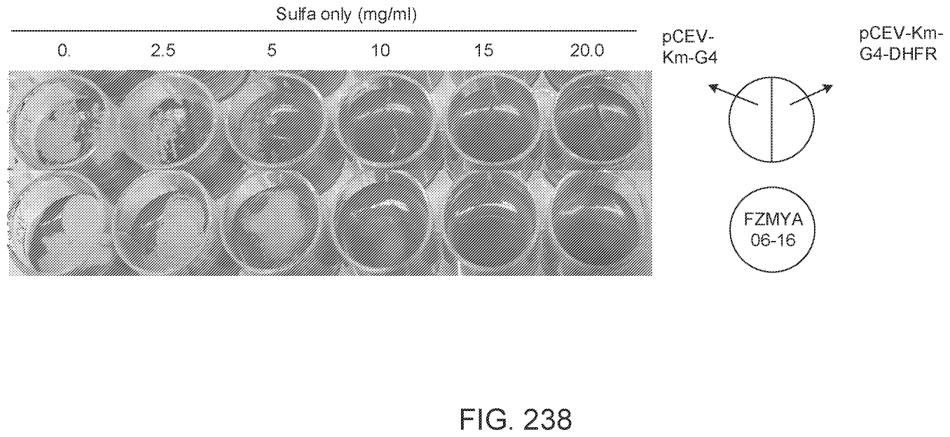

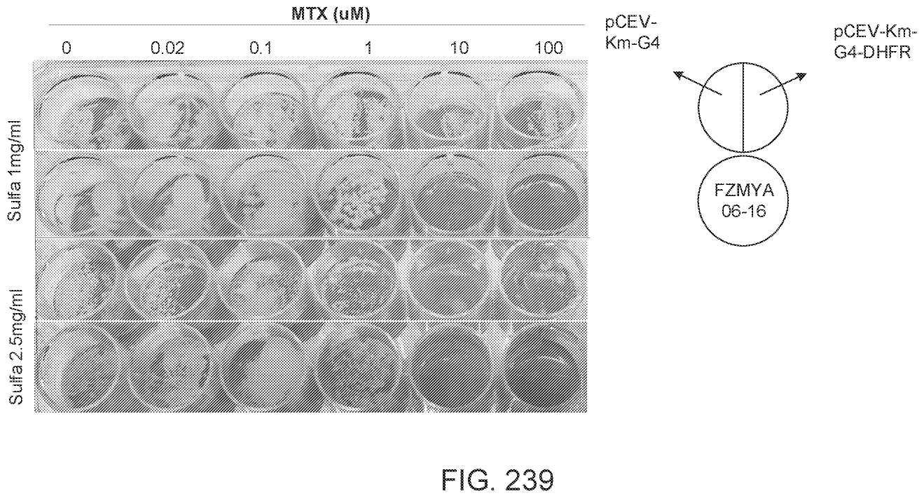

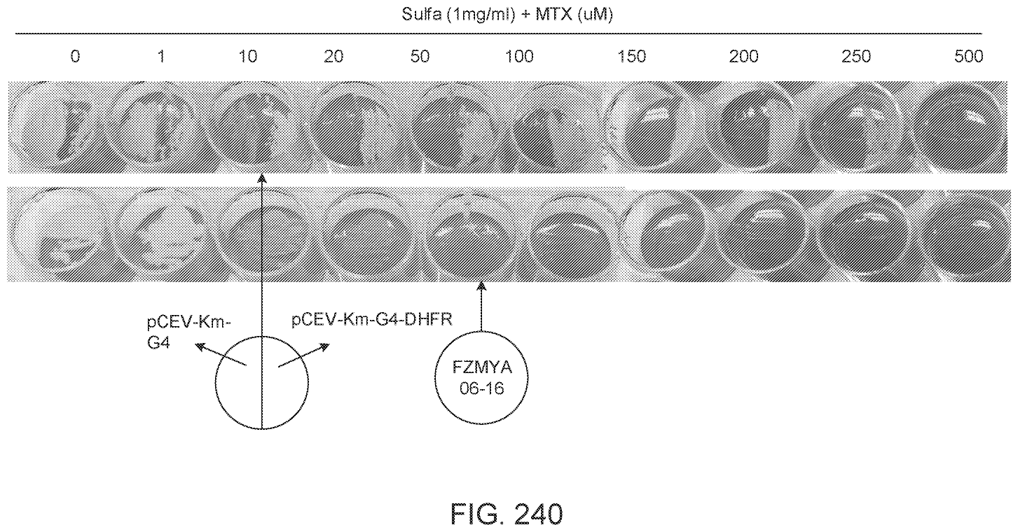

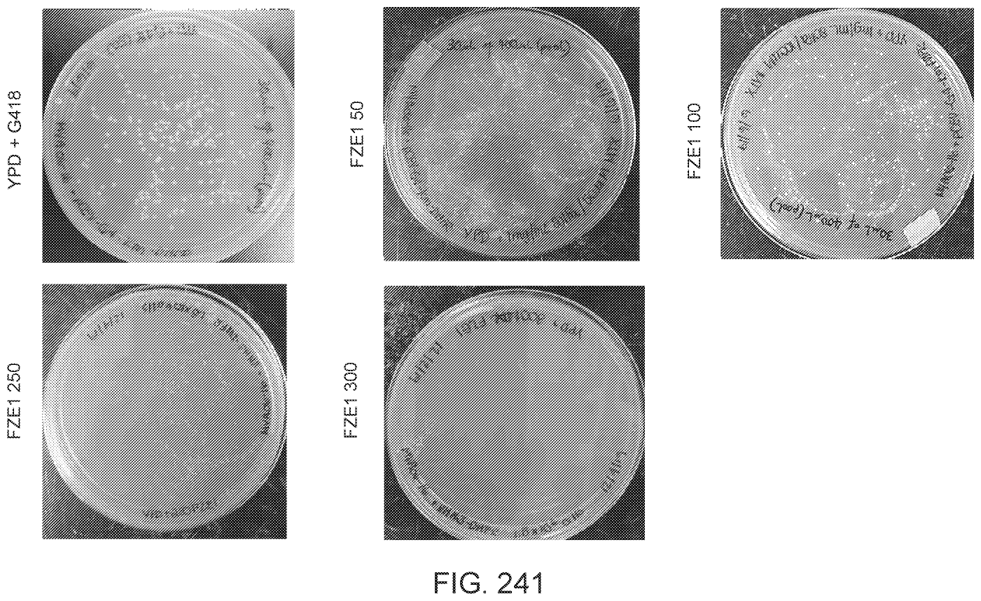

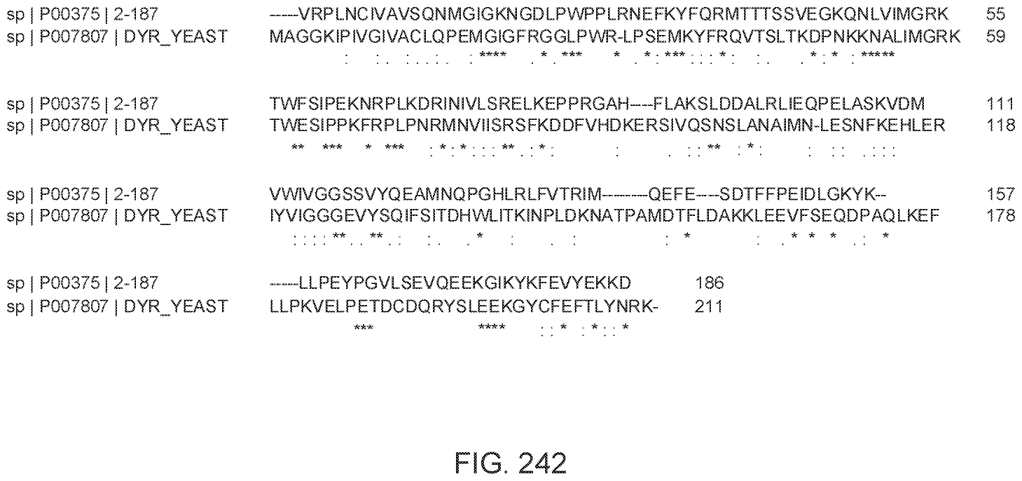

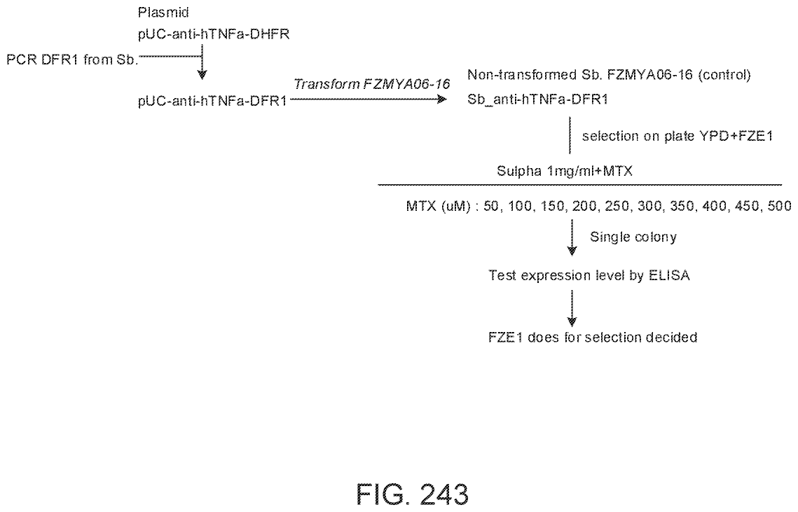

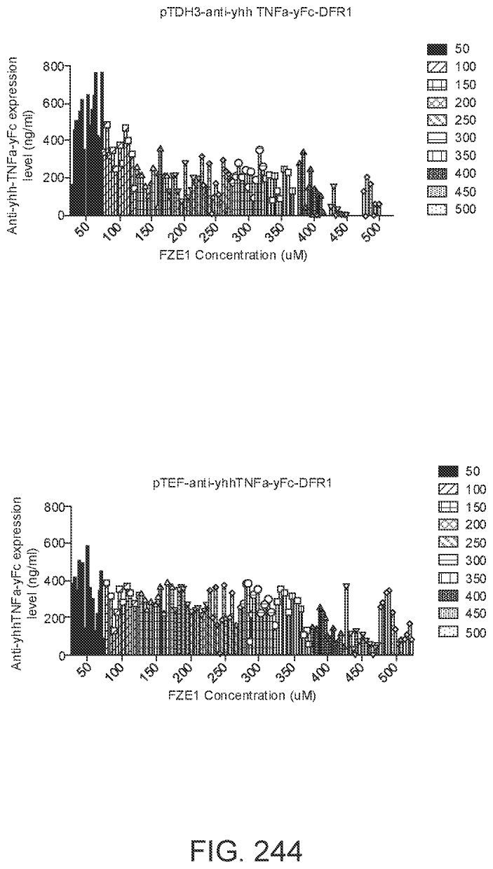

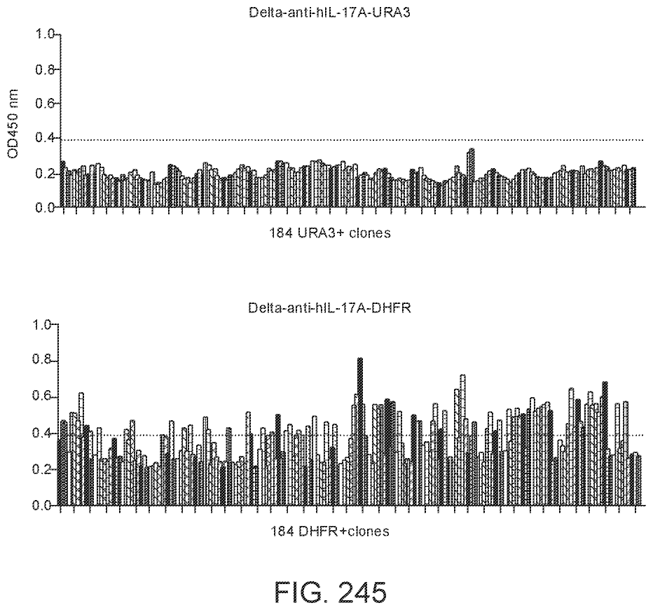

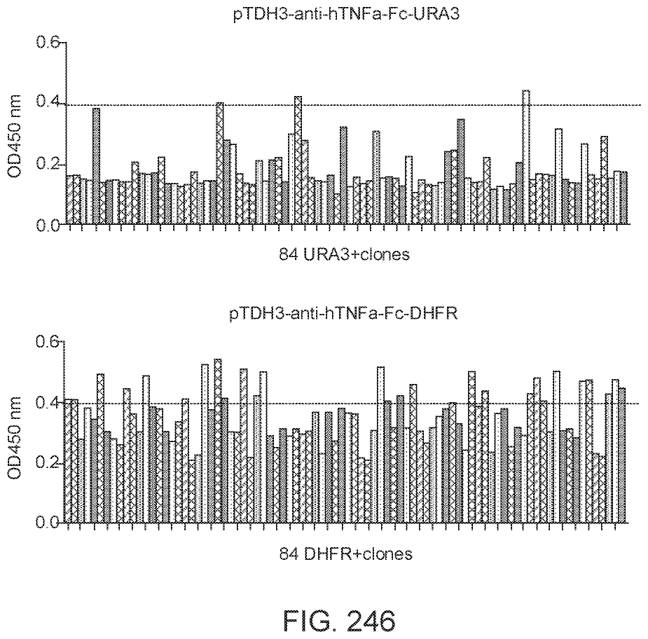

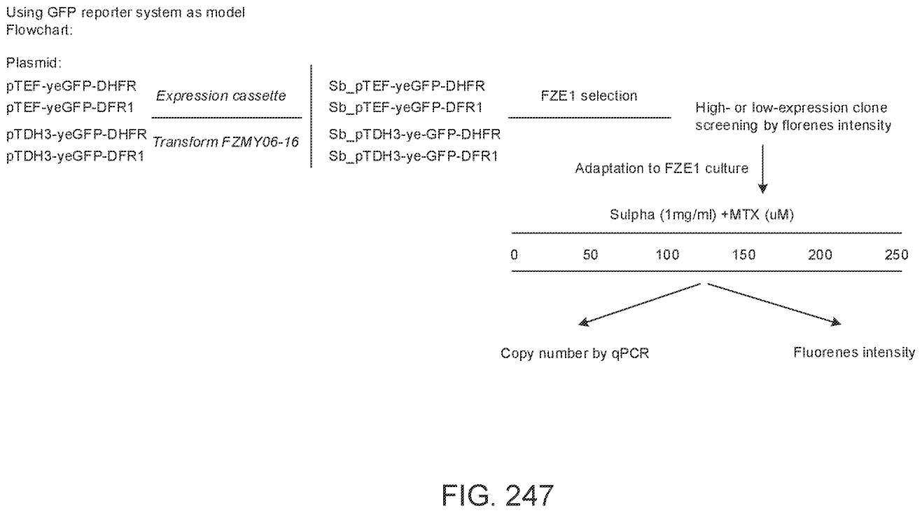

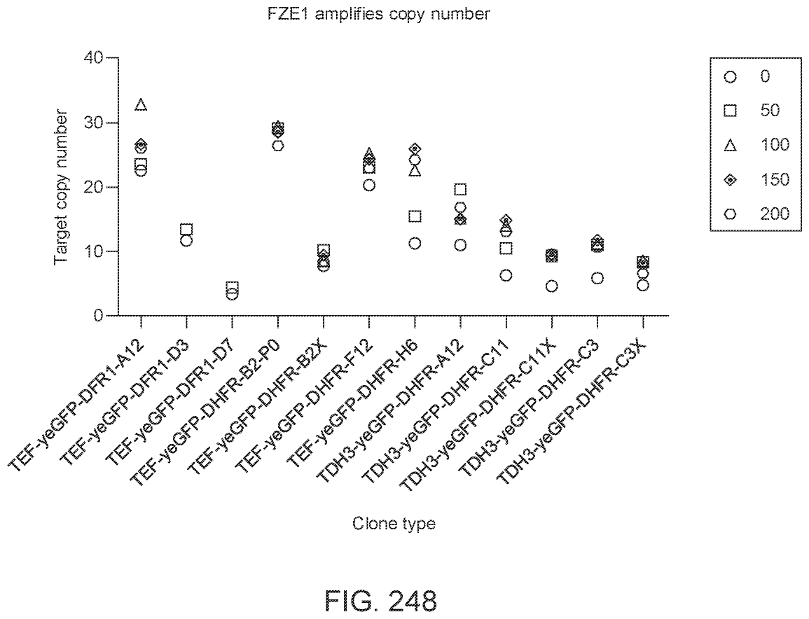

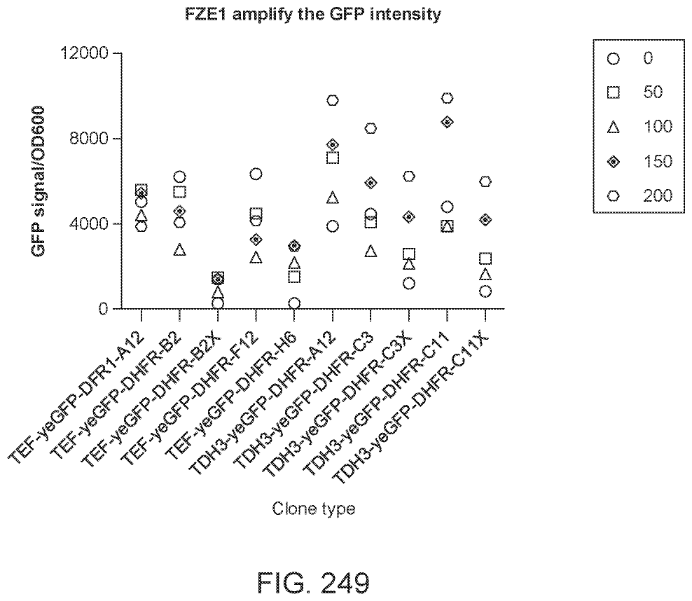

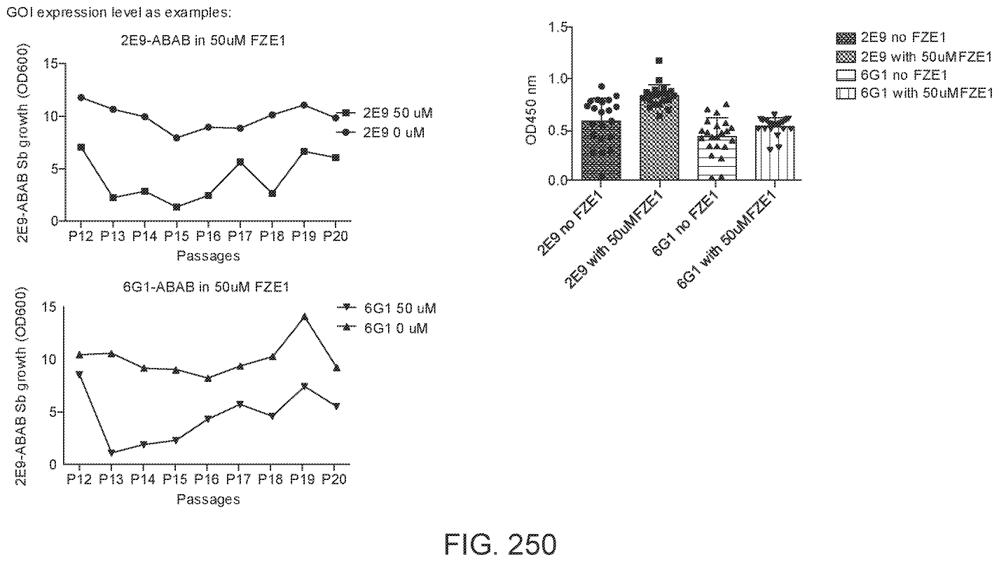

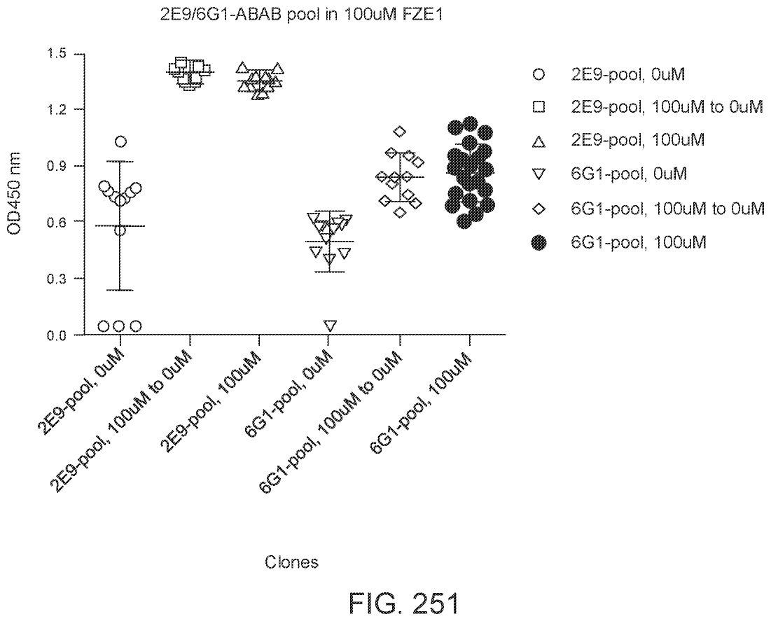

FIGS. 235-251 demonstrate development of FZE1 (methotrexate plus sulfanilamide) selection for S. boulardii.

FIG. 36 demonstrate use of deleting protease genes to increase polypeptide stability.

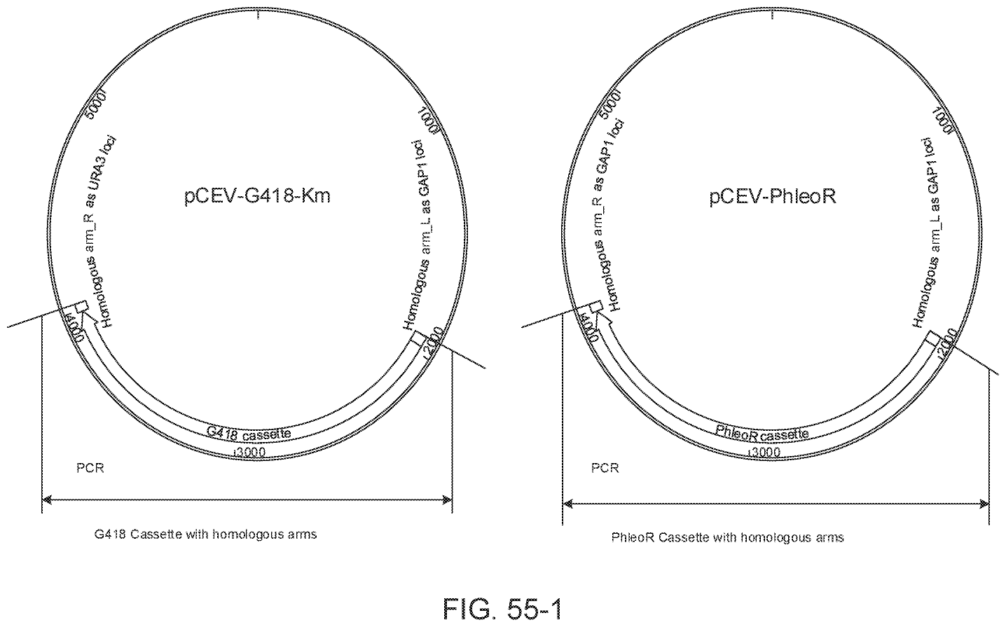

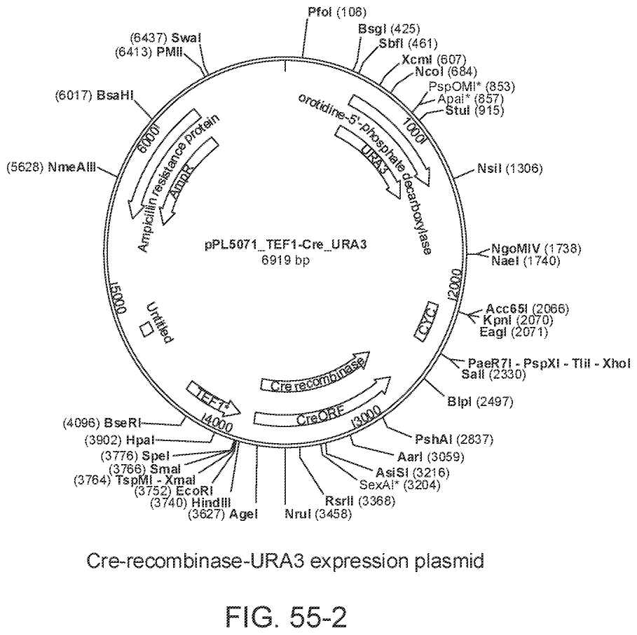

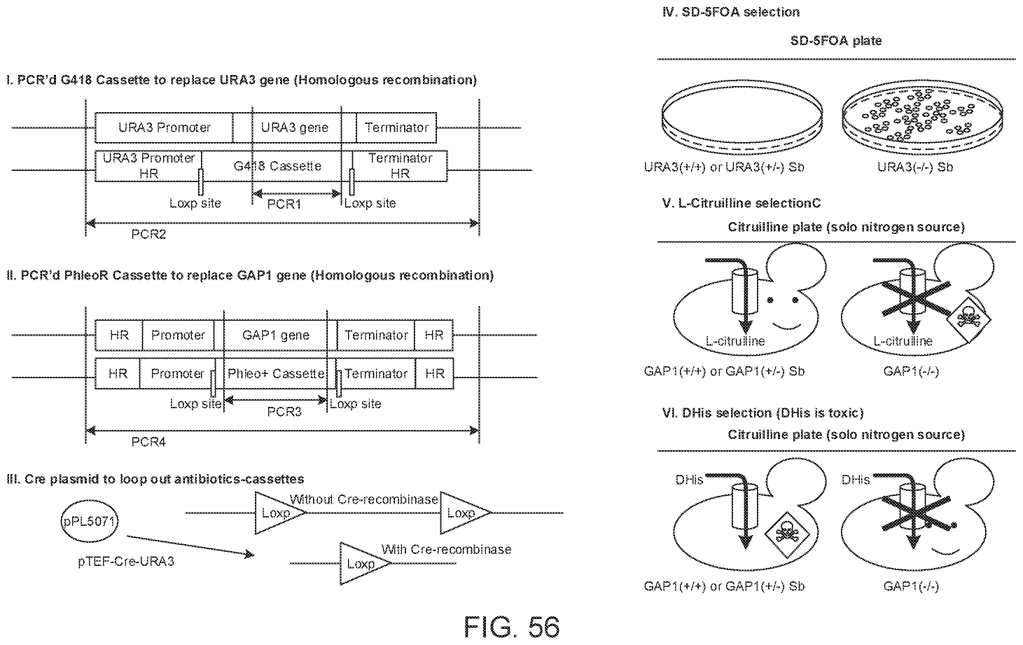

FIGS. 37-39, 55-57 shows generation of exemplary auxotrophic strains.

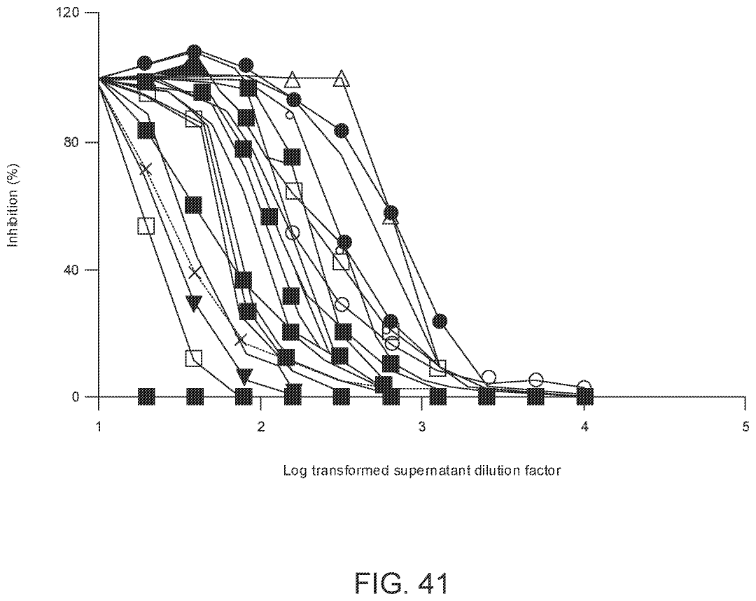

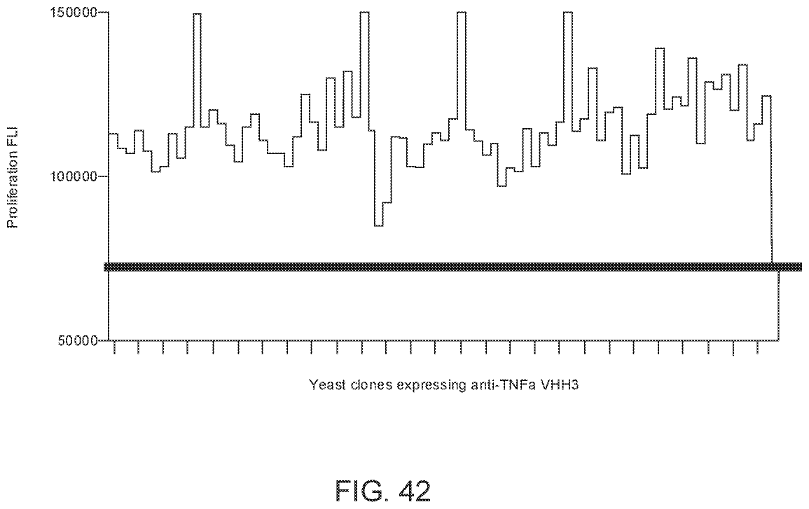

FIGS. 40-42 show exemplary engineered strains of S. boulardii to express anti-TNF-α VHH3 and characterization of same.

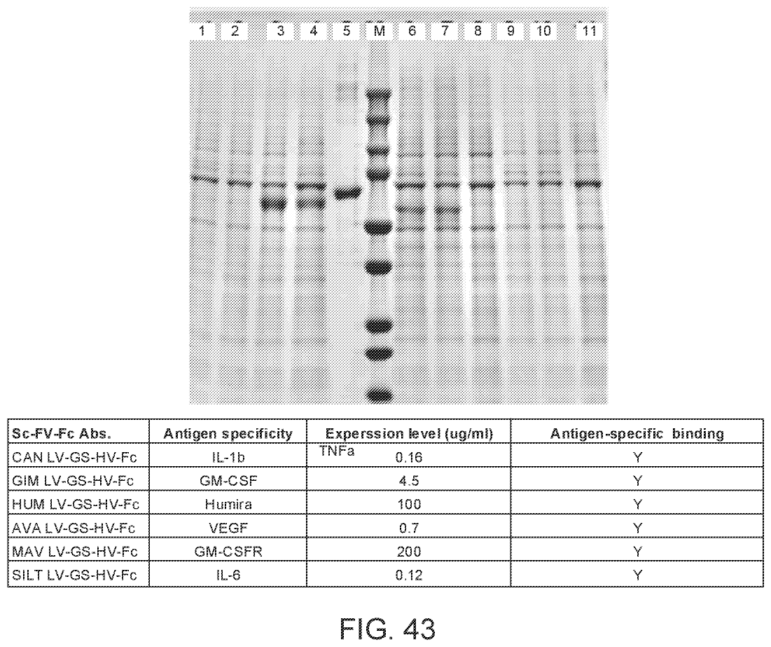

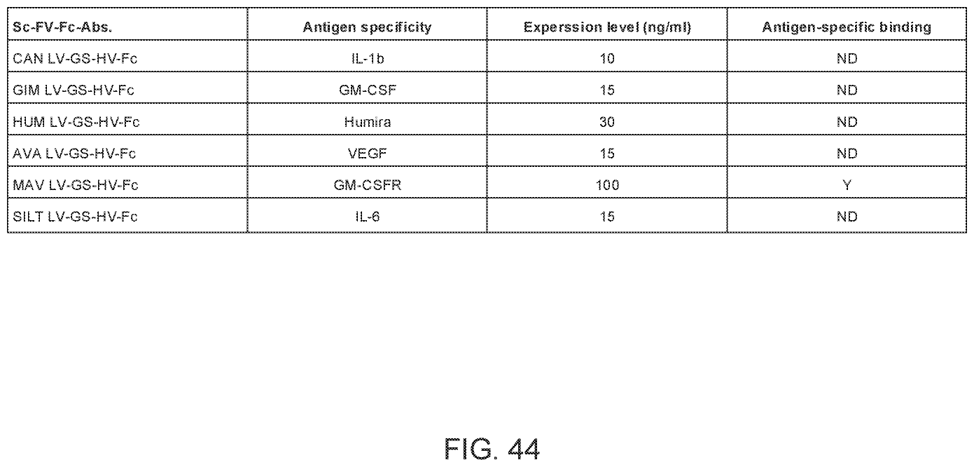

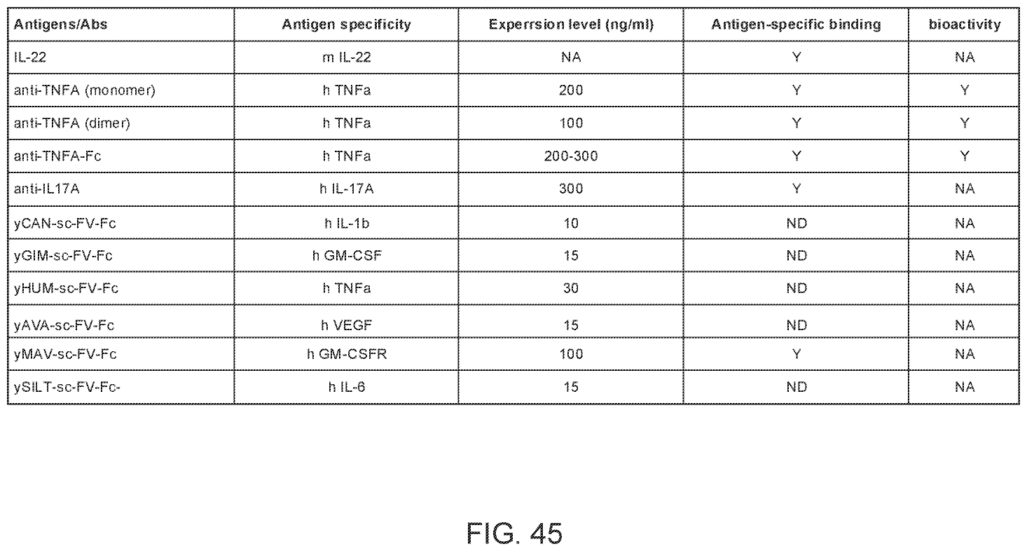

FIGS. 43-45 show expression of functional Sc-FV-Fc antibodies by S. boulardii and expression of functional VHHs, VHHs-Fc, VHH-Sc-FV, VHH-Fc-VHH antibodies by S. boulardii.

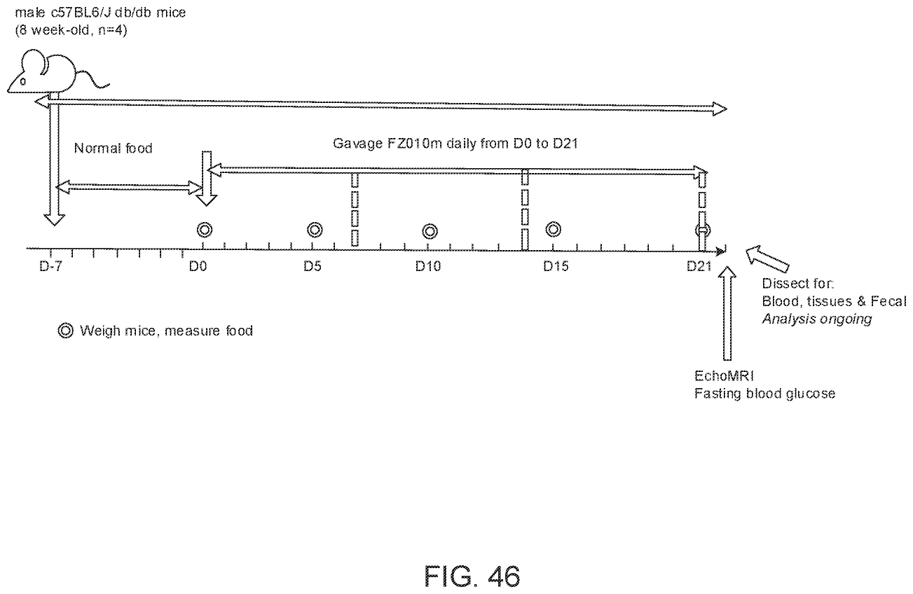

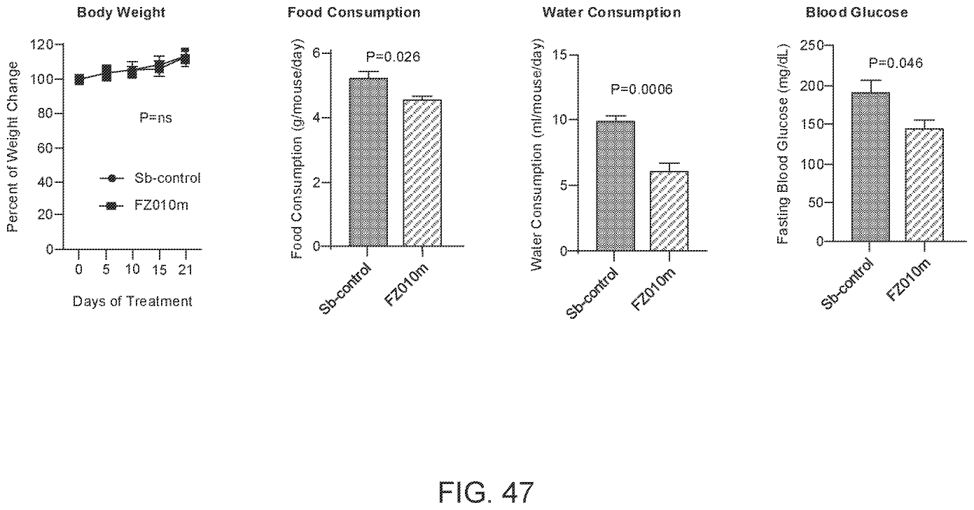

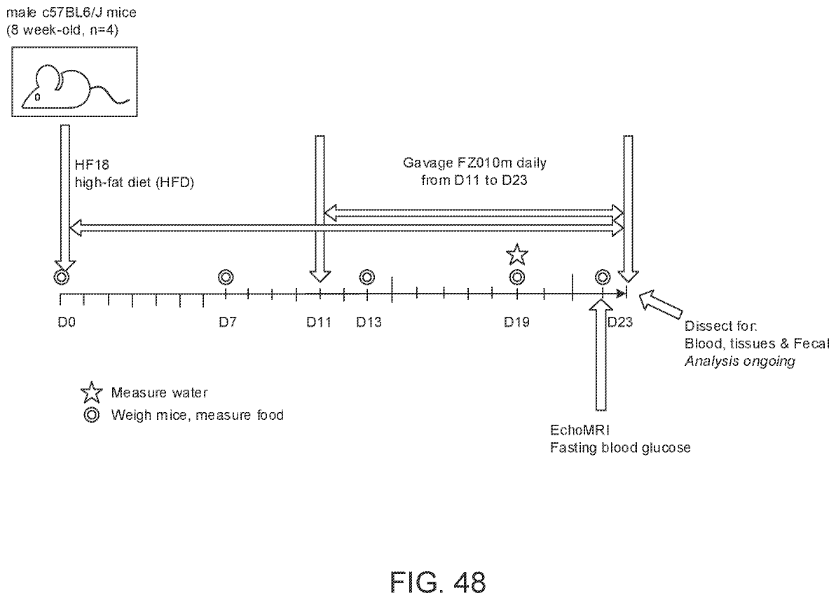

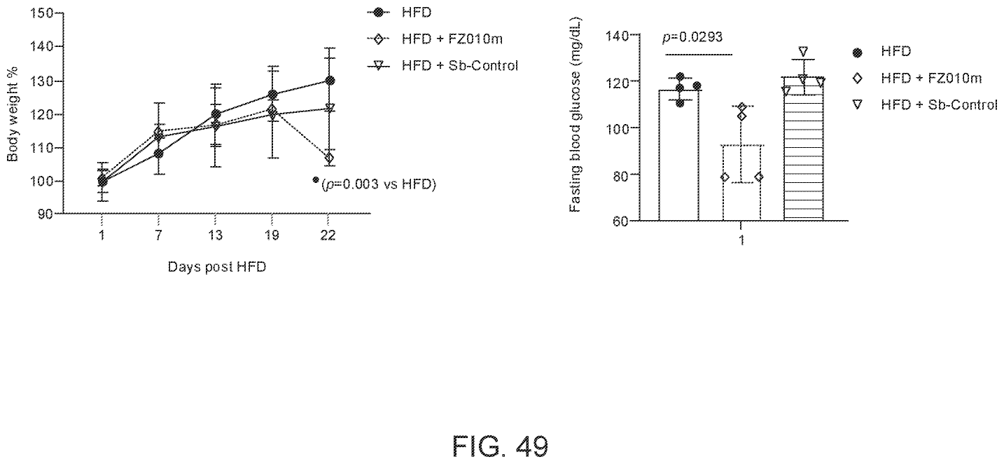

FIGS. 46-49 demonstrate exemplary use of FZ010m in mouse obesity models.

FIGS. 53-54 demonstrate exemplary evaluation of constructs for genomic insertion (expression and cell-based neutralization).

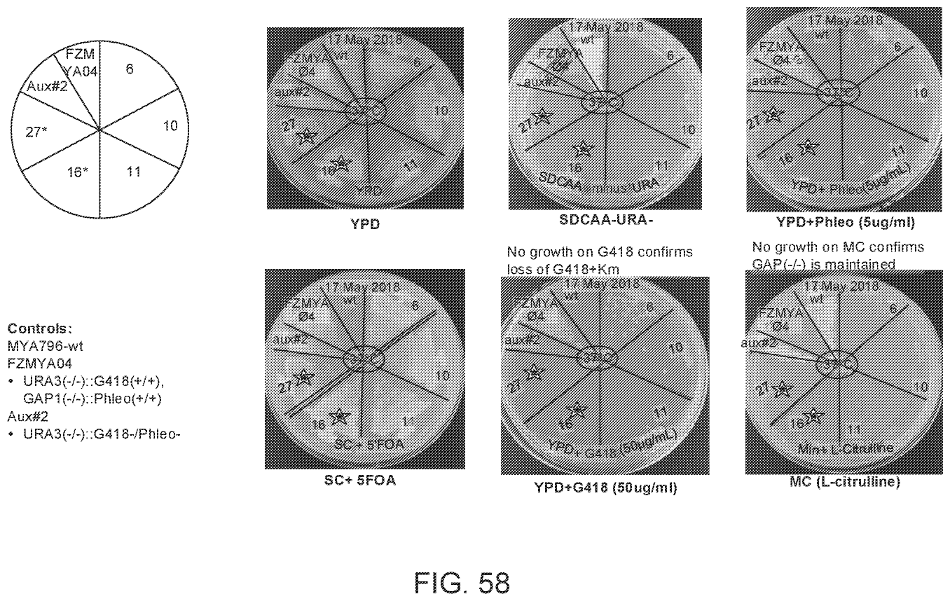

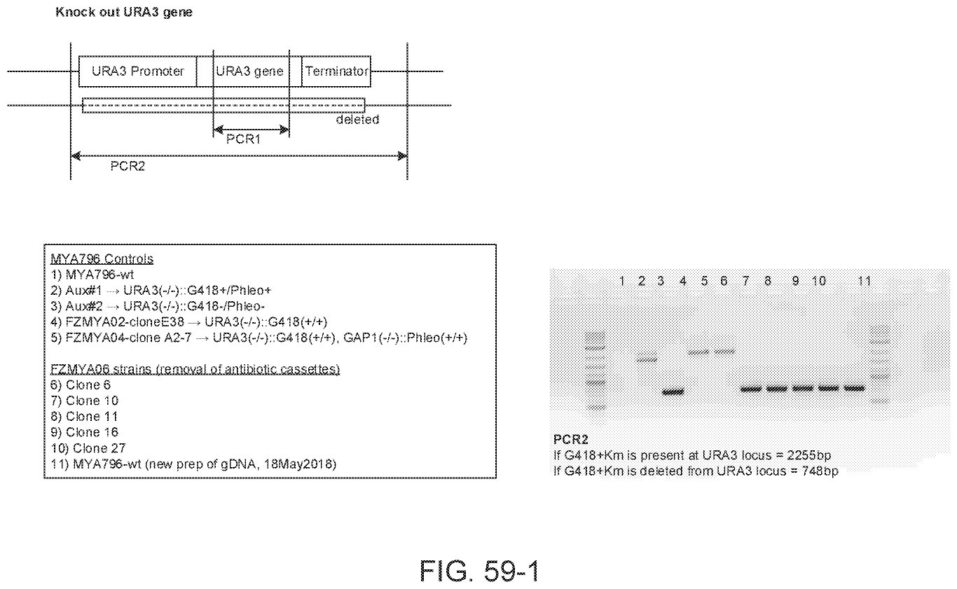

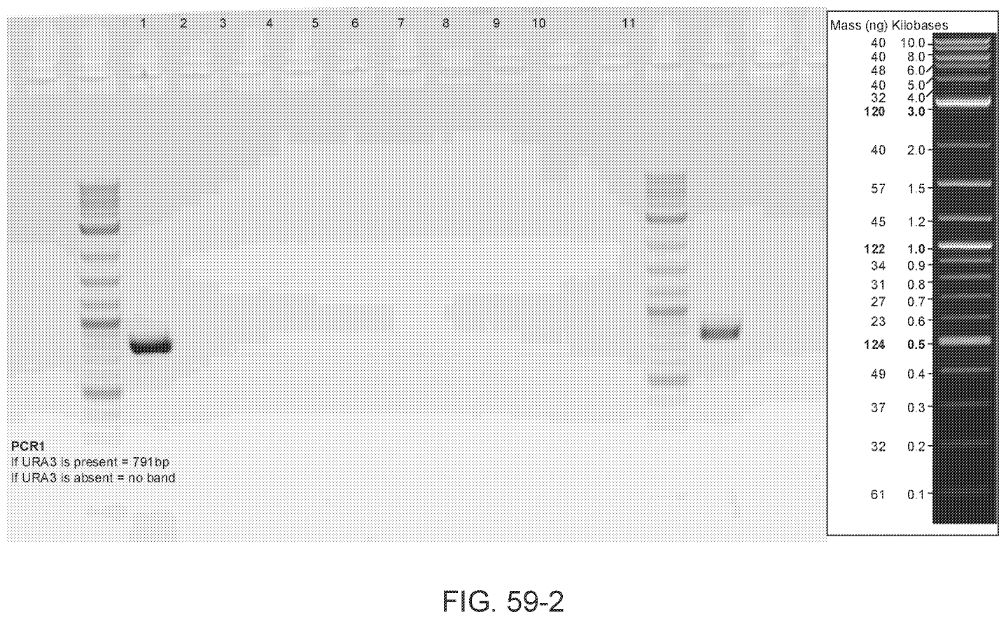

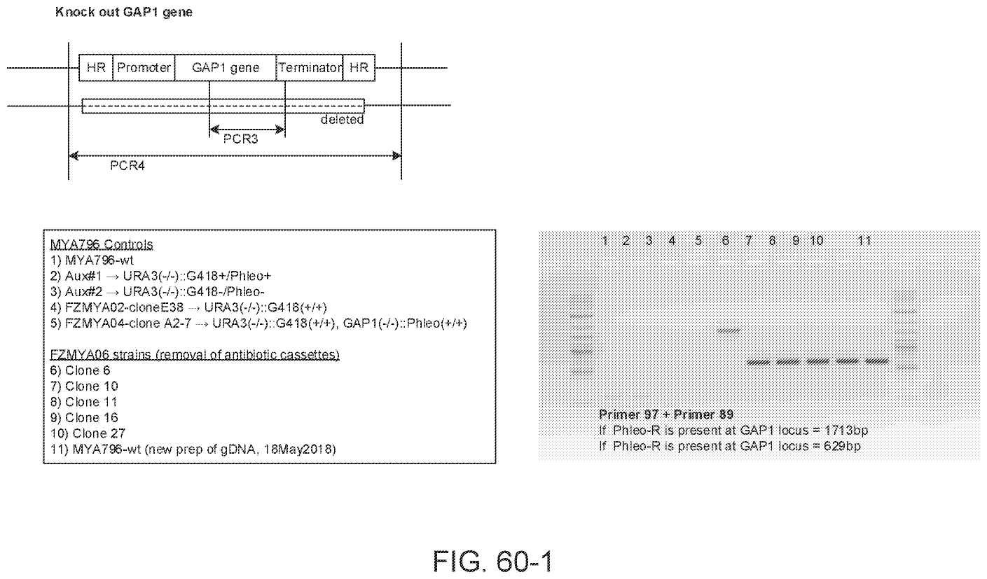

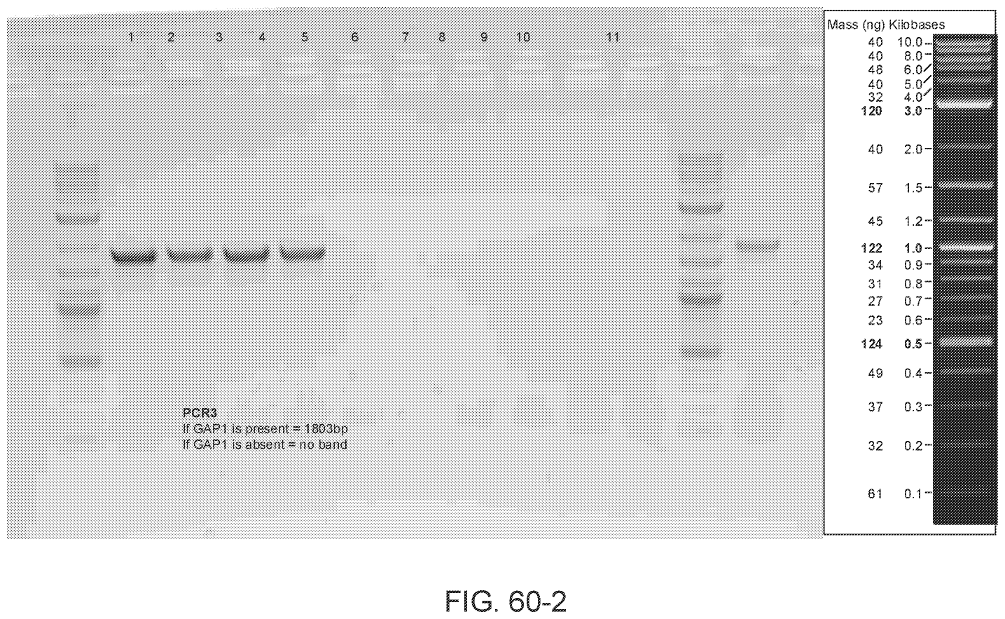

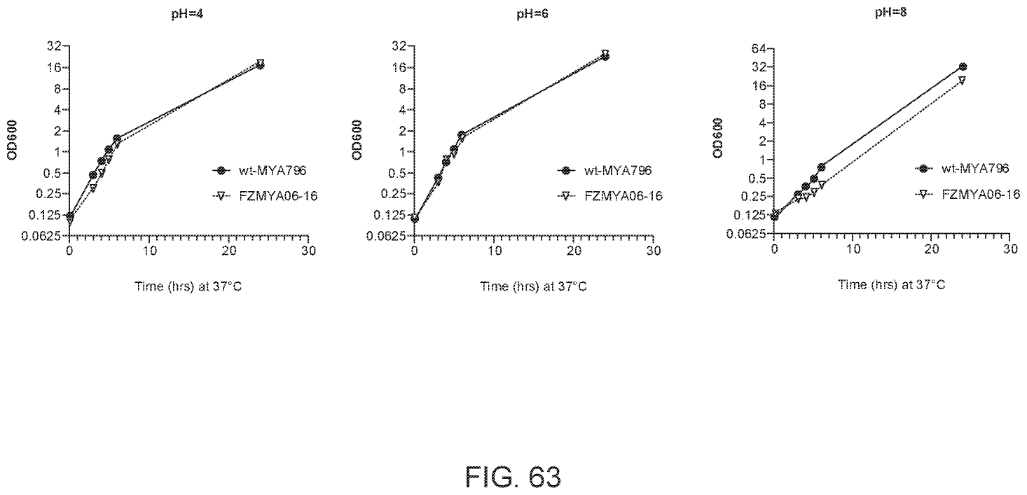

FIGS. 58-63 shows exemplary characterization of FZMYA06-16. Exemplary characterization include evaluation of growth phenotype, PCR confirmation of the site-specific deletion of antibiotics cassette, sequencing of LoxP-scar at ura3 and gap1 loci, growth curve and antibiotic resistance.

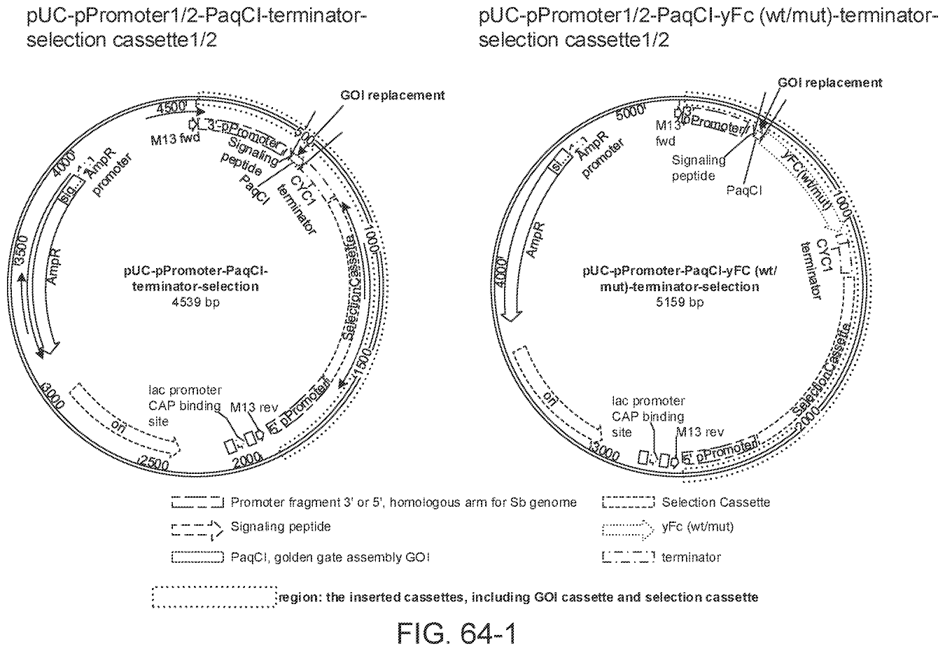

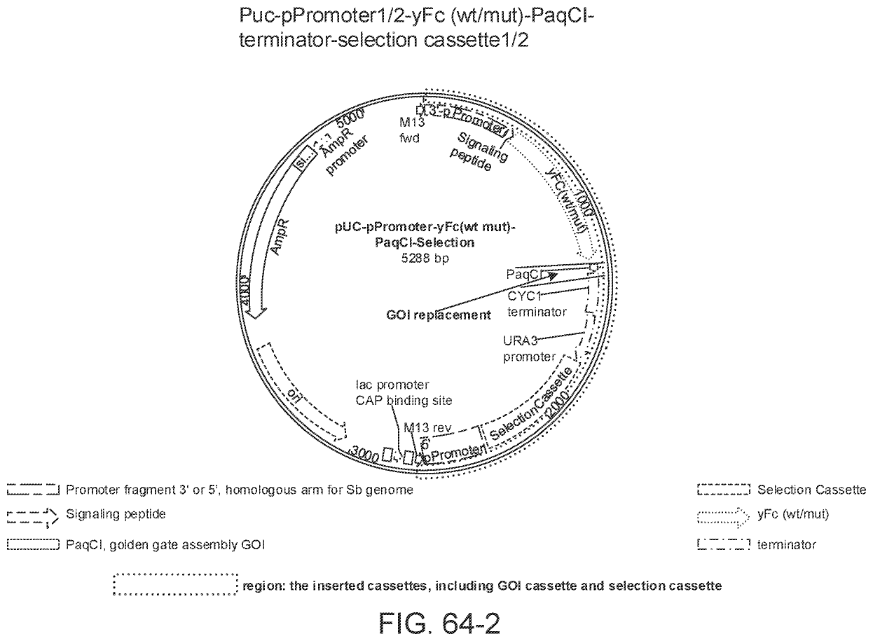

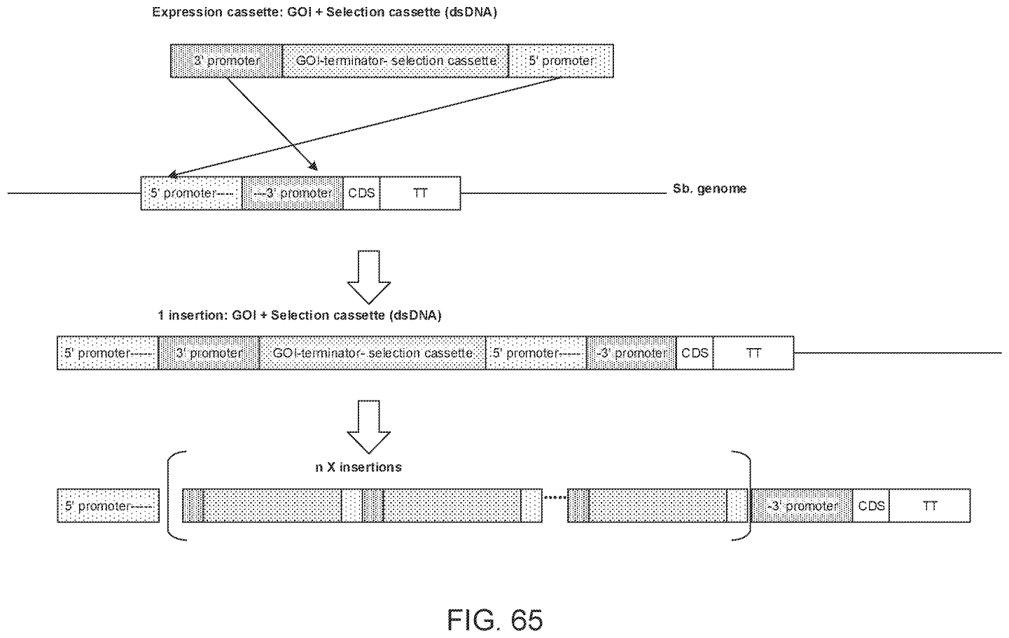

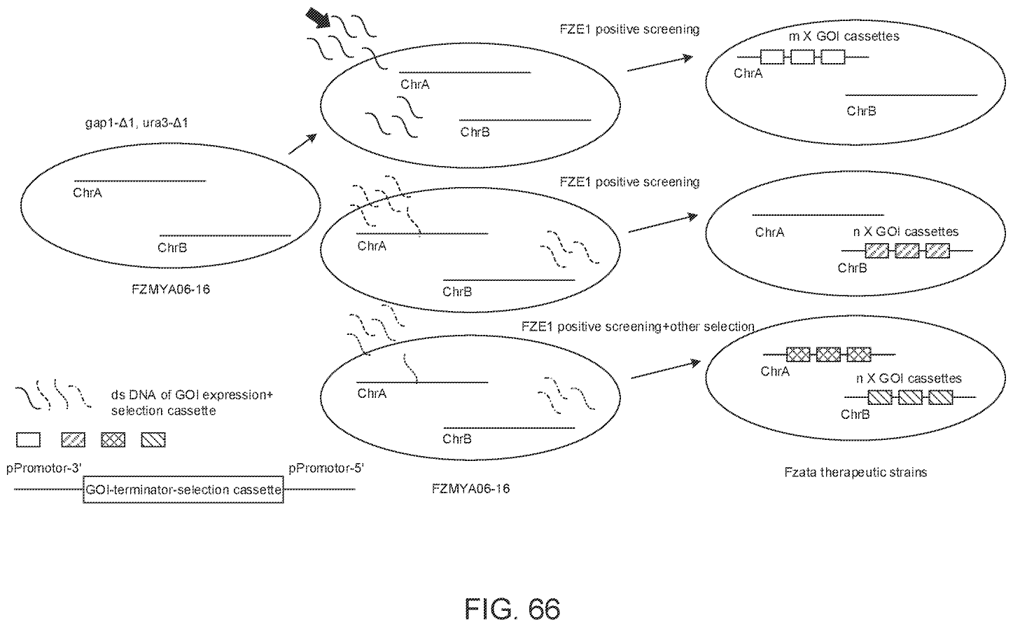

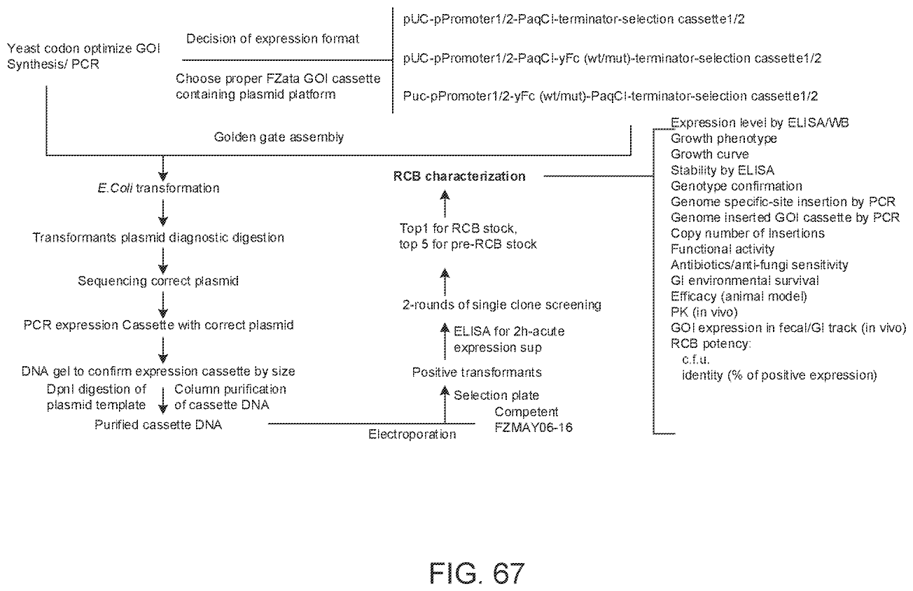

FIGS. 64-65 shows exemplary nucleic acid (e.g., plasmid containing GOI cassette) and schematic of insertion of GOI cassette.

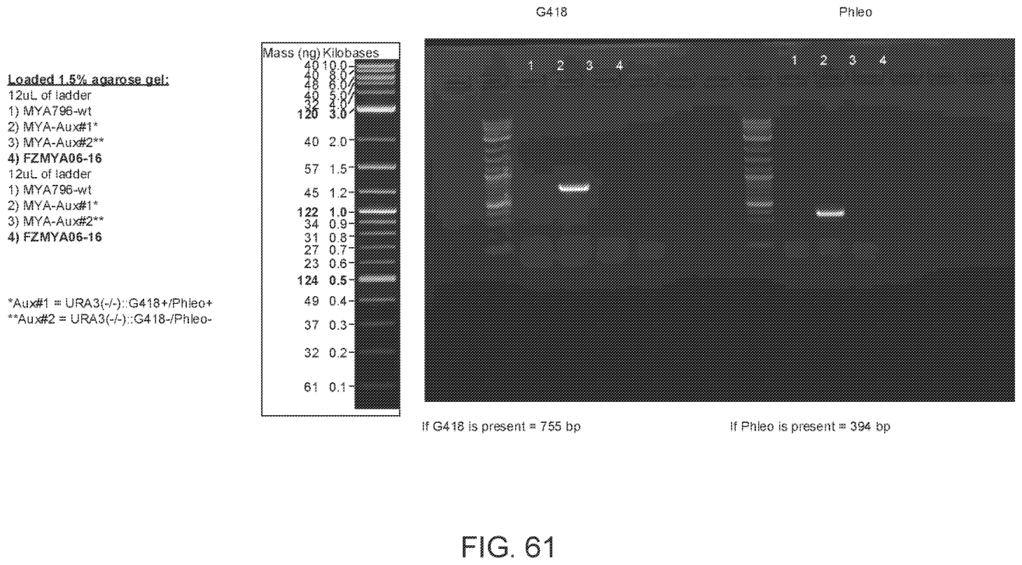

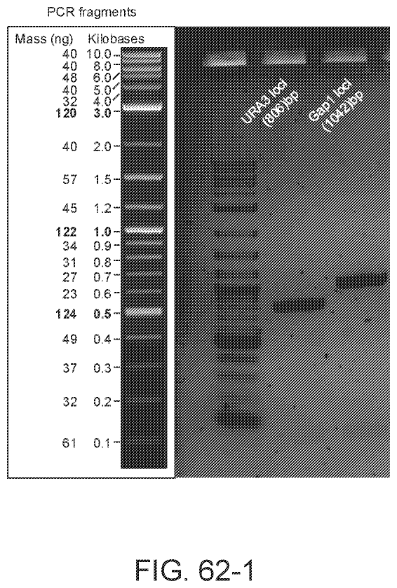

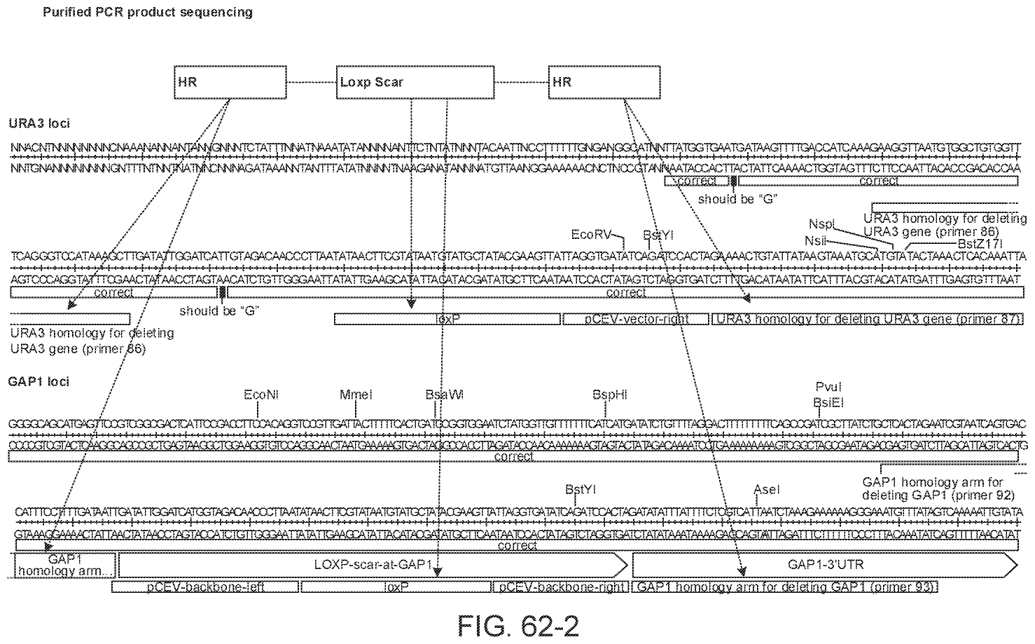

FIG. 61-62 shows exemplary generation of engineered strains of Saccharomyces yeast.

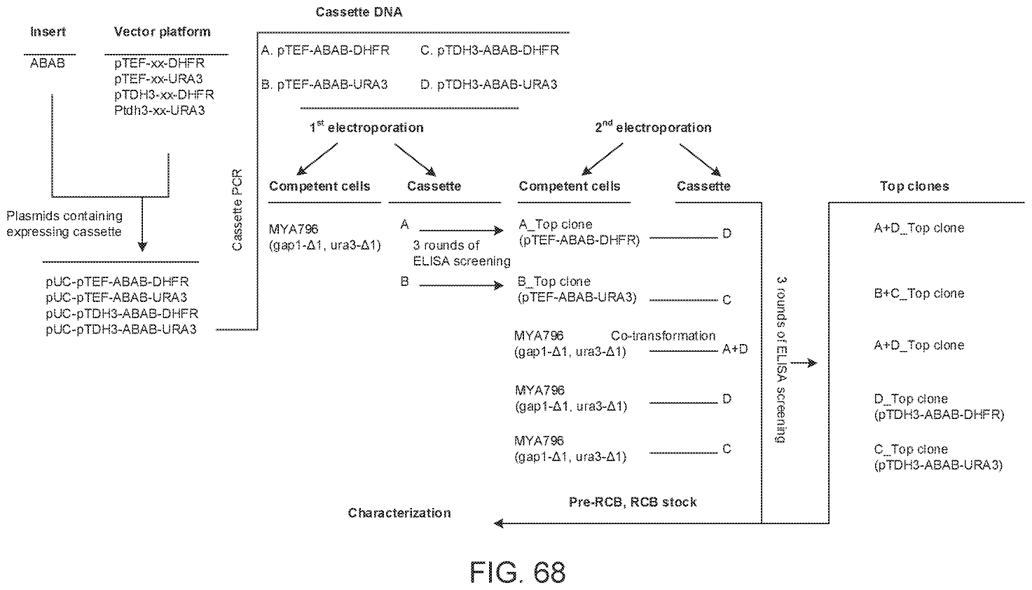

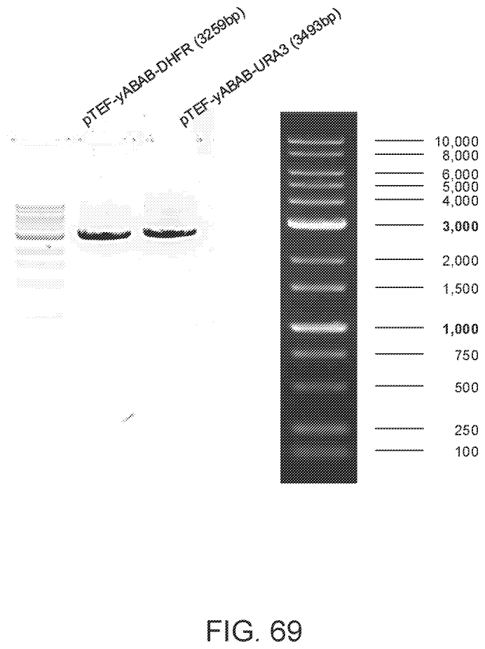

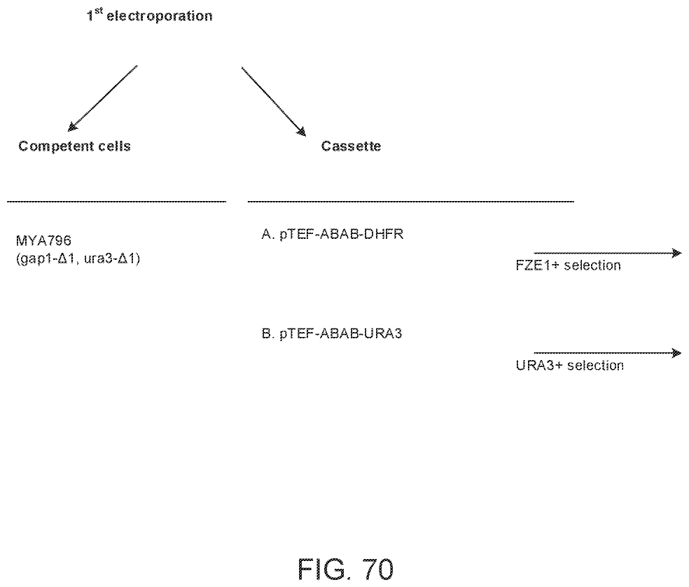

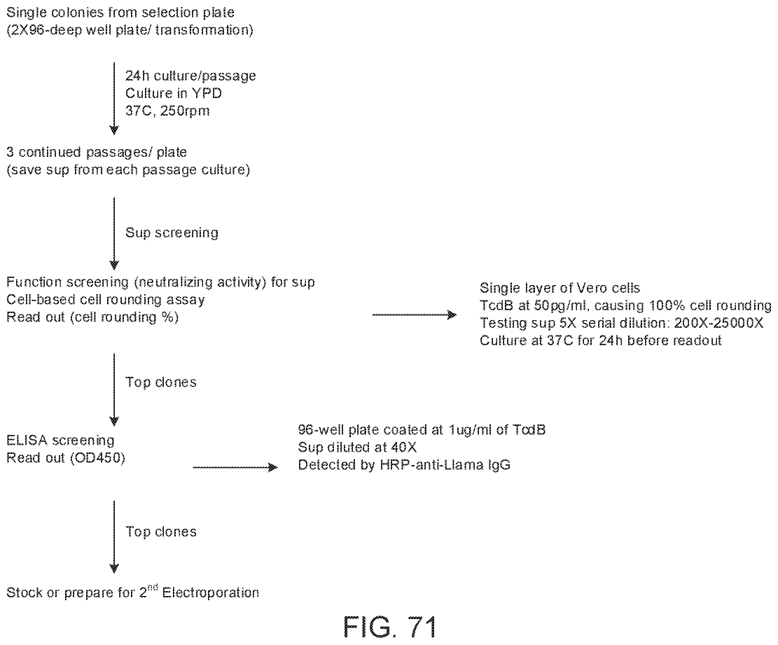

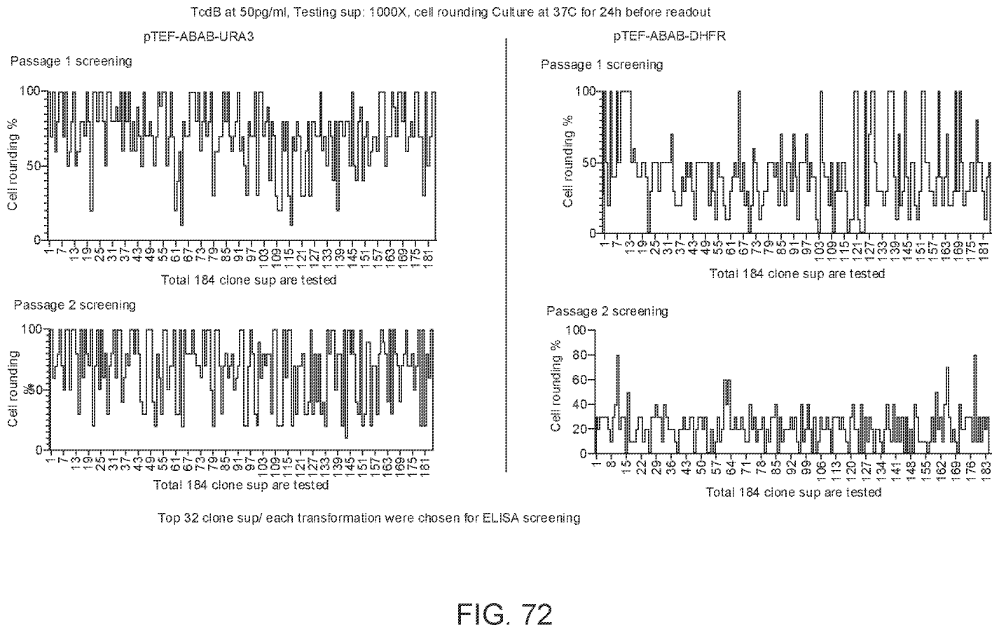

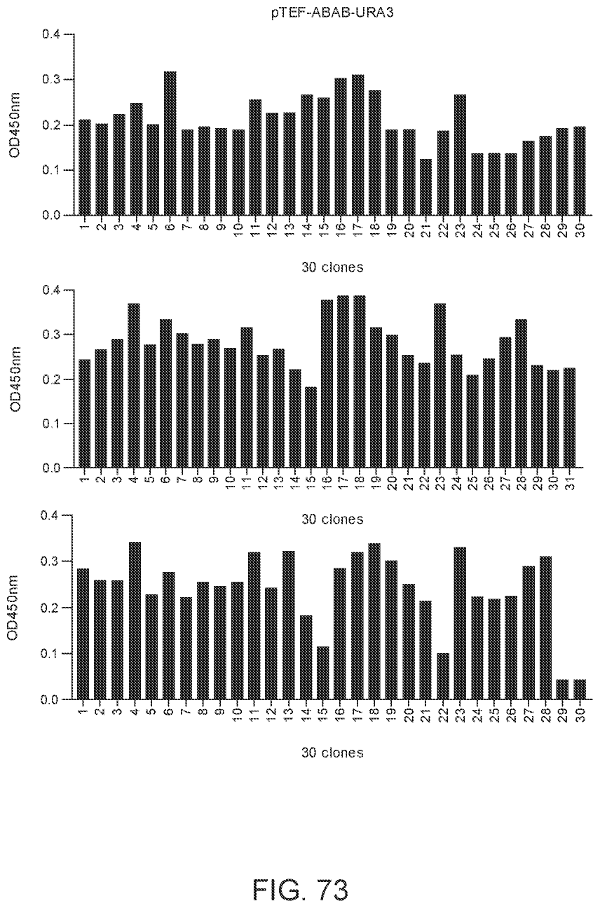

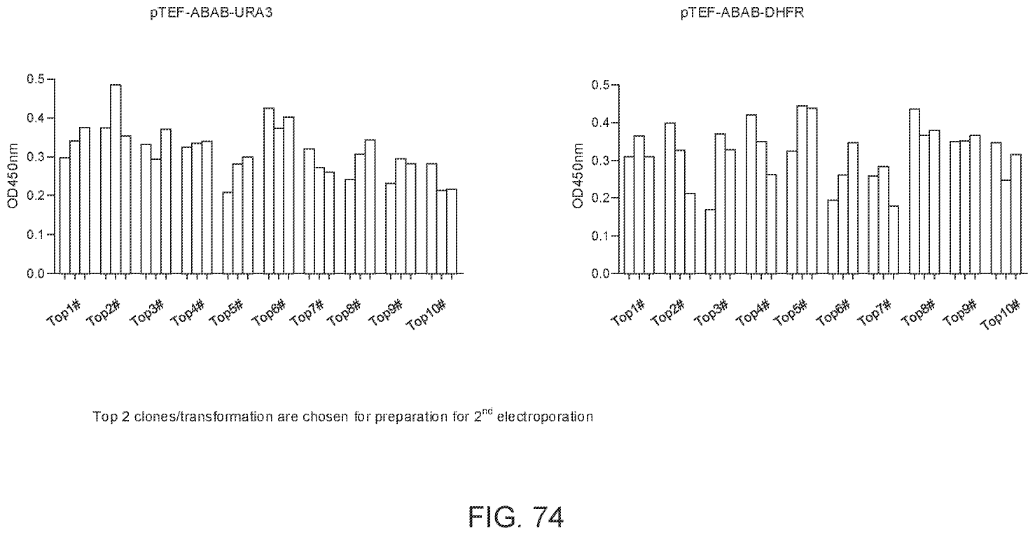

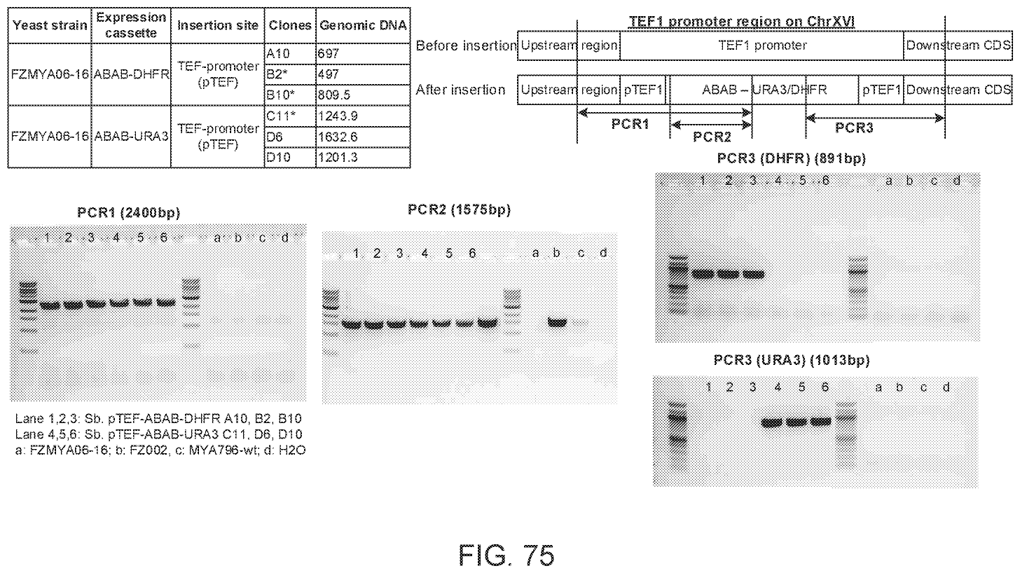

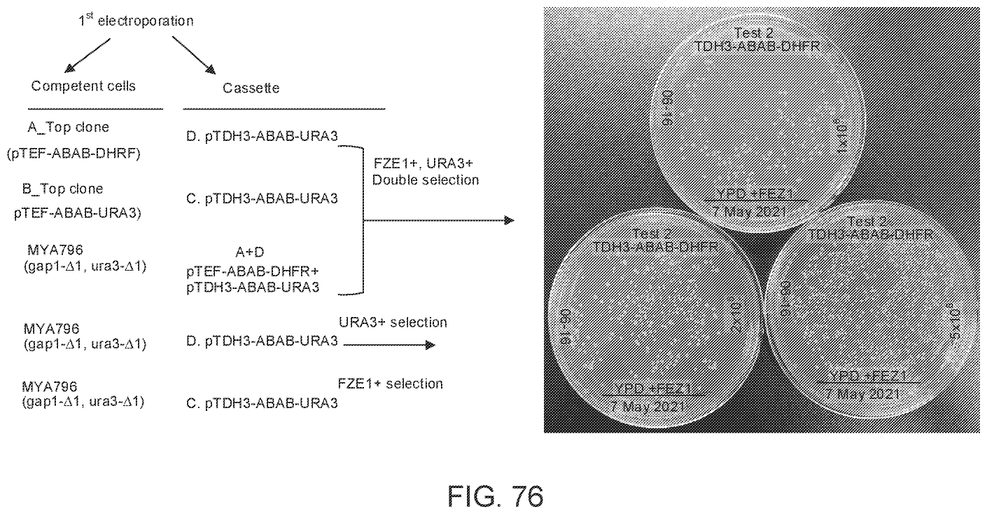

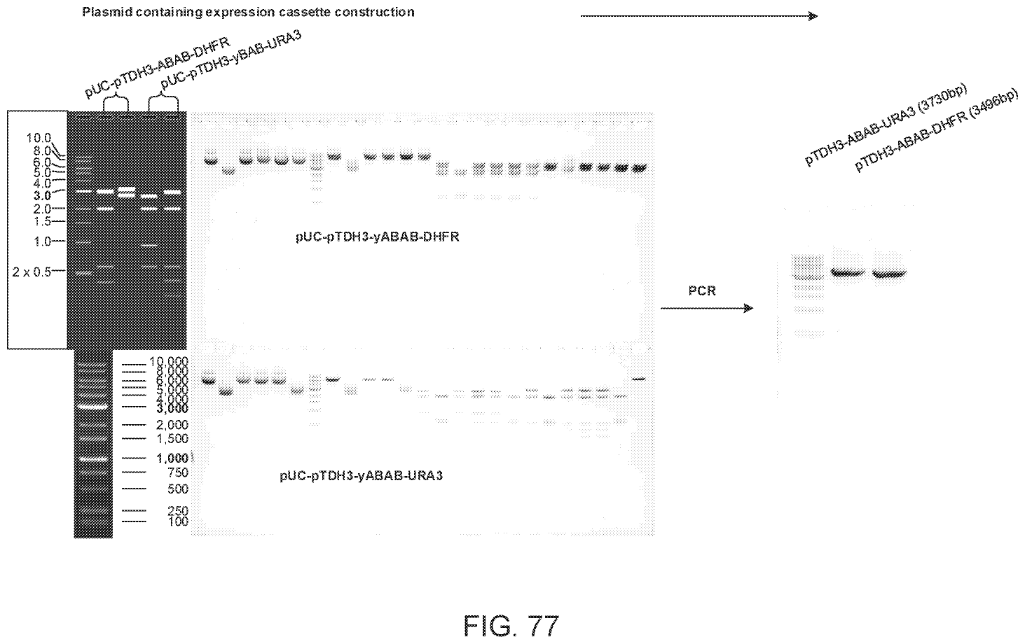

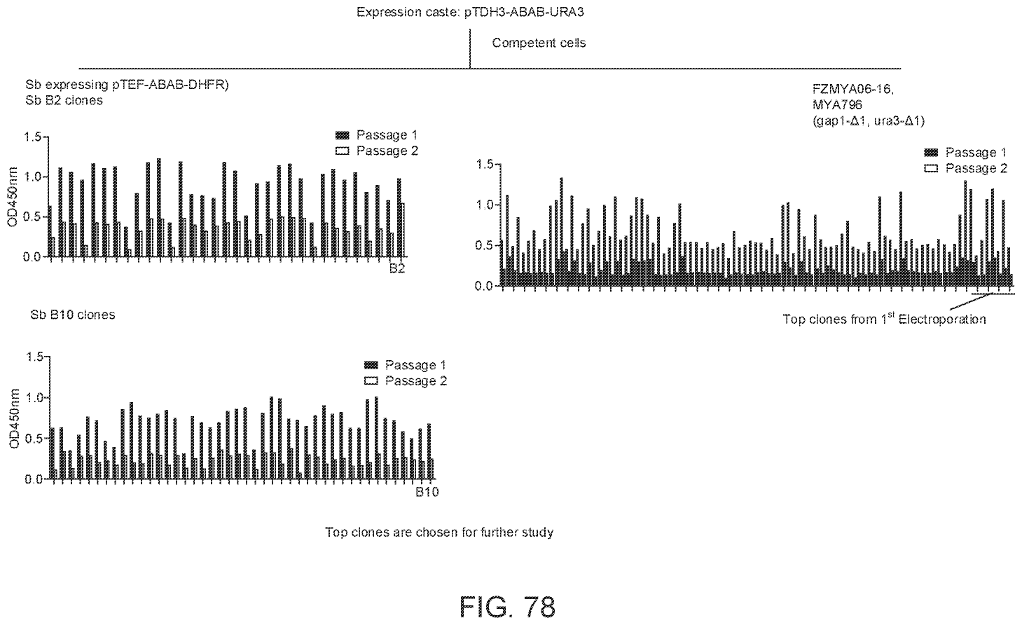

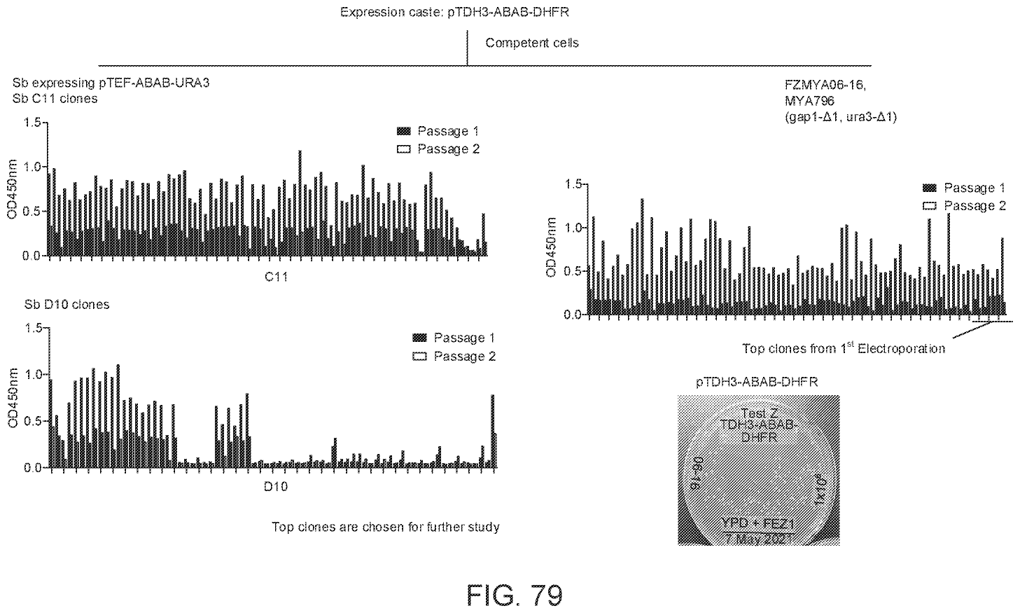

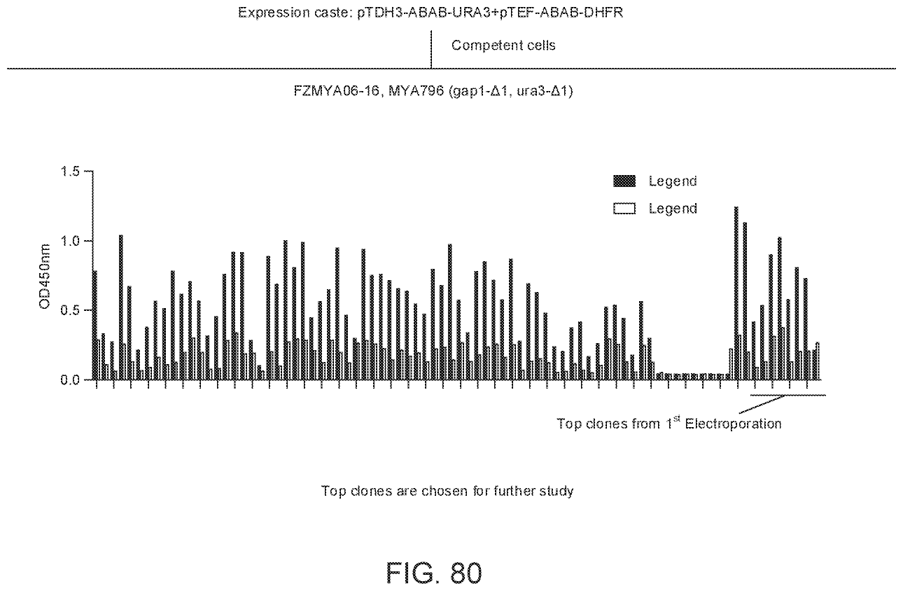

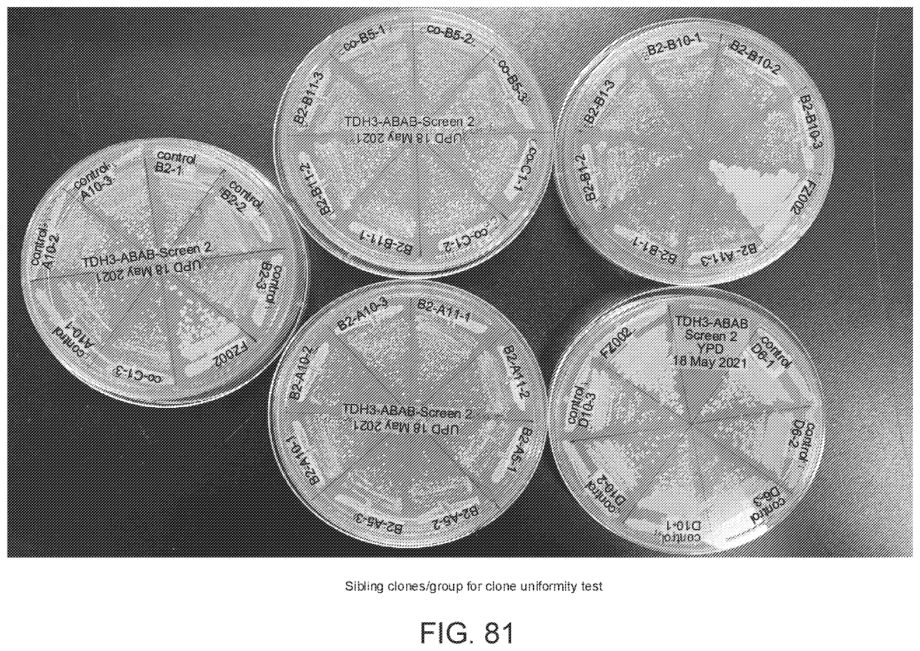

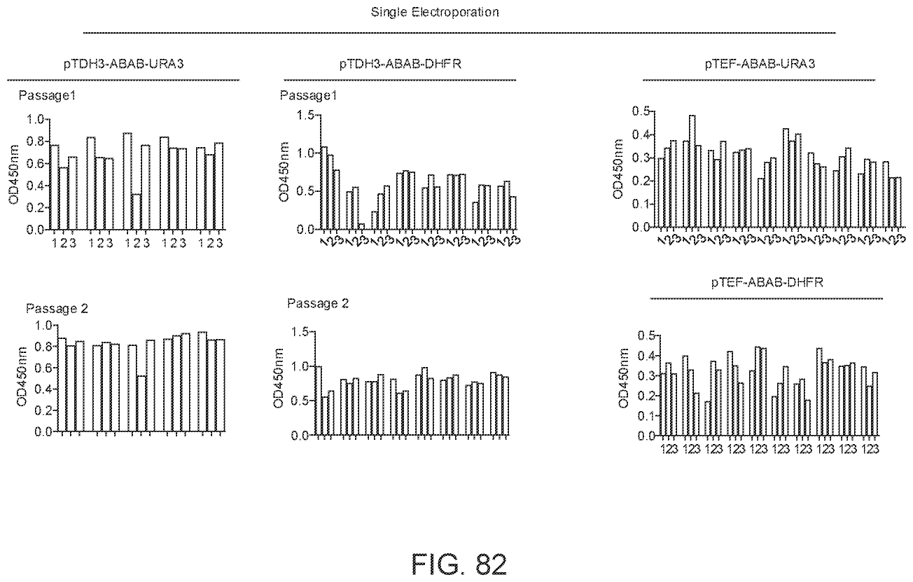

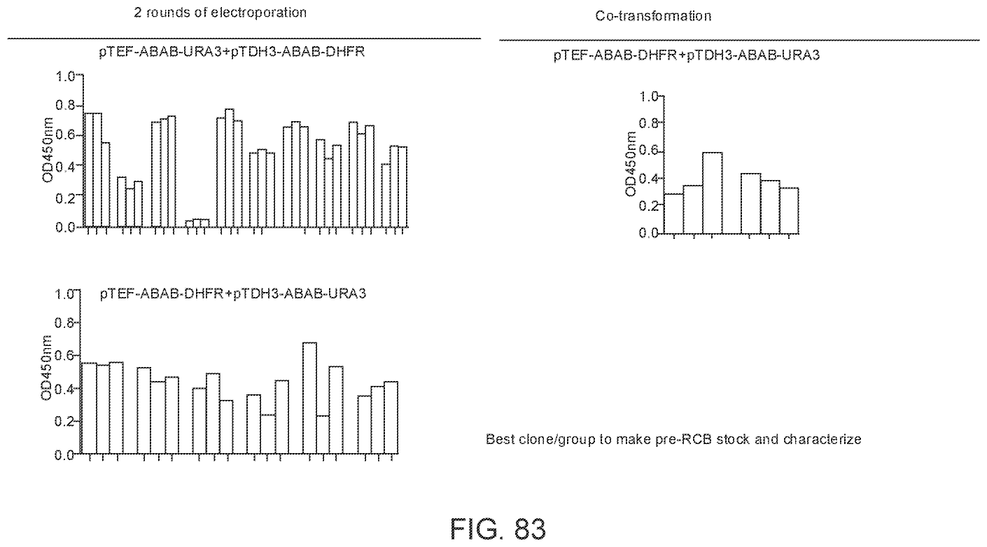

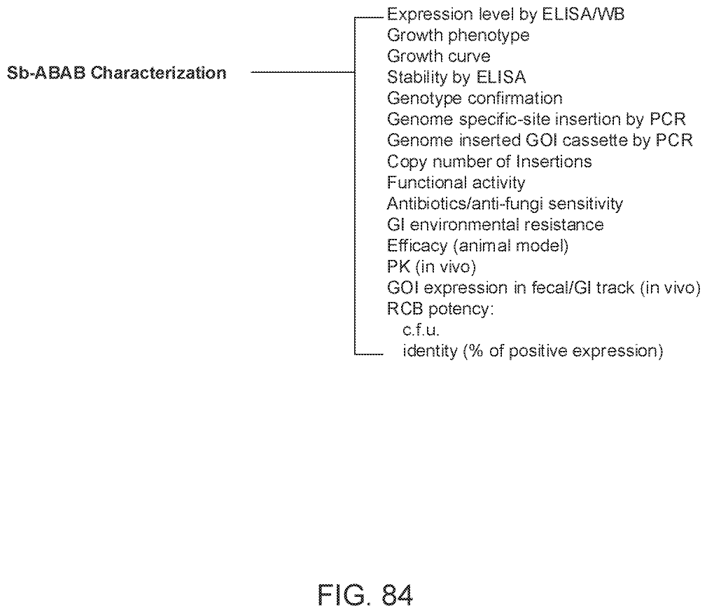

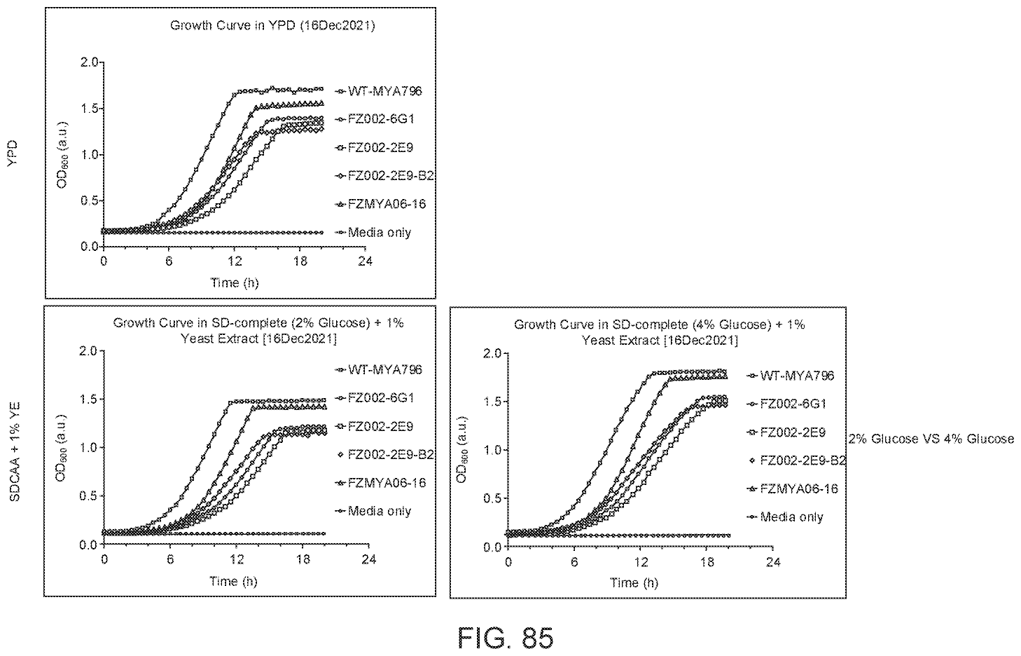

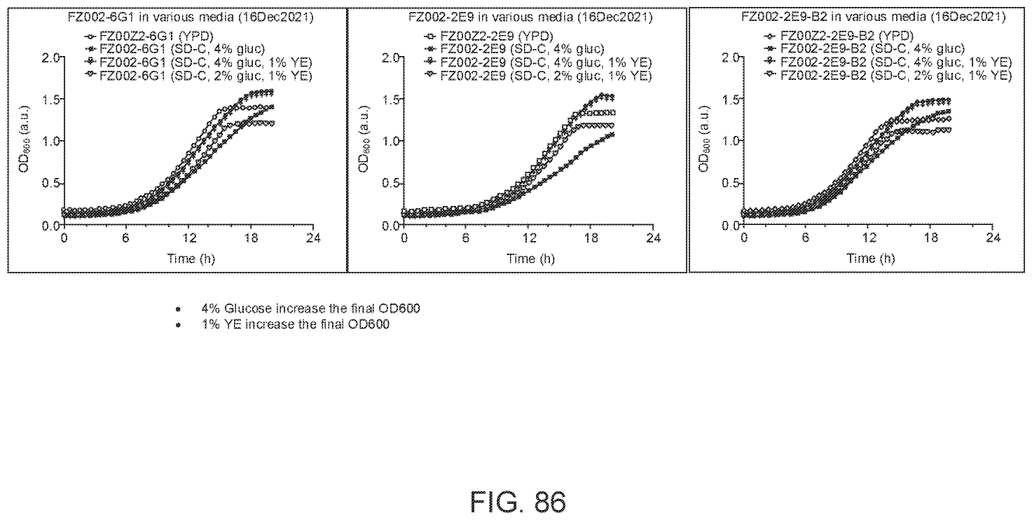

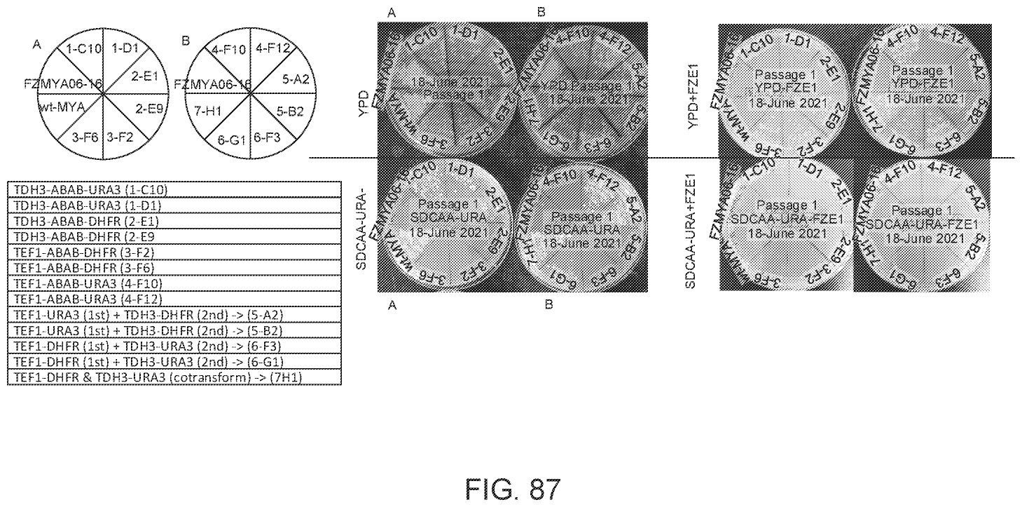

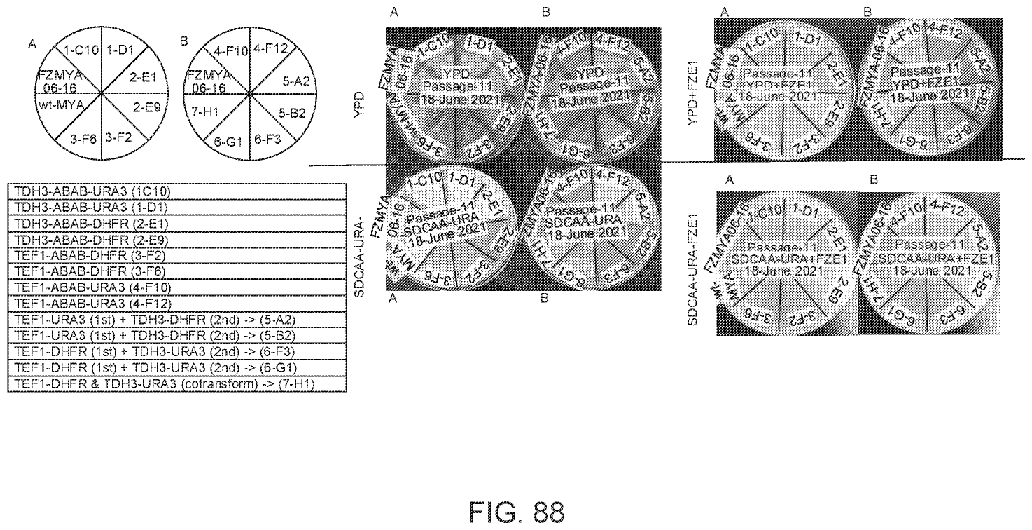

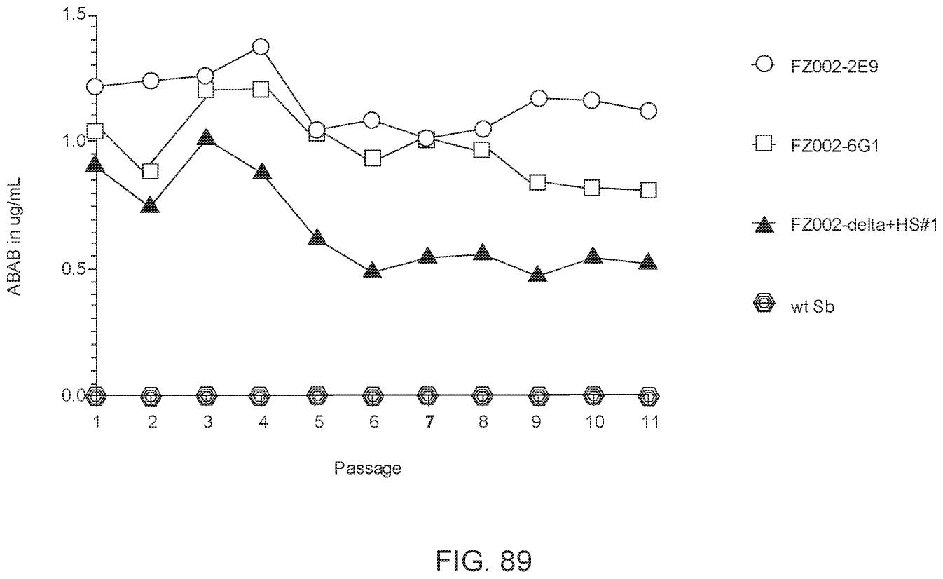

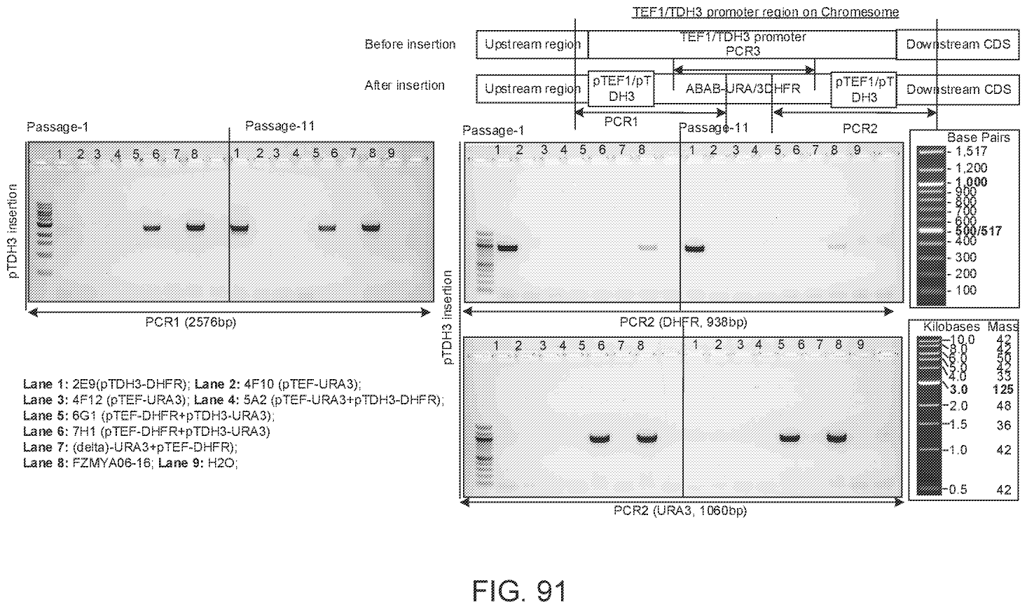

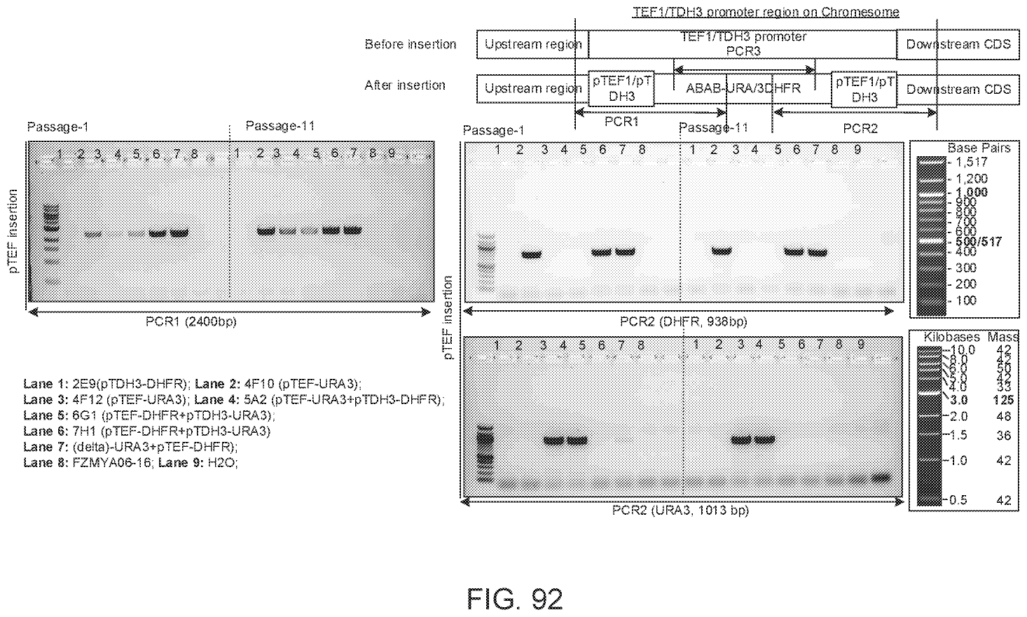

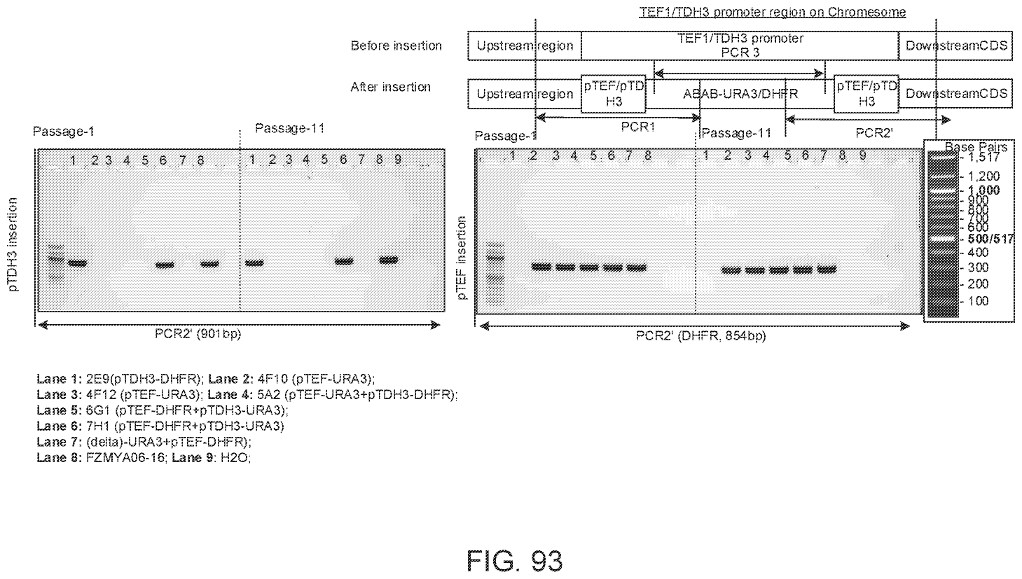

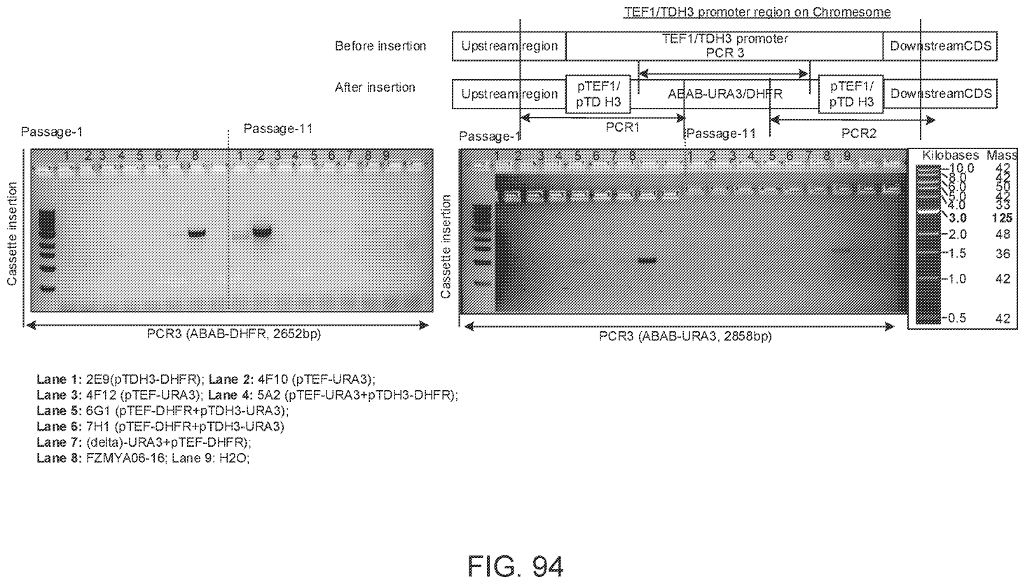

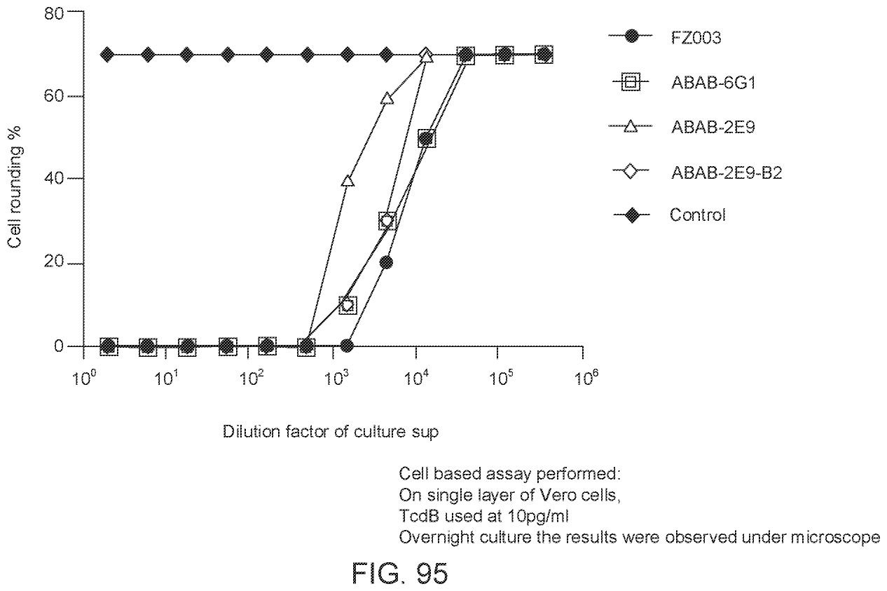

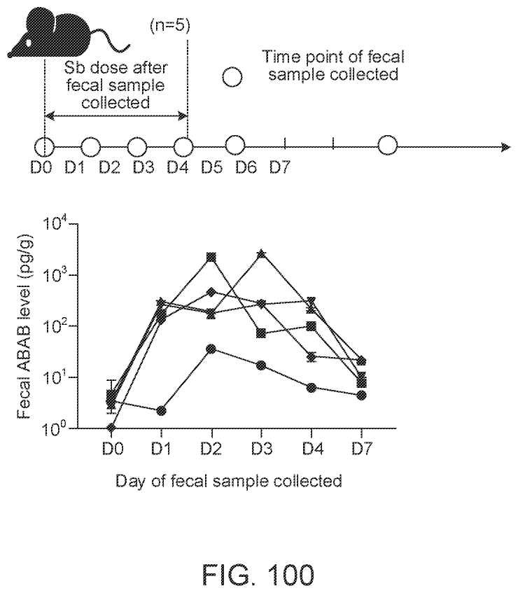

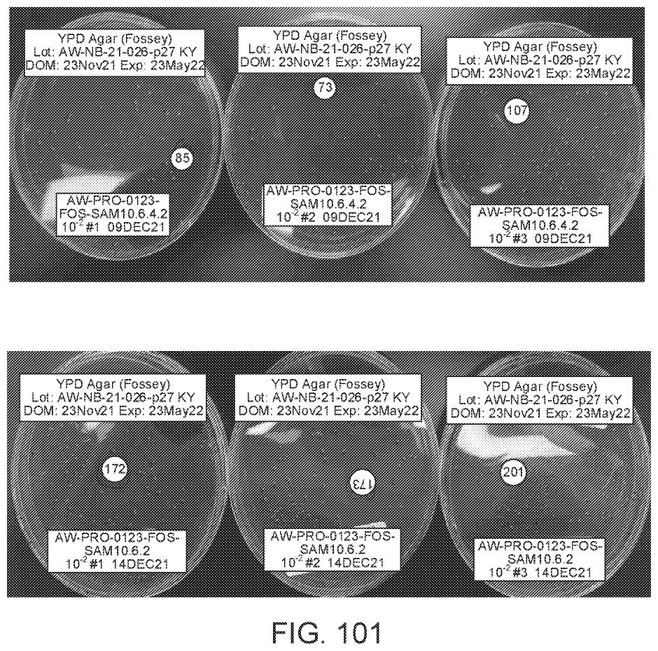

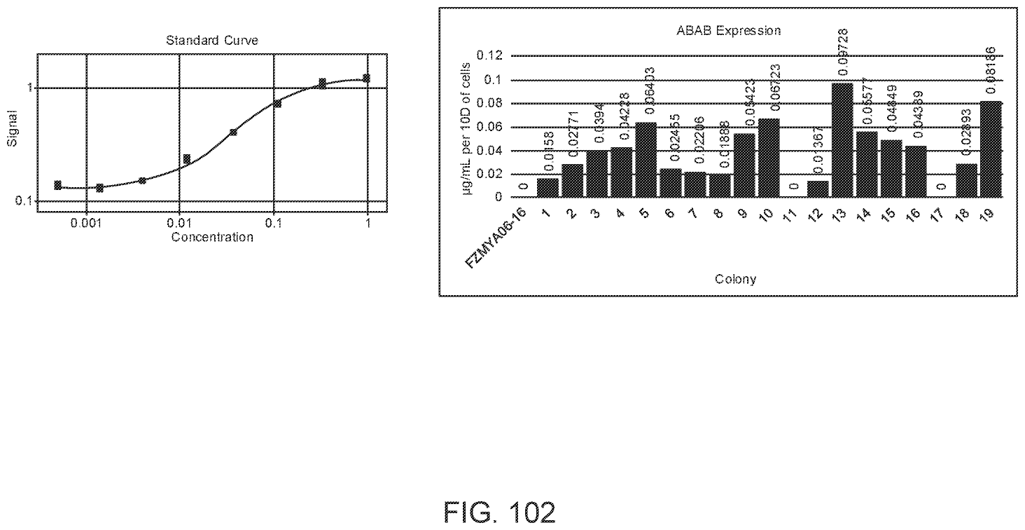

FIGS. 68-102 shows exemplary generation and characterization of engineered strain of S. boulardii expression tetra-specific antitoxin ABAB (e.g., FZ002).

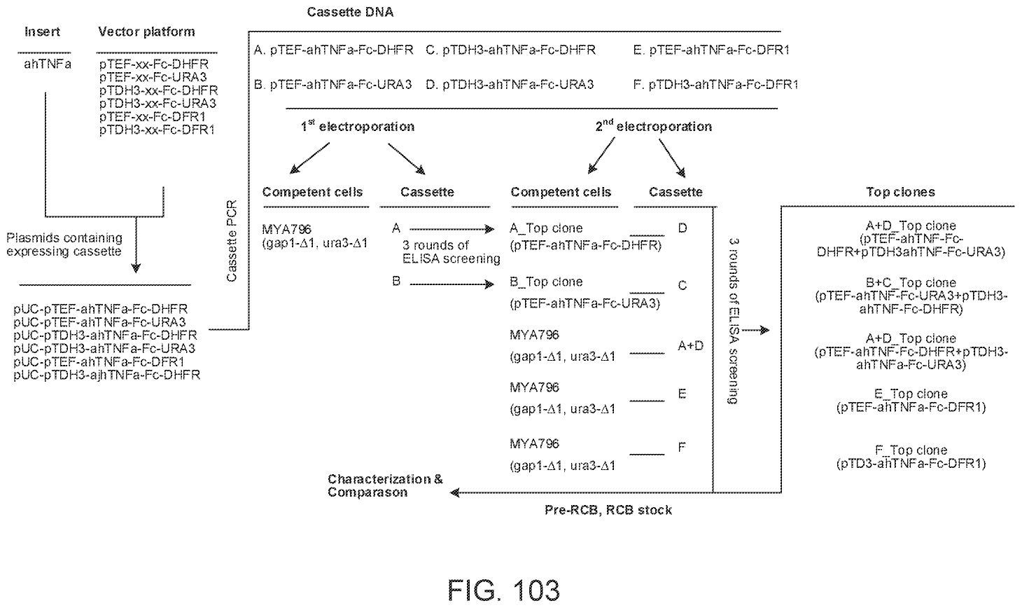

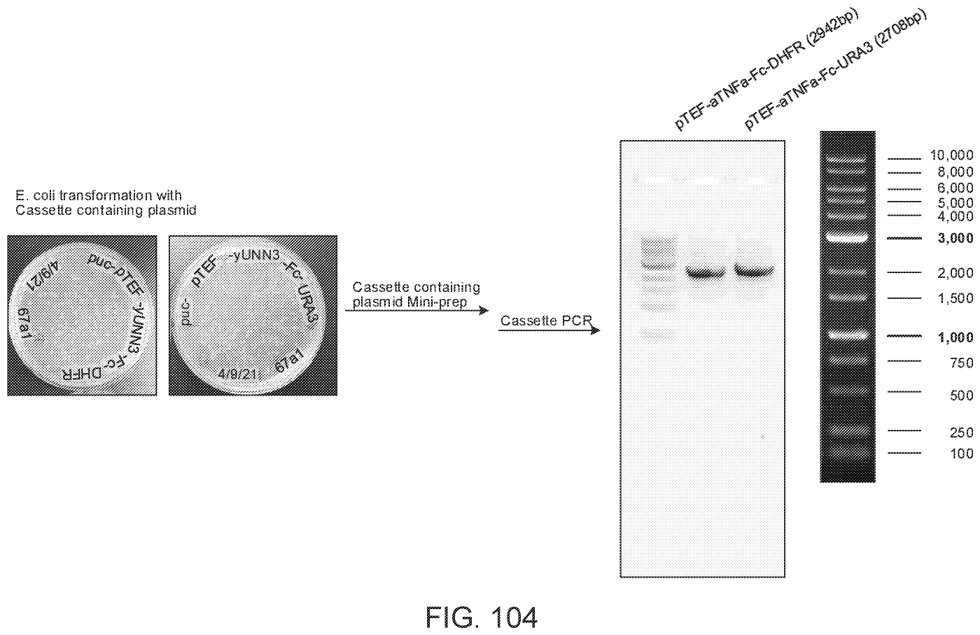

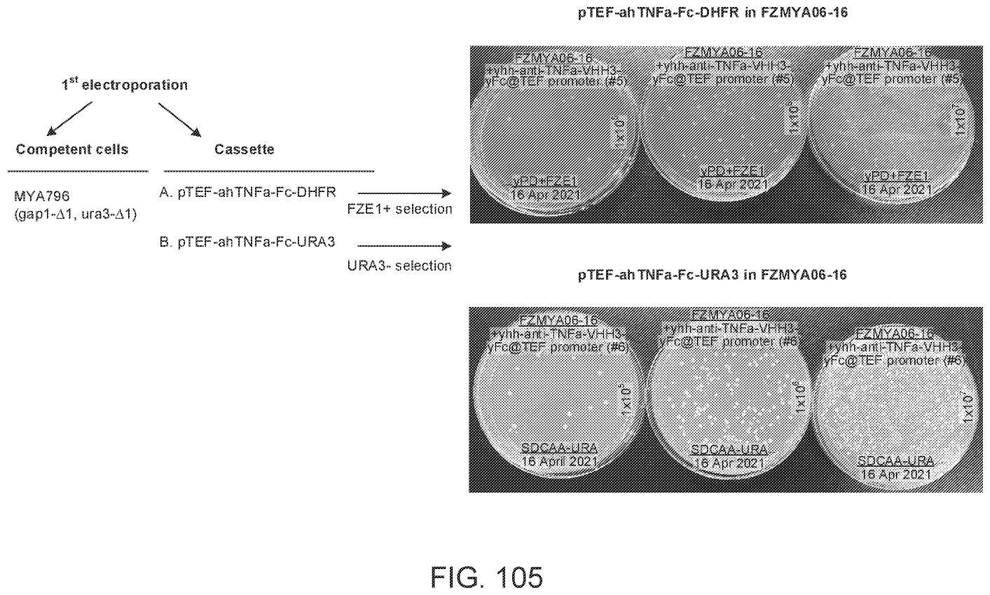

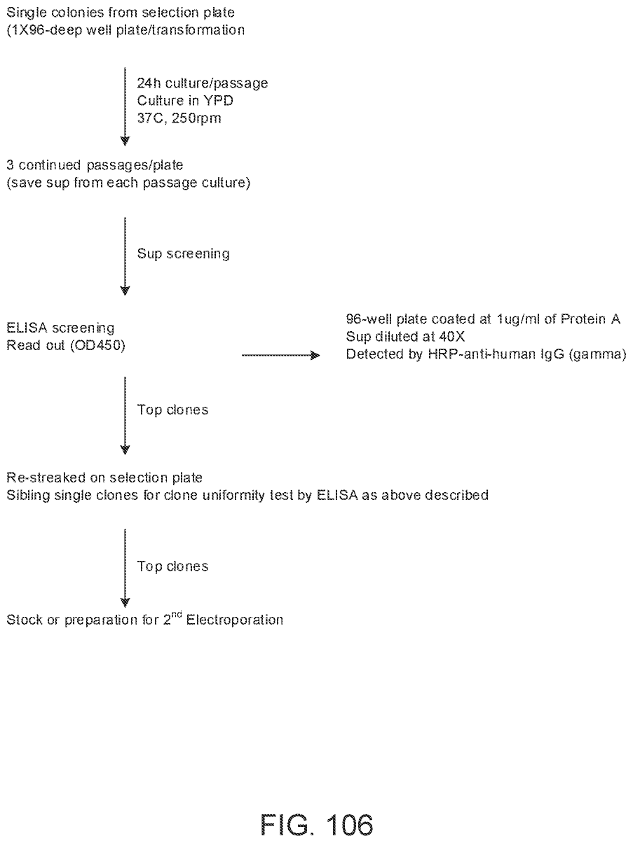

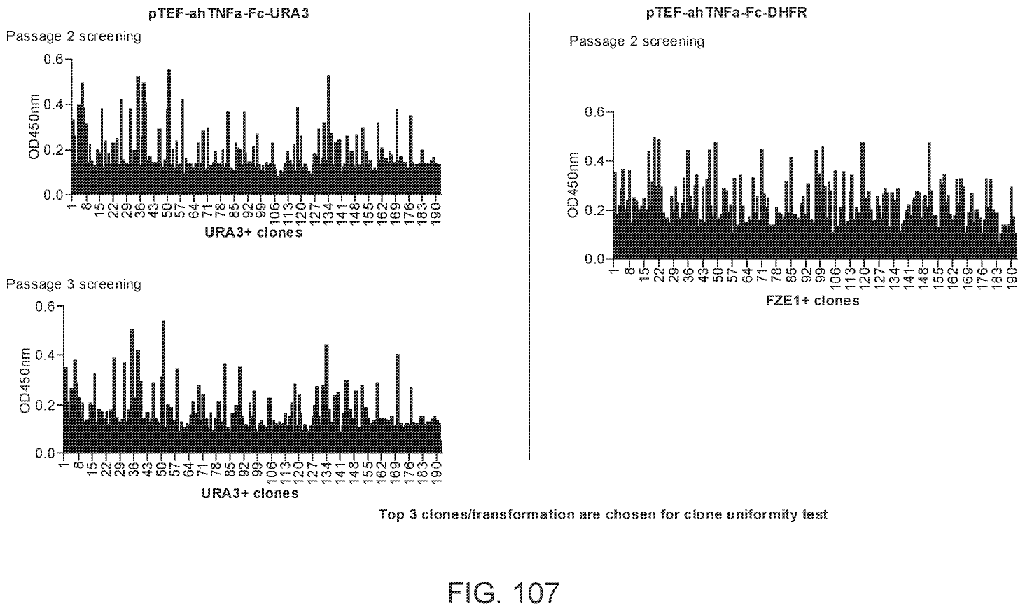

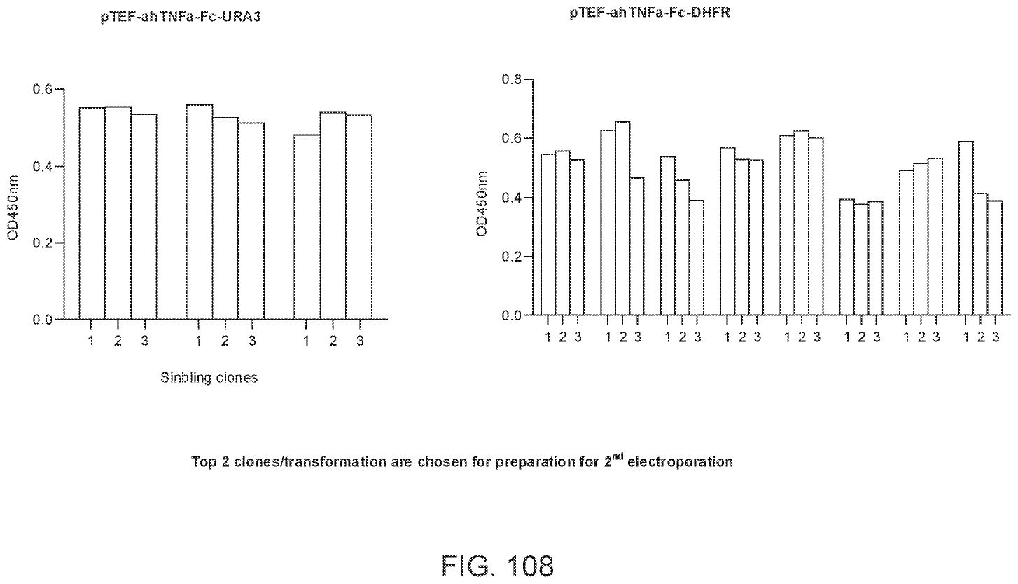

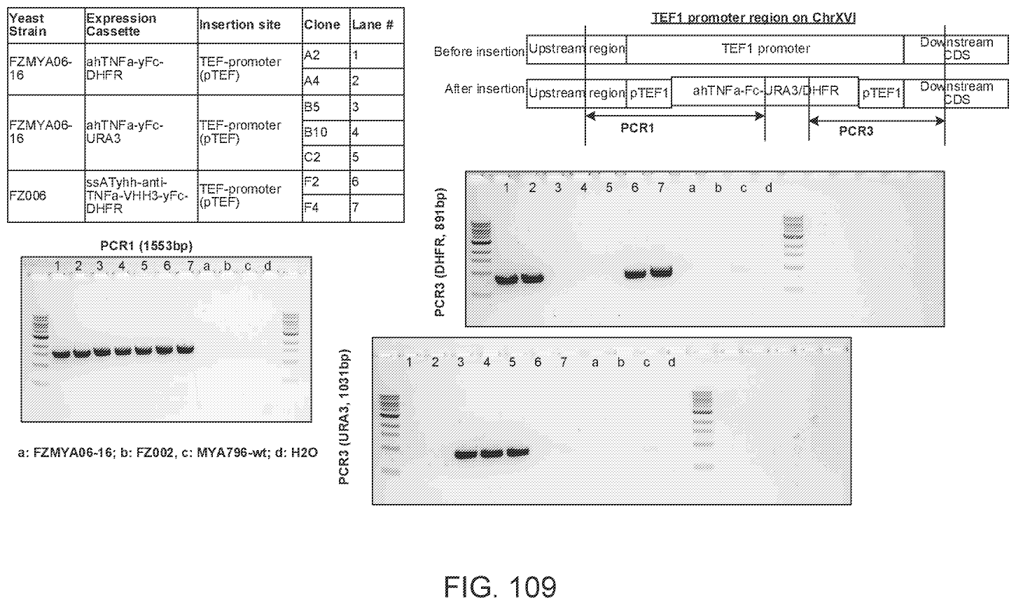

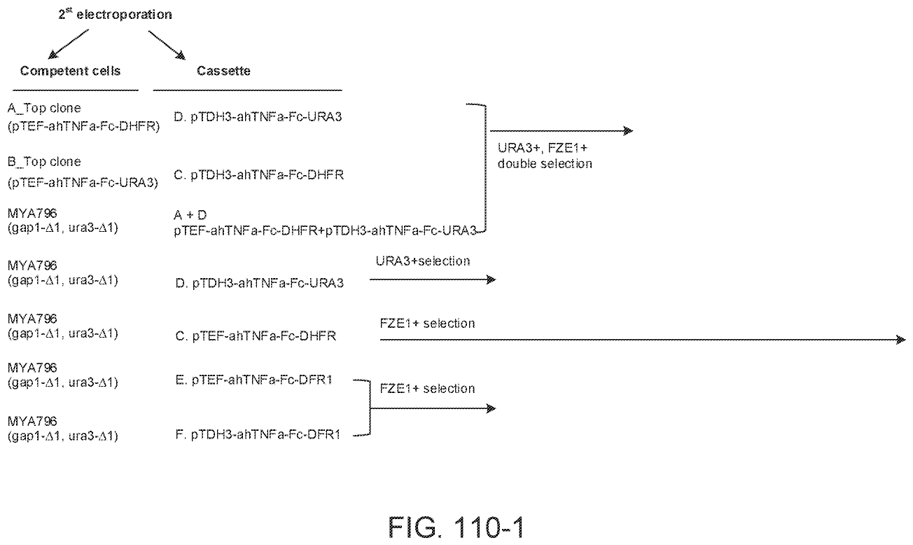

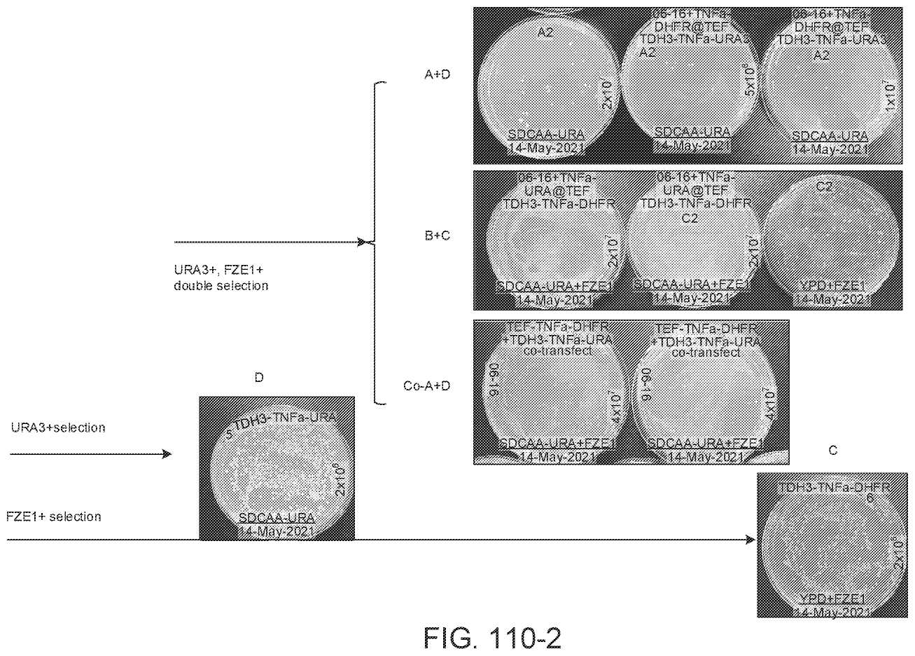

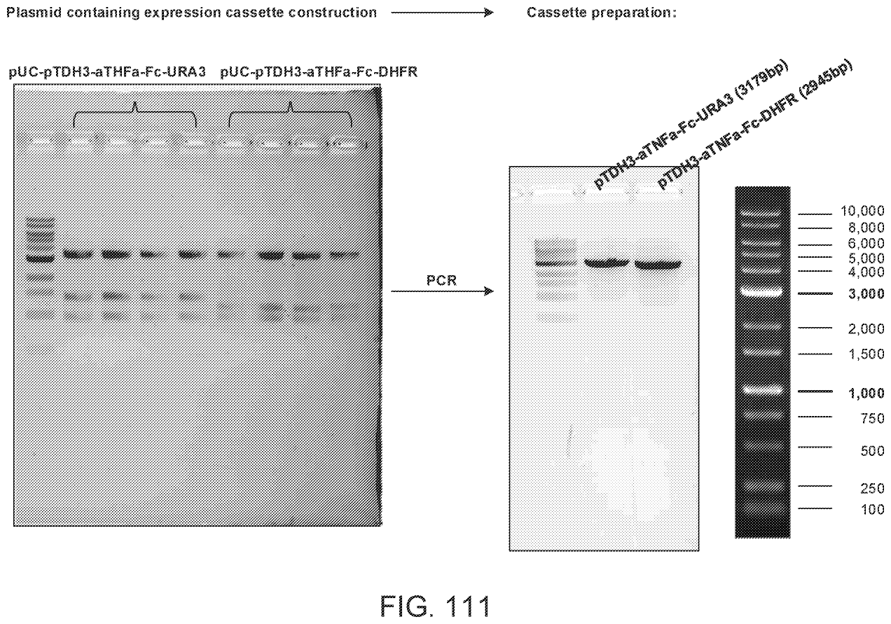

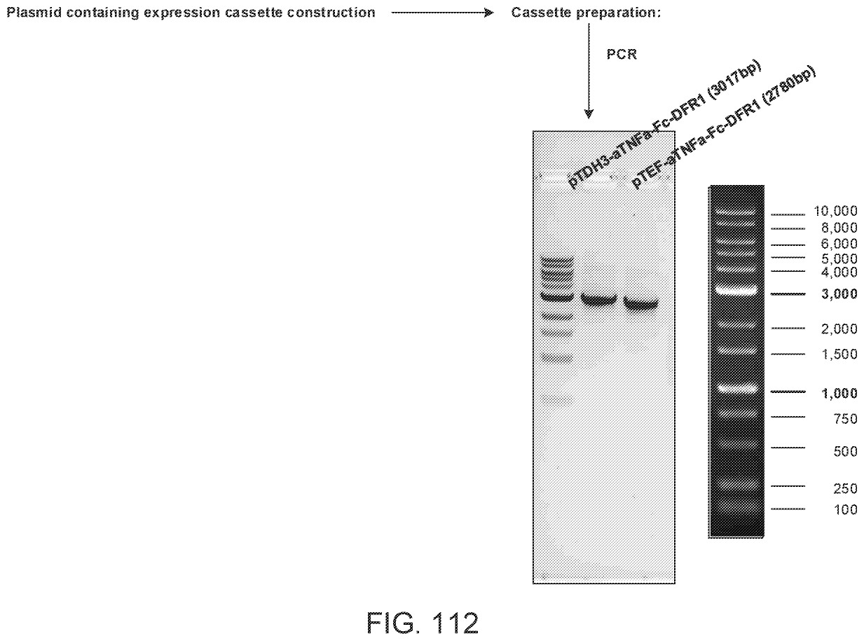

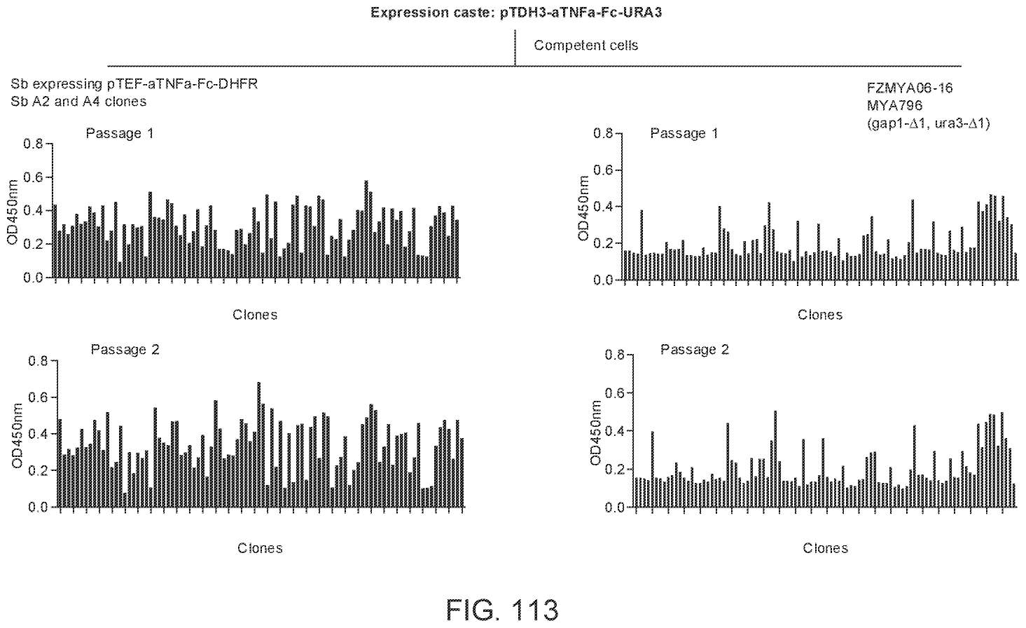

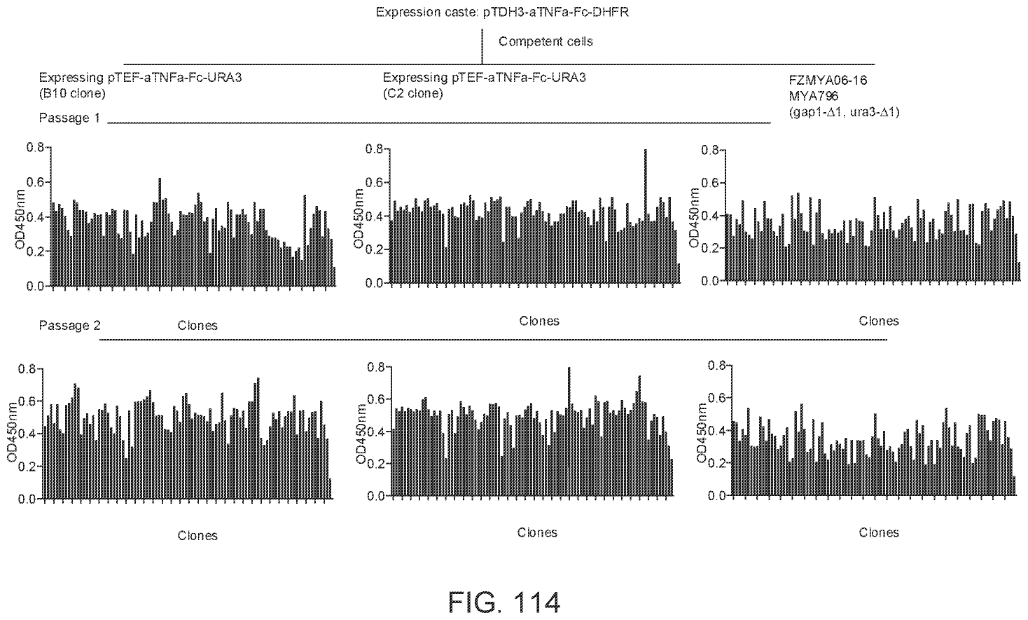

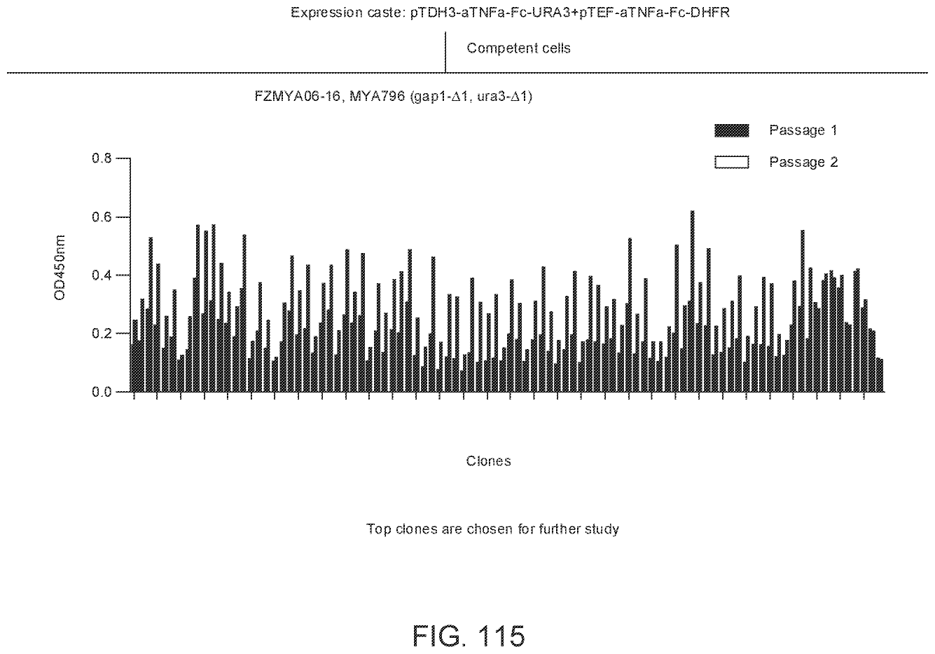

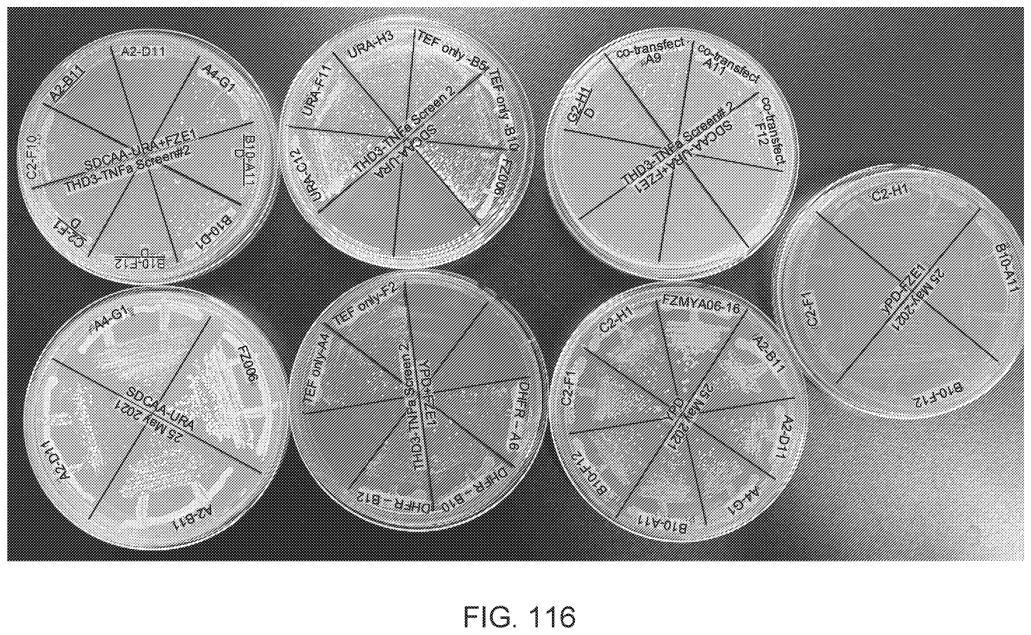

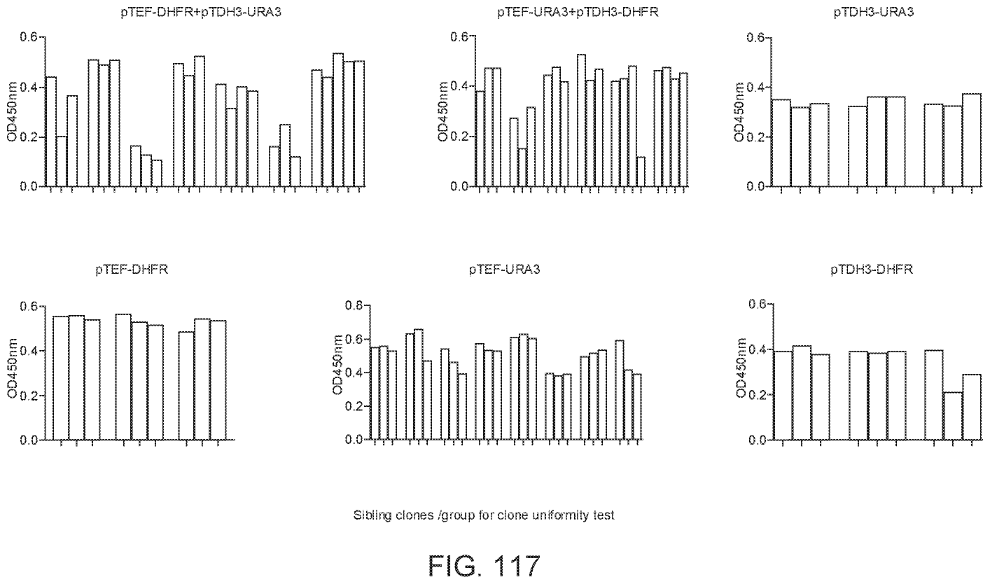

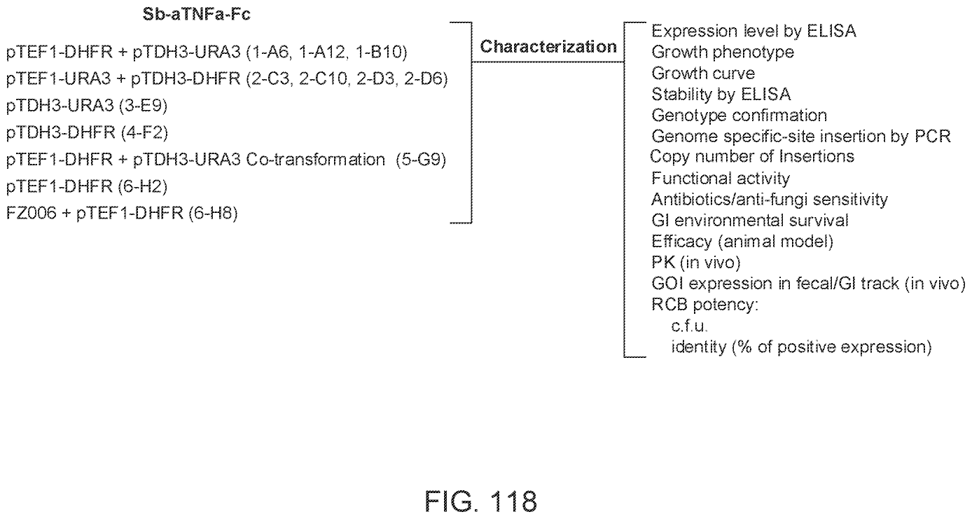

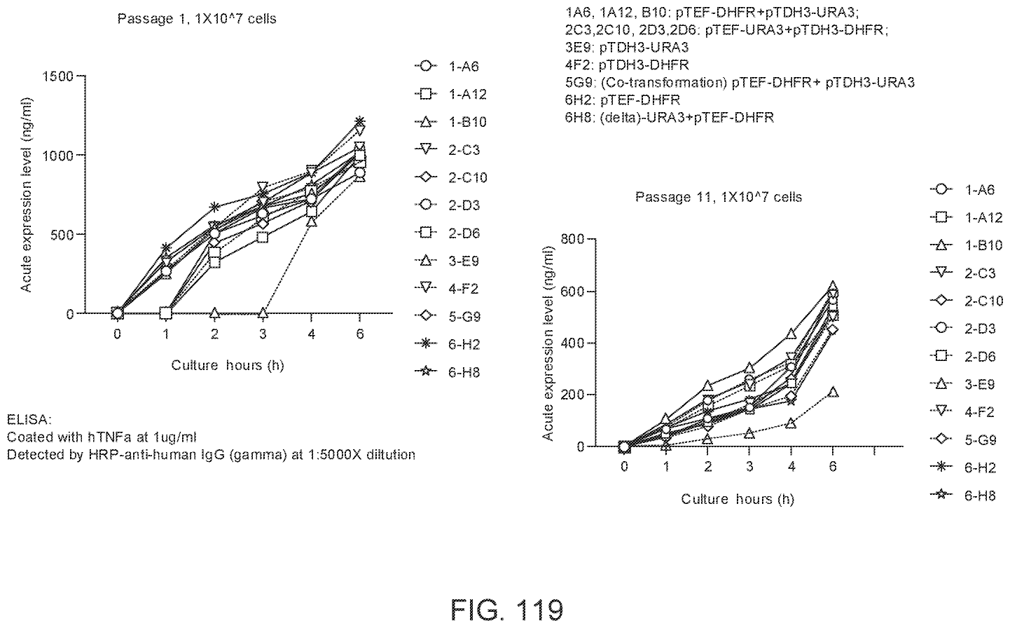

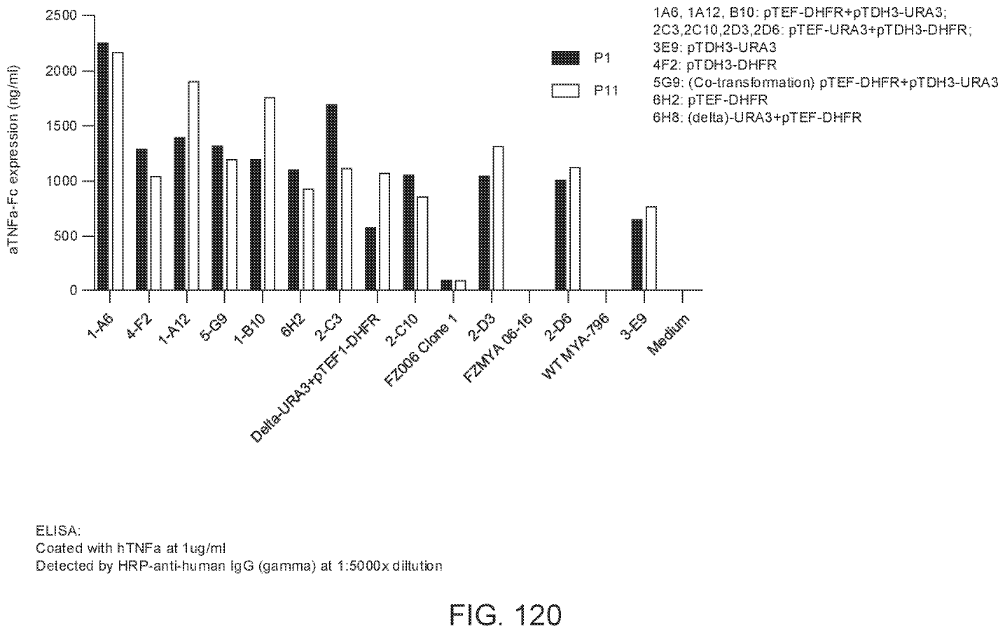

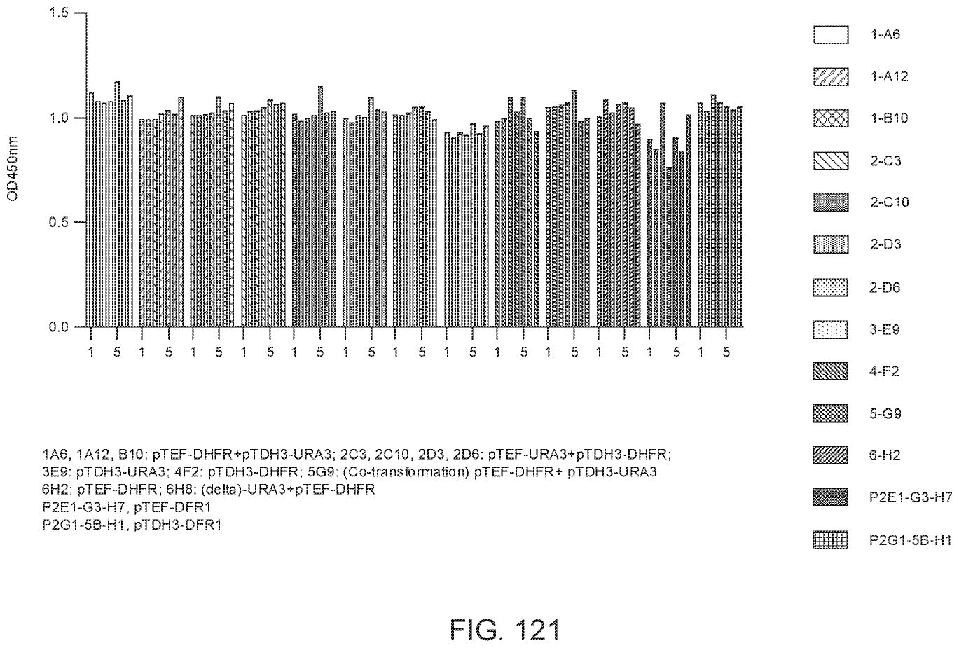

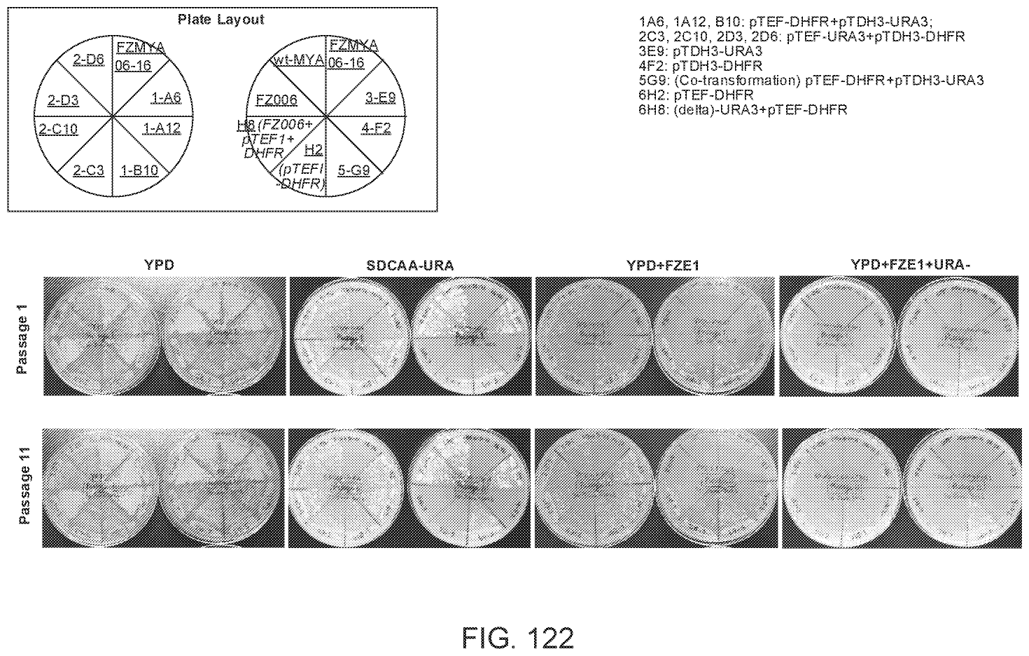

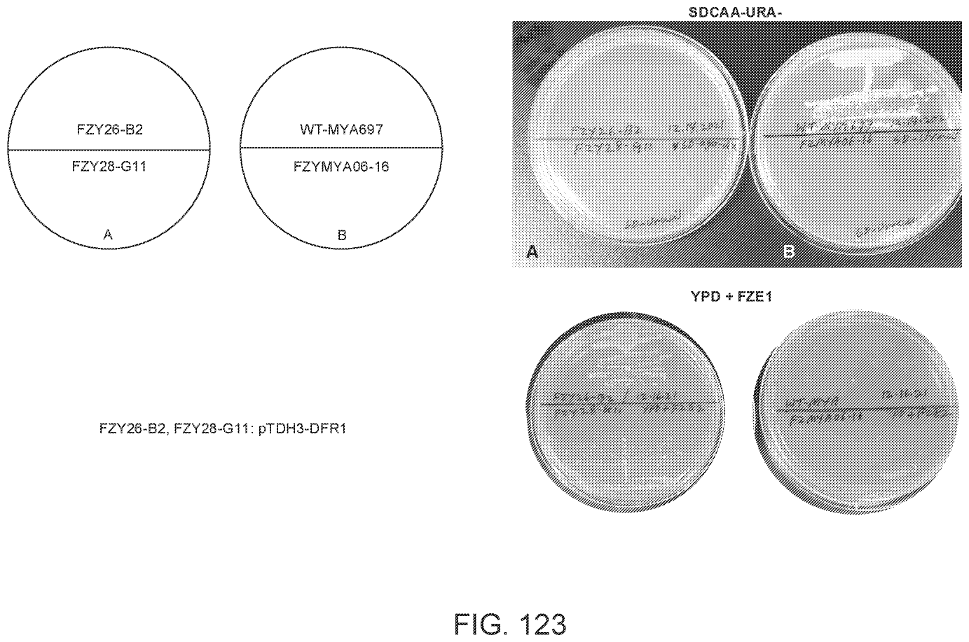

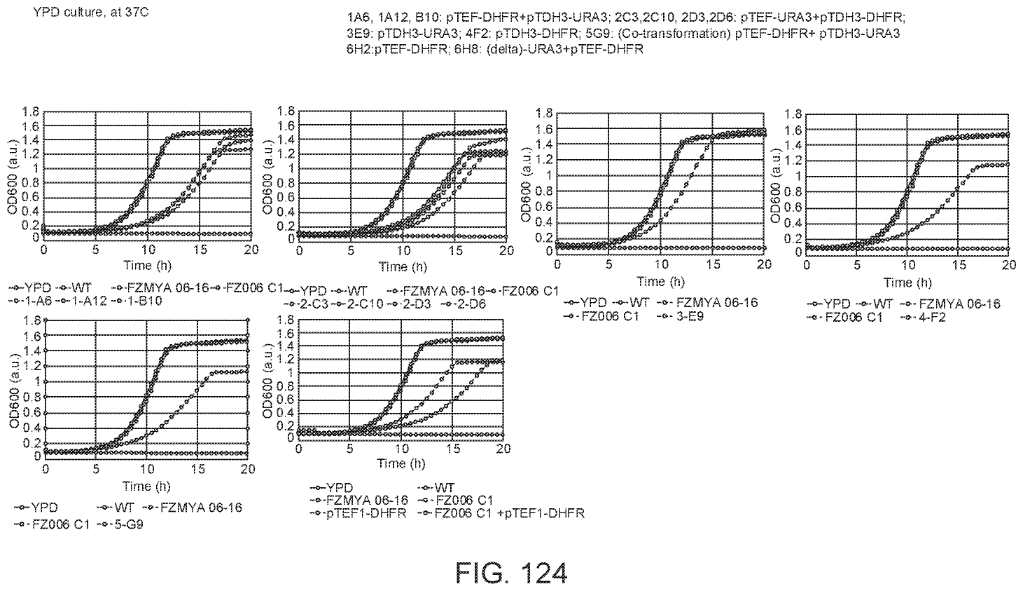

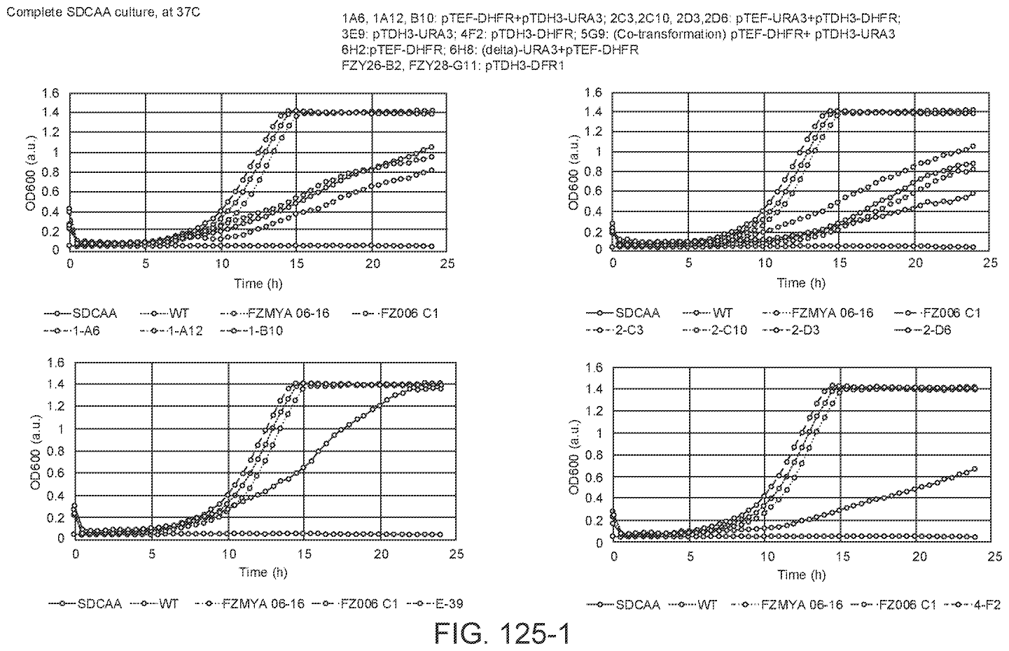

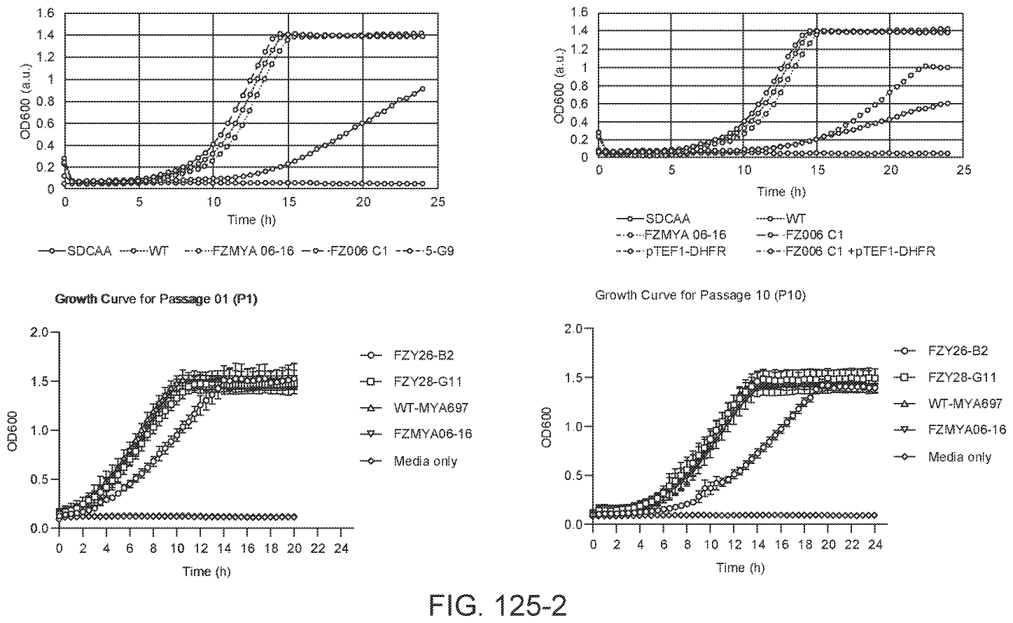

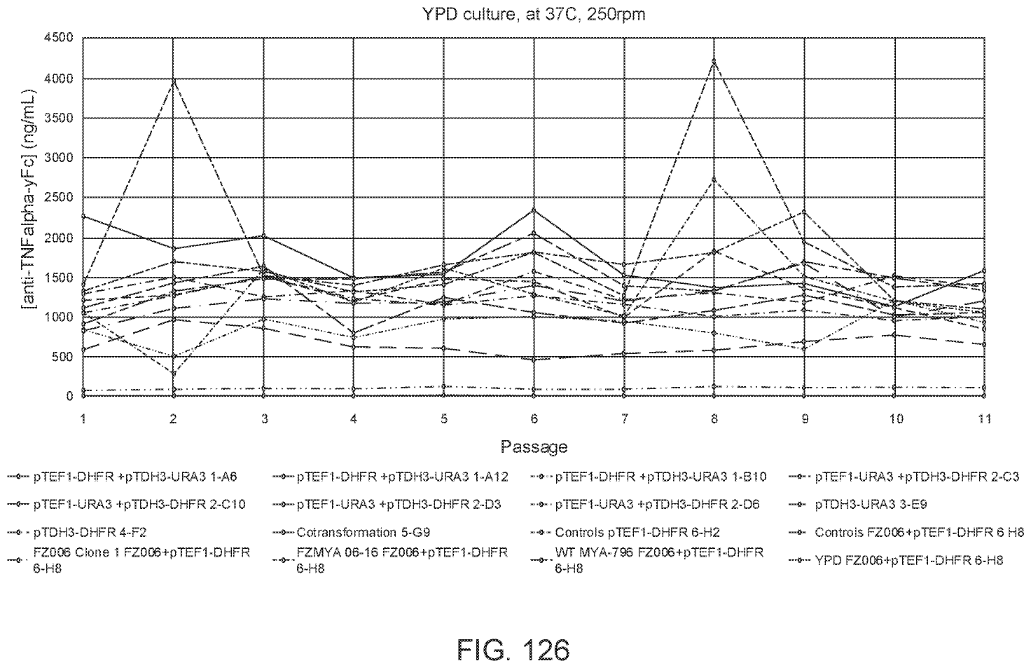

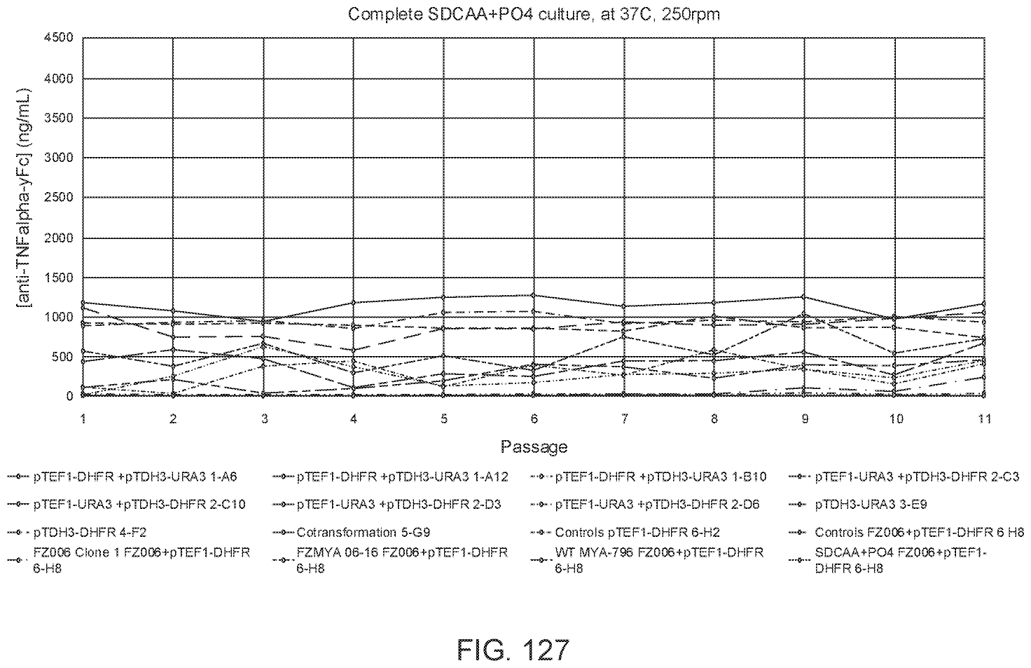

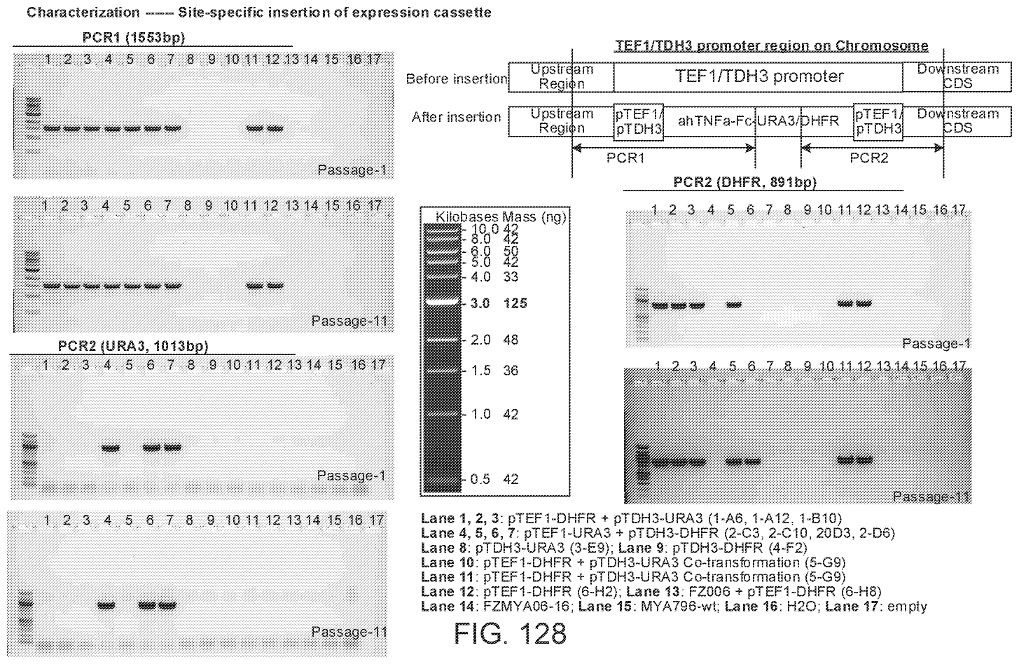

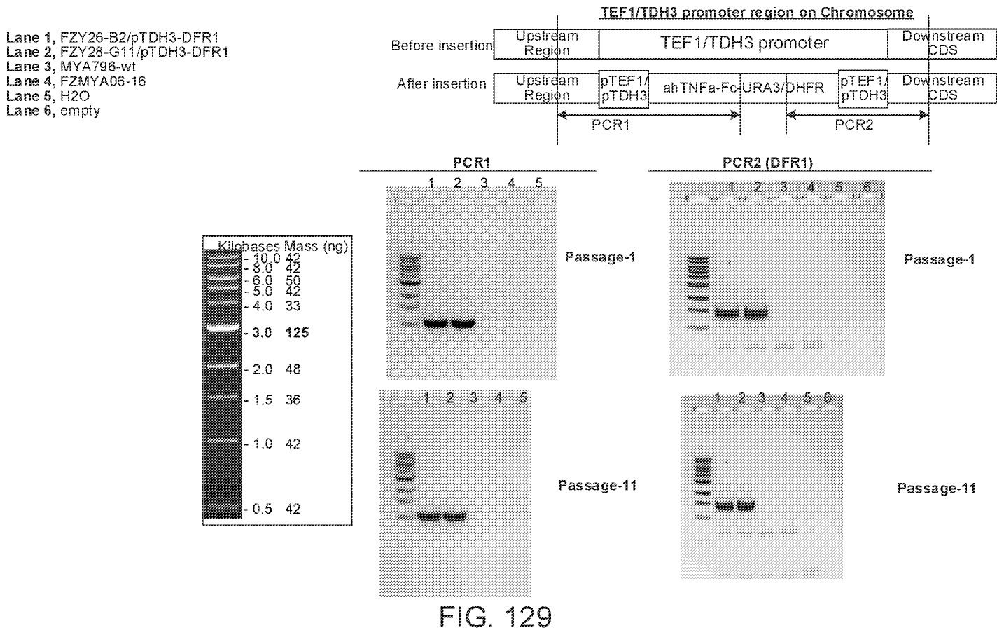

FIGS. 103-129 shows exemplary generation and characterization of engineered strain of S. boulardii constitutively expressing a single VHHs screened from yeast displayed-immune library and fused with an Fc fragment, neutralizing hTNFα, porcine TNFa, and monkey TNFα (e.g., FZ006h).

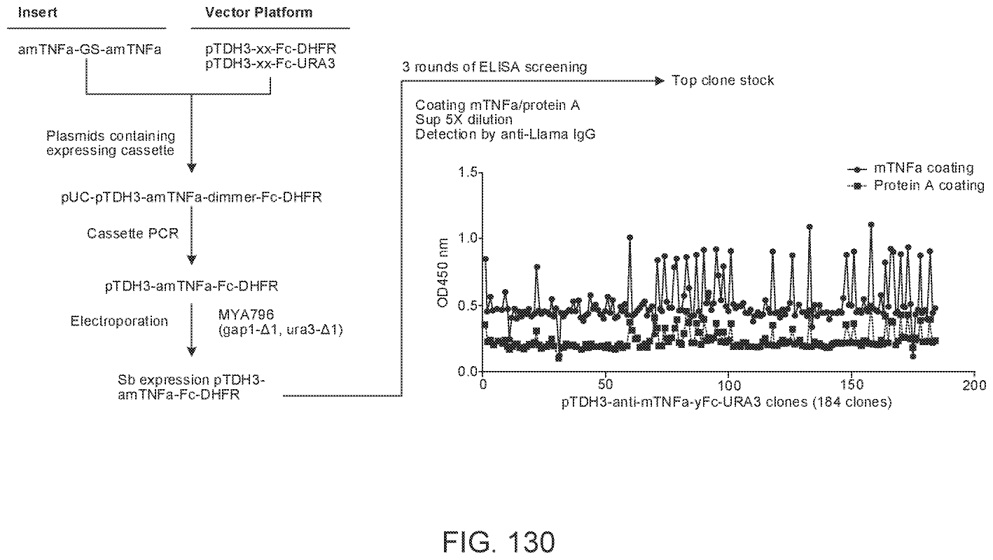

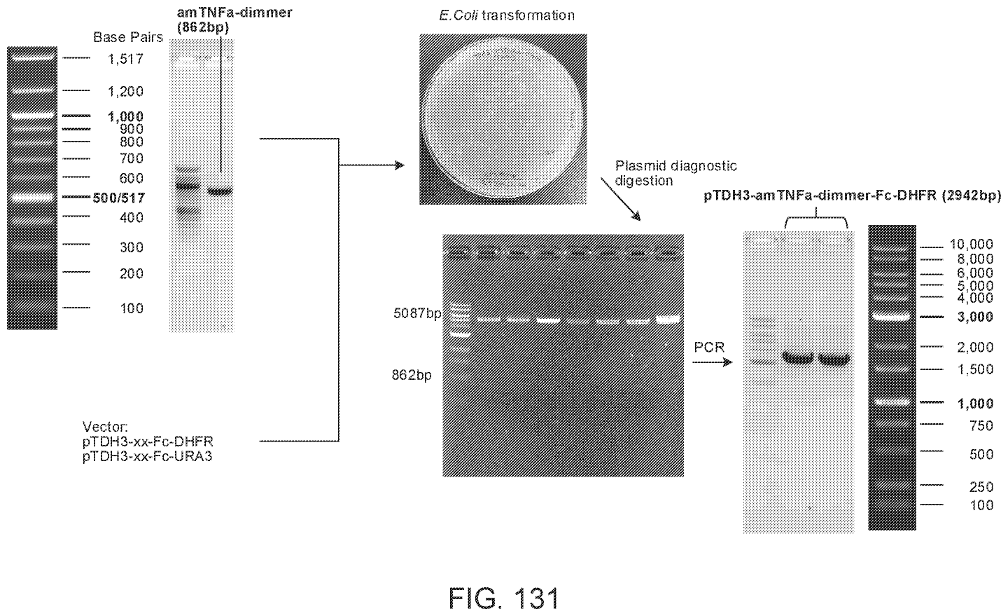

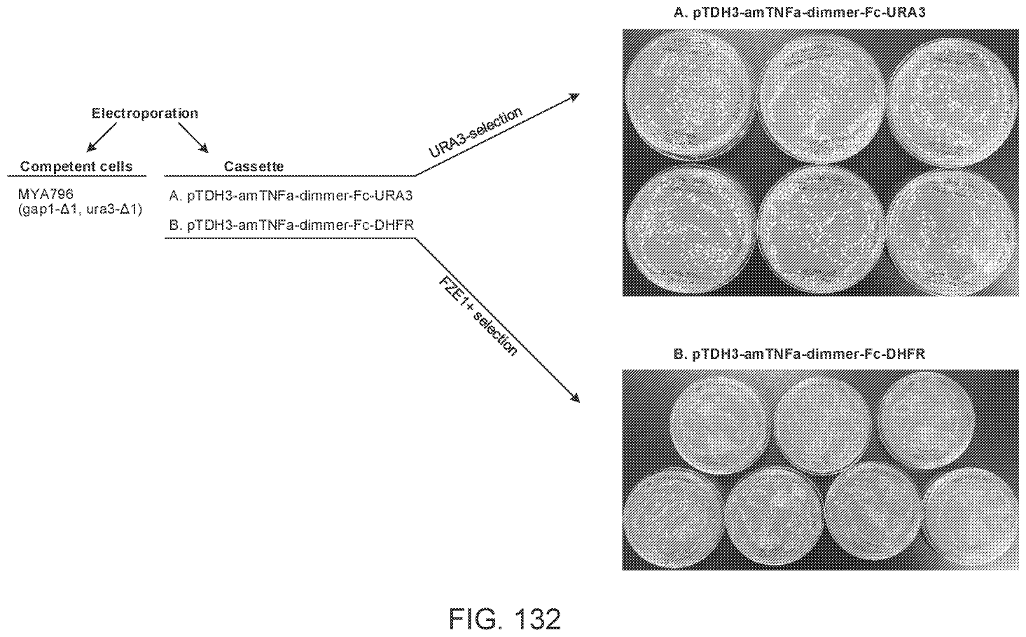

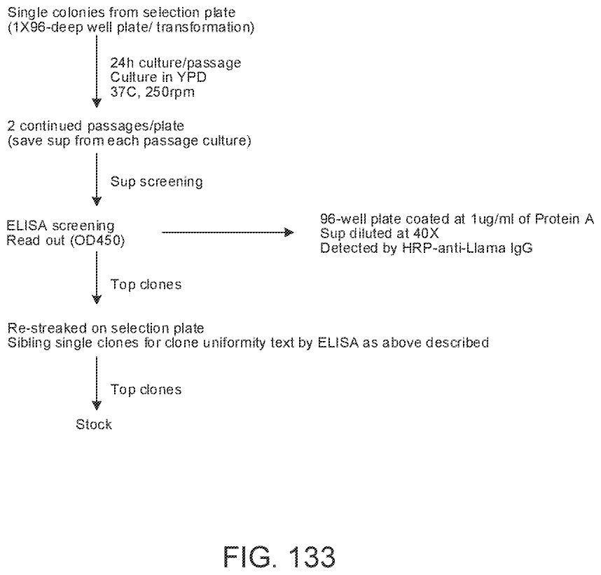

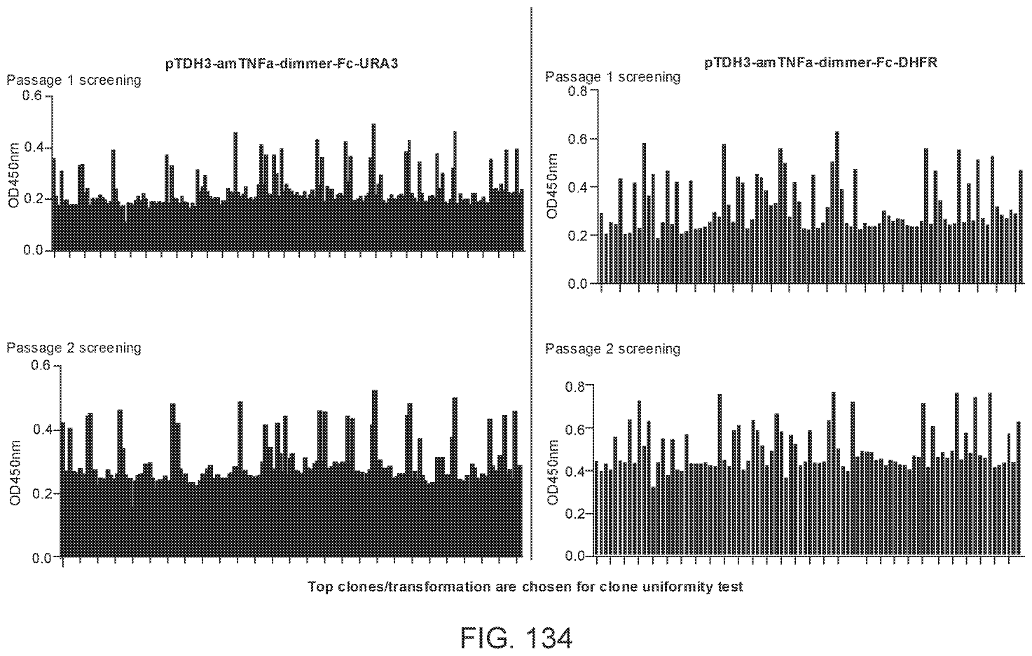

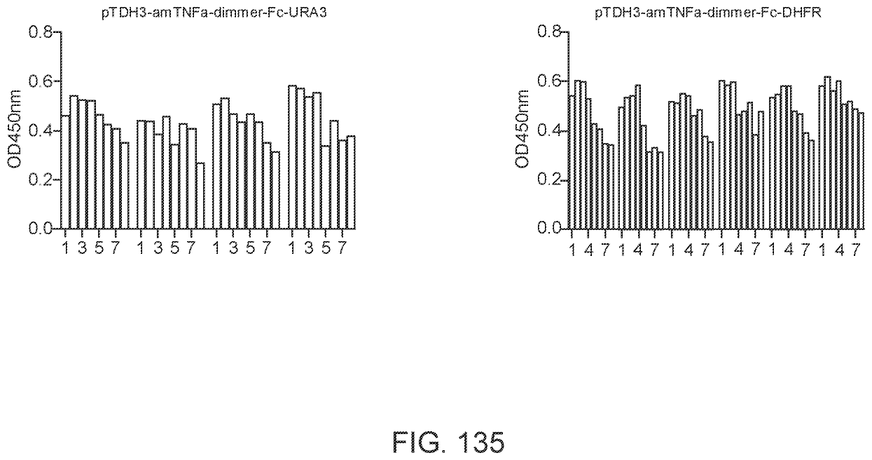

FIGS. 130-135 shows exemplary generation and characterization of engineered strain of S. boulardii constitutively expressing a VHH dimer neutralizing mTNFα and fused with an Fc fragment (e.g., FZ006m).

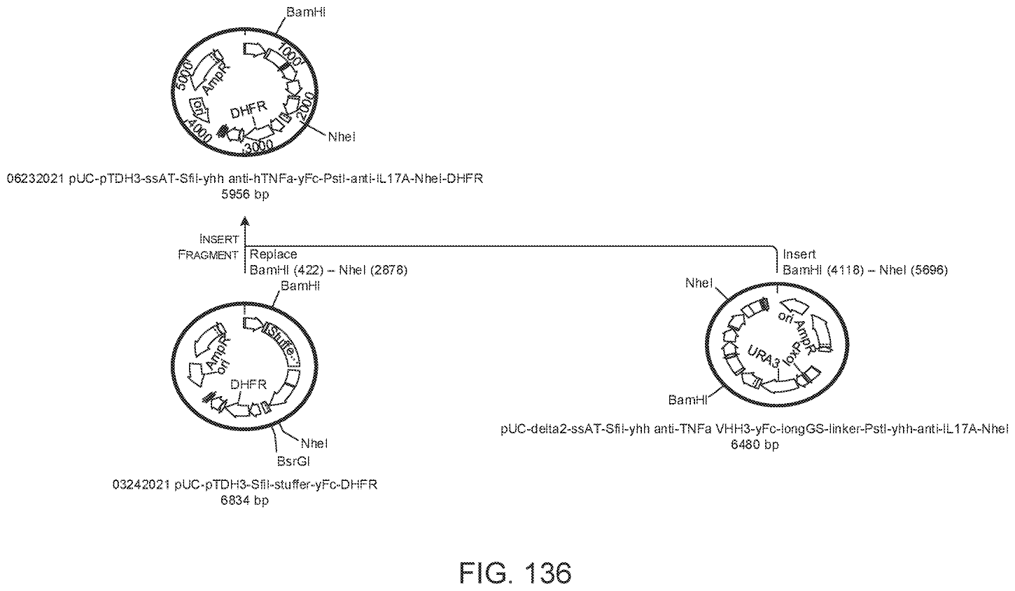

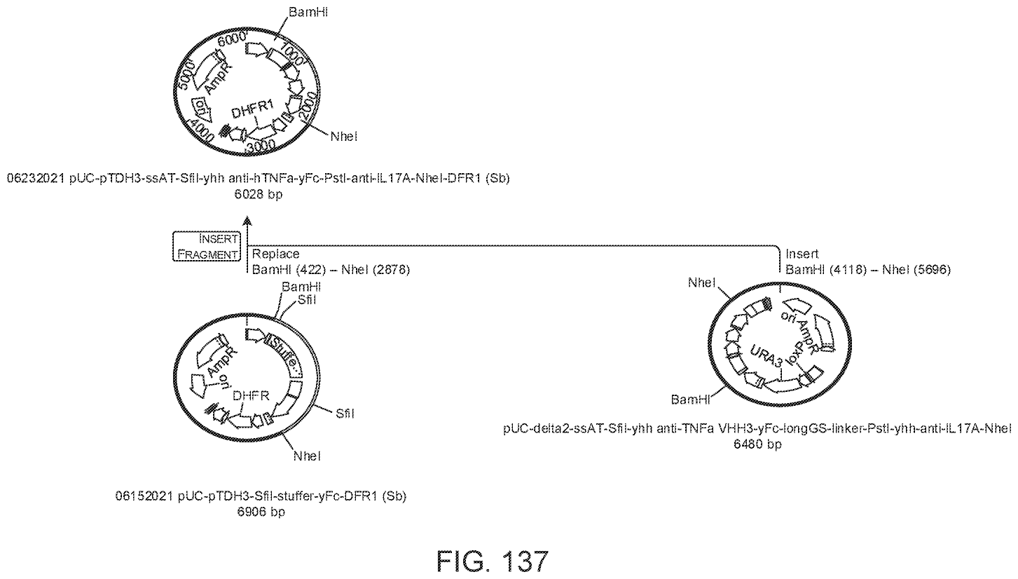

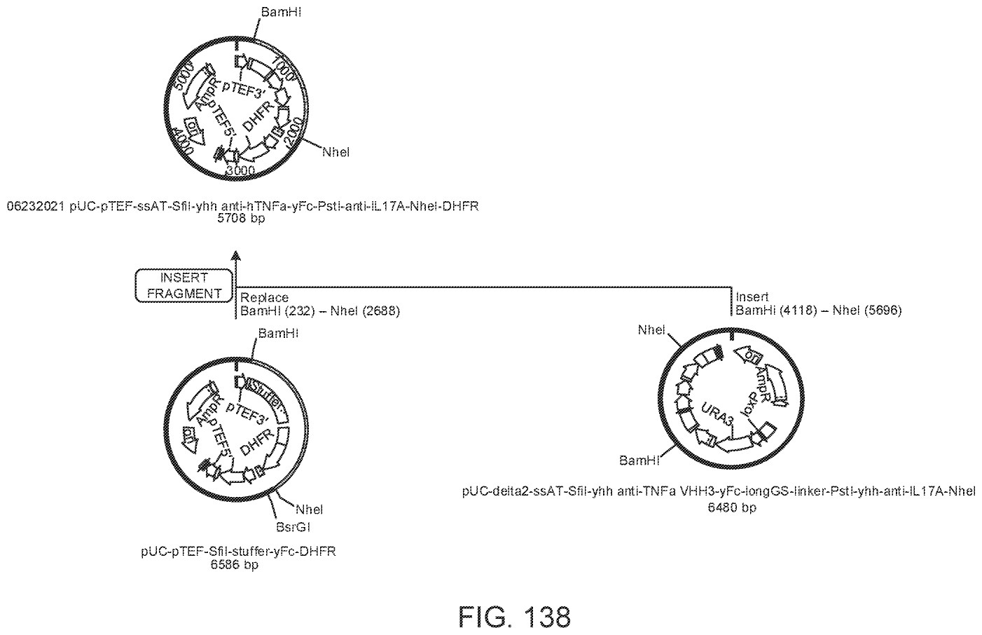

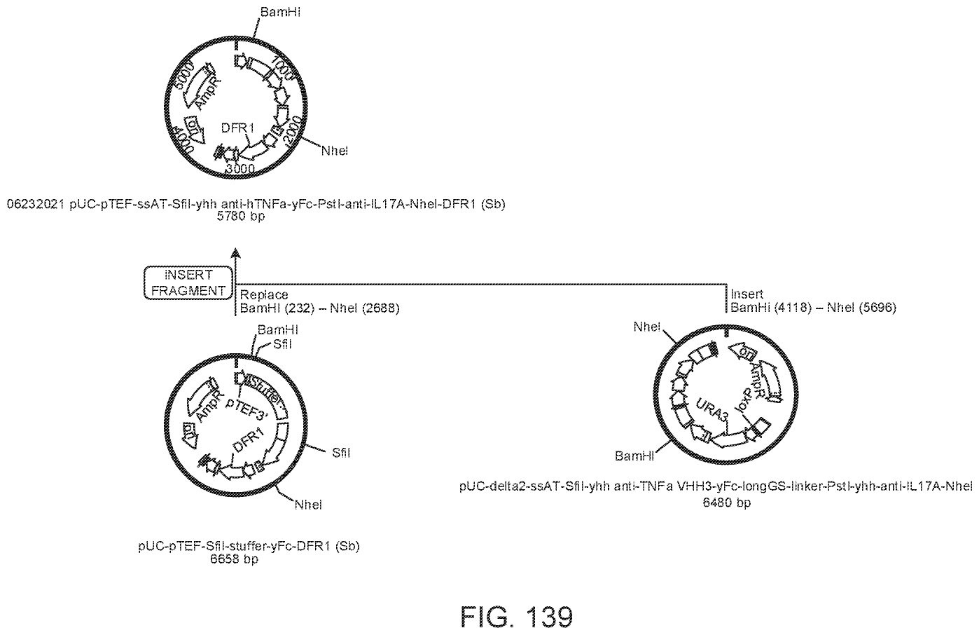

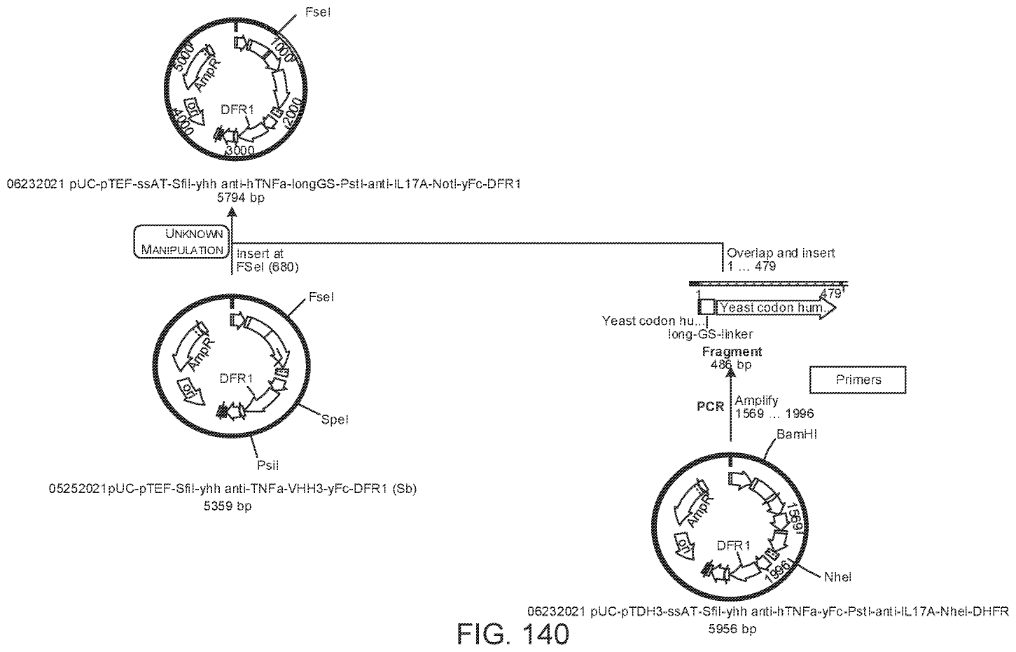

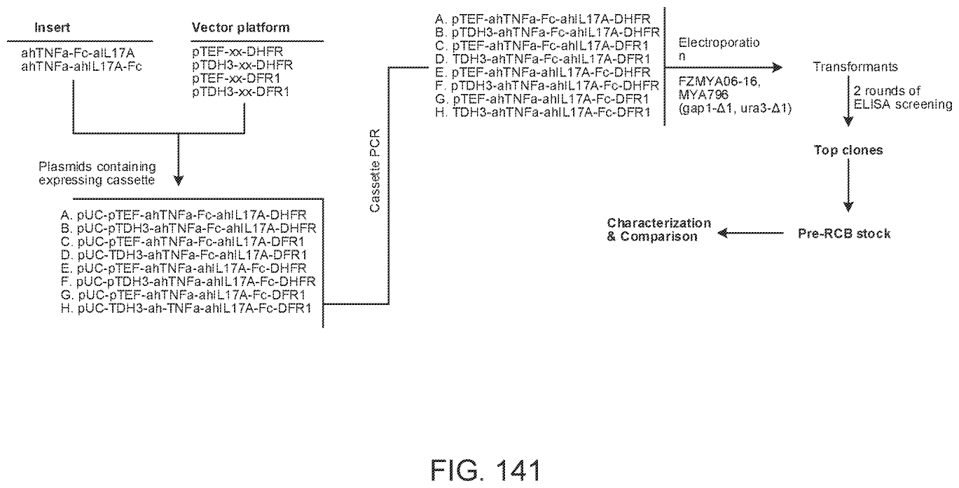

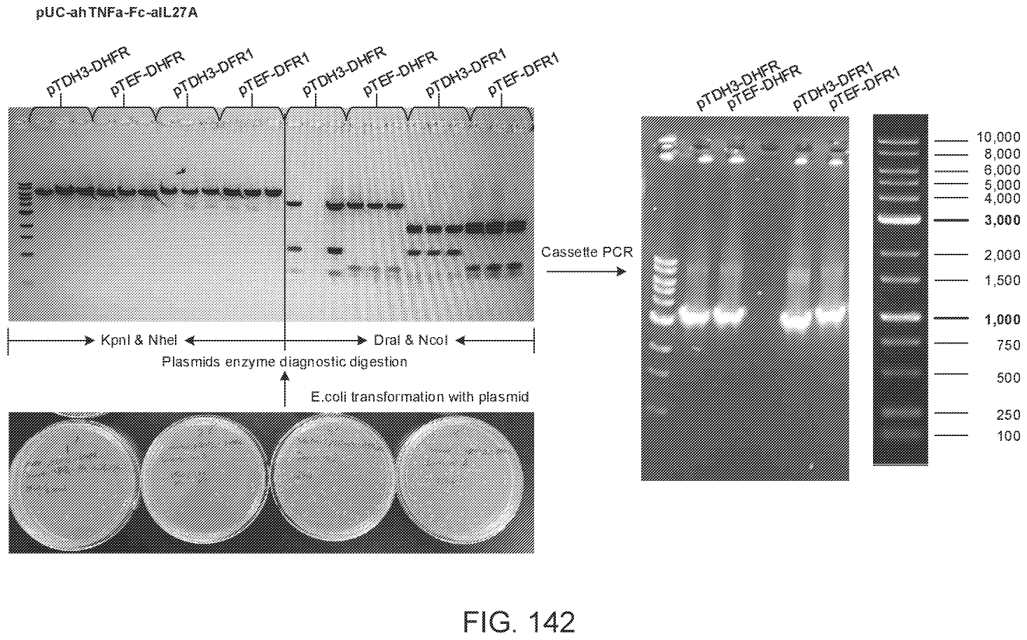

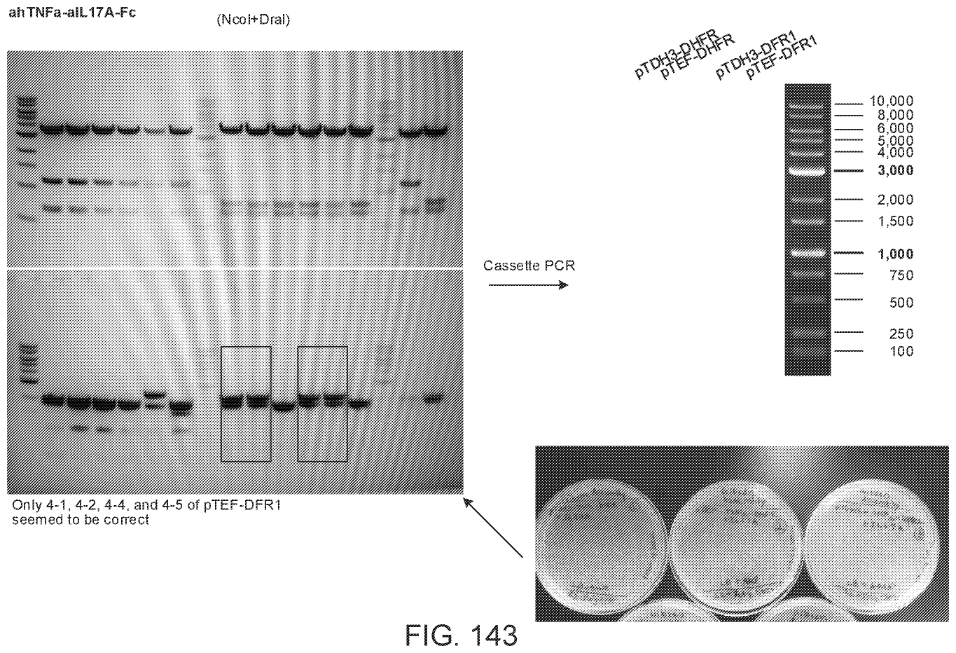

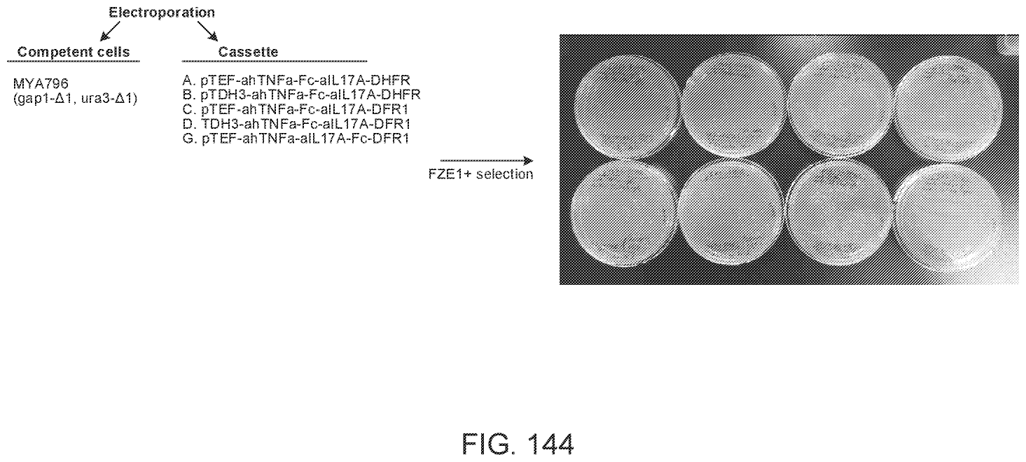

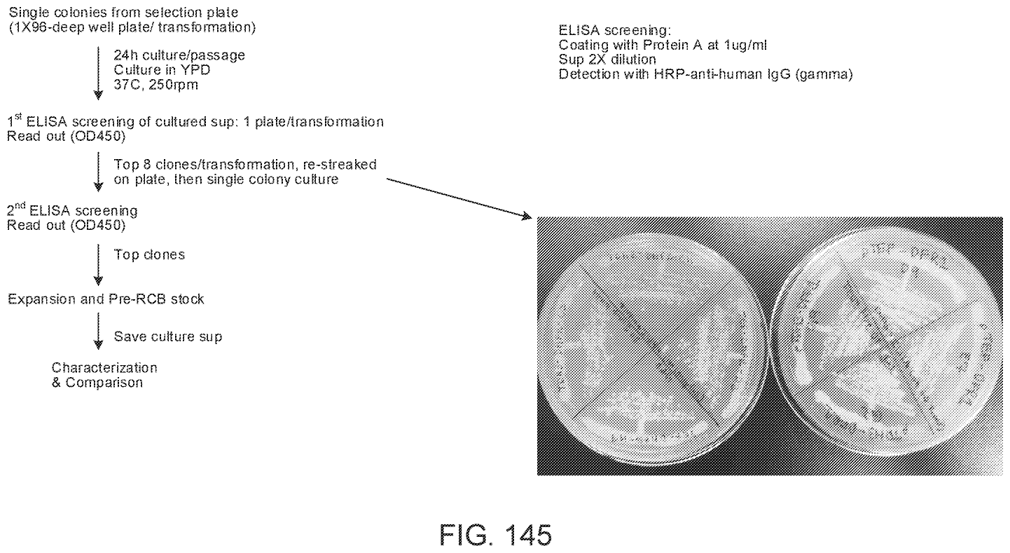

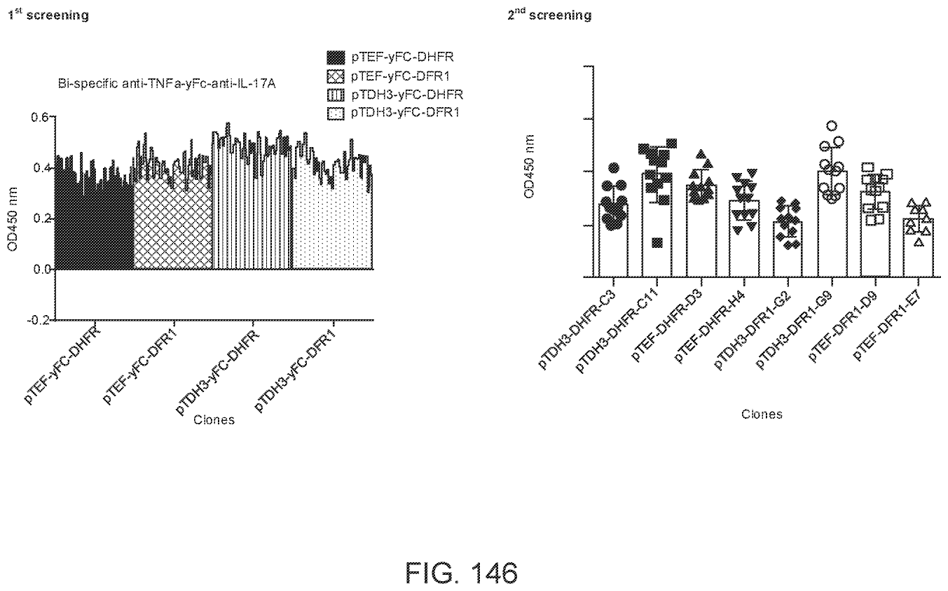

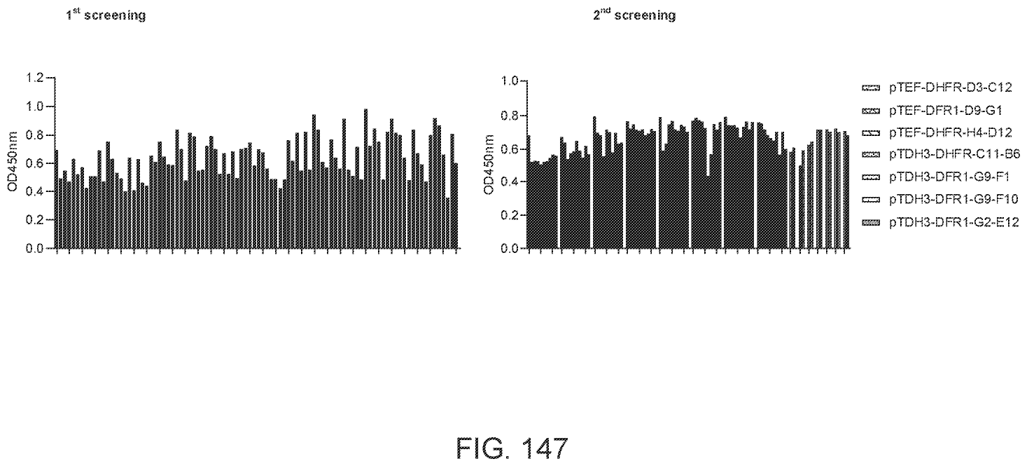

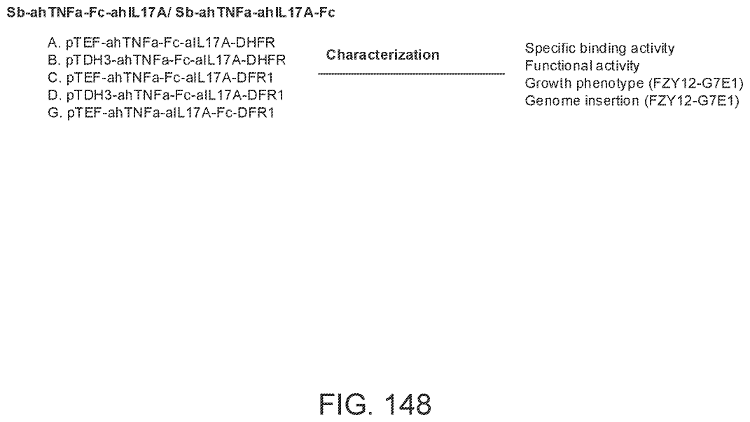

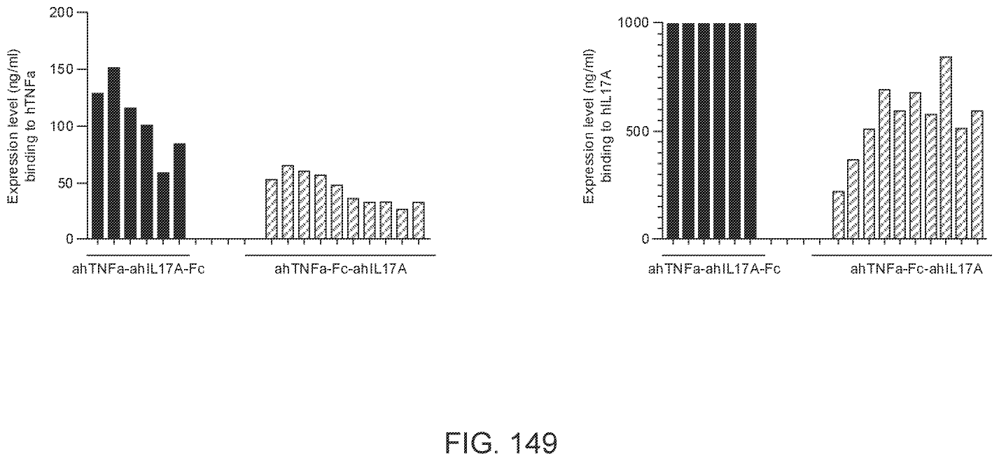

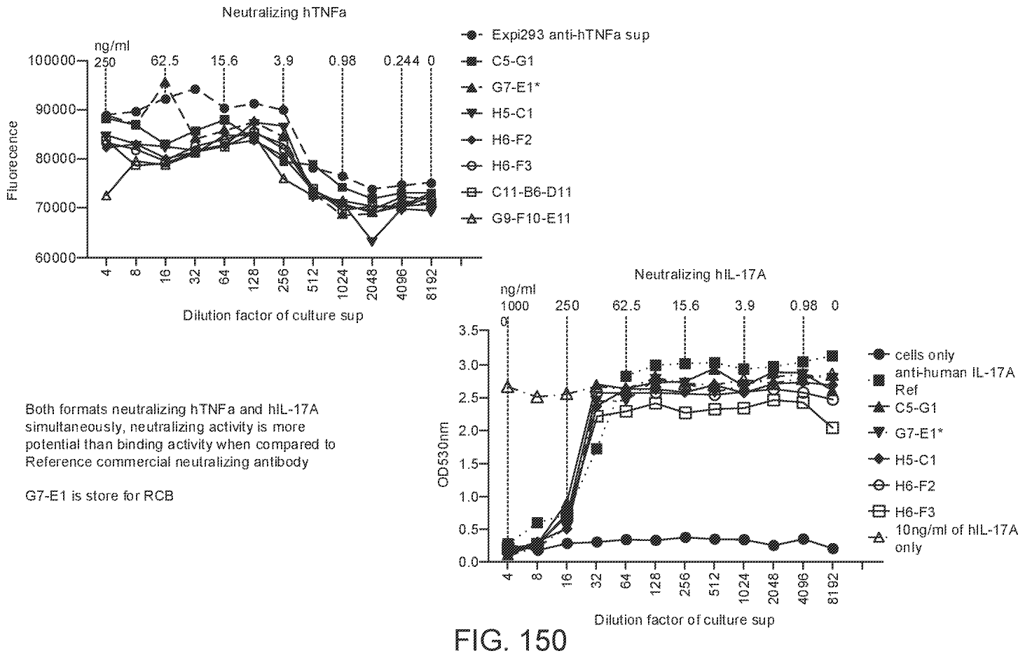

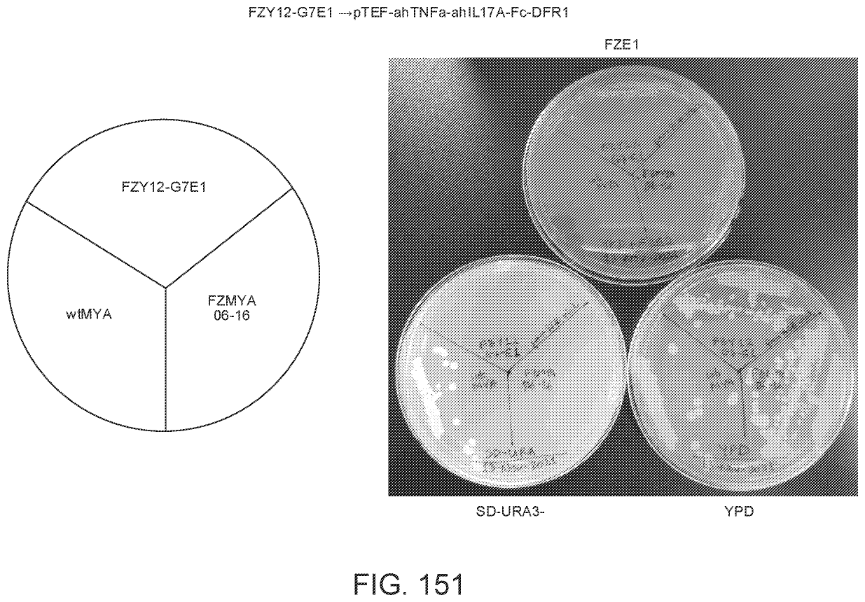

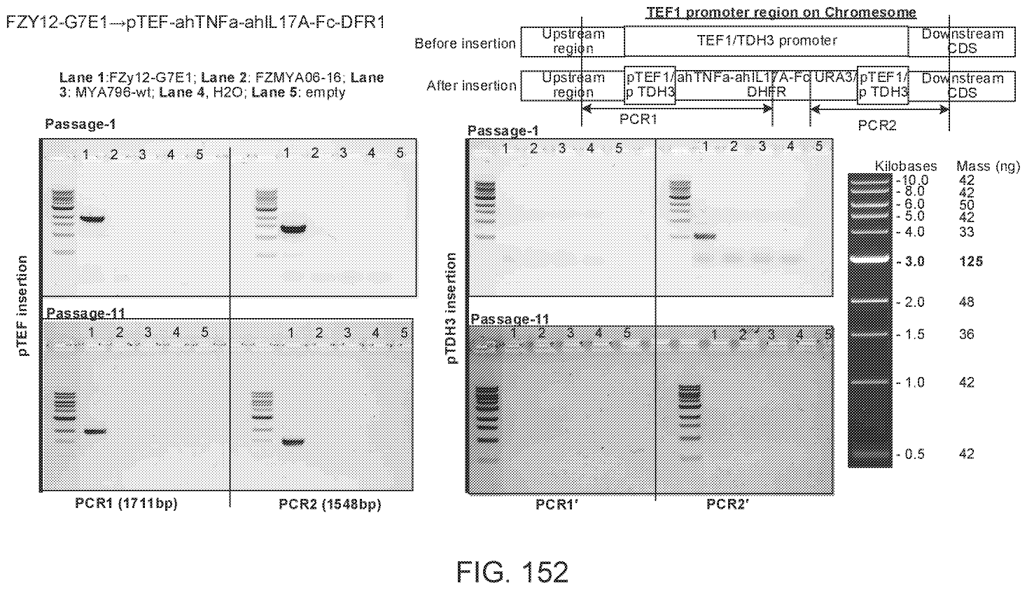

FIGS. 136-152 shows exemplary generation and characterization of engineered strain of S. boulardii constitutively expressing a bi-specific VHHs fused with Fc fragment, neutralizing hTNFα and hIL-17A (e.g., FZ008).

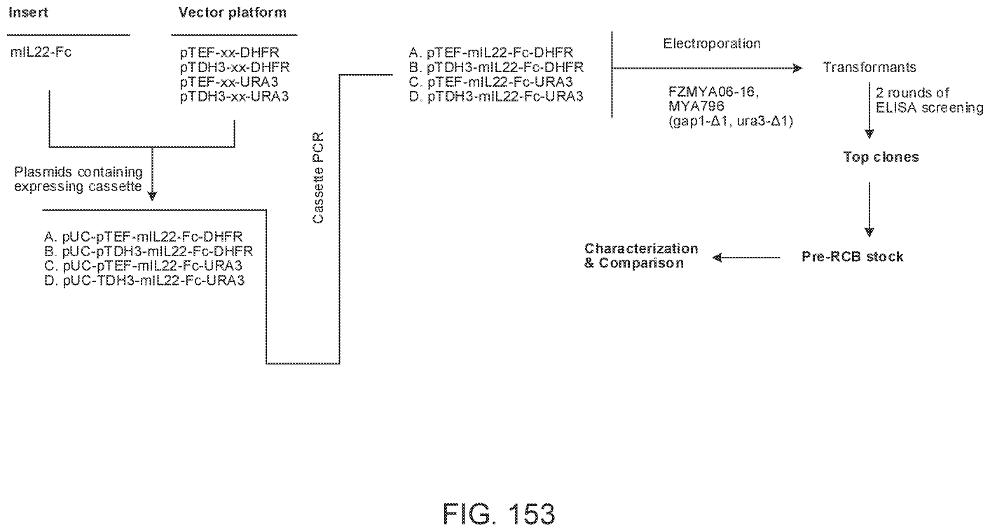

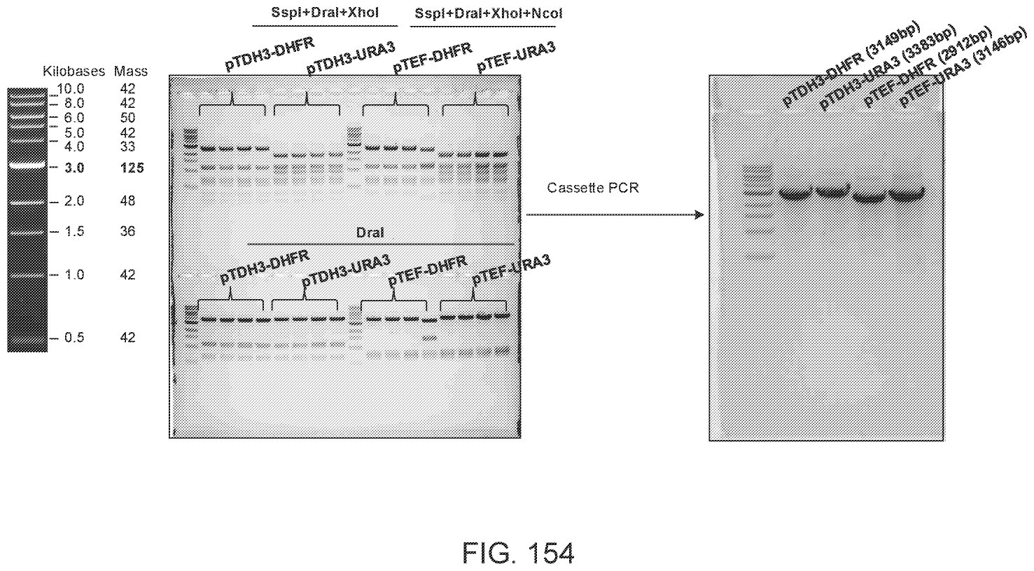

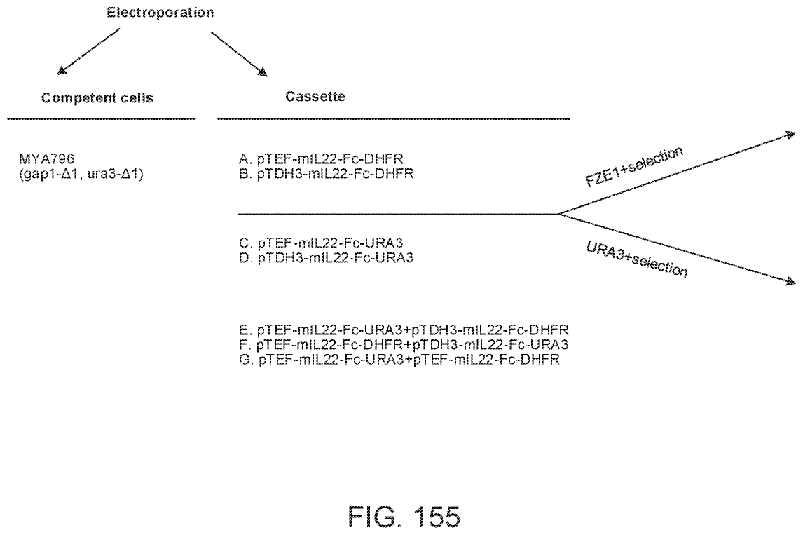

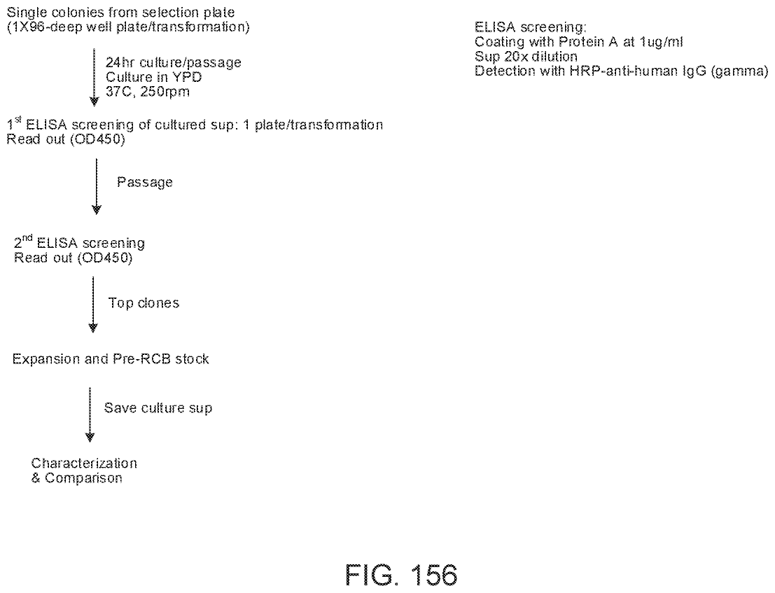

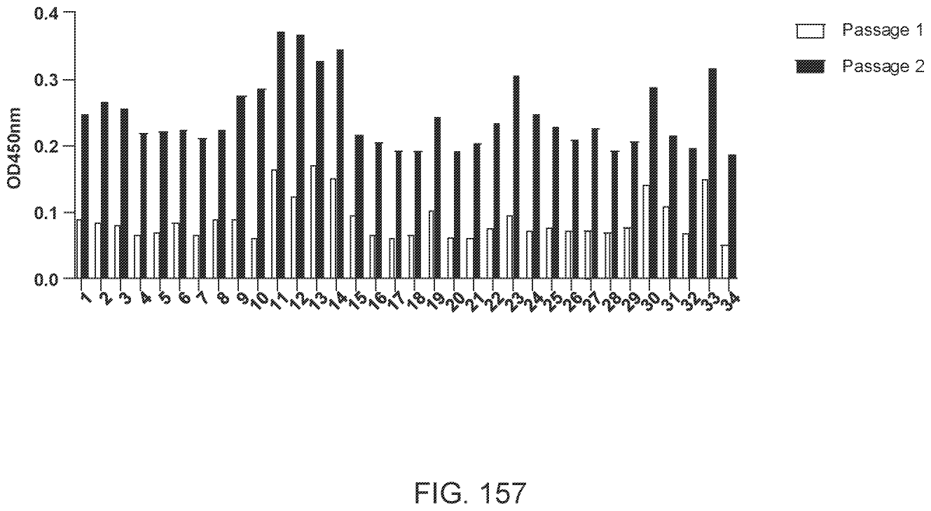

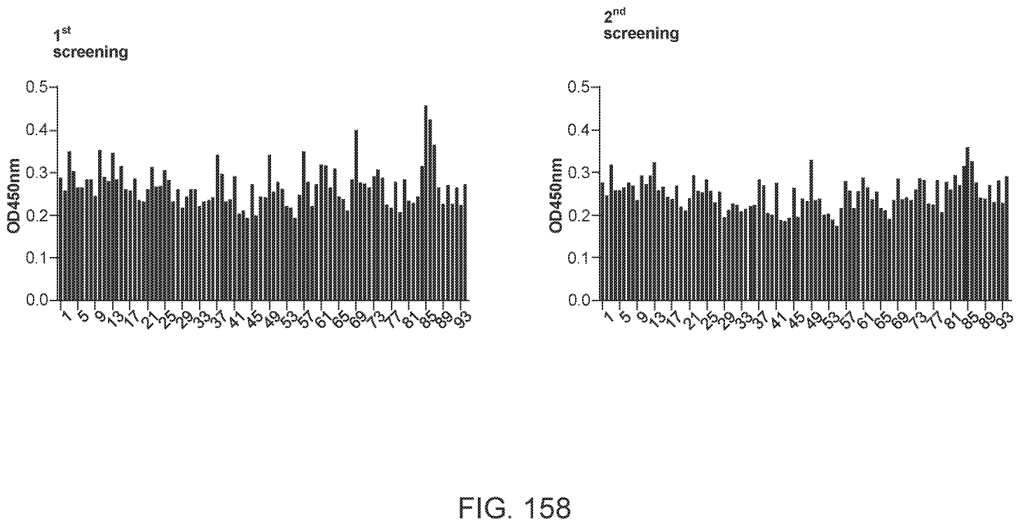

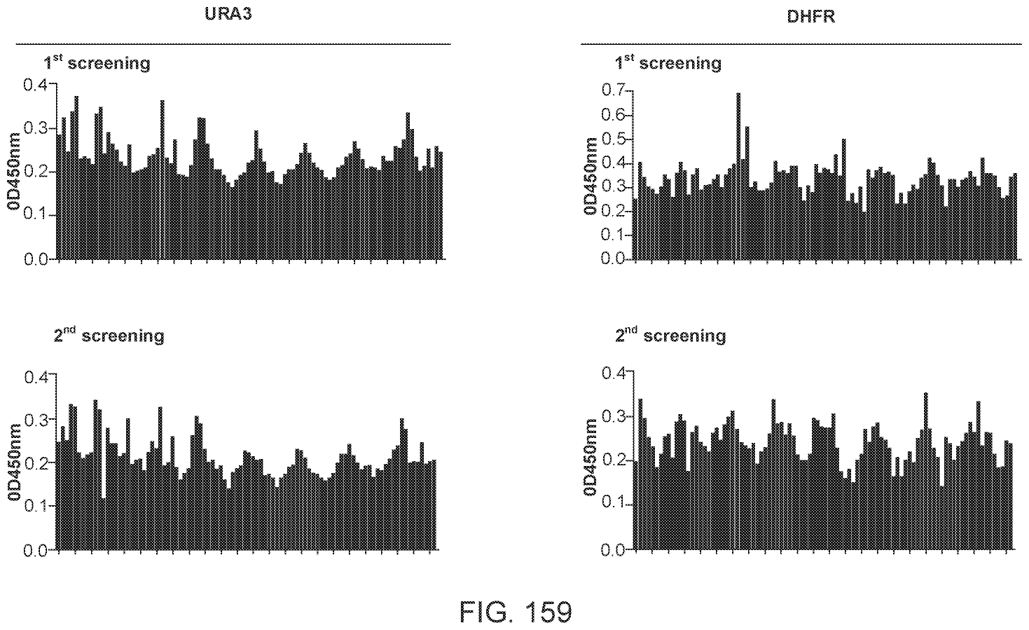

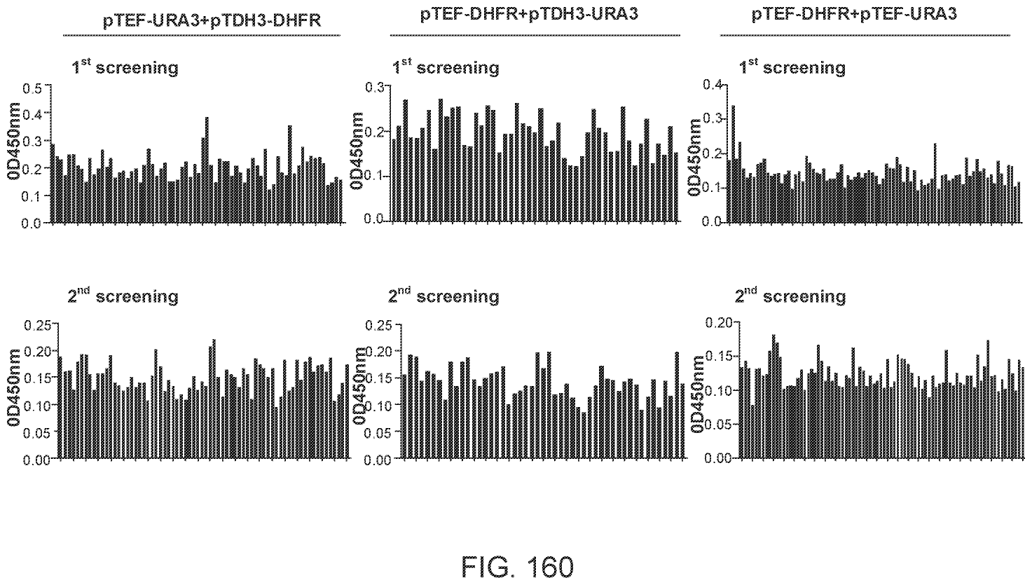

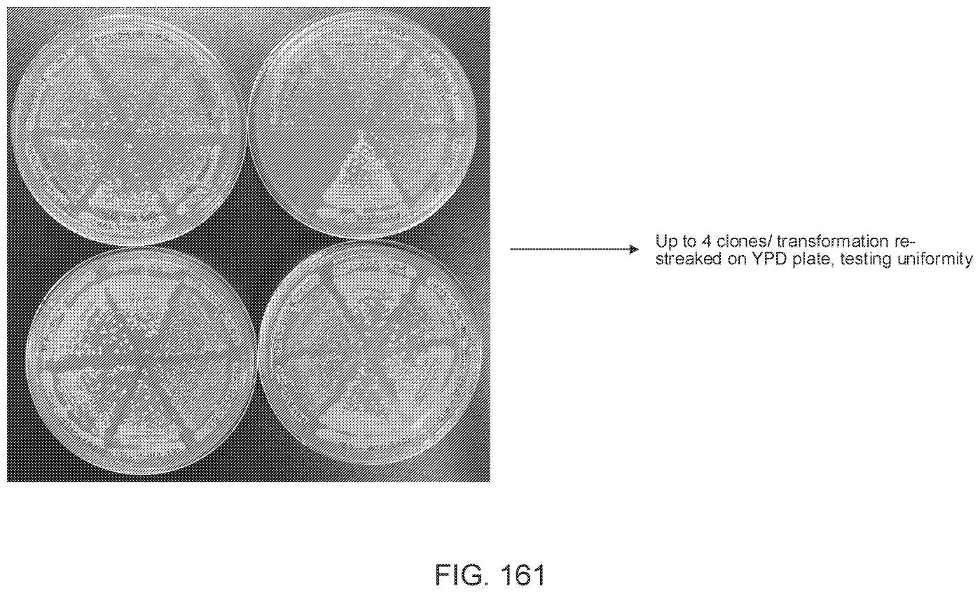

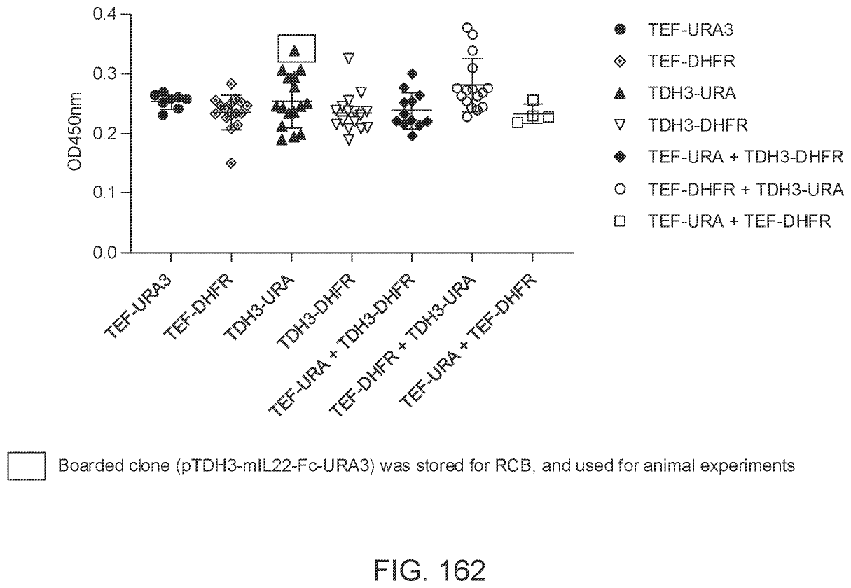

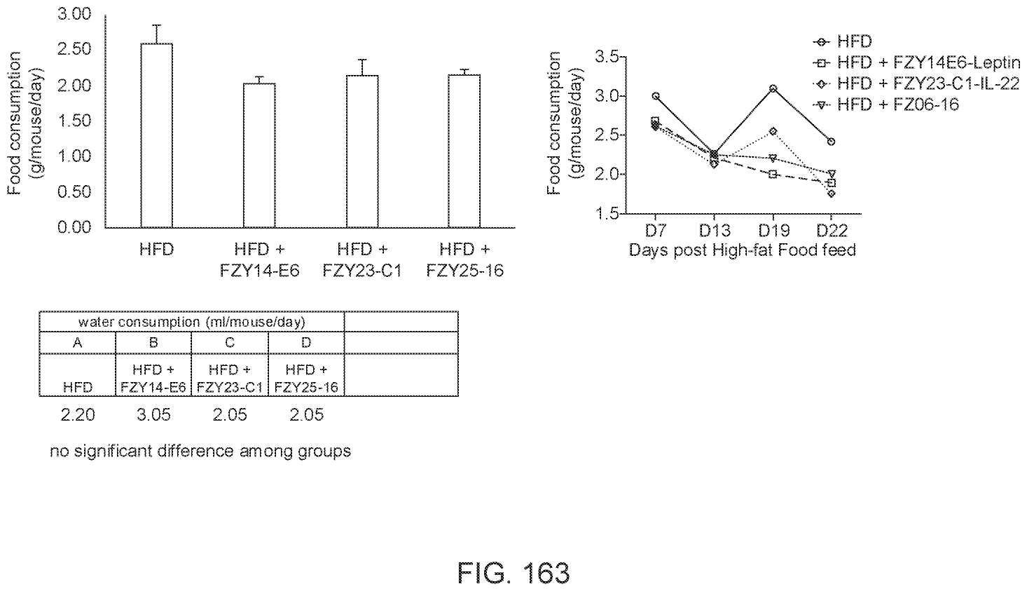

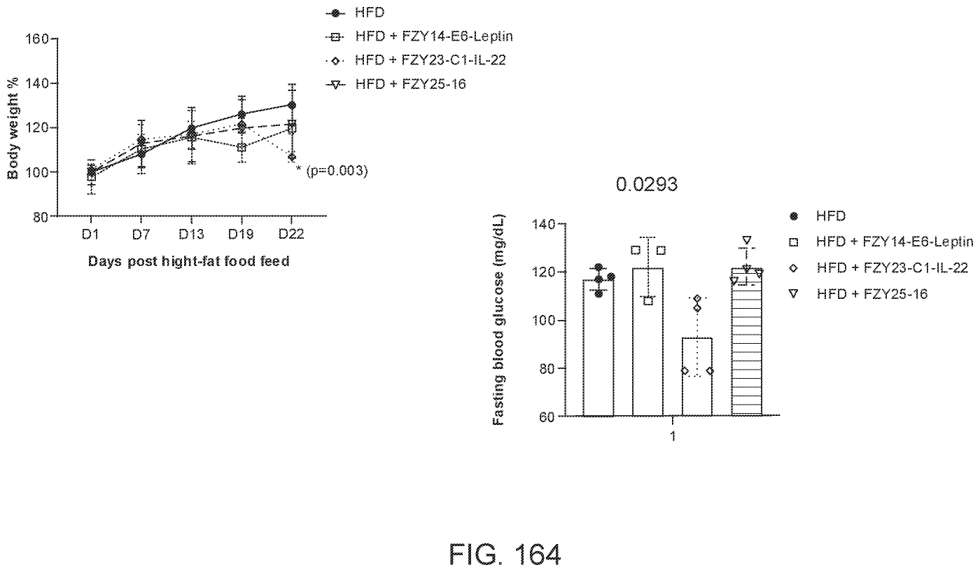

FIGS. 153-164 shows exemplary generation and characterization of engineered strain of S. boulardii constitutively expressing functional mouse IL-22 fused with Fc fragment (e.g., FZ010m).

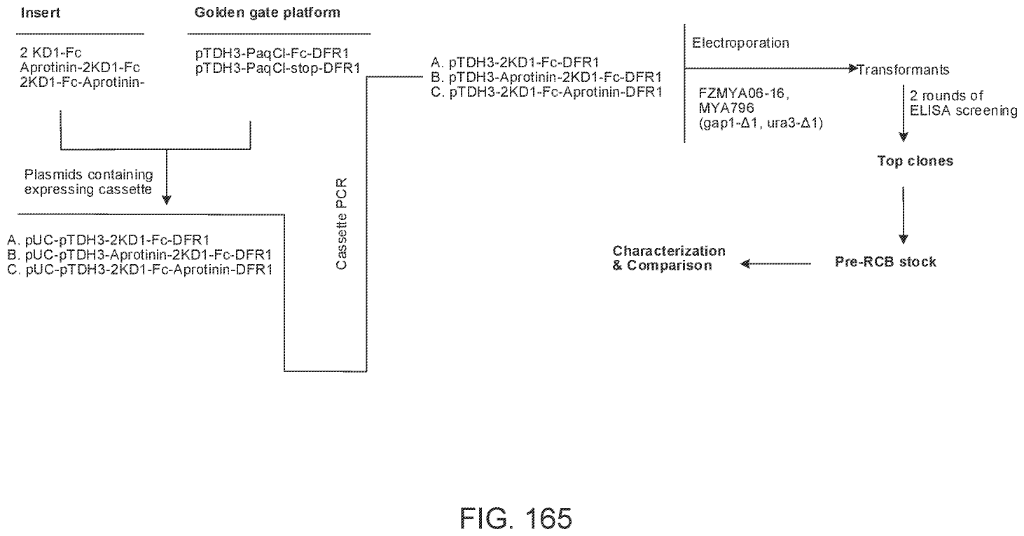

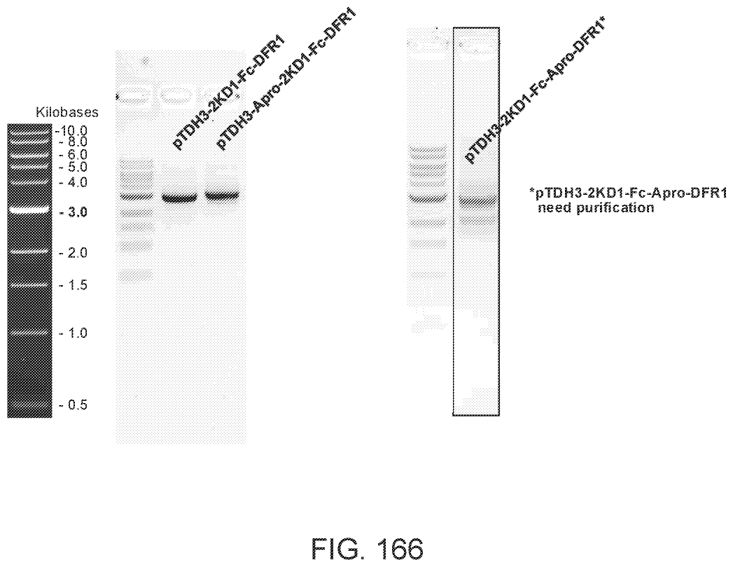

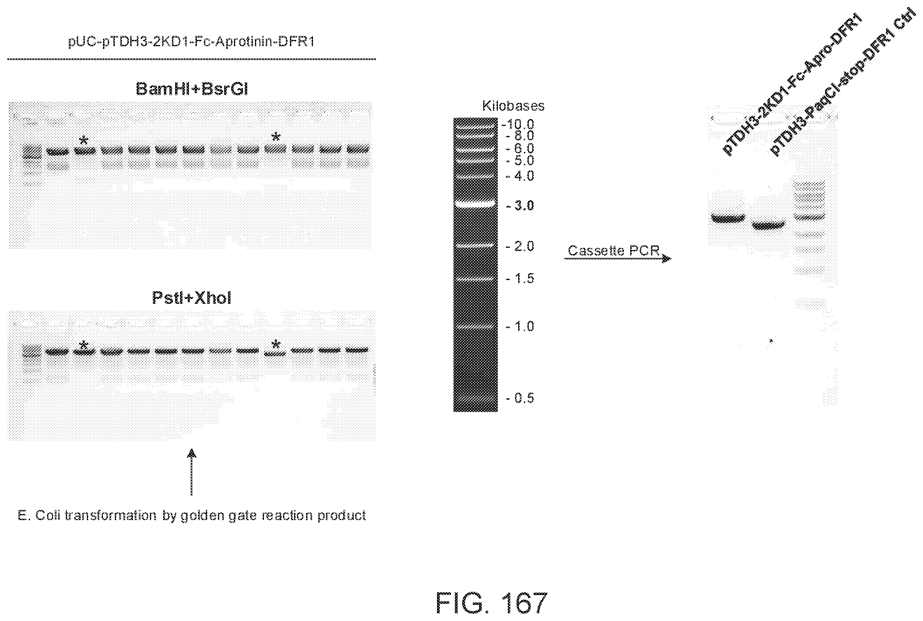

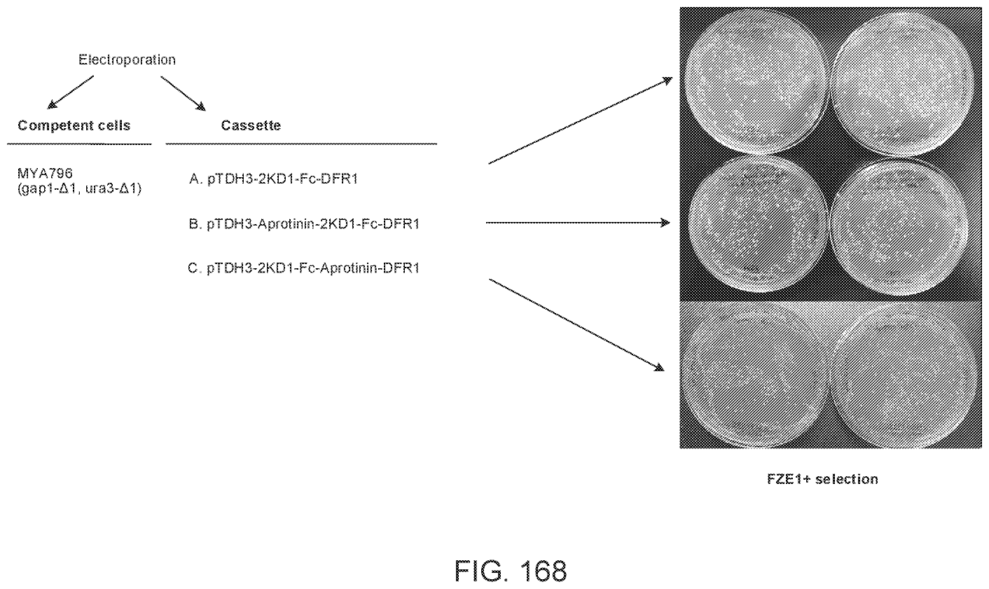

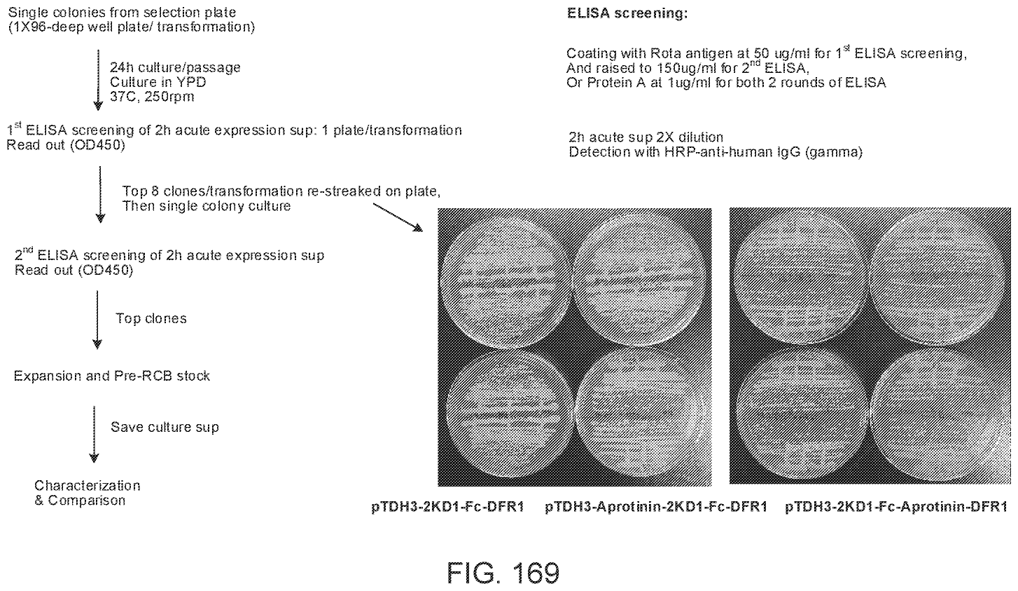

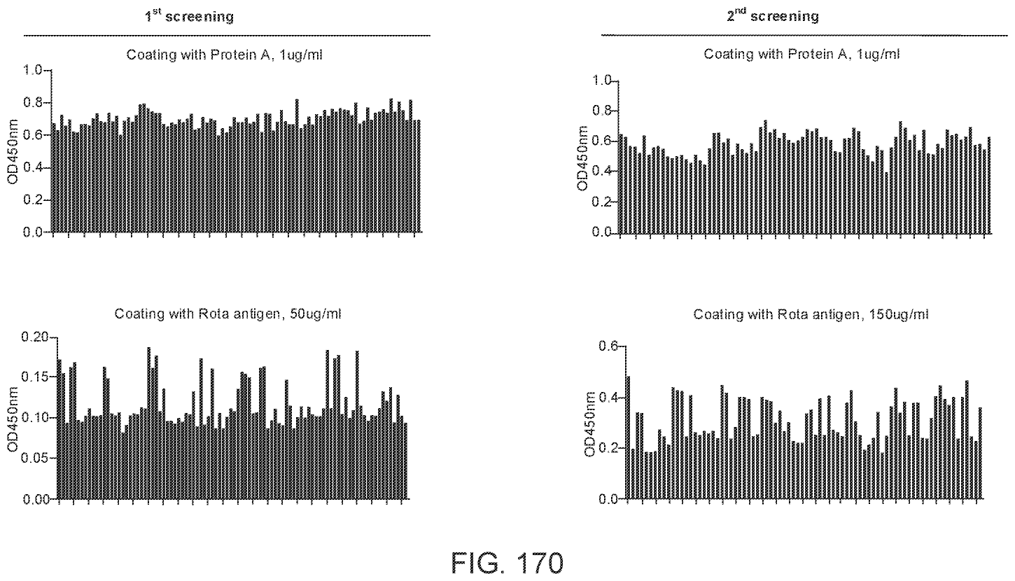

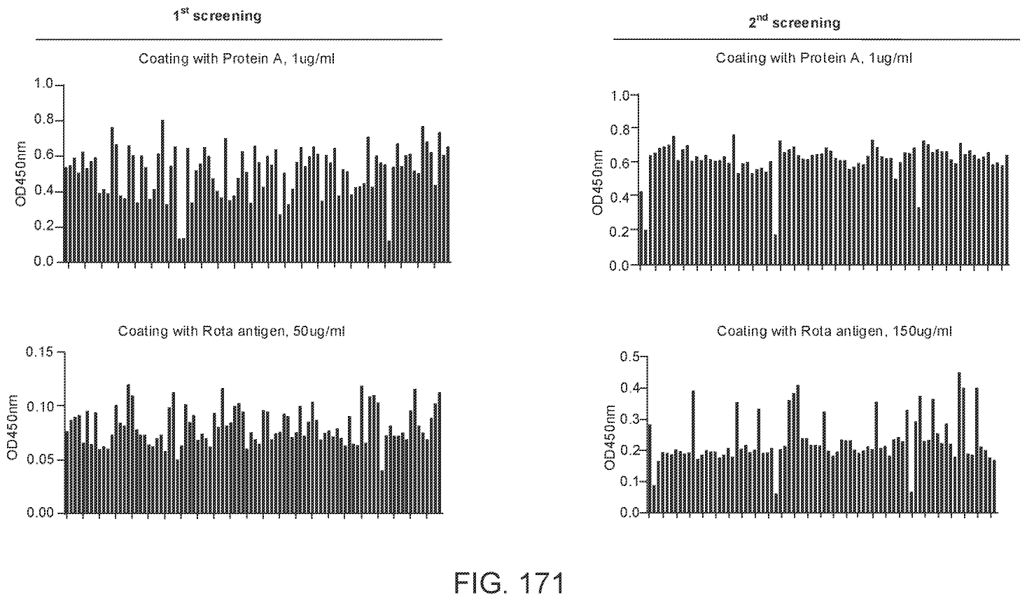

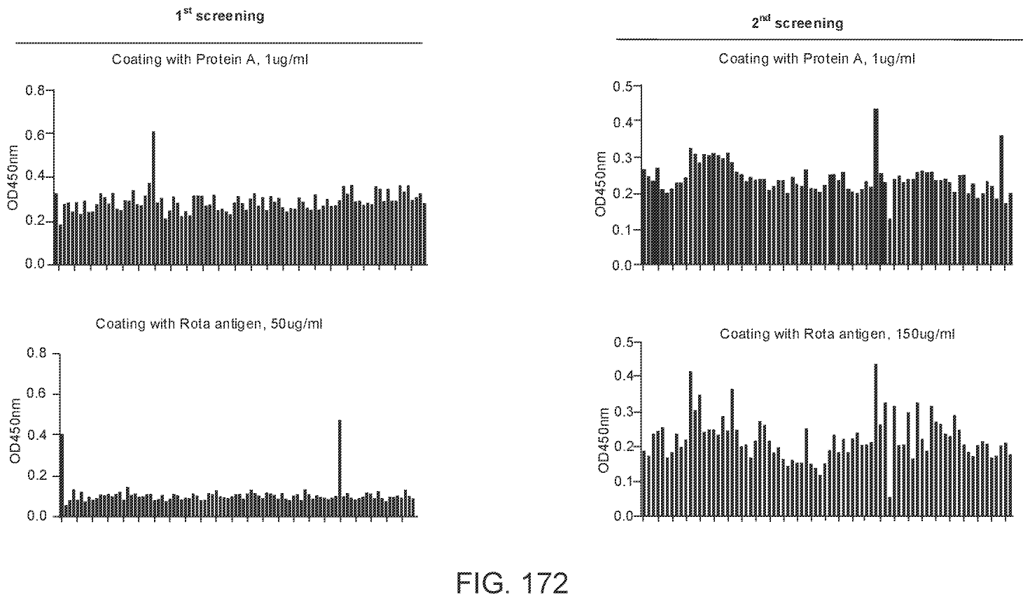

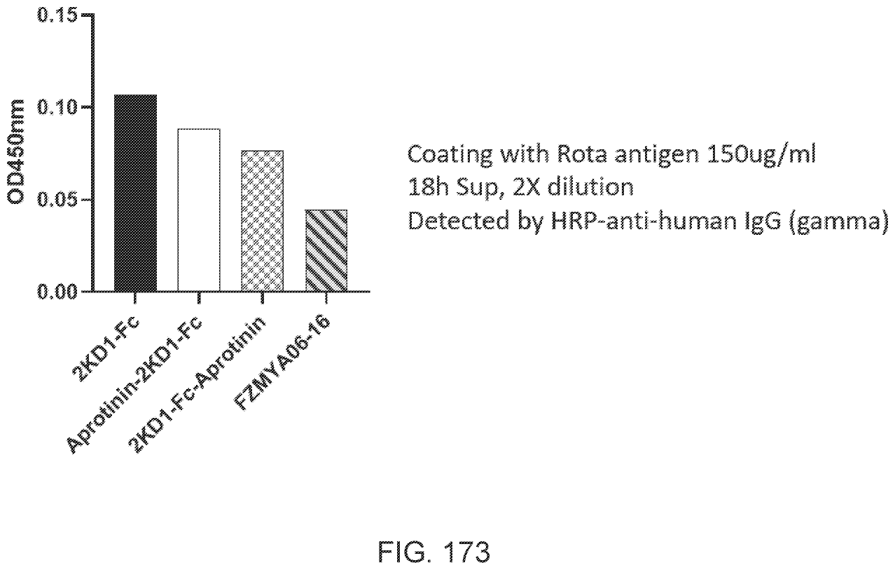

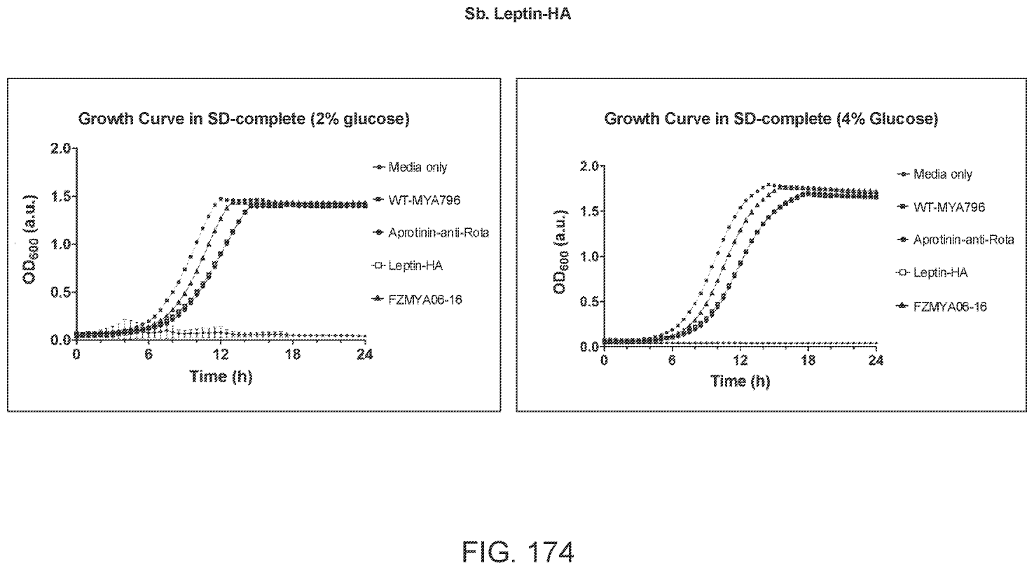

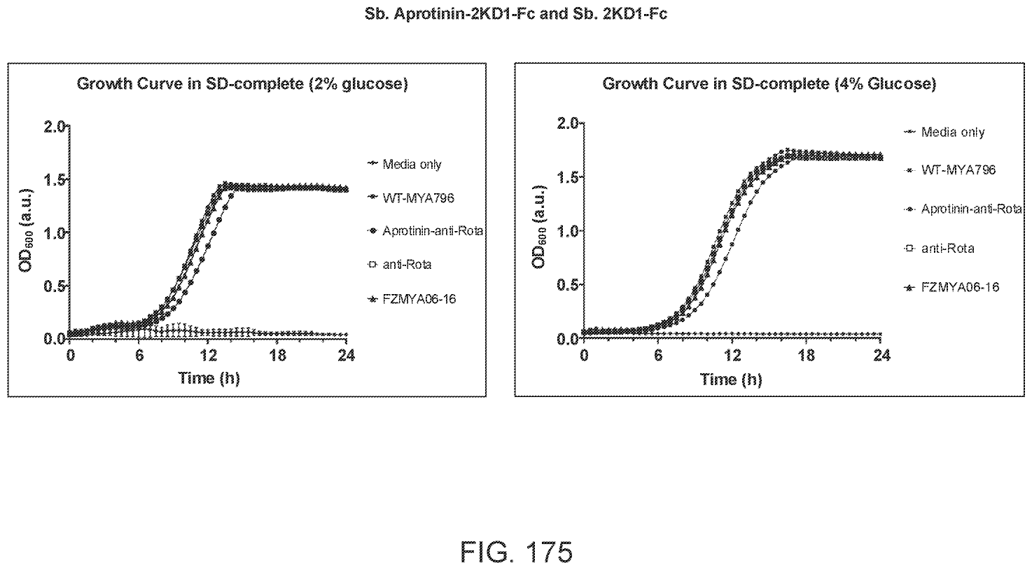

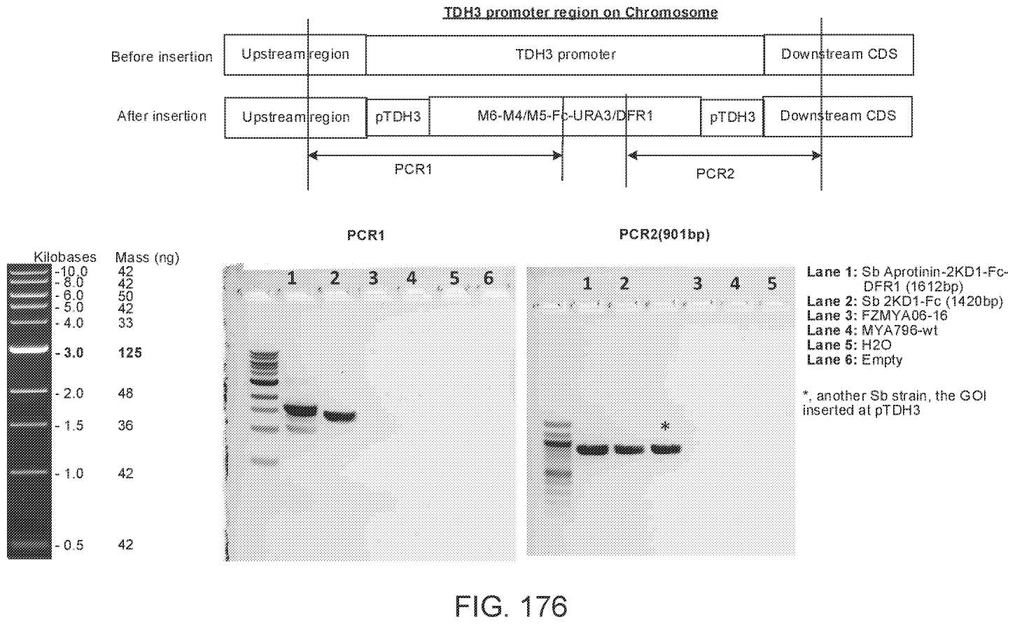

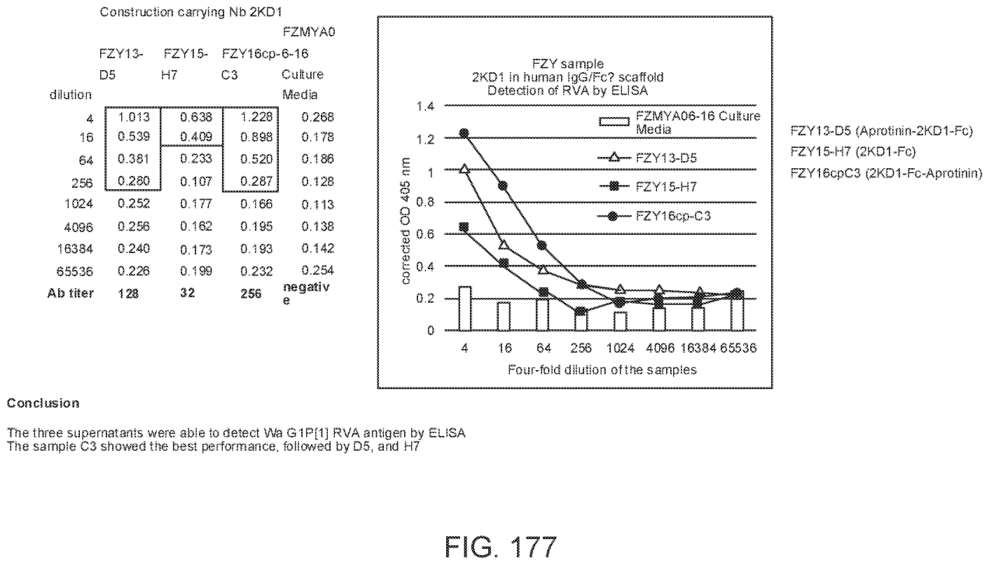

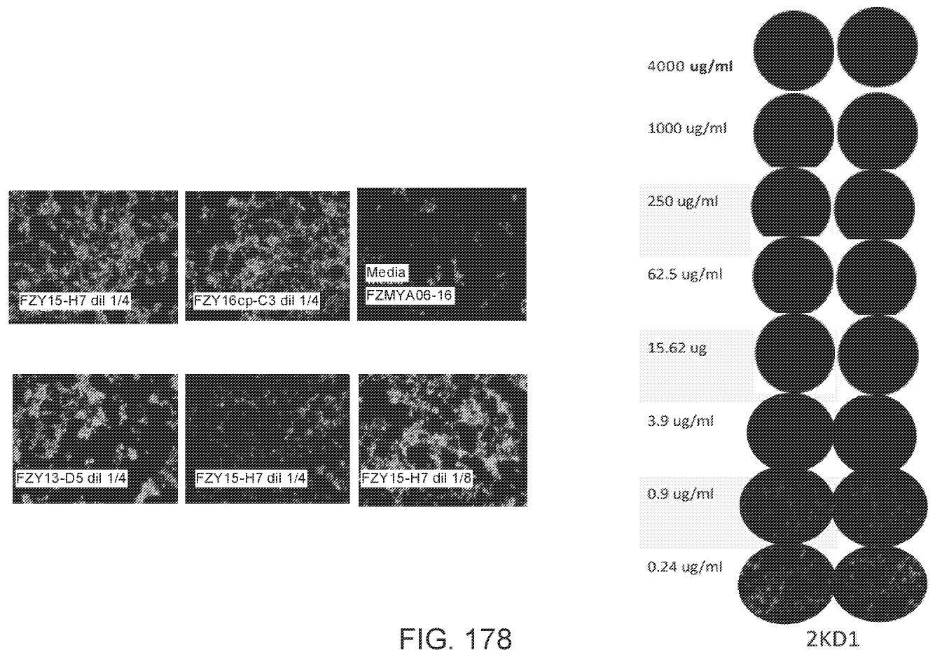

FIGS. 165-178 shows exemplary generation and characterization of engineered strain of S. boulardii constitutively expressing anti-Rotavirus VHH (2KD1) fused with Fc fragment and aprotinin (e.g., FZ014).

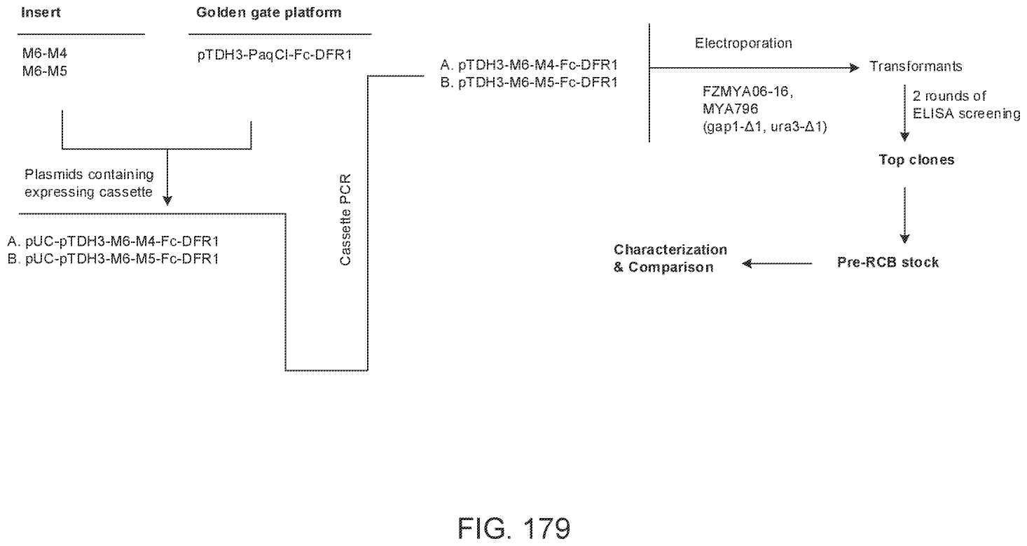

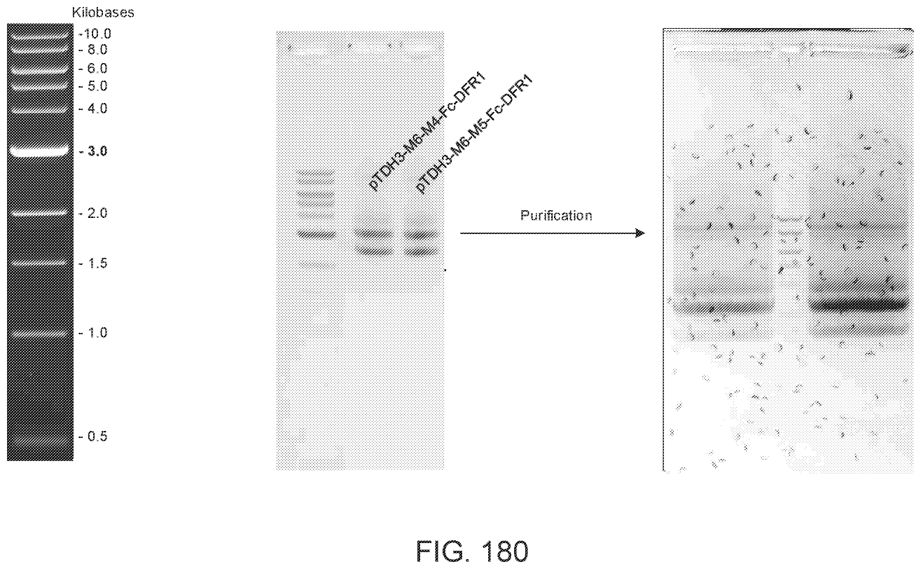

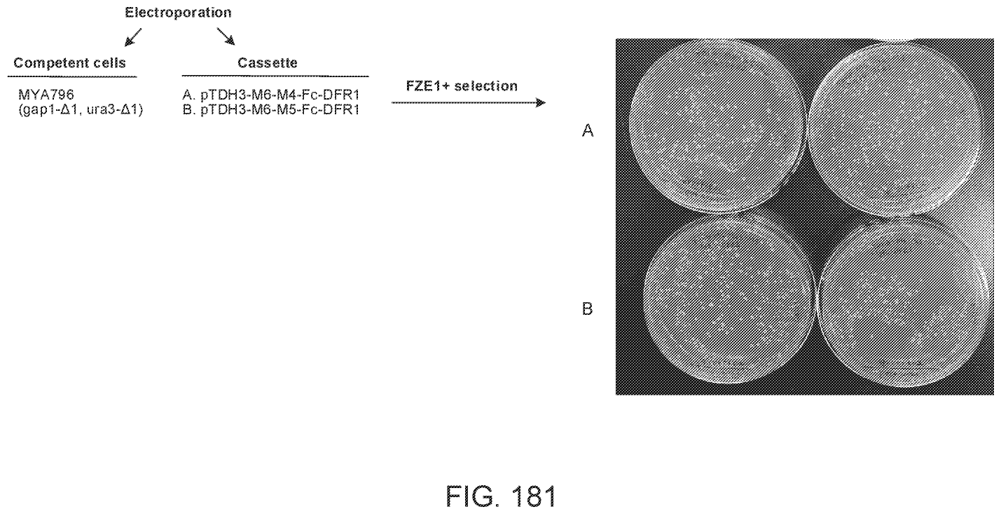

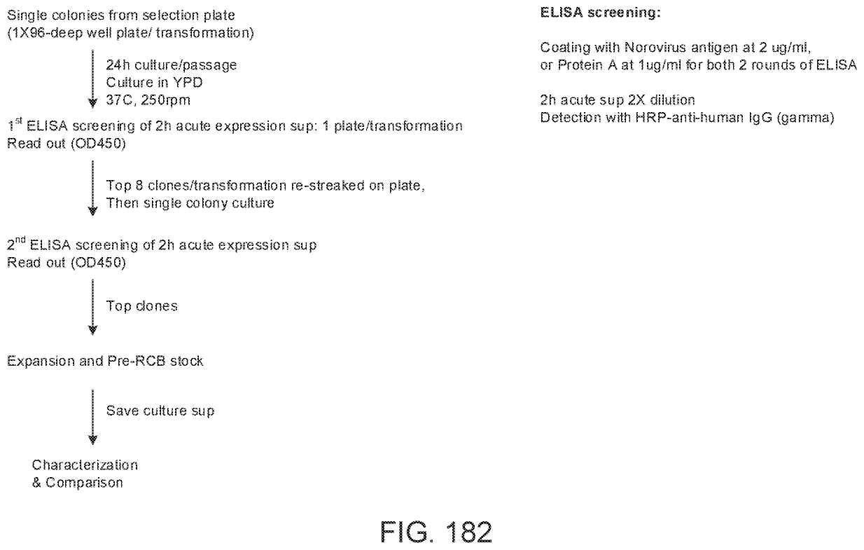

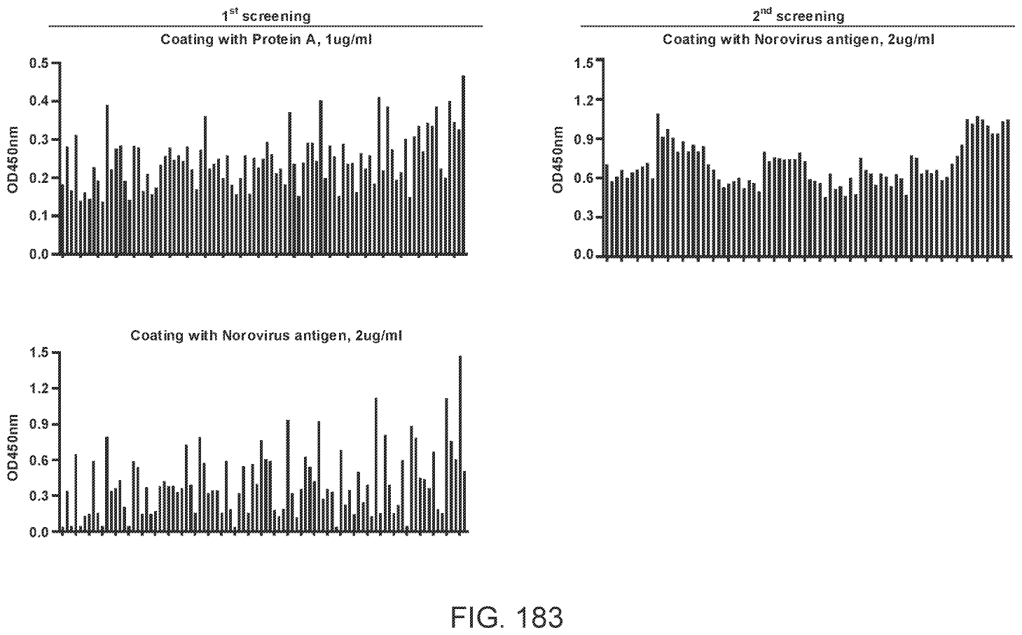

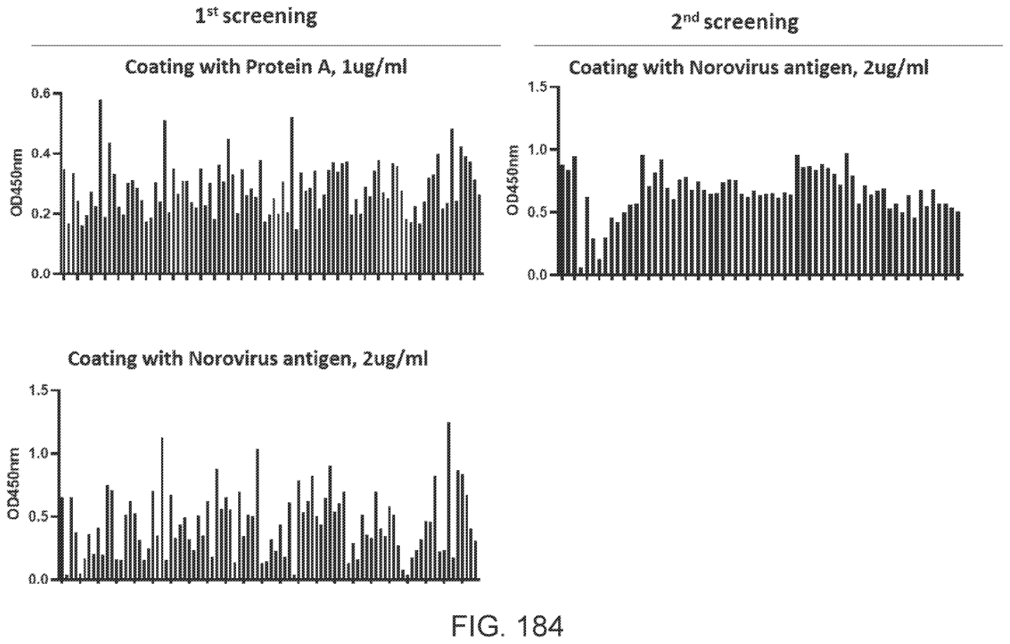

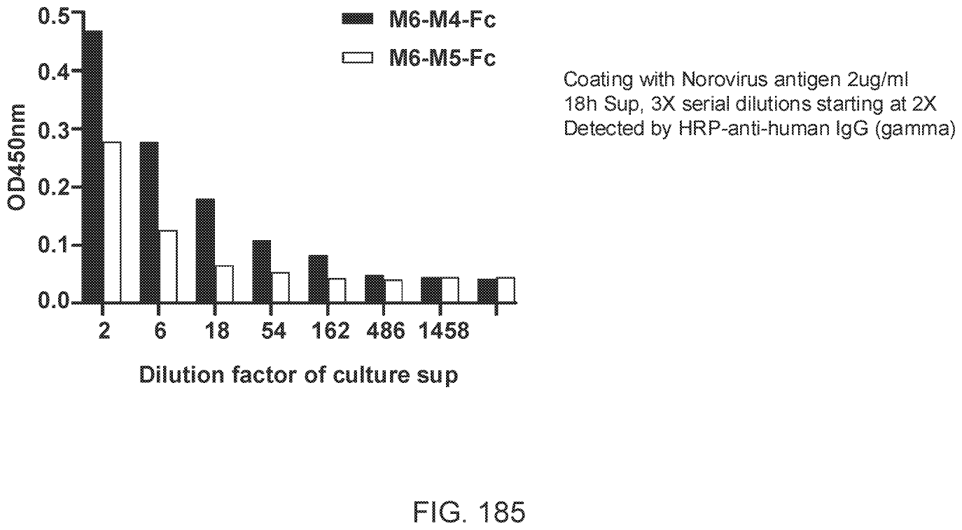

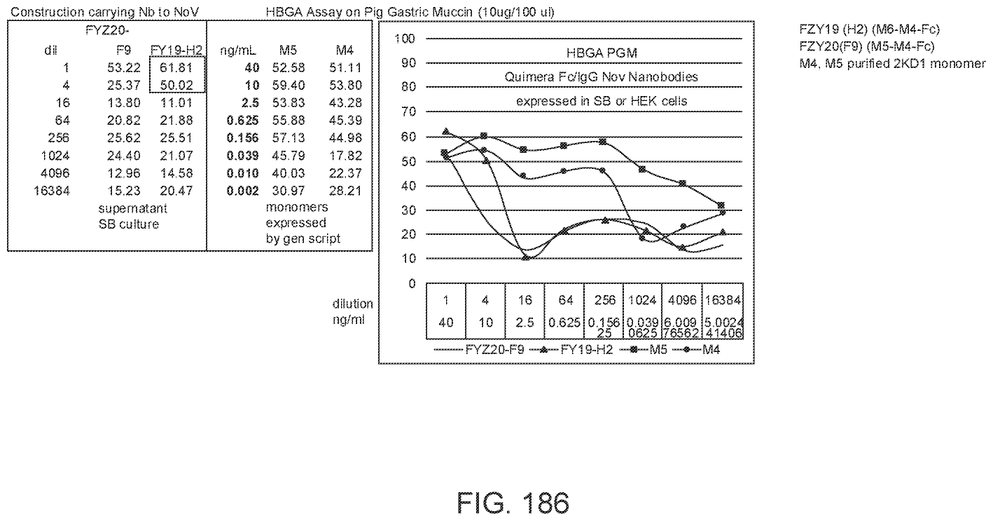

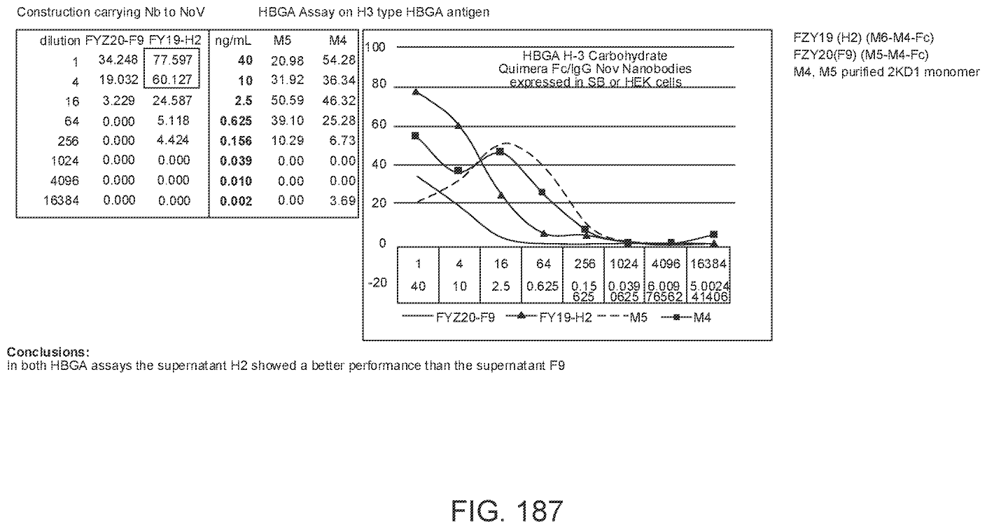

FIGS. 179-187 shows exemplary generation and characterization of engineered strain of S. boulardii constitutively expressing anti-Norovirus bi-specific VHHs fused with Fc fragment (M6M5-Fc and M6M4-Fc) against norovirus (e.g., FZ016).

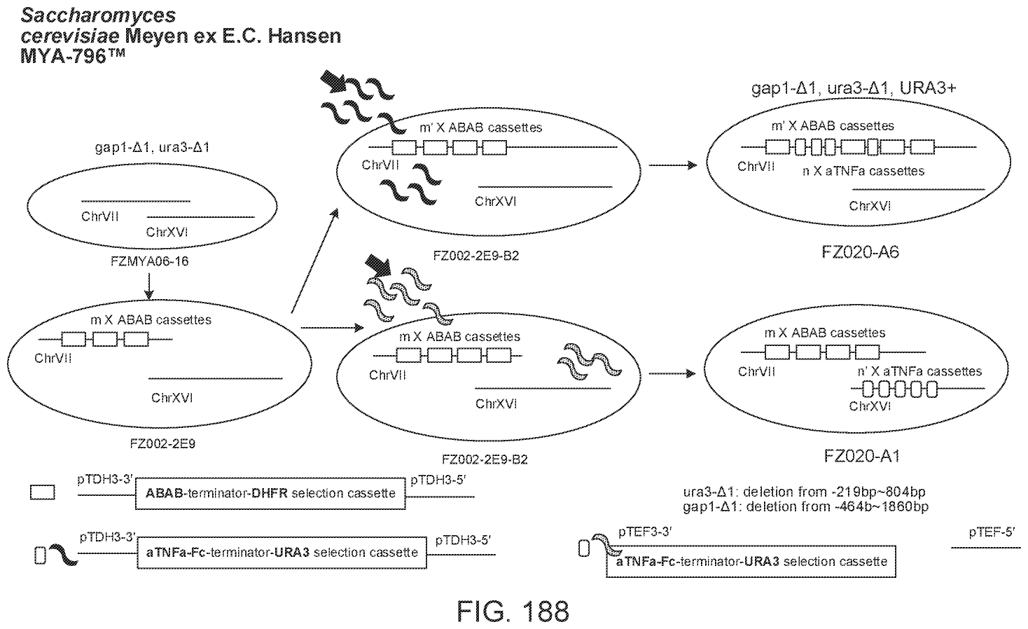

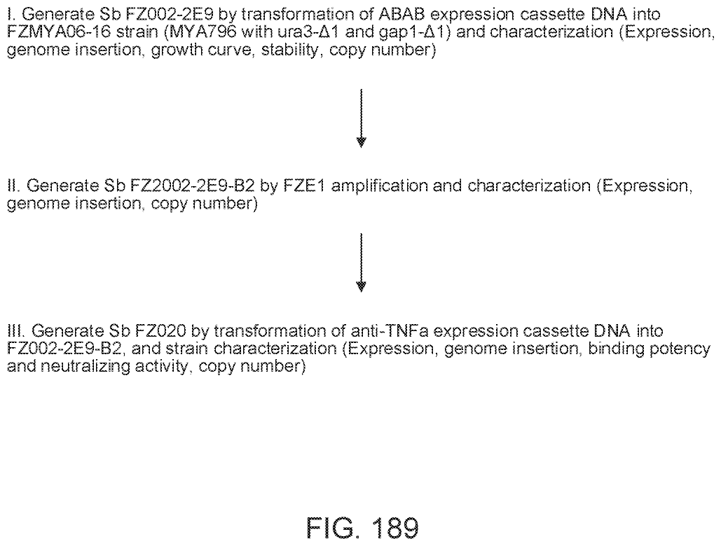

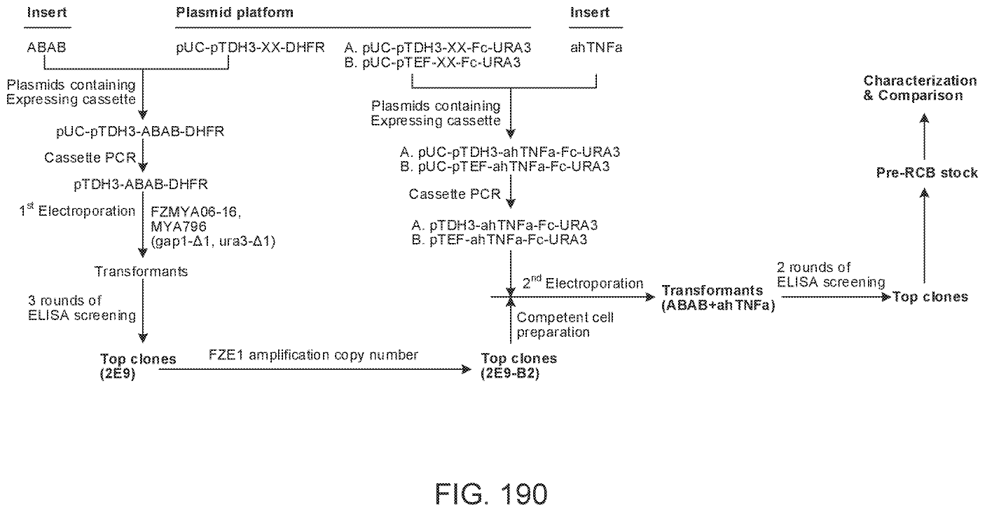

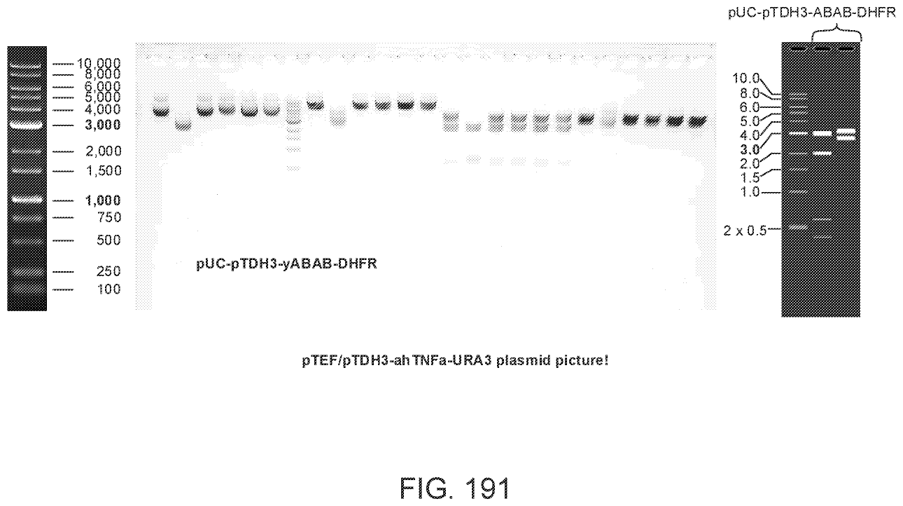

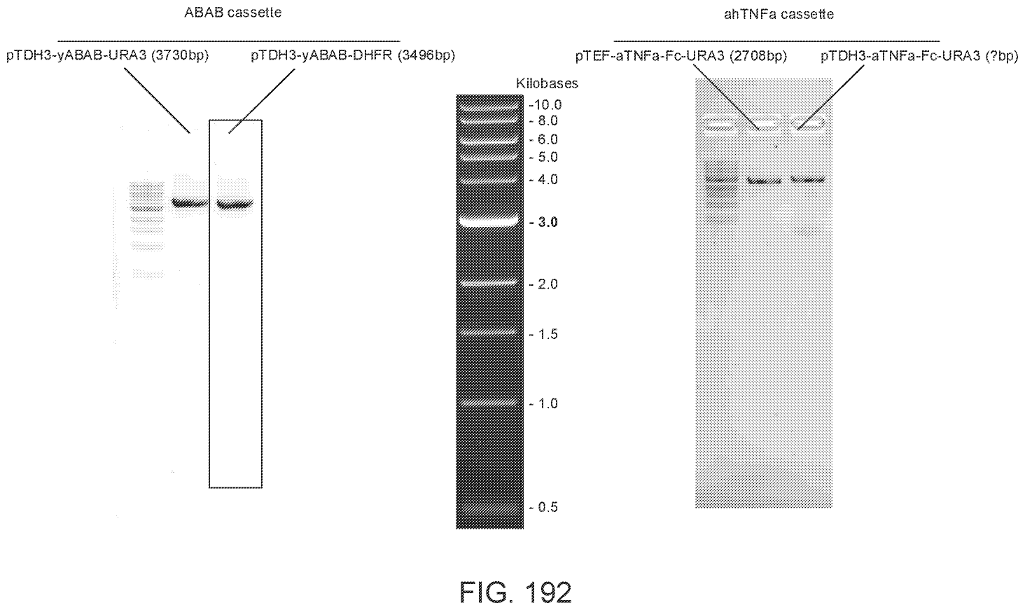

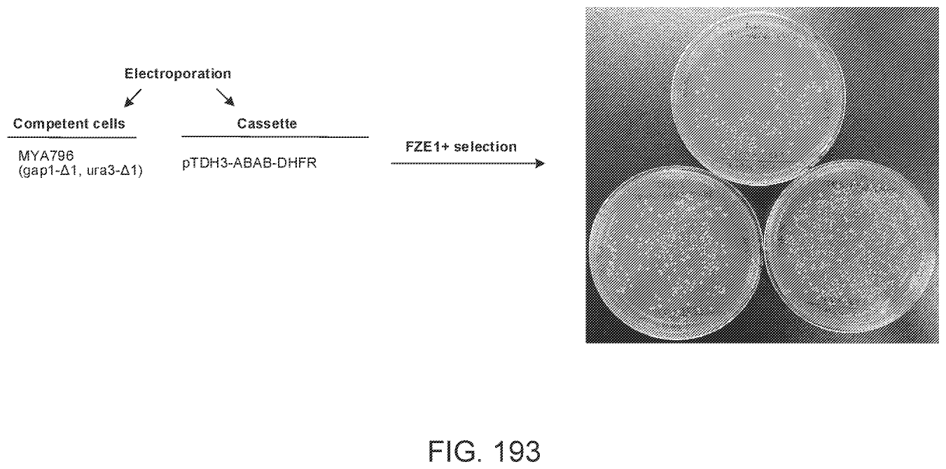

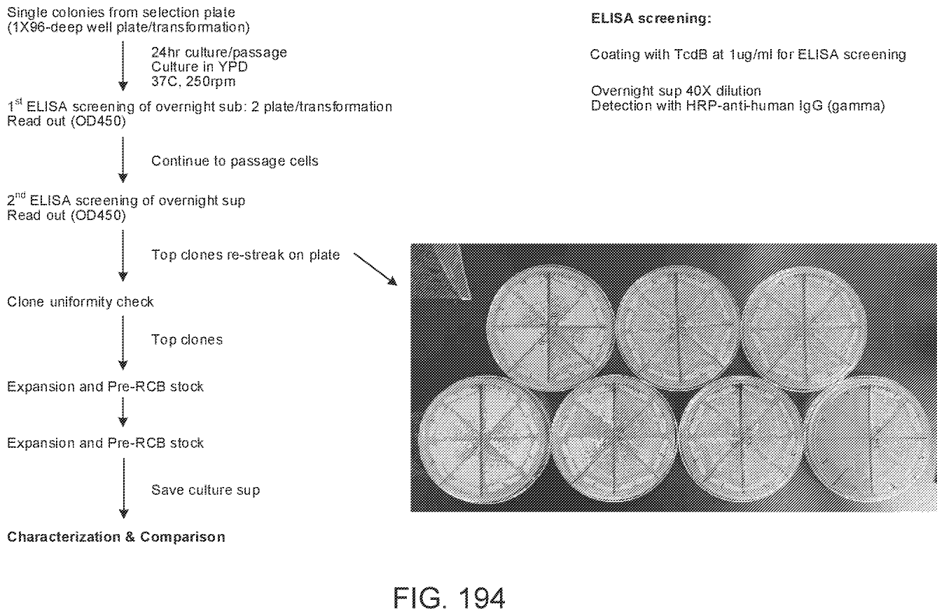

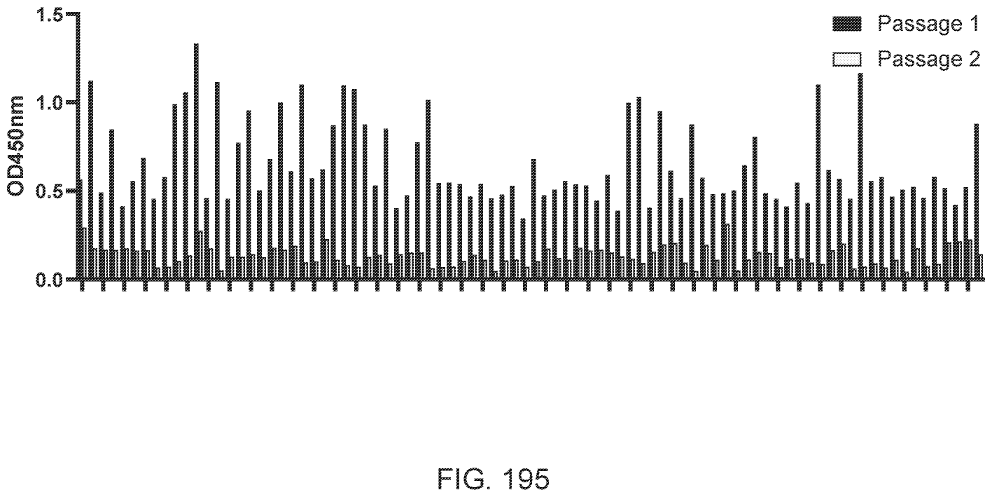

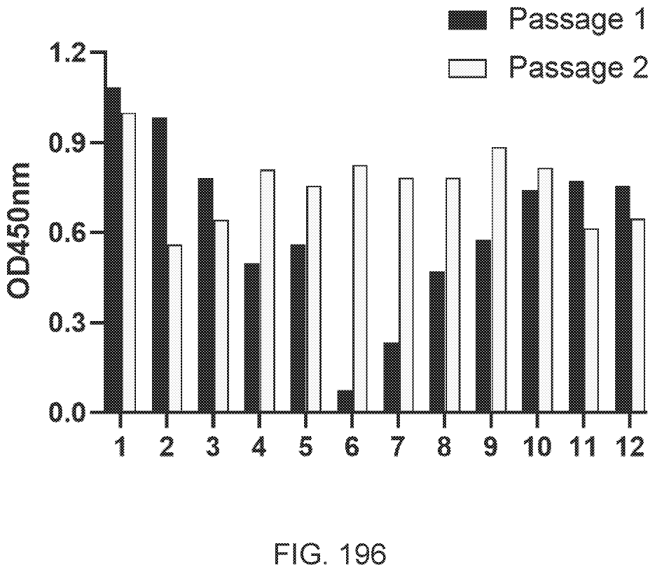

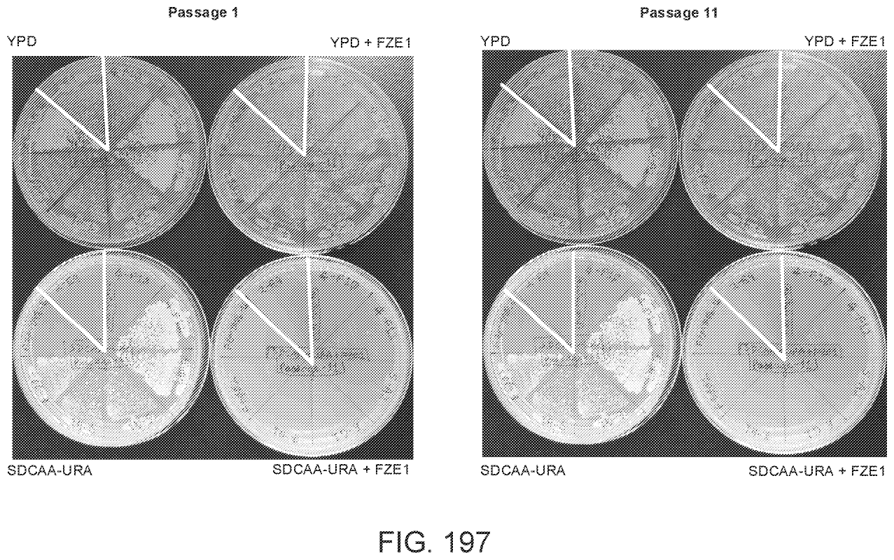

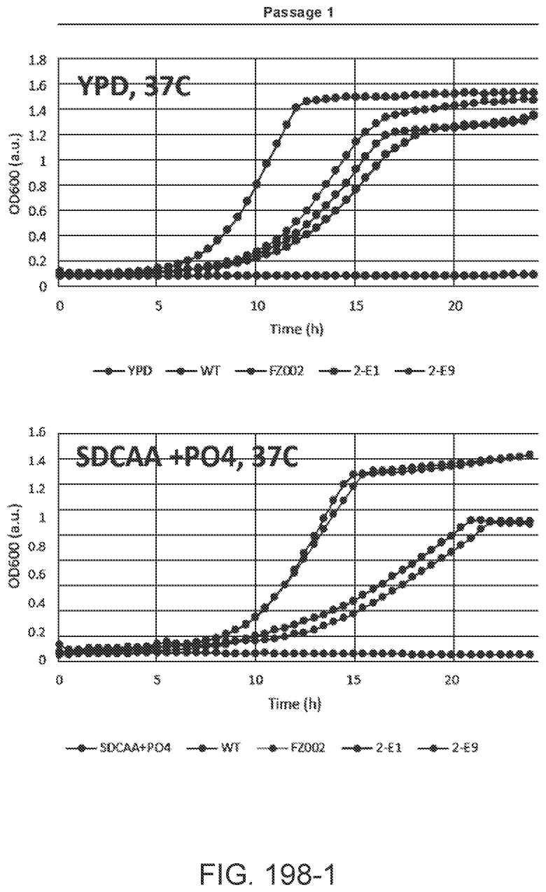

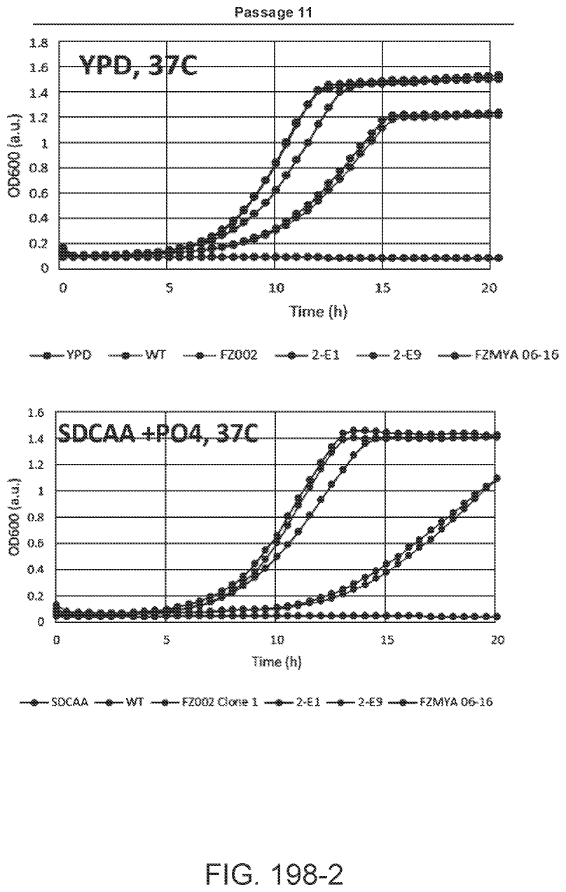

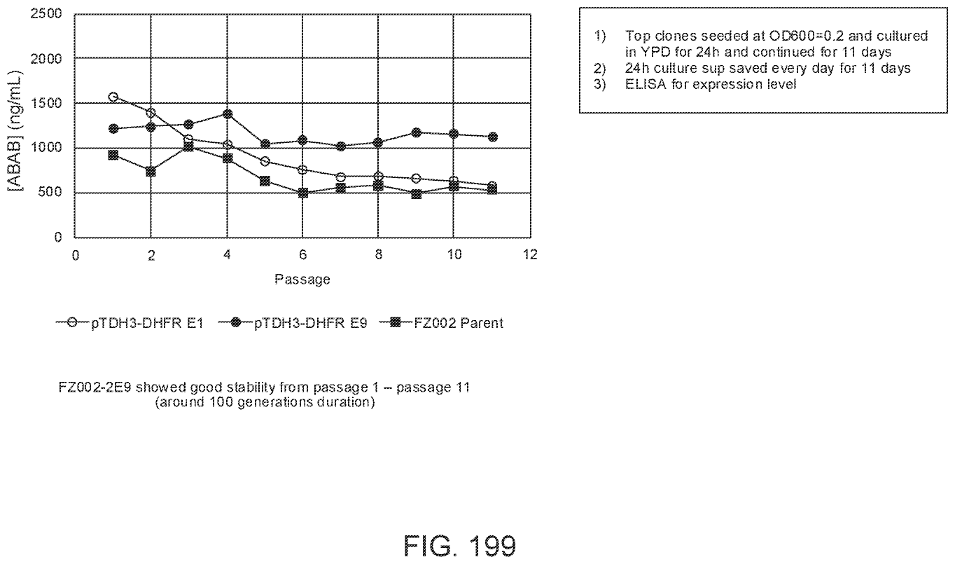

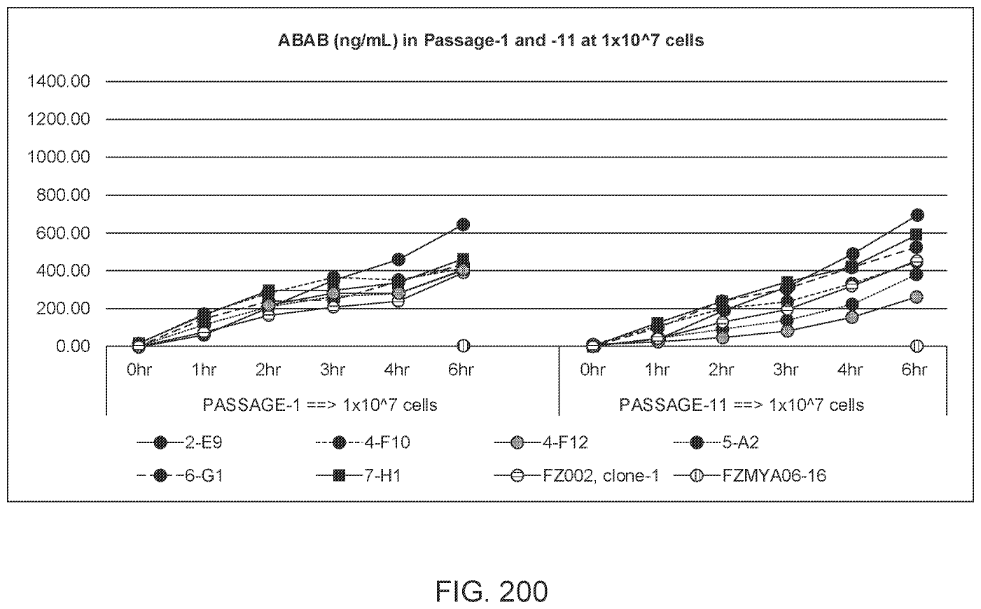

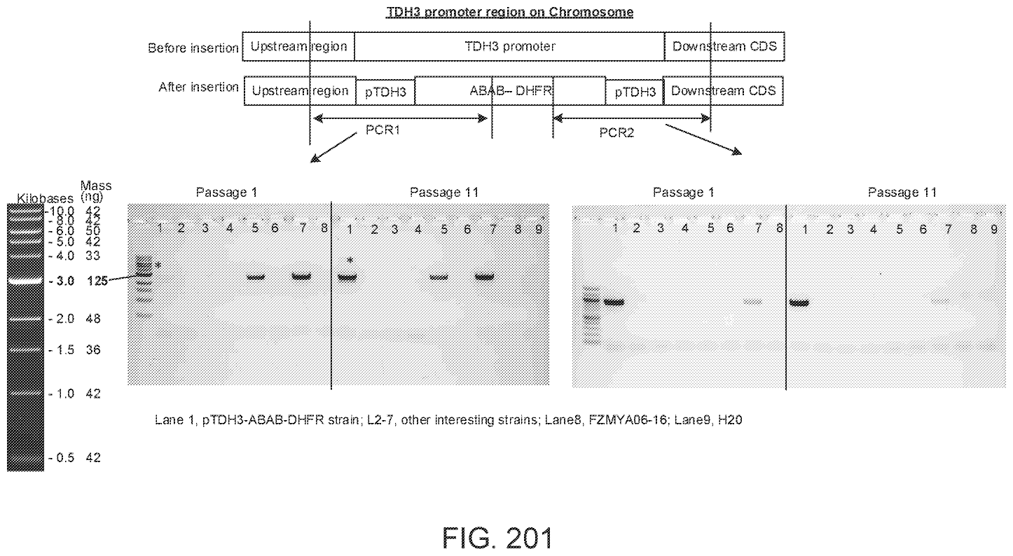

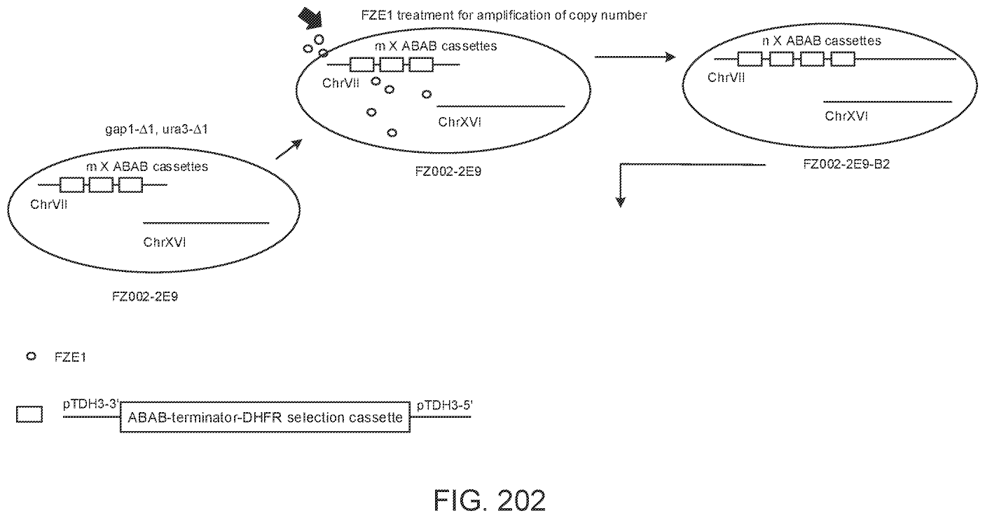

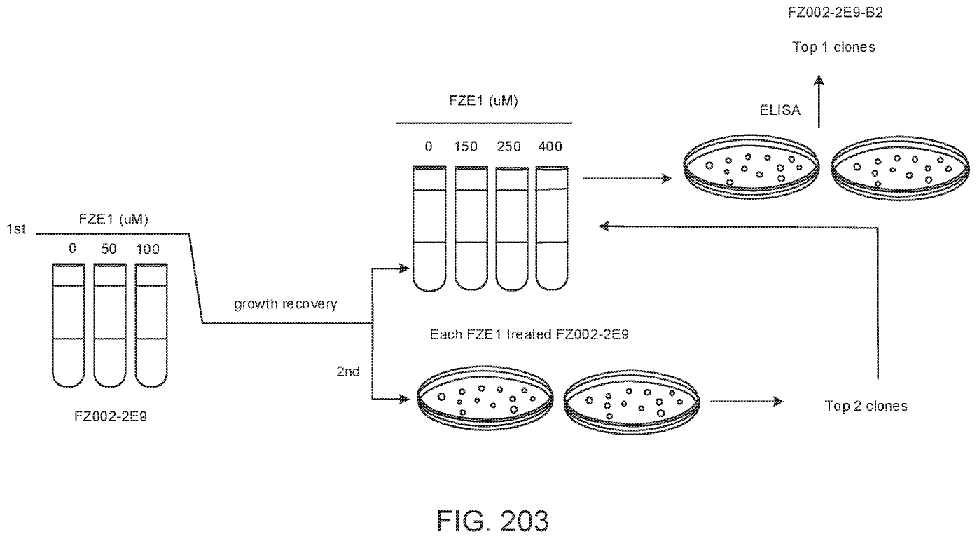

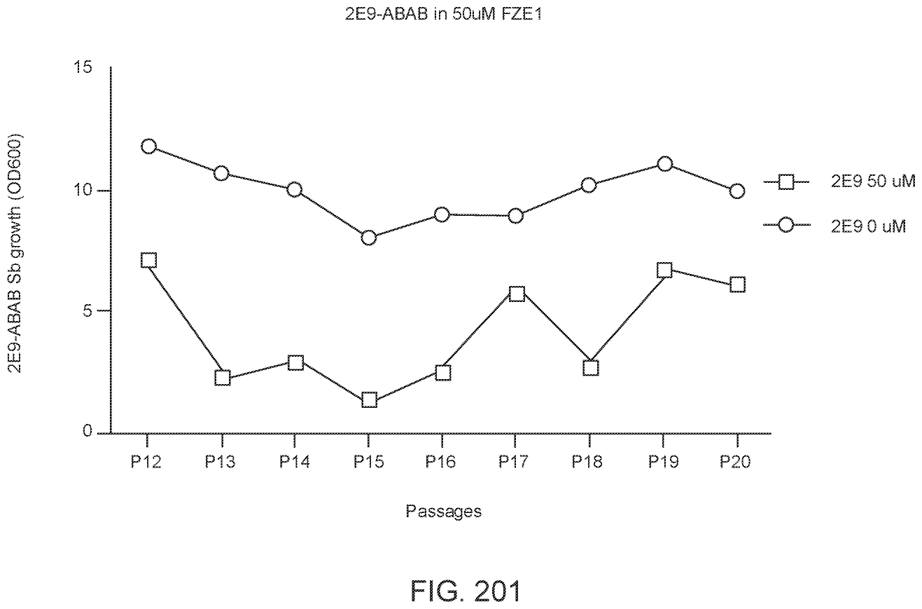

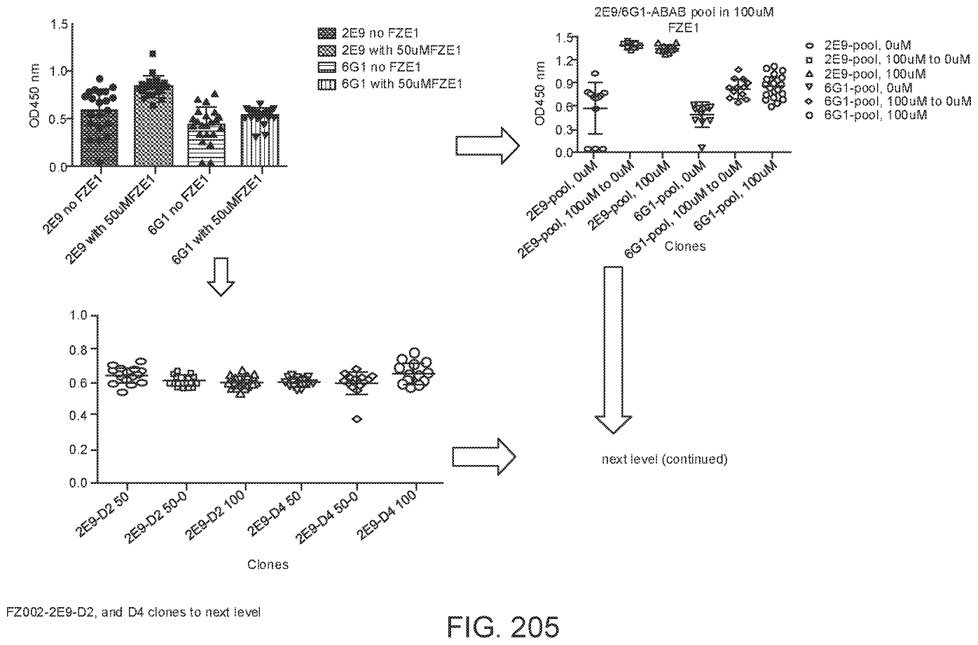

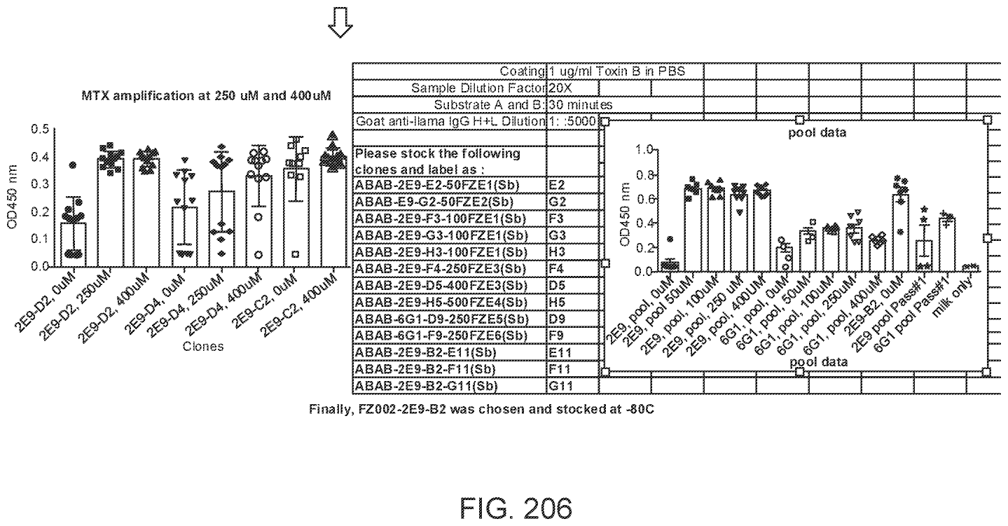

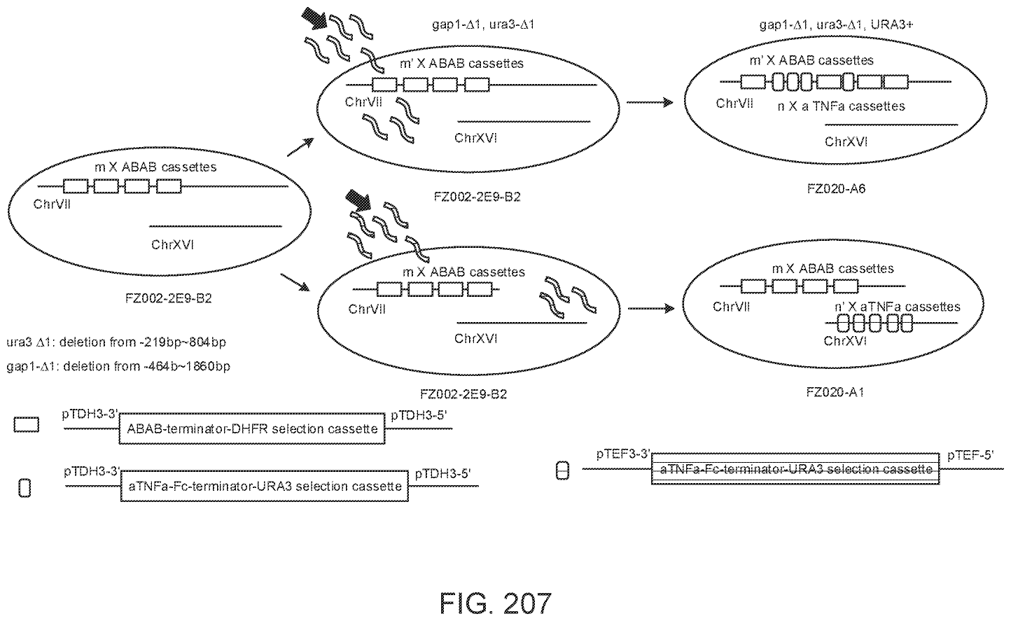

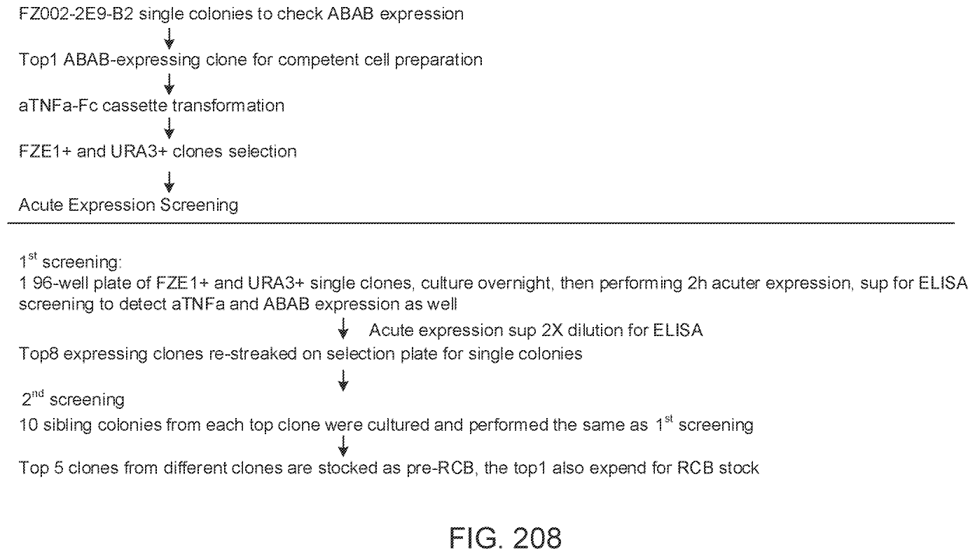

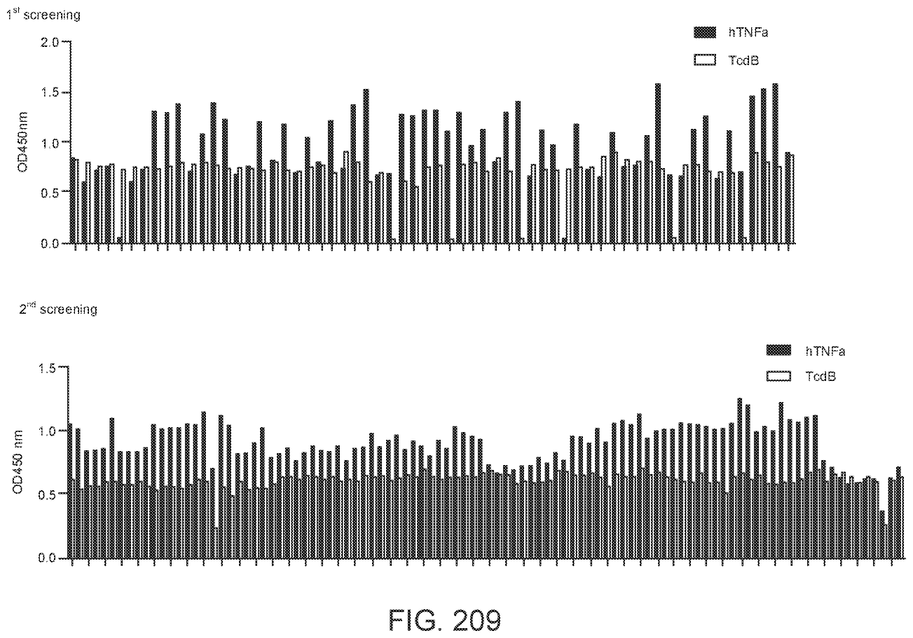

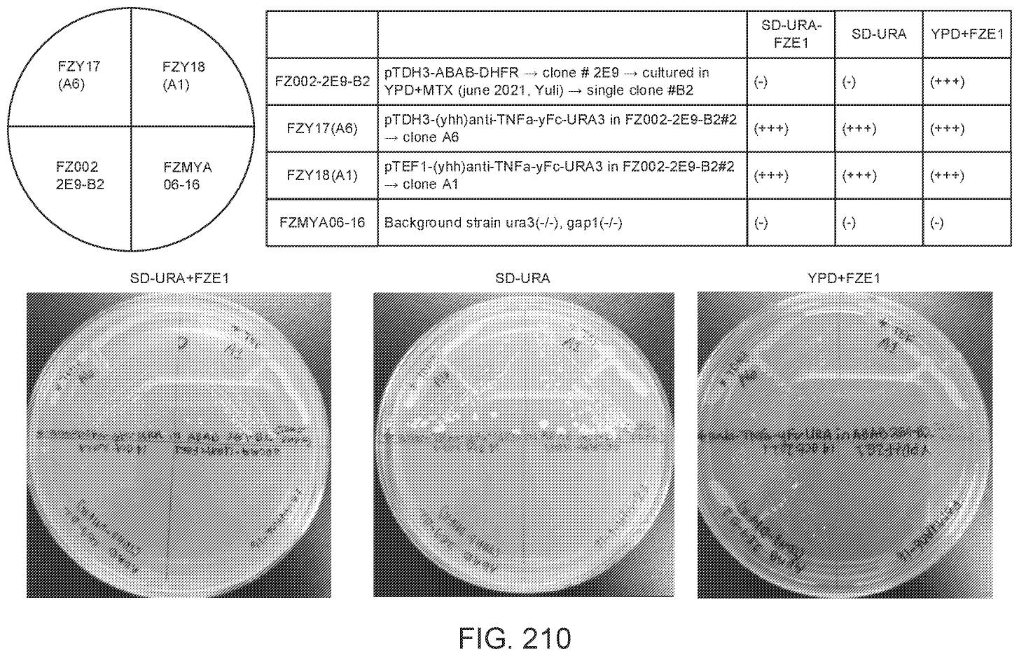

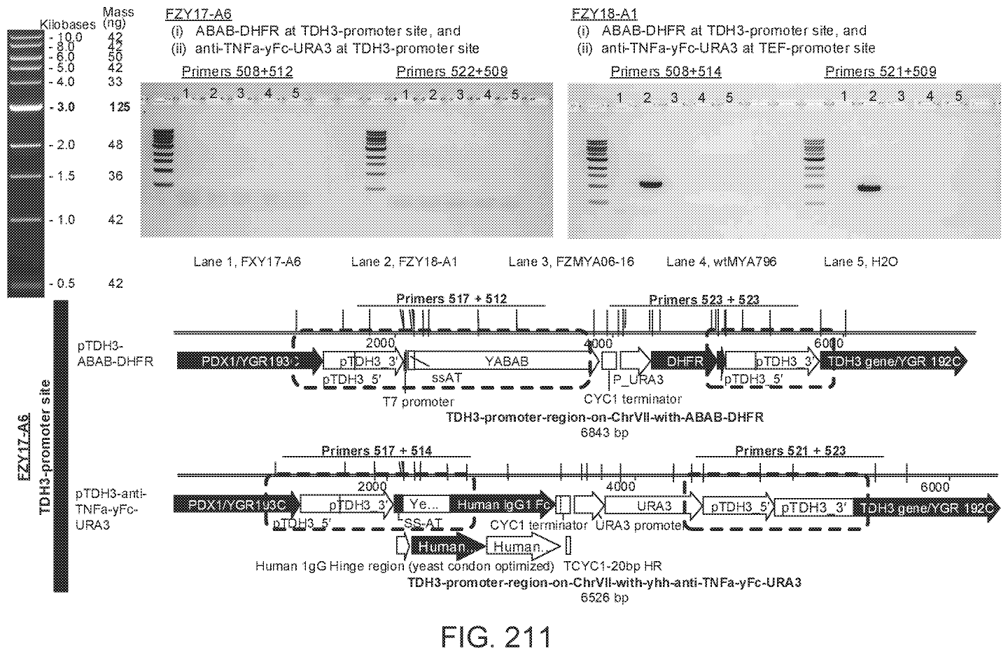

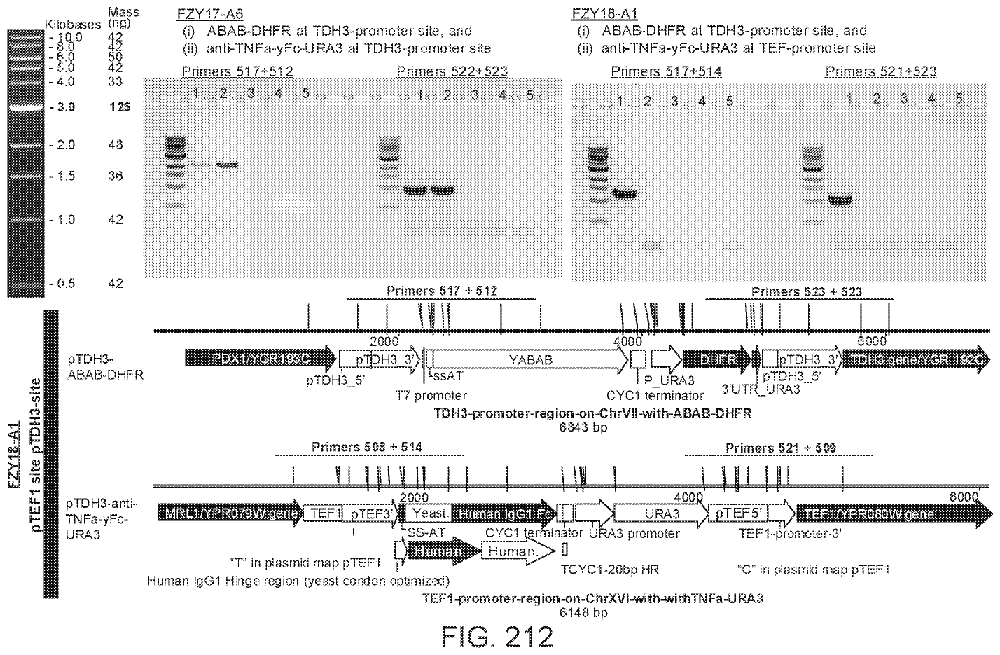

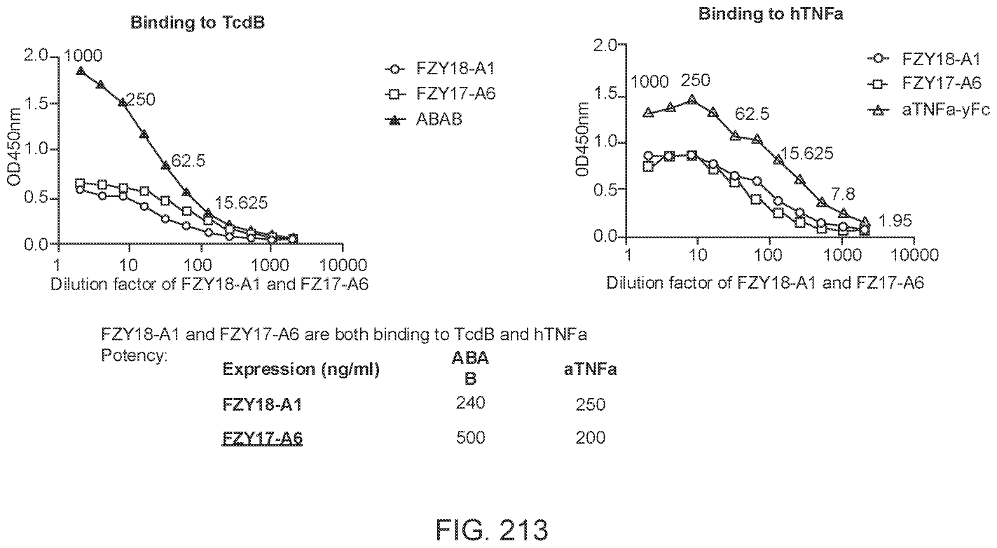

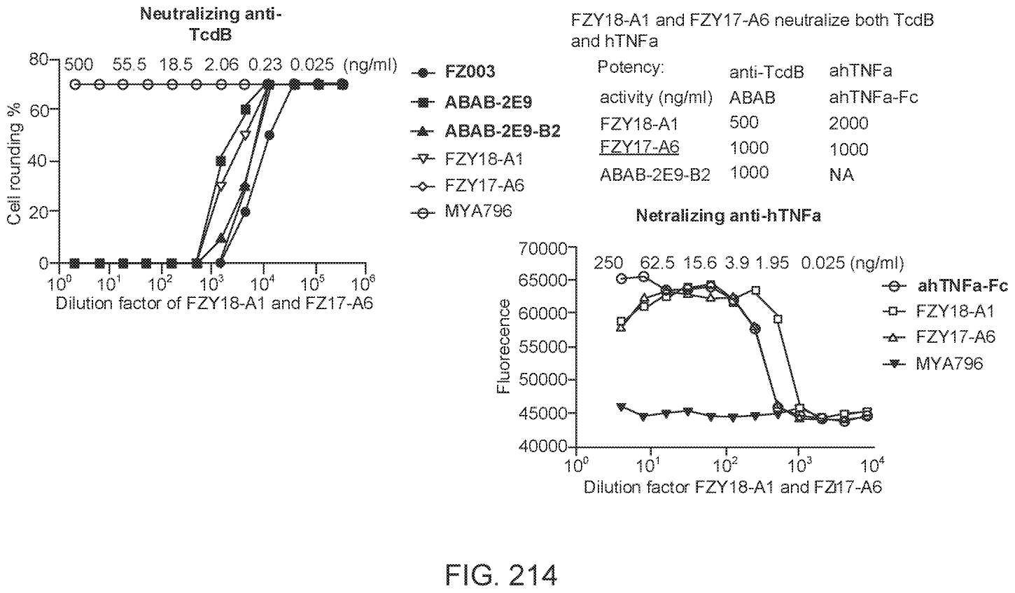

FIGS. 188-214 shows exemplary generation and characterization of engineered strain of S. boulardii constitutively expressing anti-hTNFα and antitoxin ABAB (e.g., FZ020).

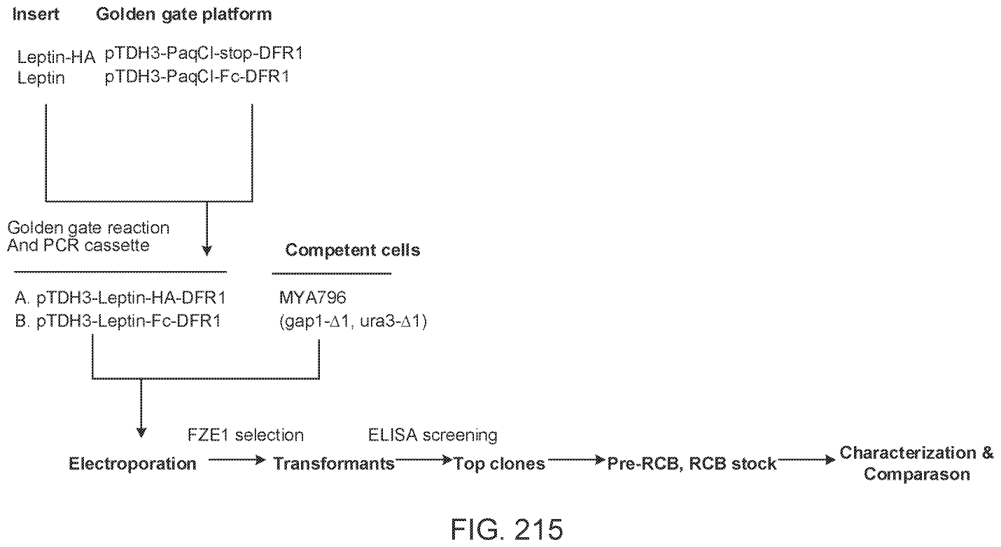

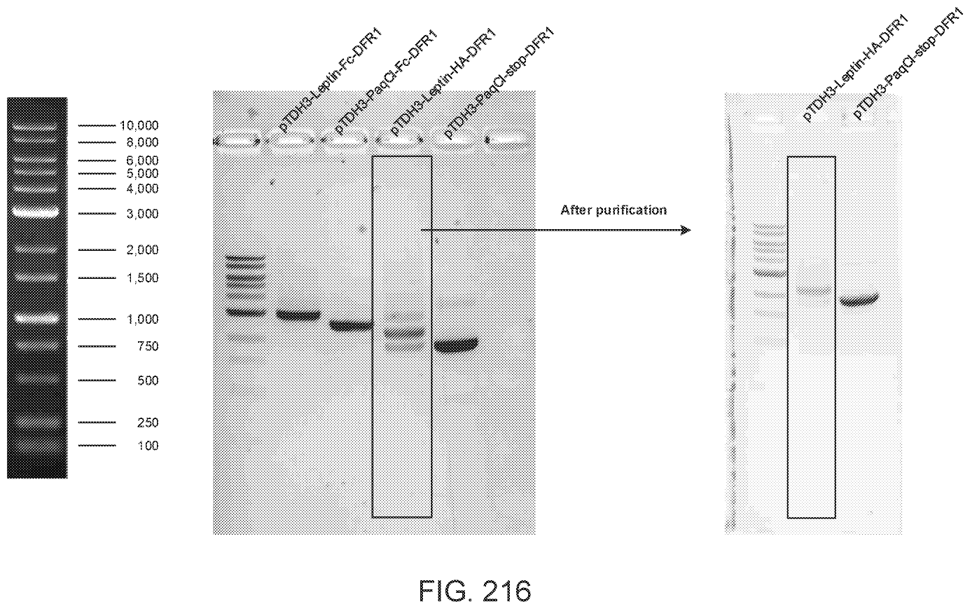

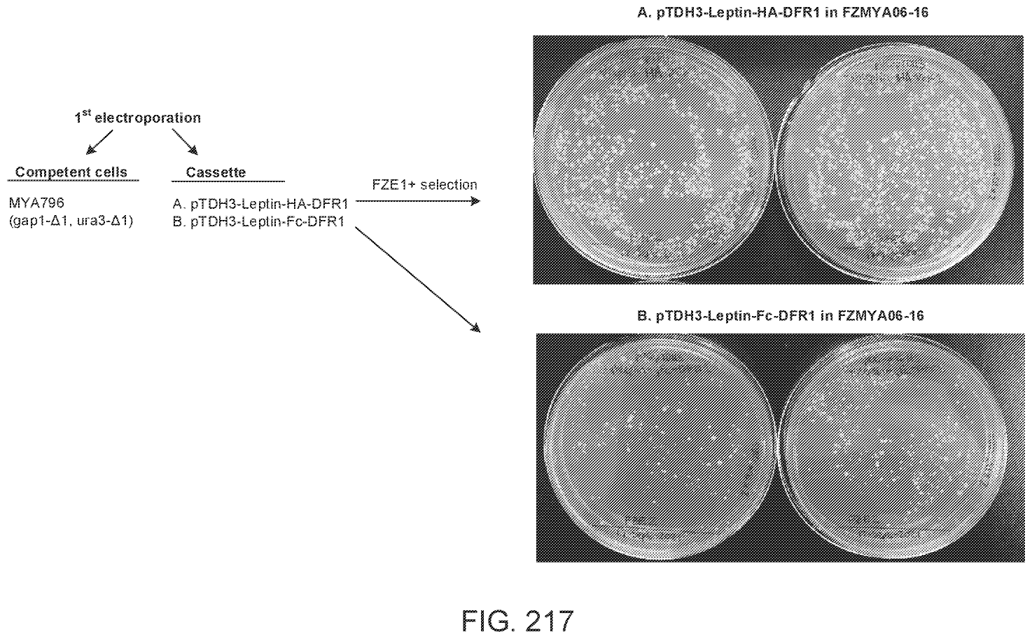

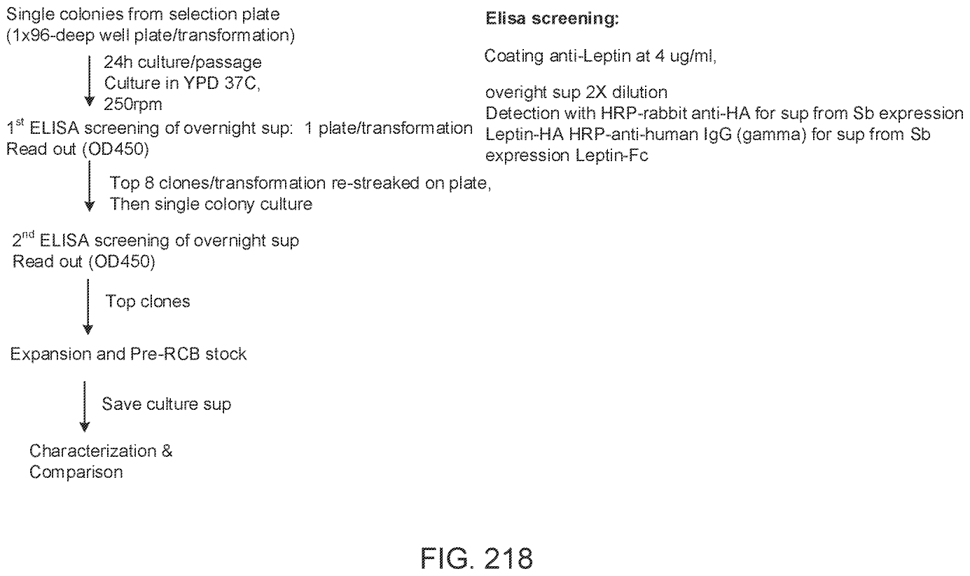

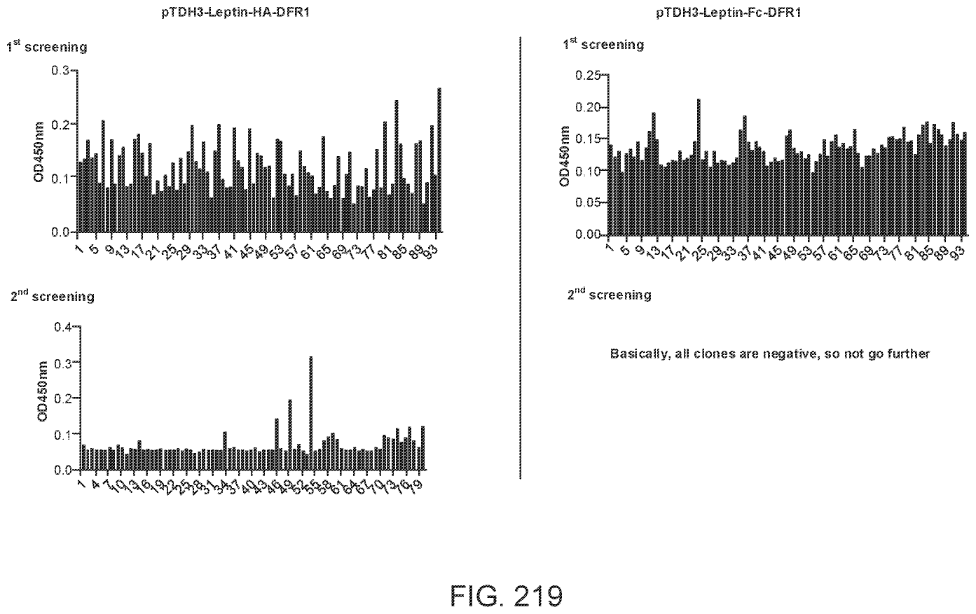

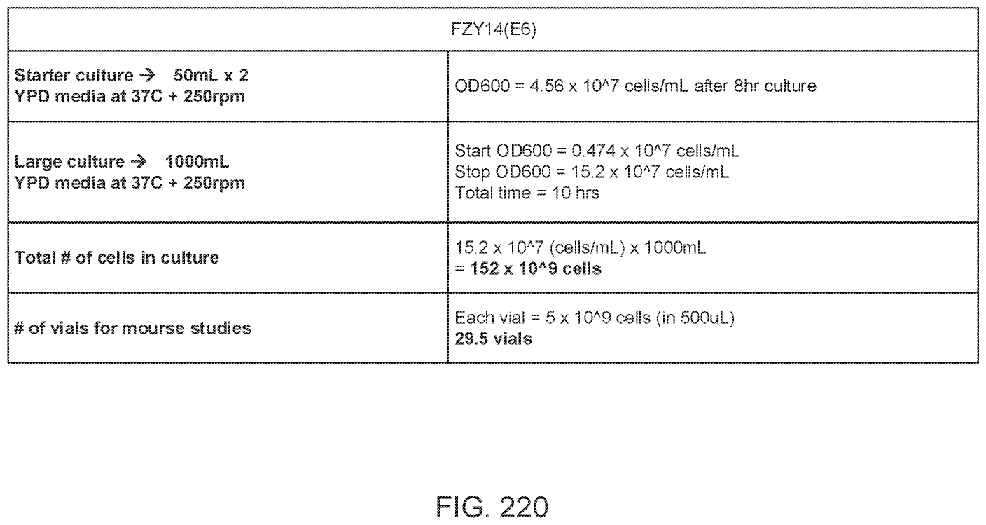

FIGS. 215-220 shows exemplary generation and characterization of engineered strain of S. boulardii constitutively expressing a leptin polypeptide (e.g., human active leptin) (e.g., FZ024).

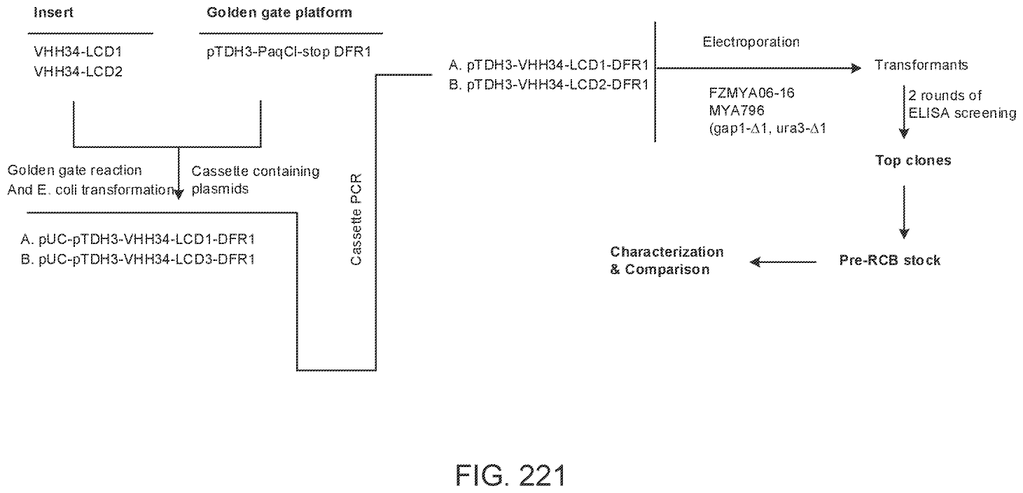

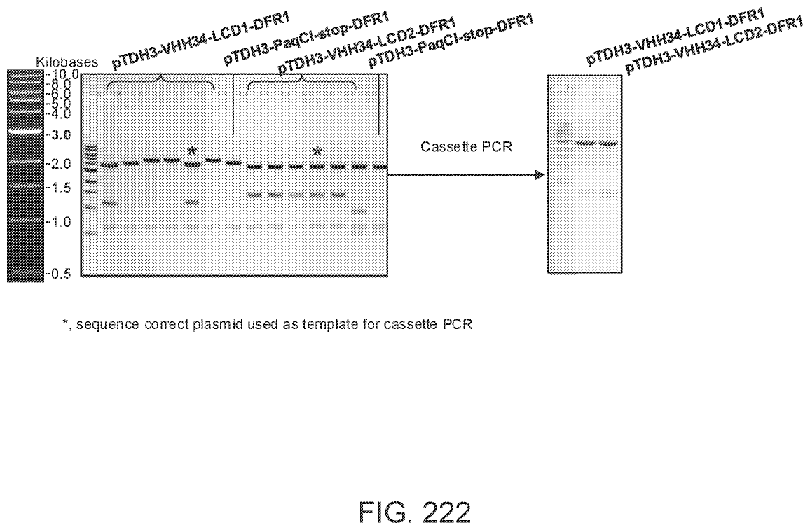

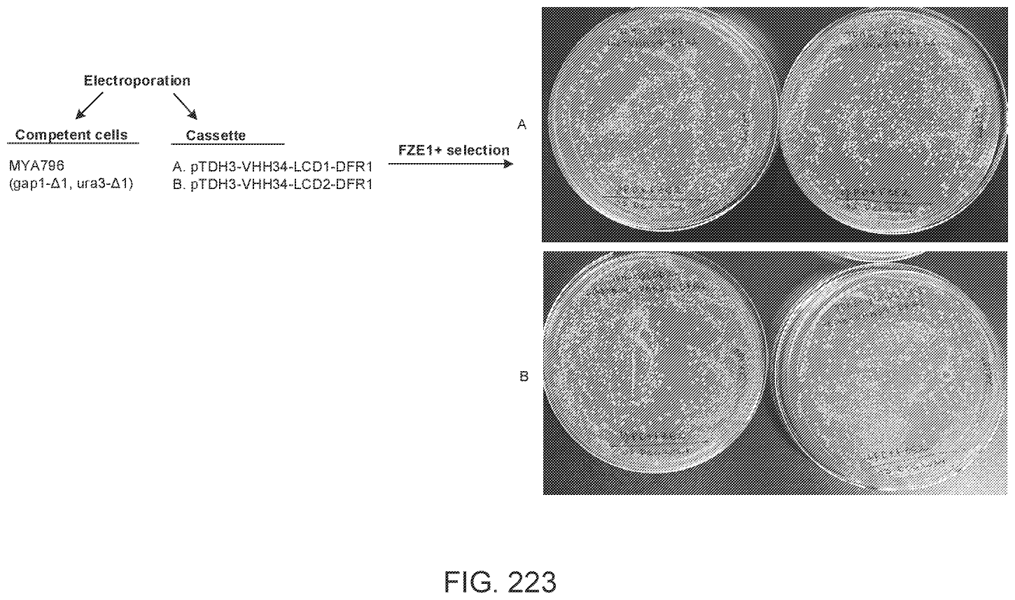

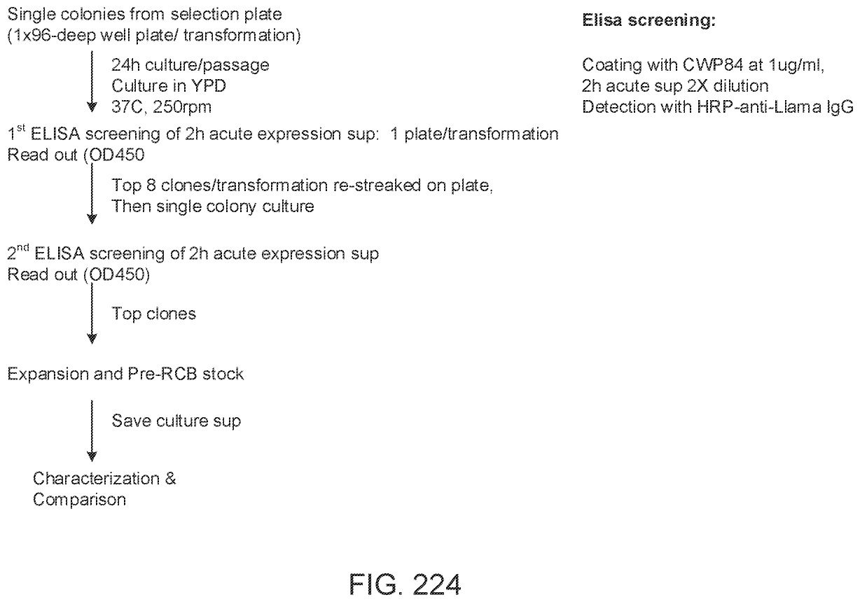

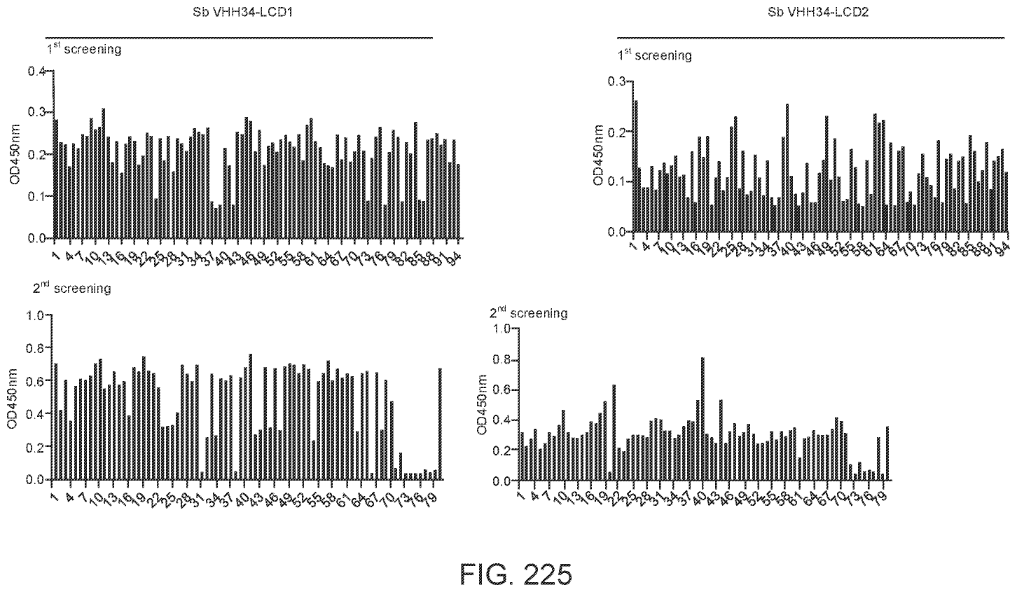

FIGS. 221-225 shows exemplary generation and characterization of engineered strain of S. boulardii constitutively expressing VHH against cwp84 and fused with lysin domain of CDI, which can specifically lyse C. difficile (e.g., FZ028).

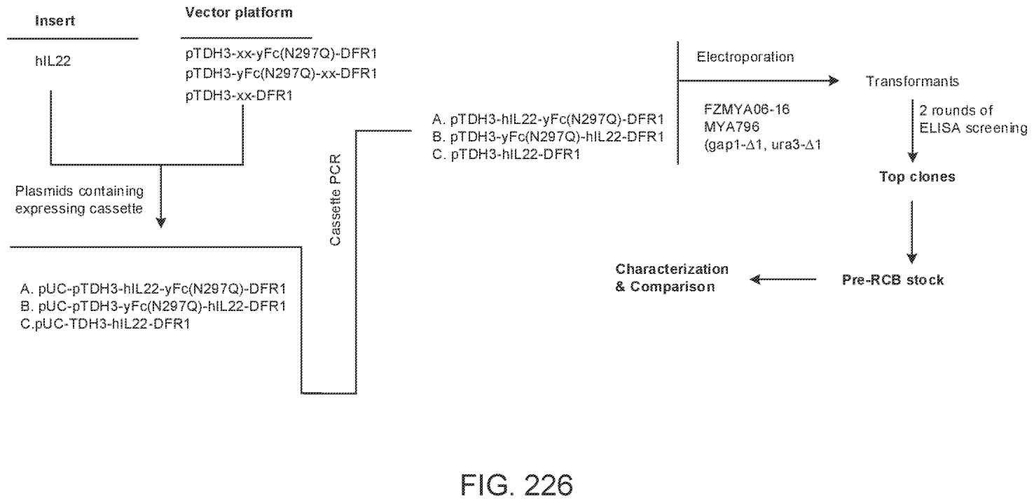

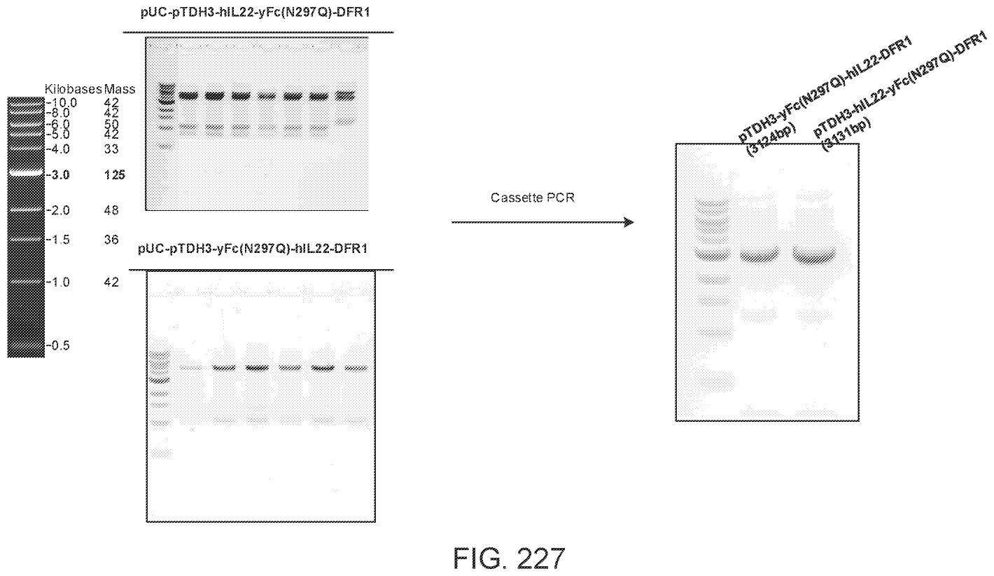

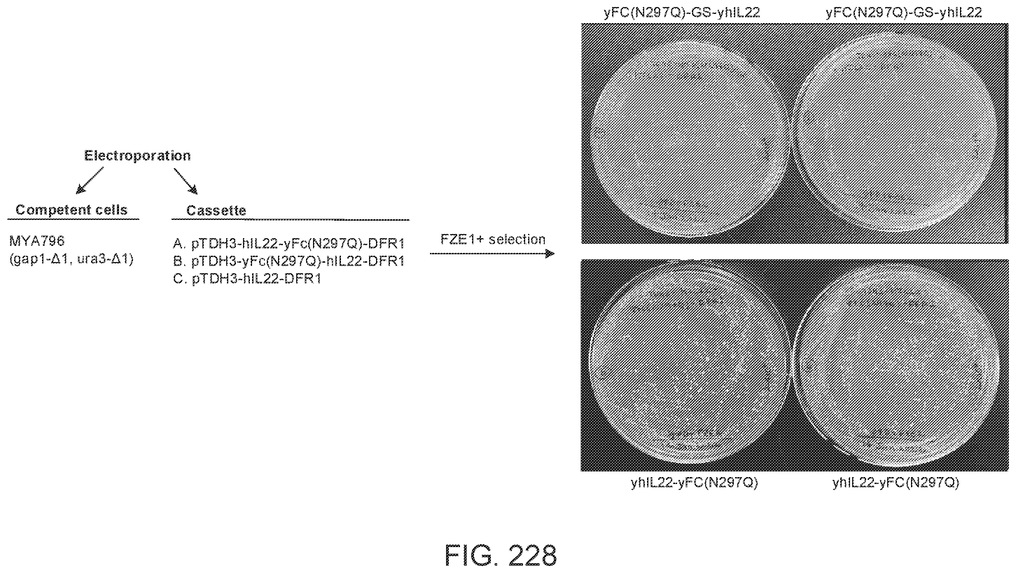

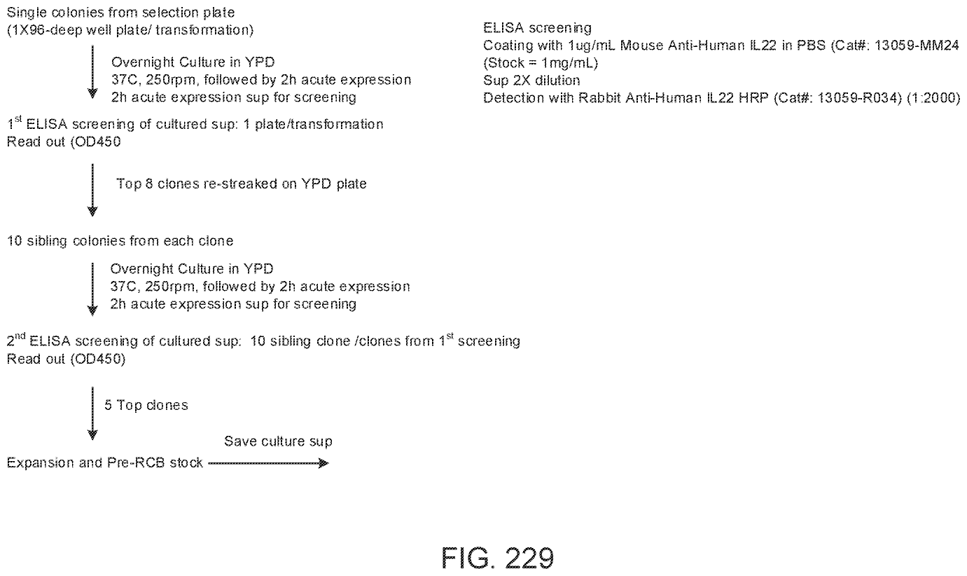

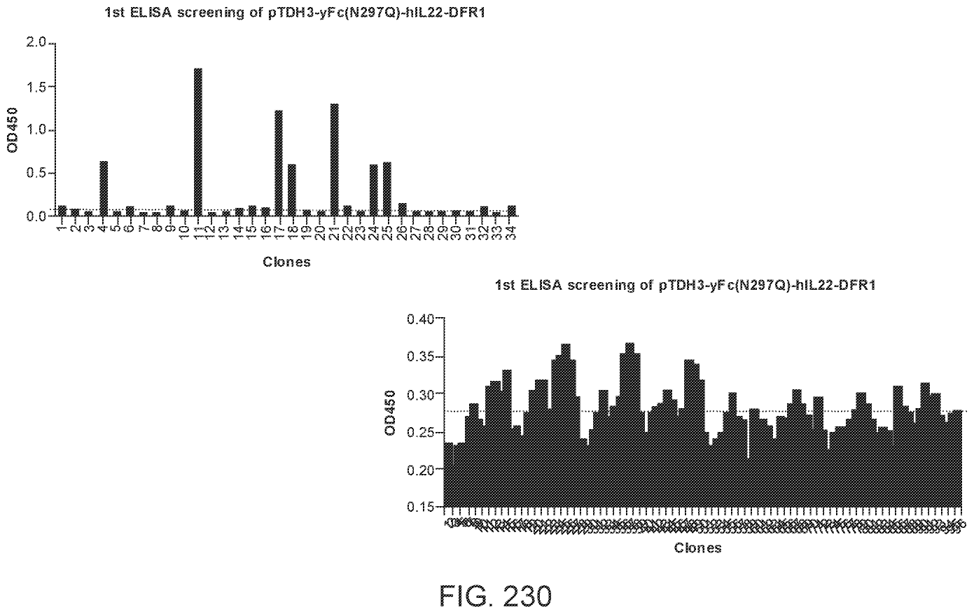

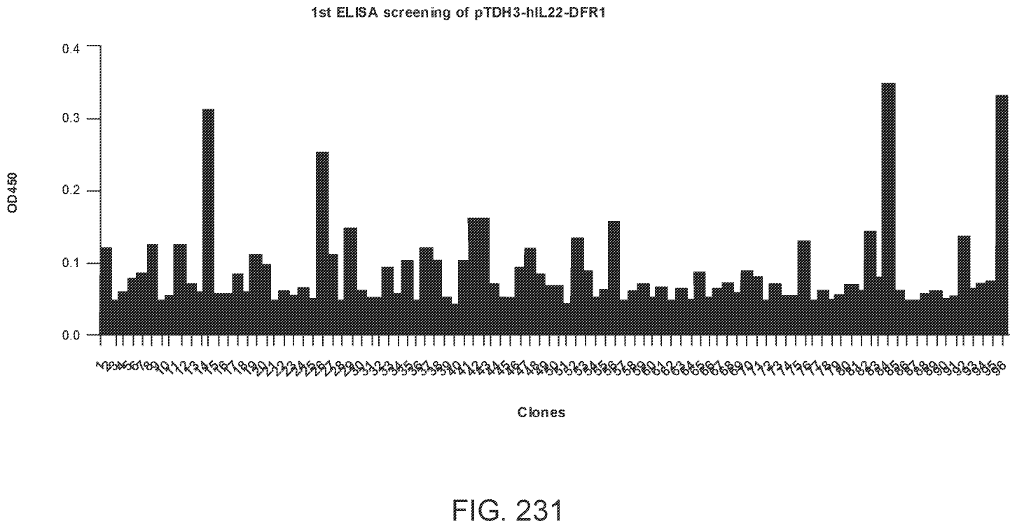

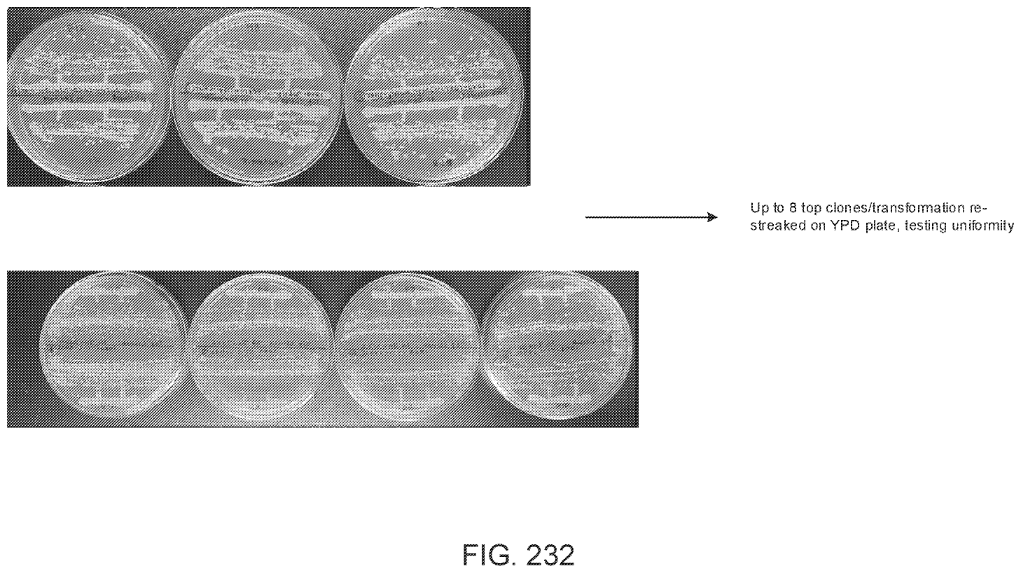

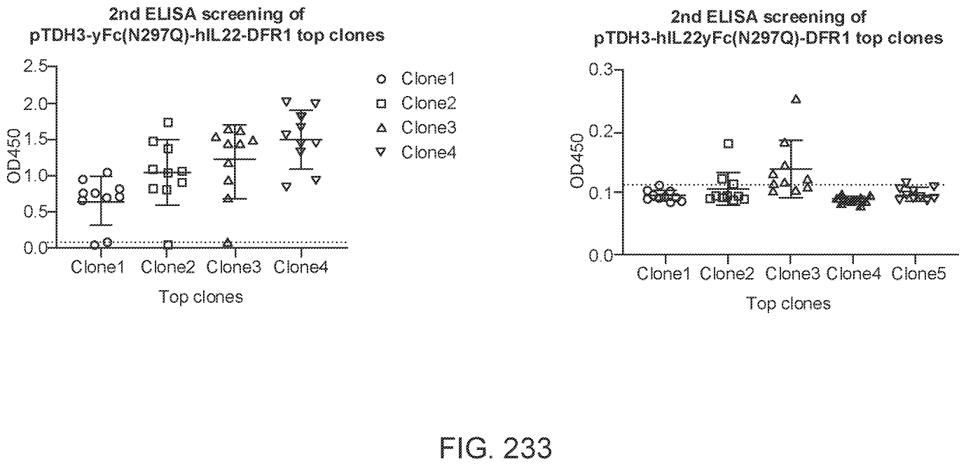

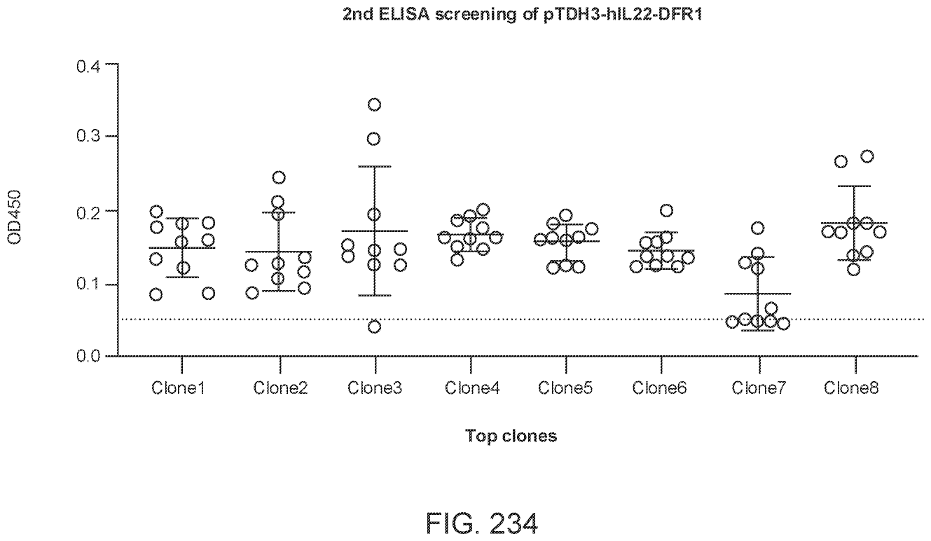

FIGS. 226-234 shows exemplary generation and characterization of engineered strain of S. boulardii constitutively expressing an IL-22 polypeptide (e.g., functional human IL-22) non-fused or fused with mutant Fc fragment at N-terminal or C-terminal (e.g., FZO10h).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed:

1. An engineered strain of Saccharomyces yeast comprising:
at least one site-specific chromosomal insertion of a nucleic acid encoding a therapeutic polypeptide,
wherein the therapeutic polypeptide is selected from a binding protein comprising an antigen-binding domain, an immunoglobulin, an antibody, a cytokine, a hormone, and a chemokine or a combination thereof.

2. The engineered strain of Saccharomyces yeast of claim 1, wherein the yeast is Saccharomyces boulardii.

3. The engineered strain of Saccharomyces yeast of claim 1 or 2 further comprising a complete or partial deletion of URA3.

4. The engineered strain of Saccharomyces yeast of any one of claims 1-3 further comprising complete or partial deletion of GAP1.

5. The engineered strain of Saccharomyces yeast of any one of claims 1-4, wherein the yeast is ura3(−/−) and gap1(−/−).

6. The engineered strain of Saccharomyces yeast of any one of claims 1-5, wherein nucleic acid encoding the therapeutic polypeptide is incorporated into at least two different positions in the yeast's genome or at least one hot spot in the yeast's genome.

7. The engineered strain of Saccharomyces yeast of any one of claims 1-6, wherein nucleic acid encoding the therapeutic polypeptide is incorporated into at least two different chromosomes.

8. The engineered strain of Saccharomyces yeast of claim 7, wherein the at least two different chromosomes comprise chromosomes VII and XVI.

9. The engineered strain of Saccharomyces yeast of any one of claims 1-8 further comprising a nucleic acid sequence encoding a dihydrofolate reductase (DHFR) incorporated into the yeast's genome, wherein the DHFR is optionally a mammalian DFHR.

10. The engineered strain of Saccharomyces yeast of any one of claims 1-9 further comprising one or more exogenous nucleic acids encoding a yeast DFR1.

11. The engineered strain of Saccharomyces yeast of any one of claims 1-10, wherein the therapeutic polypeptide is a binding protein that comprises a structure selected from VHH, Fc-VHH, VHH-Fc, VHH-VHH, VHH-VHH-VHH-VHH, Fc-VHH-VHH, VHH-Fc-VHH, and VHH-VHH-Fc, wherein each or any of the VHH or Fc domains are attached to another VHH or Fc domain via an optional linker sequence.

12. The engineered strain of Saccharomyces yeast of any one of claims 1-10, wherein the therapeutic polypeptide is a binding protein that binds to TcdA, TcdB, or a combination thereof.

13. The engineered strain of Saccharomyces yeast of claim 12, wherein the therapeutic polypeptide comprises an amino acid sequence of SEQ ID NO: 5.

14. The engineered strain of Saccharomyces yeast of any one of claims 1-10, wherein the therapeutic polypeptide is a binding protein that binds to TNF-α.

15. The engineered strain of Saccharomyces yeast of claim 14, wherein the binding protein is an IgG or comprises at least one VHH domain.

16. The engineered strain of Saccharomyces yeast of claim 14 or 15, wherein the therapeutic protein comprises an amino acid sequence selected from any one of SEQ ID NOs: 6, 7, 19, and 20.

17. The engineered strain of Saccharomyces yeast of any one of claims 1-10, wherein the therapeutic polypeptide is a binding protein that binds to IL-17A.

18. The engineered strain of Saccharomyces yeast of claim 17, wherein the binding protein is an IgG or comprises at least one VHH domain.

19. The engineered strain of Saccharomyces yeast of claim 17 or 18, wherein the therapeutic protein comprises an amino acid sequence of SEQ ID NO: 25.

20. The engineered strain of Saccharomyces yeast of any one of claims 1-10, wherein the therapeutic polypeptide is a bi-specific binding protein that binds to TNF-α and IL-17A.

21. The engineered strain of Saccharomyces yeast of claim 20, wherein the binding protein is an IgG or comprises at least two VHH domains.

22. The engineered strain of Saccharomyces yeast of claim 20 or 21, wherein the therapeutic protein comprises an amino acid sequence selected from any one of SEQ ID NOs: 8, 9, and 10.

23. The engineered strain of Saccharomyces yeast of any one of claims 1-10, wherein the therapeutic polypeptide is a binding protein that binds to a norovirus or a rotavirus.

24. The engineered strain of Saccharomyces yeast of claim 23, wherein the binding protein is an IgG or comprises at least one VHH domain.

25. The engineered strain of Saccharomyces yeast of claim 23 or 24, wherein the therapeutic protein comprises an amino acid sequence selected from any one of SEQ ID NOs: 13, 14, 15, and 16.

26. The engineered strain of Saccharomyces yeast of any one of claims 1-10, wherein the therapeutic polypeptide is a VHH that binds to cwp84 and is fused with a lysin domain.

27. The engineered strain of Saccharomyces yeast of claim 26, wherein the therapeutic protein comprises an amino acid sequence selected from any one of SEQ ID NOs: 21 and 22.

28. The engineered strain of Saccharomyces yeast of any one of claims 1-10, wherein the therapeutic polypeptide is a cytokine or chemokine.

29. The engineered strain of Saccharomyces yeast of claim 28, wherein the cytokine is IL-22 or IL-10.

30. The engineered strain of Saccharomyces yeast of claim 28 or 29, wherein the cytokine or chemokine is fused to an Fc domain.

31. The engineered strain of Saccharomyces yeast of any one of claims 28-30, wherein the therapeutic protein comprises an amino acid sequence selected from any one of SEQ ID NOs: 11, 12, and 27.

32. The engineered strain of Saccharomyces yeast of any one of claims 1-10, wherein the therapeutic polypeptide is GLP1.

33. The engineered strain of Saccharomyces yeast of claim 32, wherein the therapeutic protein comprises an amino acid sequence selected from any one of SEQ ID NOs: 17, 23, and 24.

34. The engineered strain of Saccharomyces yeast of any one of claims 1-10, wherein the therapeutic polypeptide is leptin.

35. The engineered strain of Saccharomyces yeast of claim 34, wherein the therapeutic protein comprises an amino acid sequence of SEQ ID NO: 18.

36. The engineered strain of Saccharomyces yeast of any one of claims 1-35, wherein the yeast comprises at least 2, at least 3, at least 4, at least 5, at least 6, at least 7, at least 8, at least 9, or at least 10 or more copies of the nucleic acid encoding the therapeutic polypeptide incorporated into its genome.

37. The engineered strain of Saccharomyces yeast of any one of claims 1-36 further comprising at least one site-specific chromosomal insertion of a second nucleic acid encoding a second therapeutic polypeptide,
wherein the second therapeutic polypeptide is selected from a binding protein comprising a VHH domain, an immunoglobulin, a cytokine, and a chemokine or a combination thereof.

38. A method of binding an antigen in vivo, comprising administering to a subject an engineered strain of Saccharomyces yeast of any one of claims 1-27.

39. The method of claim 38, wherein the antigen is selected from TcdA, TcdB, both TcdA and TcdB, TNF-α, IL-17A, both TNF-α and IL-17A, cwp84, a rotavirus protein, or a norovirus protein.

40. A method of treating or preventing a disease or condition, comprising administering to a subject in need thereof an effective amount of an engineered strain of Saccharomyces yeast of any one of claims 1-37.

41. The method of claim 40, wherein the disease or condition is an inflammatory condition.

42. The method of claim 41, wherein the inflammatory condition is selected from inflammatory bowel disease (IBD), gut inflammation, Crohn's disease, and ulcerative colitis.

43. The method of claim 40, wherein the disease or condition is an infection.

44. The method of claim 43, wherein the infection is a C. difficile infection, a norovirus infection, a rotavirus infection, or a combination thereof.

45. The method of claim 43 or 44, wherein the subject additionally has IBD.

46. The method of claim 40, wherein the disease or condition is irritable bowel syndrome (IBS).

47. The method of claim 40, wherein the disease or condition is neurodegenerative disease

48. The method of claim 40, wherein the disease or condition is diabetes.

49. The method of claim 40, wherein the disease or condition is obesity.

50. The method of claim 40, wherein the disease or condition is fatty liver disease.

51. The method of claim 40, wherein the disease or condition is a metabolic disease.

52. The method of claim 40, wherein the disease or condition is graft-versus-host disease (GVHD).

53. The method of claim 40, wherein the disease or condition is an autoimmune disease.

54. A method of selecting an engineered stain of Saccharomyces yeast, wherein the Saccharomyces yeast comprises a nucleic acid sequence encoding a dihydrofolate reductase (DHFR) incorporated into the yeast's genome, one or more exogenous nucleic acids encoding a yeast DFR1, or a combination thereof, the method comprising contacting the Saccharomyces yeast with methotrexate and sulfanilamide.

55. The method of claim 54, wherein the DHFR is a mammalian DHFR.

56. The method of claim 54 or 55, wherein the methotrexate is in a concentration of 1 nM to 1 mM.

57. The method of claim any one of 54-56, wherein the sulfanilamide is in a concentration of 0.1 to 10 mg/mL.

58. The method of any one of claims 54-57, wherein the engineered stain of Saccharomyces yeast comprises:
at least one site-specific chromosomal insertion of a nucleic acid encoding a therapeutic polypeptide,
wherein the therapeutic polypeptide is selected from a binding protein comprising an antigen-binding domain, an immunoglobulin, an antibody, a cytokine, a hormone, and a chemokine or a combination thereof.

59. The method of any one of claims 54-58, wherein the yeast is Saccharomyces boulardii.

60. The method of any one of claims 54-59, wherein the yeast is ura3(−/−) and gap1(−/−).

61. The method of any one of claims 58-60, wherein nucleic acid encoding the therapeutic polypeptide is incorporated into at least two different positions in the yeast's genome.

62. The method of any one of claims 58-61, wherein the yeast comprises at least 2, at least 3, at least 4, at least 5, at least 6, at least 7, at least 8, at least 9, or at least 10 or more copies of the nucleic acid encoding the therapeutic polypeptide incorporated into its genome.

63. The method of any one of claims 58-62, wherein the yeast further comprises at least one site-specific chromosomal insertion of a second nucleic acid encoding a second therapeutic polypeptide,
wherein the second therapeutic polypeptide is selected from a binding protein comprising a VHH domain, an immunoglobulin, a cytokine, and a chemokine or a combination thereof.
